SEQUENCE LISTING (52 sequences)

SEQ ID NO: 1 - protein, 5 aa, synthetic construct
HYAMN

SEQ ID NO: 2 - protein, 17 aa, synthetic construct
WINTNTGEPTYAQGFTG

SEQ ID NO: 3 - protein, 8 aa, synthetic construct
EREPGMDN

SEQ ID NO: 4 - protein, 16 aa, synthetic construct
RSSQSLVHANTNTYLE

SEQ ID NO: 5 - protein, 7 aa, synthetic construct
KVSNRFS

SEQ ID NO: 6 - protein, 9 aa, synthetic construct
FQGTHVPNT

SEQ ID NO: 7 - protein, 30 aa, synthetic construct
QIQLVQSGSELKKPGASVKVSCKASGYTFT

SEQ ID NO: 8 - protein, 14 aa, synthetic construct
WVRQAPGQGLEWMG

SEQ ID NO: 9 - protein, 32 aa, synthetic construct
RFVFSLDTSVSTAYLQISSLKAEDTAVYYCAR

SEQ ID NO: 10 - protein, 11 aa, synthetic construct
WGQGTLVTVSS

SEQ ID NO: 11 - protein, 23 aa, synthetic construct
DVVMTQSPLSLPVTLGQPASISC

SEQ ID NO: 12 - protein, 15 aa, synthetic construct
WYQQRPGQSPRLLIY

SEQ ID NO: 13 - protein, 32 aa, synthetic construct
GVPDRFSGSGSGTDFTLKISRVEAEDVGVYYC

SEQ ID NO: 14 - protein, 10 aa, synthetic construct
FGQGTKLEIK

SEQ ID NO: 15 - protein, 117 aa, synthetic construct
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSS

SEQ ID NO: 16 - protein, 112 aa, synthetic construct
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIK

SEQ ID NO: 17 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 107 aa, synthetic construct [sequence omitted]

SEQ ID NO: 19 - protein, 220 aa, synthetic construct
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 219 aa, synthetic construct
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 224 aa, synthetic construct
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 215 aa, synthetic construct
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 5 aa, synthetic construct
NSGMH

SEQ ID NO: 24 - protein, 17 aa, synthetic construct
VIWYDGSKRYYADSVKG

SEQ ID NO: 25 - protein, 4 aa, synthetic construct
NDDY

SEQ ID NO: 26 - protein, 11 aa, synthetic construct
RASQSVSSYLA

SEQ ID NO: 27 - protein, 7 aa, synthetic construct
DASNRAT

SEQ ID NO: 28 - protein, 9 aa, synthetic construct
QQSSNWPRT

SEQ ID NO: 29 - protein, 113 aa, synthetic construct [sequence omitted]

SEQ ID NO: 30 - protein, 107 aa, synthetic construct [sequence omitted]

SEQ ID NO: 31 - protein, 5 aa, synthetic construct
NYYMY

SEQ ID NO: 32 - protein, 16 aa, synthetic construct
GINPSNGGTNFNEKFK

SEQ ID NO: 33 - protein, 11 aa, synthetic construct
RDYRFDMGFDY

SEQ ID NO: 34 - protein, 15 aa, synthetic construct
RASKGVSTSGYSYLH

SEQ ID NO: 35 - protein, 7 aa, synthetic construct
LASYLES

SEQ ID NO: 36 - protein, 9 aa, synthetic construct
QHSRDLPLT

SEQ ID NO: 37 - protein, 120 aa, synthetic construct [sequence omitted]

SEQ ID NO: 38 - protein, 111 aa, synthetic construct [sequence omitted]

SEQ ID NO: 39 - protein, 120 aa, synthetic construct
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSS

SEQ ID NO: 40 - protein, 112 aa, synthetic construct
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIK

SEQ ID NO: 41 - protein, 6 aa, synthetic construct
CDNSSS

SEQ ID NO: 42 - protein, 6 aa, synthetic construct
CDQSSS

SEQ ID NO: 43 - RNA, 582 nt, synthetic construct
  source 1..582: mol_type = other RNA
atggctcgcgcccgccaggagggctcctctcctgaaccagtggagggcctcgctcgcgacggaccaagaccattcccactgggacgtcttgttccatccgccgtgtcctgcgggctgtgcgagcccggtttggctgcggcccctgccgcgccgactctgctgcccgctgcctacctctgcgcaccaacagcaccaccagccgtcaccgccgctttggggggttcccgctggcctgggggtccacgaagtcggccccgaggccctcgcccggatggtcctcagccctctctctcgctggcagagcaacatctggaatcgcccgtgcccagcgccccgggggctctggcgggcggtccaacacaagctgctccgggagtccgaggggaggaggaacaatgggccagggaaatcggggcccaacttcgtcggatggcggacgacctcaatgcacaatacgagcggcggagacaagaggagcagcagcggcatcgcccaagtccgtggcgggtgctgtacaacctgatcatgggcctactgccacttccccgtggccaccgcgctcccgagatggagcctaattga

SEQ ID NO: 44 - RNA, 327 nt, synthetic construct
  source 1..327: mol_type = other RNA
atggccaagcagccgagcgacgtgtcctccgagtgcgacagggagggccgccagctgcagccagctgagcggcccccgcaacttagacccggcgctcctacctccttgcagactgaacctcaggcttctatgcgtcaggccgagccagcggacatgcgccccgagatctggatcgcgcaggagctgcgccgcatcggtgacgagttcaatgcctactacgcgcggcgcgtttttctcaacaactatcaggccgcagaggaccacccccgcatggtgatcctgcgtctgctacgctacattgtccgcctggtgtggcgaatgcattga

SEQ ID NO: 45 - protein, 444 aa, synthetic construct
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 46 - protein, 219 aa, synthetic construct; identical to SEQ ID NO: 20

SEQ ID NO: 47 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 447 aa, synthetic construct
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 52 - protein, 219 aa, synthetic construct
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0355349 A1
(43) Pub. Date: Nov. 12, 2026

(54) LIPID-BASED NANOPARTICLE TARGETED AT ACTIVATED IMMUNE CELLS FOR THE EXPRESSION OF IMMUNE CELL INHIBITING MOLECULE AND USE THEREOF

(71) Applicant: OSE IMMUNOTHERAPEUTICS, NANTES (FR)

(72) Inventors: NICOLAS POIRIER, NANTES (FR); DAMIEN HABRANT, NANTES (FR); AURORE MORELLO, NANTES (FR)

(21) Appl. No.: 19/470,210

(22) PCT Filed: Mar. 29, 2024
(86) PCT No.: PCT/EP2024/058781

§ 371 Date: Sep. 29, 2025

(30) Foreign Application Priority Data
Mar. 30, 2023 (EP) 23305453.5
Mar. 30, 2023 (EP) 23305459.2
Jul. 6, 2023 (EP) 23306145.6
Jul. 18, 2023 (EP) 23306240.5
Oct. 31, 2023 (EP) 23306892.3

(51) Int. Cl. A61K 48/00; A61K 9/51; A61K 38/17; A61K 47/68; A61K 47/69

(52) U.S. Cl. CPC A61K 48/0033; A61K 9/5123; A61K 38/1709; A61K 47/6849; A61K 47/6929; A61K 48/005

(57) ABSTRACT

The invention relates to a lipid-based nanoparticle comprising an antigen-binding domain capable of specifically binding to a target expressed on activated immune cells surface and one or several mRNA molecule(s) encoding an activity-inhibiting protein of said activated immune cells, and to uses thereof.

### FIELD OF THE INVENTION

The invention pertains to the field of immunotherapy. The invention relates to a lipid-based nanoparticle comprising mRNA molecule(s), and its use to treat conditions such as autoimmune diseases and inflammatory diseases.

### BACKGROUND OF THE INVENTION

Both autoimmune diseases and inflammatory conditions find their origin in an inadequate response of the immune system to either cells of the self or byproducts of the inflammation process, resulting in an excessive activation of immune cells.

One strategy to inhibit or decrease immune cell activation is through direct activation of a pro-resolutive pathway, or through the suppression of inflammatory cells. However, this may lead to acute and serious adverse effects due to the non-specific suppression of immune cells.

To avoid such nonspecific deactivation of immune cells, several solutions have been developed in previous works.

One of these solutions seeks to decrease immune cell activation with the use of molecules inhibiting the activity of immune cells, the most common of these molecules being immunosuppressants. However, due to important side-effects leading to severe toxicity and limited efficacy, it has been necessary to develop strategies to overcome these drawbacks.

A solution explored in the prior art to inhibit an immune response focuses on targeting specific immune cells subsets for the delivery of inhibiting compounds.

As immune cells are notoriously difficult to transfect, there is a need for a solution to target and deliver high impact molecules to efficiently decrease immune cells activation, in particular within a specific localization.

Vaccines based on mRNA-containing lipid nanoparticles (LNPs) are a promising new delivery platform. LNPs are used to deliver mRNA to cells and have led to the expression of the encoded proteins, thus providing immune-protection to the body. Expressing a protein by delivering the encoding mRNA has many benefits over methods that use proteins, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery.

Even if LNPs have been successfully used by leading vaccines against COVID-19, many questions remain regarding the risk of systemic mRNA-LNP distribution and off-target expression of immunogens that could generate systemic cytokines, activate complement, amplify the frequency or severity of adverse events (observed in recent clinical trials) and/or impair immune response generation.

Therefore, there is a need for improving the technology for overcoming these drawbacks.

### SUMMARY OF THE INVENTION

To this end, the inventors have developed a targeted lipid-based nanoparticle aimed at activated immune cells which comprises a mRNA molecule encoding proteins inhibiting the activity of the immune cells.

The invention ensures a specific and localized delivery of mRNA encoding activity-inhibiting protein to particular immune cells populations, for example located in a chronically inflamed tissue, for a maximal efficiency with limited or no systemic side effects.

The immune cell inhibiting protein can therefore be selected even among proteins exhibiting severe side effects, as their action is circumscribed to a precise location, and the protein will not be expressed systemically and causes off-target side effects.

The invention also allows the person skilled in the art to easily adjust the mRNA cargo to replace or combine the effect of different immune cells inhibiting proteins, to tailor the invention according to the need of the patient, without the necessity to assess and prove the safety of the delivery lipid-based nanoparticle.

The inventors provide herein a method to specifically and selectively target and inhibit a selected subset of immune cells, instead of using LNP to target cells located in an inflamed tissue to deliver inhibitory compounds or of targeting every cells of a given type and haphazardly deactivate them.

Furthermore, the target can be selected based on the location of the immune cell harbouring it. For example, CD80 and CD86 are expressed in pro-inflammatory M1 macrophages.

Targeting CD80 and/or CD86 positive immune cells allows to deliver mRNAs to the immune cells located in the most needed location, ensuring a very effective and specific impact. In comparison, targeting an antigen expressed on numerous cell types with naïve or inactivated cells, like CD3 or CD5, does not confer the same level of precision.

As the protein encoded by the mRNA envisioned herein may be selected among the most potent effectors, it is therefore crucial for the patient's safety that the risks of off-targets are kept as low as possible, or even purely suppressed.

In addition, a further aspect of the invention is to provide LNPs with very low uptake by capturing organs, in particular the liver, but also optionally other organs such as the spleen and/or the lungs. In this aspect, an increased free distribution of the LNPs throughout the body, especially in the blood or other biological fluid, could be achieved, allowing the delivery of the LNPs to the area of interest (e.g., the site of inflammation).

In a first aspect, the invention relates to a lipid-based nanoparticle comprising an antigen-binding domain capable of specifically binding to a target expressed on activated immune cells surface and one or several mRNA molecule(s) encoding an activity-inhibiting protein of said activated immune cells, wherein the mRNA molecule encodes i) an intracellular protein having an intracellular effect on the activated immune cell and/or ii) a transmembrane protein that is preferably not a chimeric associated receptor (CAR).

Particularly, the invention concerns a lipid-based nanoparticle comprising an antigen binding domain capable of specifically binding to a target expressed on activated immune cells surface and one or several mRNA molecule(s) encoding an activity-inhibiting protein of said activated immune cells, wherein the activity-inhibiting protein is i) an intracellular protein having an intracellular effect on the activated immune cell and/or ii) a transmembrane protein, wherein the antigen binding domain is an antibody or an antigen binding fragment thereof.

In an aspect, the lipid-based nanoparticle comprises at least two mRNA molecules, wherein one of said at least two mRNA molecules encodes a transmembrane protein that is a receptor and another of said at least two mRNA molecules encodes a secreted protein that is a ligand of said receptor.

In an aspect, the activated immune cells are selected from the group consisting of activated T cells, activated B cells, activated myeloid cells such as activated macrophages and activated dendritic cells.

In an aspect, the target expressed on activated immune cells surface is selected from the group consisting of PD-1, BCMA/TNFRSF17, BTLA, CD101/IGSF2, CD103, CD119, CD137/4-1BB/TNFRSF9, CD150, CD153, CD154, CD223, CD226, CD25, CD254, CD26, CD27, CD275, CD39/ENTPD1, CD40L, CD44, CD45RO, CD45RC, LGR6, CD69, GPR18, GPR35, FPR2, CD80, CD83, CD86, CD95, CMKLR1, CRTAM, CST7, CTLA4, CXCR3, CXCR4, CXCR5, CXCR6, FasL/TNFSF6, GITR/TNFRSF18, GPR32, TIM3/HAVCR2, ICOS, IL18R1/CXCR1/CD218a, ITGAE, LAG3, TRAILR, OX40L, LY108/SlamF6, NKG2D, OX40/TNFRSF4, PTPN22, RGS1, LOX1, SIGLEC 6, TACI/TNFRSF13B, TIGIT, CD163, CD206, LTBR/CD70, TNFSF14, SLAMF1, SLAMF7, NKG2A, KIR2DL2, CD96, CD112R, CD28H, IL2RB, TRAIL, CD48, CD53, CD164, CD138 (SDC1), CD38, CD39, FCRL4, CD30/TNFRSF8, CD78, TRAF1, TRAF2, TRAF3/CD40BP, TRAF3IP1, TRAF4, TRAF7, TRAP1, TNFR1/TNFRSF1A/CD120A, TRAP100/MED24, TNFR2/TNFRSF1811/CD120B, CDCR3/TNFRSF6B, TNFRSF12A/FN14/TWEAKR, BAFFR/TNFRSF13C/CD268, HVEM/TNFRSF14/CD270, GITR/TNFRSF8/CD357, RELT/TNFRSF19L, TNFRSF19/TROY, TNFRSF21/DR6, TNFRSF25/DR3/TNFRSF12, CD301, IL4R, CLEC-1A, CD21, CLEC-9A, CD180, CD59, CD54, CD71, CD35, CD218a, CD74, CD165, 4-1BBL/CD137L, ICOSL, CD160, CD127 and SIRPa.

In an aspect, the target expressed on activated immune cells surface is selected from the group consisting of BCMA/TNFRSF17, BTLA, CD101/IGSF2, CD103, CD119, CD137/4-1BB/TNFRSF9, CD150, CD153, CD154, CD223, CD226, CD25, CD254, CD26, CD27, CD275, CD39/ENTPD1, CD40L, CD44, CD45RO, CD45RC, LGR6, CD69, GPR18, GPR35, FPR2, CD80, CD83, CD86, CD95, CMKLR1, CRTAM, CST7, CTLA4, CXCR3, CXCR4, CXCR5, CXCR6, FasL/TNFSF6, GITR/TNFRSF18, GPR32, TIM3/HAVCR2, ICOS, IL18R1/CXCR1/CD218a, ITGAE, LAG3, TRAILR, OX40L, LY108/SlamF6, NKG2D, OX40/TNFRSF4, PDCD1, PTPN22, RGS1, LOX1, SIGLEC 6, TACI/TNFRSF13B, TIGIT, CD163, CD206, LTBR/CD70, TNFSF14, SLAMF1, SLAMF7, NKG2A, KIR2DL2, CD96, CD112R, CD28H, IL2RB, TRAIL, CD48, CD53, CD164, CD138 (SDC1), CD38, CD39, FCRL4, CD30/TNFRSF8, CD78, TRAF1, TRAF2, TRAF3/CD40BP, TRAF3IP1, TRAF4, TRAF7, TRAP1, TNFR1/TNFRSF1A/CD120A, TRAP100/MED24, TNFR2/TNFRSF1811/CD120B, CDCR3/TNFRSF6B, TNFRSF12A/FN14/TWEAKR, BAFFR/TNFRSF13C/CD268, HVEM/TNFRSF14/CD270, GITR/TNFRSF8/CD357, RELT/TNFRSF19L, TNFRSF19/TROY, TNFRSF21/DR6, TNFRSF25/DR3/TNFRSF12, CD301, IL4R, CLEC-1A, CD21, CLEC-9A, CD180, CD59, CD54, CD71, CD35, CD218a, CD74, CD165, 4-1BBL/CD137L, ICOSL and CD160.

Preferably, the target expressed on activated immune cells surface is selected from the group consisting of PD-1, CD127, SIRPa and CLEC-1A.

In an aspect, the target expressed on activated immune cells surface is PD-1.

In an aspect, the antigen binding domain is an anti-PD-1 binding domain comprising:
a) (i) a VH comprising HCDR1, HCDR2 and HCDR3, and (ii) a VL comprising LCDR1, LCDR2 and LCDR3, wherein:
   the heavy chain CDR1 (HCDR1) comprises or consists of an amino acid sequence of SEQ ID NO: 1;
   the heavy chain CDR2 (HCDR2) comprises or consists of an amino acid sequence of SEQ ID NO: 2;
   the heavy chain CDR3 (HCDR3) comprises or consists of an amino acid sequence of SEQ ID NO: 3;
   the light chain CDR1 (LCDR1) comprises or consists of an amino acid sequence of SEQ ID NO: 4;
   the light chain CDR2 (LCDR2) comprises or consists of an amino acid sequence of SEQ ID NO: 5, and
   the light chain CDR3 (LCDR3) comprises or consists of an amino acid sequence of SEQ ID NO:6; or
b) i) a VH comprising or consisting of an amino acid sequence of SEQ ID NO: 15; and ii) a VL comprising or consisting of an amino acid sequence of SEQ ID NO: 16.

In an aspect, the antigen binding domain is an anti-PD-1 binding domain comprising:
a) (i) a VH comprising HCDR1, HCDR2 and HCDR3, and (ii) a VL comprising LCDR1, LCDR2 and LCDR3, wherein:
   the heavy chain CDR1 (HCDR1) comprises or consists of an amino acid sequence of SEQ ID NO: 31;
   the heavy chain CDR2 (HCDR2) comprises or consists of an amino acid sequence of SEQ ID NO: 32;
   the heavy chain CDR3 (HCDR3) comprises or consists of an amino acid sequence of SEQ ID NO: 33;
   the light chain CDR1 (LCDR1) comprises or consists of an amino acid sequence of SEQ ID NO: 34;
   the light chain CDR2 (LCDR2) comprises or consists of an amino acid sequence of SEQ ID NO: 35, and
   the light chain CDR3 (LCDR3) comprises or consists of an amino acid sequence of SEQ ID NO: 36; or
b) i) a VH comprising or consisting of an amino acid sequence of SEQ ID NO: 37; and ii) a VL comprising or consisting of an amino acid sequence of SEQ ID NO: 38.

In an aspect, the antigen binding domain is an anti-PD-1 binding domain comprising:
a) (i) a VH comprising HCDR1, HCDR2 and HCDR3, and (ii) a VL comprising LCDR1, LCDR2 and LCDR3, wherein:
   the heavy chain CDR1 (HCDR1) comprises or consists of an amino acid sequence of SEQ ID NO: 23;
   the heavy chain CDR2 (HCDR2) comprises or consists of an amino acid sequence of SEQ ID NO: 24;
   the heavy chain CDR3 (HCDR3) comprises or consists of an amino acid sequence of SEQ ID NO: 25;
   the light chain CDR1 (LCDR1) comprises or consists of an amino acid sequence of SEQ ID NO: 26;
   the light chain CDR2 (LCDR2) comprises or consists of an amino acid sequence of SEQ ID NO: 27, and
   the light chain CDR3 (LCDR3) comprises or consists of an amino acid sequence of SEQ ID NO: 28; or
b) i) a VH comprising or consisting of an amino acid sequence of SEQ ID NO: 29; and ii) a VL comprising or consisting of an amino acid sequence of SEQ ID NO: 30.

Typically, the antigen binding domain disclosed herein comprises a Fc domain, preferably an IgG Fc domain.

Preferably, the antigen binding domain is not covalently bound to any of the lipids of the lipid-based nanoparticle or does not comprise any modification for coupling or grafting the antigen binding domain to a lipid. Particularly, the lipid-based nanoparticle does not comprise an anchoring moiety comprising a lipidation peptide or motif.

In an aspect, the lipid-based nanoparticle comprises an additional antigen binding domain capable of specifically binding to another target expressed on activated immune cells surface.

Preferably, the additional antigen binding domain is i) not covalently bound to any of the lipids of the lipid-based nanoparticle, ii) does not comprise any modification for coupling or grafting the antigen binding domain to a lipid and/or iii) is not covalently bound to a lipidation peptide or motif.

In an aspect, the mRNA molecule encodes an intracellular protein having an intracellular effect on the activated immune cells selected from the group consisting of a cytoplasmic protein, an intracellular signalling protein, an enzyme, a transcription factor, an intrabody, a dominant negative receptor or an antagonist engineered protein such as an engineered blocking protein.

Preferably, the one or several mRNA molecule(s) encode(s) for a molecule selected from the group consisting of:
a) a protein selected from the group consisting of TIM3, ENTPD1, LAG3, PD-1, PD-L1, TIGIT, FOXO1, MLH1, MSH2, MSH6, APC, CDKN2A, CASPASE, FAS, TRAIL, Bax, Bak, Bok, Bad, Bid et Bim, BIM, BIM-S, FADD, FASL, TRAILR, TNFR, ATG5, LC3, GABARAP, GATE16, ATG 5/7/10/12, NOXA, PUMA, P53, FOXP3, TOX, EOMES, BCL6 and BACH2, HIFa, PKCq and VHL, IDO-1, IDO-2, ARG1; TDO, mTOR/DAPTOR/RAPTOR, SHP, SMAD, SHIP-1, SHP-1/2, PTEN, PTP1B, IKAROS, EGR2/3, CREM, P27 (KIP1), CD107a, RAC1, AP2, RB7, M6P, MPR, BBS10, BBS12, TCP1, HSP, APC, Laminin, Actin, Vimentin, DEF1, Dynein, Kinesin, TAPASIN, LMP7, Erp57, Cbl-b, CHEMR23, GPR37, GPR32, GPR18, FPR2, GPR35, HDAC, HMT, SIRT1, DOT1L, PRDME, PRMT1, PRMT3, PRMT5, PRMT6, SETD7, SETD8, SMYD2, SMYD3 or SUV420H2, preferably selected from the group consisting of TIM3, LAG3, PD-L1, TIGIT, FAS, TRAIL, PUMA, BIM-S, FOXP3, TOX, IDO-1, IDO-2, ARG1, CHEMR23 and FPR2, and any combination thereof,
b) a cytokine, preferably selected from the group consisting of IFNG, IFNa, IL2, IL13, IL4 and IL-10, and any combination thereof,
c) a cytokine receptor, preferably selected from the group consisting of IL-1R, IL-4R, IL-6R, IL-8R, IL-10R, IL-11R, IL-17R, IL-18R, IL-21R, IL-23R, IL-35R and any combination thereof, and
d) a chemokine receptor, preferably selected from the group consisting of CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6 and XCR1, and any combination thereof.

In an aspect, the one or several mRNA molecule(s) encode(s) for CCR4, CCR10, CXCR3, CCR5, CCR2, CX3CR1, CCR7, CXCR4, CXCR5, IL10R, TGFR, TNFR, ILR1A, GCSFR, IL4R, CXCR1, CXCR2, CXCR6, CCR1, CCR3, CCR6, CCR8, CCR9, CCR11, XCR1, CD72, DCIR, MICL, CLEC-1, TGFB, IL13, IL4, IL-10, IL35, IL37, IL38 IL-6R, IL17R, IL23R, IL35R, IL21R, IFNa R, BTLA, CTLA4, B7H1, B7H3, B7H4, PD1H, LAIR1, TIM1, TIM3, TIM4, 2B4, PD-1, PDL1, PDL2, galectin9, CD48, HVEM, B7DC, CD155, CD113, CD112, ILT2, ILT3, NKG2A, LILRB1, IRp60, KIR, CD22, CD5, CD66a, PIR-B, KIR2DL1, KIR2DL2/3, ILT4, KIR2DL5, KIR3DL1, KIR3DL2, KIR3DL3, TIGIT, LAG3 or A2AR, SOCS, RIPK1 and any member of the STAT family or any combination thereof.

Preferably, the one or several mRNA molecule(s) encode(s) for BIM-S and/or PUMA.

In some aspects, the lipid-based nanoparticle comprises at least two mRNA molecules, wherein one of the least two mRNA molecules encodes a transmembrane protein that is a receptor and another of the least two mRNA molecules encodes a secreted protein that is a ligand of said receptor.

In some aspects, the lipid-based composition of the lipid based nanoparticle comprises or consists of a cationic or ionizable lipid, a helper lipid, a sterol and a PEG-lipid.

Preferably, the ionizable lipid is selected from the group consisting of [(4-hydroxybutyl)azanediyl]di(hexane-6,1-diyl) bis(2-hexyldecanoate) (ALC-0315), 1,2-dioleoyl-3-trimethylammonium propane (DOTAP); N,N-dimethyl-2,3-dioleyloxypropylamine (DODMA), 1,2-di-O-octadecenyl-3-trimethylammoniumpropane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammoniumpropanes; 1,2-dialkyloxy-3-dimethylammoniumpropanes; dioctadecyldimethylammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (+)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (+)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminiiim (DOBAQ), 2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98Niz-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), 9-Heptadecanyl 8-{(2-hydroxyethyl)[6-oxo-6-(undecyloxy)hexyl]amino}octanoate (SM-102), bis[2-(4-{2-[4-(cis-9-octadecenoyloxy)phenylacetoxy]ethyl}piperidinyl)ethyl]disulfide (SS-OP; e.g., CASnumber2377474-67-2) bis{2-[4-(α-D-tocopherolhemisuccinateethyl)piperidyl]ethyl}disulfide (SS-EC) and any mixtures thereof, preferably is ALC-0315, SM-102, Dlin-MC3-DMA or SS-OP, more preferably ALC-0315 or SS-OP.

Preferably, the sterol is selected from the group consisting of cholesterol, fecosterol, sitosterol, ergosterol, campesterol, stigmasterol, brassicasterol, tomatidine, ursolic acid, alpha-tocopherol, and any mixtures thereof, preferably is cholesterol.

Preferably, the helper lipid is selected from DOPE, DOPS, DODMA, DOTAP, DODAP, DDAB, POPE, DSPC, DOPC, DEPC and DSPE, and any mixtures thereof preferably is DOPE or DSPC.

Preferably, the PEG-lipid is selected from PEG-DMG, PEG-DSPE, PEG-c-DOMG, DMG-PEG-2000, PEG-DLPE, PEG-DMPE, PEG-DPPC, PEG-DPPE, PEG-DAG and PEG-c-DMA, ALC-0159, and any mixture thereof preferably is PEG-DMG, PEG-DSPE or a mixture thereof.

Particularly, the PEG has a size of between about 2000 Daltons and about 5000 Daltons, preferably is DSPE-PEG-2000, DMG-PEG-2000, DSPE-PEG-5000, DMG-PEG-5000 or a mixture thereof.

In some instances, the lipid-based composition of the lipid-based nanoparticle is selected from the group consisting of:
a) ALC-0315, DOPE, cholesterol and DMG-PEG,
b) ALC-0315, DDAB, cholesterol and DMG-PEG,
c) ALC-0315, POPE, cholesterol and DMG-PEG,
d) ALC-0315, DOPE, cholesterol and DSPE-PEG,
e) ALC-0315, DSPC, cholesterol and DMG-PEG,
f) ALC-0315, DSPC, cholesterol and ALC-0159;
g) SM-102, DSPC, cholesterol and DMG-PEG,
h) Dlin-MC3-DMA, DSPC, cholesterol and DMG-PEG,
i) ALC-0315, DOPE, cholesterol, DMG-PEG and DSPE-PEG,
j)SS-OP, DOPE, cholesterol and DMG-PEG;
k)SS-OP, DSPC, cholesterol and DSPE-PEG; and
1)SS-OP, DOPC, cholesterol and DMG-PEG.

In some aspects, the lipid-based nanoparticle comprises from about 35 mol % to about 55 mol % of a cationic or ionizable lipid, from about 5 mol % to about 20 mol % of a helper lipid, from about 30 mol % to about 60 mol % of a sterol, and from about 0.5 mol % to about 4 mol % of a PEG-lipid. Preferably, the lipid-based nanoparticle comprises from about 45 mol % to about 55 mol % of a cationic or ionizable lipid, from about 5 mol % to about 15 mol % of a helper lipid from about 35 mol % to about 45 mol % of a sterol, and from about 0.5 mol % to about 2.5 mol % of a PEG-lipid.

In an aspect, the mRNA molecule comprises a modified nucleotide, preferably selected from the group comprising alternative uracils, alternative cytosine, alternative guanine and alternative adenine, preferably selected from the group comprising pseudouridine, 1-methylpseudouridine, 1-ethyl pseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine and 2′-O-methyl uridine.

In an aspect, the mRNA molecule comprises a 5′UTR, a 5′ cap structure, a Kozak sequence, an IRES sequence, a chain terminating nucleotide, a stem loop, a 3′UTR, a poly A sequence and/or a polyadenylation signal.

The invention also relates to a pharmaceutical composition comprising at least one lipid-based nanoparticle according to the present invention and optionally a pharmaceutically acceptable carrier or excipient.

In an aspect, the pharmaceutical composition further comprises an additional lipid-based nanoparticle comprising an antigen binding domain capable of specifically binding to a target expressed on activated immune cells surface and comprising one or several mRNA molecule(s) encoding an immune cell activity inhibiting protein.

The invention also relates to the lipid-based nanoparticle according to the present invention, or the pharmaceutical composition according to the present invention, for use as a medicament.

In an aspect, the lipid-based nanoparticle or the pharmaceutical composition are for use in the treatment of a disease in a subject in need thereof, wherein the disease is selected from the group consisting of disease wherein the resolution of inflammation is delayed or disrupted, and/or a disease selected from the group of inflammatory diseases, in particular acute inflammatory diseases, chronic inflammatory diseases such as chronic inflammatory pulmonary diseases (e.g asthma), keratoconjunctivitis, periodontal disease, eczema, inflammatory bowel disease, in particular Crohn's disease or colitis, in particular ulcerative colitis or spontaneous colitis, cystic fibrosis, cutaneous inflammation; autoimmune diseases such as diabetes, NASH, in particular type I diabetes, psoriasis, lupus, rheumatoid arthritis, multiple sclerosis, Sjögren's syndrome, celiac disease, vasculitis, myasthenia gravis; infection diseases such as sepsis, severe viral indications with severe inflammatory conditions, such as coronavirus (e.g. COVID-19), peritonitis; degenerative diseases; wound healing disorders, dry eye syndrome; cancer diseases, in particular solid and liquid cancers, metastatic cancers, in particular carcinoma, in particular mammary carcinoma or colon carcinoma, or colorectal cancer or lung cancer or mesothelioma, or myeloid cancer, in particular leukemia; fibrosis (notably lung and hepatic fibrosis) and ANCA (Anti neutrophil Cytoplasmic Autoantibodies) pathologies (vasculitis).

The present invention also relates to the use of the lipid-based nanoparticle or the pharmaceutical composition according to the present invention for the manufacture of a medicament for the treatment of a disease as listed herein in a subject in need thereof. It further relates to a method for treating a disease as listed herein in a subject in need thereof comprising administering a therapeutic effective amount of the lipid-based nanoparticle or the pharmaceutical composition according to the present invention to said patient.

The invention also relates to an in vitro method for decreasing immune cells activity, comprising the step of contacting activated immune cells with a lipid-based nanoparticle according to the present invention or with the pharmaceutical composition according to the present invention.

### DETAILED DESCRIPTION OF THE INVENTION

### Definitions

In order that the present invention may be more readily understood, certain terms are defined hereafter. Additional definitions are set forth throughout the detailed description.

Unless otherwise defined, all terms of art, notations and other scientific terminology used herein are intended to have the meanings commonly understood by those of skill in the art to which this invention pertains.

As used herein, the term “antibody” describes a type of immunoglobulin molecule and is used in its broadest sense. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. The heavy-chain constant domains that correspond to the different classes of immunoglobulins are called alpha, delta, epsilon, gamma, and mu, respectively. The term “antibody” particularly refers to immunoglobulin molecules and immunologically active portions of immunoglobulin molecules, i.e., molecules that contain an antigen binding domain that specifically binds an antigen. As such, the term antibody encompasses whole antibody molecules such as four-chain antibodies comprising 2 heavy chains and 2 light chains, such as polyclonal antibodies, monoclonal antibodies or recombinant antibodies, but also any antibody fragments thereof that comprise an antigen binding domain.

The terms “antigen-binding fragment”, “antibody fragment” or “antigen-binding domain” of an antibody, as used herein, refers to one or more fragments or derivatives of an antibody that retain the ability to specifically bind to an antigen (e.g., PD-1).

Examples of binding fragments encompassed within the term “antigen-binding fragment” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain, or any fusion proteins comprising such antigen-binding fragments. Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody.

These antibody fragments are obtained using conventional techniques known to those of skill in the art, and the fragments are screened for utility in the same manner as are intact antibodies.

As used herein, the term “isolated” indicates that the recited material (e.g., antibody, polypeptide, nucleic acid, etc.) is substantially separated from, or enriched relative to, other materials with which it occurs in nature. Particularly, an “isolated” antibody is one which has been identified and separated and/or recovered from a component of its natural environment.

As used herein, the term “treatment”, “treat” or “treating” refers to any act intended to ameliorate the health status of patients such as therapy, prevention, prophylaxis and retardation of the disease. In certain aspects, such term refers to the amelioration or eradication of a disease or symptoms associated with a disease, such as according to the present disclosure, the disruption or the delay in the resolution of the inflammation leading to inflammation associated disease. In other aspects, this term refers to minimizing the spread or worsening of the disease resulting from the administration of one or more therapeutic agents to a subject with such a disease.

As used herein, the “sequence identity” between two sequences is described by the parameter “sequence identity”, “sequence similarity” or “sequence homology”. For purposes of the present invention, the “percentage identity” between two sequences (A) and (B) is determined by comparing the two sequences aligned in an optimal manner, through a window of comparison. The percent identity between the two sequences is particularly a function of the number of identical positions shared by the sequences (i.e., % identity=number of identical positions/total number of positions×100), taking into account the number of gaps, and the length of each gap, which need to be introduced for optimal alignment of the two sequences. Said alignment of sequences can be carried out by well-known methods in the art, for example, using the algorithm for global alignment of Needleman-Wunsch. Protein analysis software matches similar sequences using measures of similarity assigned to various substitutions, deletions, and other modifications, including conservative amino acid substitutions. Once the total alignment is obtained, the percentage of identity can be obtained by dividing the full number of identical amino acid residues aligned by the full number of residues contained in the longest sequence between the sequence (A) and (B). Sequence identity is typically determined using sequence analysis software. For comparing two amino acid sequences, one can use, for example, the tool “Emboss needle” for pairwise sequence alignment of proteins providing by EMBL-EBI and available on:
www.ebi.ac.uk/Tools/services/web/toolform. ebi?tool=emboss_needle&context=protein, for example using default settings: (I) Matrix: BLOSUM62, (ii) Gap open: 10, (iii) gap extend: 0.5, (iv) output format: pair, (v) end gap penalty: false, (vi) end gap open: 10, (vii) end gap extend: 0.5.

The percent identity between two amino acid sequences or nucleotide sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4.

Alternatively, the percent identity between two amino acid sequences or nucleotide sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. The percent identity between two nucleotide or amino acid sequences may also be determined using for example algorithms such as the BLASTN program for nucleic acid or amino acid sequences using as defaults a word length (W) of 11, an expectation (E) of 10, M=5, N=4, and a comparison of both strands.

Alternatively, sequence identity can also be typically determined using sequence analysis software Clustal Omega using the HHalign algorithm and its default settings as its core alignment engine. The algorithm is described in Soding, J. (2005) ‘Protein homology detection by HMM-HMM comparison’. Bioinformatics 21, 951-960, with the default settings.

“Eu numbering” (also known as Eu index) refers to the antibody numbering system (Kabat et al., 1991, Sequences of Proteins of Immunological Interest, 5th Ed., United States Public Health Service, National Institutes of Health, Bethesda), which is based on the sequential numbering of the first human IgG1 sequenced (the Eu antibody; Edelman, et al., 1969, Proc Natl Acad Sci USA 63: 78-85).

By “amino acid change” or “amino acid modification” is meant herein a change in the amino acid sequence of a polypeptide. “Amino acid modifications” include substitution, insertion and/or deletion in a polypeptide sequence. By “amino acid substitution” or “substitution” herein is meant the replacement of an amino acid at a particular position in a parent polypeptide sequence with another amino acid. By “amino acid insertion” or “insertion” is meant the addition of an amino acid at a particular position in a parent polypeptide sequence. By “amino acid deletion” or “deletion” is meant the removal of an amino acid at a particular position in a parent polypeptide sequence. The amino acid substitutions may be conservative. A conservative substitution is the replacement of a given amino acid residue by another residue having a side chain (“R-group”) with similar chemical properties (e.g., charge, bulk and/or hydrophobicity). As used herein, “amino acid position” or “amino acid position number” are used interchangeably and refer to the position of a particular amino acid in an amino acids sequence, generally specified with the one letter codes for the amino acids. The first amino acid in the amino acids sequence (i.e., starting from the N terminus) should be considered as having position 1.

A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art. For instance, conservative substitutions can be defined by substitutions within the groups of amino acids reflected in the following tables:

TABLE A
Amino Acid Residue

| Amino Acid groups | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE B
Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE C
Further Alternative Physical and Functional
Classifications of Amino Acid Residues

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, T, V, W, and Y |
| Negatively charged residues | D and E |
| Polar residues | C, D, E, H, K, N, Q, R, S, and T |
| Small residues | A, C, D, G, N, P, S, T, and V |
| Very small residues | A, G, and S |
| Residues involved in turn | A, C, D, E, G, H, K, N, Q, R, S, P, and T |
| formation | |
| Flexible residues | E, Q, T, K, S, G, P, D, E, and R |

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to +/−10% (e.g., +/−0.5%, +/−1%, +/−1.5%, +/−2%, +/−2.5%, +/−3%, +/−3.5%, +/−4%, +/−4.5%, +/−5%, +/−5.5%, +/−6%, +/−6.5%, +/−7%, +/−7.5%, +/−8%, +/−8.5%, +/−9%, +/−9.5%). The use of the term “about” at the beginning of a string of values modifies each of the values (i.e., “about 1, 2 and 3” refers to about 1, about 2 and about 3). Further, when a listing of values is described herein (e.g., about 50%, 60%, 70%, 80%, 85% or 86%) the listing includes all intermediate and fractional values thereof (e.g., 54%, 85.4%).

The term “essentially” as used herein in connection with any given biological sequence means said biological sequence varies from the reference sequence contained in the sequence listing by up to 10% of the biological sequence length. In particular, by “consists essentially of” is intended that the biological sequence consists of that sequence, but it may also include 1, 2, 3, 4, 5, 6, 7, 8, 9 or 10 substitutions, additions, deletions or a mixture thereof, preferably 1, 2, 3, 4, or 5 substitutions, additions, deletions or a mixture thereof, with the proviso that said biological sequence varies from the reference sequence contained in the sequence listing by up to 10% of the biological sequence length.

### Lipid-Based Nanoparticles

The present disclosure relates to a lipid-based nanoparticle comprising an antigen-binding domain capable of specifically binding to a target expressed on activated immune cells surface and one or several mRNA molecule(s) encoding an activity-inhibiting protein of said activated immune cells.

The invention particularly concerns a lipid-based nanoparticle comprising an antigen binding domain capable of specifically binding to a target expressed on activated immune cells surface and one or several mRNA molecule(s) encoding an activity-inhibiting protein of said activated immune cells, wherein the activity-inhibiting protein is i) an intracellular protein having an intracellular effect on the activated immune cell and/or ii) a transmembrane protein, wherein the antigen binding domain is an antibody or an antigen binding fragment thereof.

The lipid-based nanoparticle according to the invention is particularly formulated either as a liposome or a lipid nanoparticle (LNP), especially a lipid nanoparticle comprising a mixture of lipids.

The lipid-based nanoparticle also encompasses similar nanoparticles such as but not limited to micelles and nano-emulsions. Lipid based nanoparticles also include Hybrid nanoparticles comprising polymers-lipids hybrid compounds, such as polamines-polaxamers, in particular as described herein.

The lipid-based nanoparticle according to the invention is preferably a t-LNP. As used herein, the term “t-LNP” refers to a targeted lipid nanoparticle, i.e., a lipid nanoparticle comprising an antigen binding domain. Alternatively, a “nt-LNP” refers to a lipid nanoparticle devoid of antigen binding domain.

Lipid-based nanoparticles of the invention typically comprise helper lipid, sterol and/or PEG lipid components along with the mRNA of interest. The elements of a LNP may be selected based on a particular application or target, and/or based on the efficacy, toxicity, expense, ease of use, availability, or other feature of one or more elements. Similarly, the particular formulation of a lipid-based nanoparticle may be selected for a particular application or target according to, for example, the efficacy and toxicity of particular combination of elements.

The lipid-based nanoparticles of the disclosure can particularly be generated using components, compositions, and methods as generally known in the art, for example such as disclosed in WO2017049245; WO2017112865; WO2017218704; WO2015164674; WO2017031232; WO2017099823; WO2016118724; WO2016118724; WO2017223135; WO2014152211; WO2015038892; WO2017049074; WO2013090648; WO2017180917; WO2017075531 and WO2017117528 all of which are incorporated by reference herein in their entirety.

The manufacture of LNPs is well described in the art, for example in U.S. Patent Application Publication No. US20120276209, Semple et al., 2010, Nat Biotechnol., 28(2): 172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

In some aspects, the method for obtaining the lipid-based nanoparticles of the invention is as described under the “Examples” section below, in particular in Examples 1-3, or as described in FIG. 19J-K. In particular, the method for obtaining the lipid-based nanoparticles of the invention is as described in PCT/EP2024/058775.

In a particular aspect, the lipid-based nanoparticle comprises one or more ionizable or cationic lipid(s), one or more helper lipid(s), one or more sterol(s), and/or one or more polyethylene glycol (PEG)-modified lipid(s).

In a very particular aspect, the lipid-based nanoparticle comprises one or more ionizable or cationic lipid(s) (also referred as component No 1), one or more helper lipid(s) (also referred as component No 2), one or more sterol(s) (also referred as component No 3), and/or one or more polyethylene glycol (PEG)-modified lipid(s) (also referred as component No 4).

In some aspects, the lipid-based nanoparticle according to the invention comprises one or more ionizable or cationic lipid(s). As used herein, the term “ionizable or cationic lipid” refers to a lipid molecule positively charged in an acidic environment.

Ionizable or cationic lipids particularly promotes nucleic acid molecule(s) delivery and transfection efficiency. Their mechanism of action is based on complexing the nucleic acid by electrostatic interactions. Several properties, such as the charge or lipid shape, as well as the protein corona formation, have been described as important factors to consider when understanding structure-activity relationship studies, and thus, the design of new ionizable lipids.

In one aspect, the ionizable or cationic lipid comprises a head group which includes at least one nitrogen atom (N) which is positively charged or capable of being protonated.

In some aspects, the ionizable or cationic lipid is selected from the group consisting of [(4-hydroxybutyl)azanediyl]di(hexane-6,1-diyl) bis(2-hexyldecanoate) (ALC-0315), 1,2-dioleoyl-3-trimethylammonium propane (DOTAP); N,N-dimethyl-2,3-dioleyloxypropylamine (DODMA), 1,2-di-O-octadecenyl-3-trimethylammoniumpropane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammoniumpropanes; 1,2-dialkyloxy-3-dimethylammoniumpropanes; dioctadecyldimethylammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (+)-N-(3-aminopropyl)-N,N-dimethyl-2,3-bis(cis-9-tetradecenyloxy)-1-propanaminium bromide (GAP-DMORIE), (+)-N-(3-aminopropyl)-N,N-dimethyl-2,3-bis(dodecyloxy)-1-propanaminium bromide (GAP-DLRIE), (+)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminiiim (DOBAQ), 2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98Niz-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), 9-Heptadecanyl 8-{(2-hydroxyethyl)[6-oxo-6-(undecyloxy)hexyl]amino}octanoate (SM-102), bis[2-(4-{2-[4-(cis-9-octadecenoyloxy)phenylacetoxy]ethyl}piperidinyl)ethyl]disulfide (SS-OP; e.g., CAS number 2377474-67-2) bis{2-[4-(α-D-tocopherolhemisuccinateethyl)piperidyl]ethyl}disulfide (SS-EC) and any mixtures thereof, preferably is ALC-0315, SM-102, Dlin-MC3-DMA or SS-OP, more preferably ALC-0315 or SS-OP.

Ionizable or cationic lipids can be selected from the group consisting of 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N— (N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (+)-N-(3-aminopropyl)-N,N-dimethyl-2,3-bis(cis-9-tetradecenyloxy)-1-propanaminium bromide (GAP-DMORIE), (+)-N-(3-aminopropyl)-N,N-dimethyl-2,3-bis(dodecyloxy)-1-propanaminium bromide (GAP-DLRIE), (+)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminiiim (DOBAQ), 2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), Nl-[2-((lS)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98Niz-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), [(4-hydroxybutyl)azanediyl]di(hexane-6,1-diyl) bis(2-hexyldecanoate) (ALC-0315), 9-Heptadecanyl 8-{(2-hydroxyethyl)[6-oxo-6-(undecyloxy)hexyl]amino}octanoate (SM-102) and any mixtures thereof.

Additional examples of ionizable or cationic lipids are described in WO 2016/021683, WO 2015/011633, WO 2011/153493, WO 2013/126803, WO 2010/054401, WO 2010/042877, WO 2016/104580, WO 2015/005253, WO 2014/007398, WO 2017/117528, WO 2017/075531, WO 2017/00414, WO 2015/199952, US 2015/0239834, WO2019/131839 all of which are incorporated by reference herein in their entirety.

In addition, synthetic ionizable or cationic lipids (e.g., K-E12, H-A12, Y-E12, G-O12, K-A12, R-A12, cKK-E12, cPK-E12, PK1K-E12, PK500-E12, cQK-E12, cKK-A12, KK-A12, PK-4K-E12, cWK-E12, PK500-012, PK1K-O12, cYK-E12, cDK-E12, cSK-E12, cEK-E12, cMK-E12, cKK-O12, cIK-E12, cKK-E10, cKK-E14, and cKK-E16, preferably, cKK-E12, cKK-E14) described in Dong et al. (Proc Natl Acad Sci USA. 2014 Apr. 15; 111(15):5753, the disclosure thereof being incorporated herein by reference), and the synthetic ionizable or cationic lipid (e.g., C14-98, C18-96, C14-113, C14-120, C14-120, C14-110, C16-96 and C12-200, preferably C14-110, C16-96 and C12-200) described in Love K T et al. (Proc Natl Acad Sci USA. 2010 May 25; 107(21):9915, the disclosure thereof being incorporated herein by reference) can be also envisioned.

In a preferred aspect, the lipid-based nanoparticle according to the invention comprises an ionizable or cationic lipid such as described in WO 2016/021683 or WO 2019/131839 which are incorporated herein by reference in their entirety.

Particularly, the lipid composition of the lipide-based nanoparticle according to the invention comprises an ionizable or cationic lipid selected from the group consisting of [(4-hydroxybutyl)azanediyl]di(hexane-6,1-diyl) bis(2-hexyldecanoate) (ALC-0315), heptatriaconta-6,9,28,31-tetraen-19-yl-4-(dimethylamino)butanoate (DLin-MC3-DMA), 9-Heptadecanyl 8-{(2-hydroxyethyl)[6-oxo-6-(undecyloxy)hexyl]amino}octanoate (SM-102), and bis[2-(4-{2-[4-(cis-9-octadecenoyloxy)phenylacetoxy]ethyl}piperidinyl)ethyl]disulfide (SS-OP) and any mixtures thereof.

In some aspects, the ionizable or cationic lipid is selected from the group consisting of ALC-0315, SM-102, Dlin-MC3-DMA and SS-OP.

Preferably, the ionizable or cationic lipid comprised in the lipid-based nanoparticle is ALC-0315. Alternatively, the ionizable or cationic lipid comprised in the lipid-based nanoparticle is SS-OP. In some aspects, the ionizable or cationic lipid represents from about 10 mol % to about 100 mol %, about 20 mol % to about 100 mol %, about 30 mol % to about 100 mol %, about 40 mol % to about 100 mol %, or about 50 mol % to about 100 mol % of the total lipids present in the lipid-based nanoparticle according to the invention.

In some aspects, the ionizable or cationic lipid, preferably ALC-0315 or SS-OP, represents from about 45 mol % to about 55 mol % of the total lipids present in the lipid composition of the lipid-based nanoparticle according to the invention. Particularly, the ionizable or cationic lipid, preferably ALC-0315 or SS-OP, represents from about 48 mol % to about 52 mol % of the total lipids present in the lipid composition of the lipid-based nanoparticle according to the invention.

More particularly, the ionizable or cationic lipid, preferably ALC-0315 or SS-OP, represents about 50 mol % of the total lipids present in the lipid composition of the lipid-based nanoparticle according to the invention.

In some aspects, the lipid-based nanoparticle according to the invention comprises a helper lipid. As used herein, the term “helper lipid” refers to a class of lipid molecules that increases particle stability, fluidity tolerability and/or biodistribution of lipid-based nanoparticles.

Helper lipids are also constituents of LNPs, playing an important role in terms of stability and fusogenicity. These lipids are mainly phospholipids (such as DOPE, DSPC, DEPC, DSPE), which forms the main skeleton of LNPs. Helper lipids modulate nanoparticle fluidity and enhance efficacy by promoting lipid phase transitions that aid membrane fusion with the endosome. Helper lipids are generally saturated phospholipids, which can increase the phase transition temperature of cationic liposomes, support the formation of lamellar lipid bilayers and stabilize their structural arrangement.

For instance, the helper lipid can be selected from the group consisting of 1,2-distearoyl-sn-glycero-3-phosphocholine (DSPC), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1-stearoyl-2-oleoyl-sn-glycero-3-phosphocholine (SOPC), ethyl phosphatidylcholine (EPC), 1-oleoyl-2-hydroxy-sn-glycero-3-phosphocholine (18:1 Lyso PC), 1,2-dioleoyl-sn-glycero-3-phosphoethanolamine (DOPE), 1,2-diphytanoyl-sn-glycero-3-phosphoethanolamine (ME 16:0 PE), 1-hexadecyl-2-(9Z-octadecenoyl)-sn-glycero-3-phosphoethanolamine (C16-18:1), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1-palmitoyl-2-oleoyl-sn-glycero-3-phosphoethanolamine (POPE), 1,2-di-O-phytanyl-sn-glycero-3-phosphoethanolamine (4ME), 1-stearoyl-2-oleoyl-sn-glycero-3-phosphoethanolamine (SOPE), 1,2-dielaidoylsn-glycero-3-phosphoethanolamine (DEPE), N-(7-nitrobenz-2-oxa-1,3-diazol-4-yl)-phosphatidylethanolamine (NBD-PE), N-(lisamineRhodamine B sulfonyl)-phosphatidylethanolamine (Rh-PE), 1-oleoyl-2-hydroxy-sn-glycero-3-phosphoethanolamine (18:1 Lyso PE), 1,2-dioleoyl-sn-glycero-3-phosphoethanolamine-N-methyl (18:1 Monomethyl PE), 1,2-dioleoyl-sn-glycero-3-phosphoethanolamine-N,N-dimethyl (18:1 Dimethyl PE), 1,2-dioleoyl-sn-glycero-3-phosphoethanolamine-N-(hexanoylamine) (18:1 Caproylamine PE), 1,2-dioleoyl-sn-glycero-3-phosphoethanolamine-N-(biotinyl) (18:1 Biotinyl PE), sn-(3-oleoyl-2-hydroxy)-glycerol-1-phospho-sn-1′-(3′-oleoyl-2′-hydroxy)-glycerol (BMP-S,S), sn-(3-(9Z-octadecenoyl)-2-hydroxy)-glycerol-1-phospho-sn-3′-(1′-(9Z-octadecenoyl)-2′-hydroxy)-glycerol (BMP-S,R), 1,2-dioleoyl-sn-glycero-3-phospho-rac-(1-glycerol) sodium salt (DOPG), 1,2-Diacyl-sn-glycero-3-phospho-L-serine (DSPS), L-α-phosphatidylserine (PS), 1,2-dioleoyl-sn-glycero-3-phosphate (PA), 1,2-dioleoyl-sn-glycero-3-phospho-(1′-rac-glycerol) (PG), 1,2-dioleoyl-sn-glycero-3-phosphomethanol (18:1 Phosphatidymethanol), 1,2-dioleoyl-sn-glycero-3-phosphoethanol (18:1 Phosphatidyethanol), 1,2-dioleoyl-sn-glycero-3-phosphopropanol (18:1 Phosphatidypropanol), 1,2-dioleoyl-sn-glycero-3-phospho-L-serine (18:1 PS, DOPS), 1,2-distearoyl-sn-glycero-3-phospho-L-serine (18:0 PS), N-oleoyl-D-erythro-sphingosine (Ceramide), Sphingomyelin (SM), Phosphatidylinositol (PI), 9A1P9, 1,2-dioleoyl-3-trimethylammonium-propane (DOTAP), 1,2-di-O-octadecenyl-3-trimethylammonium propane (DOTMA), Dimethyldioctadecylammonium (18:0 DDAB), and any mixtures thereof.

Particularly, the helper lipid can be selected from the group consisting of 1,2-distearoyl-sn-glycero-3-phosphocholine (DSPC), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1-stearoyl-2-oleoyl-sn-glycero-3-phosphocholine (SOPC), ethyl phosphatidylcholine (EPC), 1-oleoyl-2-hydroxy-sn-glycero-3-phosphocholine (18:1 Lyso PC), 1,2-dioleoyl-sn-glycero-3-phosphoethanolamine (DOPE), 1,2-diphytanoyl-sn-glycero-3-phosphoethanolamine (ME 16:0 PE), 1-hexadecyl-2-(9Z-octadecenoyl)-sn-glycero-3-phosphoethanolamine (C16-18:1), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1-palmitoyl-2-oleoyl-sn-glycero-3-phosphoethanolamine (POPE), 1,2-di-O-phytanyl-sn-glycero-3-phosphoethanolamine (4ME), 1-stearoyl-2-oleoyl-sn-glycero-3-phosphoethanolamine (SOPE), 1,2-dielaidoylsn-glycero-3-phosphoethanolamine (DEPE), N-(7-nitrobenz-2-oxa-1,3-diazol-4-yl)-phosphatidylethanolamine (NBD-PE), N-(lisamineRhodamine B sulfonyl)-phosphatidylethanolamine (Rh-PE), 1-oleoyl-2-hydroxy-sn-glycero-3-phosphoethanolamine (18:1 Lyso PE), 1,2-dioleoyl-sn-glycero-3-phosphoethanolamine-N-methyl (18:1 Monomethyl PE), 1,2-dioleoyl-sn-glycero-3-phosphoethanolamine-N,N-dimethyl (18:1 Dimethyl PE), 1,2-dioleoyl-sn-glycero-3-phosphoethanolamine-N-(hexanoylamine) (18:1 Caproylamine PE), 1,2-dioleoyl-sn-glycero-3-phosphoethanolamine-N-(biotinyl) (18:1 Biotinyl PE), sn-(3-oleoyl-2-hydroxy)-glycerol-1-phospho-sn-1′-(3′-oleoyl-2′-hydroxy)-glycerol (BMP-S,S), sn-(3-(9Z-octadecenoyl)-2-hydroxy)-glycerol-1-phospho-sn-3′-(1′-(9Z-octadecenoyl)-2′-hydroxy)-glycerol (BMP-S,R), 1,2-dioleoyl-sn-glycero-3-phospho-rac-(1-glycerol) sodium salt (DOPG), 1,2-Diacyl-sn-glycero-3-phospho-L-serine (DSPS), L-α-phosphatidylserine (PS), 1,2-dioleoyl-sn-glycero-3-phosphate (PA), 1,2-dioleoyl-sn-glycero-3-phospho-(1′-rac-glycerol) (PG), 1,2-dioleoyl-sn-glycero-3-phosphomethanol (18:1 Phosphatidymethanol), 1,2-dioleoyl-sn-glycero-3-phosphoethanol (18:1 Phosphatidyethanol), 1,2-dioleoyl-sn-glycero-3-phosphopropanol (18:1 Phosphatidypropanol), 1,2-dioleoyl-sn-glycero-3-phospho-L-serine (18:1 PS, DOPS), 1,2-distearoyl-sn-glycero-3-phospho-L-serine (18:0 PS), N-oleoyl-D-erythro-sphingosine (Ceramide), Sphingomyelin (SM), Phosphatidylinositol (PI), 9A1P9, 1,2-dioleoyl-3-trimethylammonium-propane (DOTAP), 1,2-di-O-octadecenyl-3-trimethylammonium propane (DOTMA), Dimethyldioctadecylammonium (18:0 DDAB), 1,2-dioleyloxy-3-dimethylaminopropane (DODMA), 1,2-dioleoyl-3-dimethylammonium-propane (DODAP), 1,2-dierucoyl-sn-glycero-3-phosphocholine (DEPC), 1,2-distearoyl-sn-glycero-3-phosphoethanolamine (DSPE) and any mixtures thereof.

Preferably, the helper lipid is selected from the group consisting of DOPE, DOPS, DODMA, DOTAP, DODAP, DDAB, POPE, DSPC, DOPC, DEPC and DSPE and any mixtures thereof, preferably from the group consisting of DOPE, DOPC, DDAB, POPE and DSPC and any mixtures thereof.

In some embodiments, the helper lipid is DOPE. Alternatively, the helper lipid is DSPC.

In some aspects, the helper lipid represents from about 5 mol % to about 100 mol %, from about 10 mol % to about 100 mol %, about 20 mol % to about 100 mol %, about 30 mol % to about 100 mol %, about 40 mol % to about 100 mol %, or about 50 mol % to about 100 mol % of the total lipids present in the lipid-based nanoparticle of the invention.

In some aspects, the helper lipid, preferably DOPE or DSPC, represents from about 5 mol % to about 15 mol % of the total lipids present in the lipid-based nanoparticle of the invention.

In some aspects, the helper lipid, preferably DOPE or DSPC, represents from about 8 mol % to about 12 mol % of the total lipids present in the lipid-based nanoparticle of the invention.

In some aspects, the helper lipid, preferably DOPE or DSPC, represents about 10 mol % of the total lipids present in the lipid-based nanoparticle of the invention.

In some aspects, the lipid of the lipid-based nanoparticle disclosed herein comprises one or more molecules comprising polyethylene glycol (PEG). Accordingly, the lipid-based nanoparticle may comprise PEG or PEG-modified lipids.

As used herein, the term “PEG lipid” may refer to polyethylene glycol (PEG)-modified lipids.

PEG lipid stabilizes lipid nanoparticles, regulates nanoparticle size by limiting lipid fusion and increases nanoparticle half-life by reducing nonspecific interactions with macrophages, improving colloidal stability and preventing the formation of the protein corona. PEG lipid phospholipids that are located on the surface of nanoparticles, improve their hydrophilicity, avoid rapid clearance by the immune system, prevent particle aggregation, and increase stability.

As used herein, the term “PEG lipid” may refer to polyethylene glycol (PEG)-modified lipids. Non-limiting examples of PEG lipids include PEG-modified phosphatidylethanolamine and phosphatidic acid, PEG-ceramide conjugates (e.g., PEG-CerCI4 or PEG-CerC20), PEG-modified dialkylamines and PEG-modified 1,2-diacyloxypropan-3-amines. Such lipids are also referred to as PEGylated lipids. In some aspects, a PEG lipid can be PEG-c-DOMG, PEG-DMG, PEG-DLPE, PEG-DMPE, PEG-DPPC, or a PEG-DSPE lipid. In some aspects, the PEG-modified lipids are a modified form of PEG-DMG. A PEG lipid may particularly be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some aspects, a PEG lipid is selected from the group consisting of PEG-c-DOMG, PEG-DMG, DMG-PEG-2000, PEG-DLPE, PEG-DMPE, PEG-DPPC, and PEG-DSPE lipid.

In some aspects, the PEG-lipid includes, but is not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some aspects, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA) and ALC-0159 (N,N-dimyristylamide of 2-hydroxyacetic acid, O-pegylated to a PEG), and any mixture thereof.

In one aspect, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

For example, such PEG is selected from the group consisting of 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), PEG-c-DOMG, PEG-DMG, DMG-PEG-2000, PEG-DLPE, PEG-DMPE, PEG-DPPC, and PEG-DSPE.

Preferably, the PEG-lipid is selected from the group consisting of PEG-DMG, PEG-DSPE, PEG-c-DOMG, DMG-PEG-2000, DSPE-PEG-2000, PEG-DLPE, PEG-DMPE, PEG-DPPC, PEG-DPPE, PEG-DAG and PEG-c-DMA, ALC-0159 (N,N-dimyristylamide of 2-hydroxyacetic acid, O-pegylated to a PEG), and any mixture thereof; particularly from the group consisting of PEG-DMG, PEG-DSPE, ALC-0159 and any mixture thereof.

Preferably, the PEG is comprised between 2000 Daltons and 5000 Daltons (i.e., PEG-2000 to PEG-5000), preferably is DSPE-PEG-2000, DMG-PEG-2000, DSPE-PEG-5000, DMG-PEG-5000 or any mixture thereof.

In a particular aspect, the LNP comprises a PEG that is not functionalized, which is a PEG that does not comprises any reactive species at its end, said reactive species being usable to conjugate a target moiety, such as an antibody or a fragment thereof, to the PEG.

In some aspects, the lipid moiety of the PEG-lipids includes those having lengths of from about C14 to about C22, preferably from about C14 to about C16.

Whereas the abundant prior art focuses on a high variety of structures and ratio of the cationic lipid and of the helper lipids in the LNP, the applicant has tested different structures and lengths of PEG-lipid (component no 4) to determine their impact on the biodistribution of the LNP in different areas of the body. A typical biodistribution of LNP particles that has been observed so far was with a high ratio in the liver, and much less in other organs that contain immune cells such as the spleen. Due to said liver uptake, the circulation of LNP towards various other tissues, and in particular towards tumoral tissues, is variable. An increased targeting of liver cells is for example described in WO2022/261101. However, in other clinical situations, a high or excessive uptake of LNP by capturing organs (mostly the liver, and potentially also the spleen and lungs notably) is not advantageous. Therefore, there is a need to provide LNP formulations that can escape the liver and reach other organs such as the spleen.

The inventors observed that the PEG-lipid has an impact on the biodistribution of the LNPs. The choice of the PEG-lipid can modify the uptake of the LNPs by the capturing organs (mostly liver, and optionally spleen and/or lungs). For instance, a PEG-C14 lipid seems to favour liver uptake whereas a PEG-C18 lipid decreases liver uptake and may optionally favour spleen targeting. Optionally, the PEG-lipid and the amount thereof in the LNP can be selected so as favouring free distribution of the LNP throughout the circulation of the body, in particular in direction of an area of interest for a specific targeted therapeutic treatment.

As an illustrative advantage, targeted LNP carrying targeting ligand such as antibodies, are designed to escape the uptake by organs and target activated immune cells (such as PD1+ T lymphocytes). The mRNA carried by the LNP and transfected into said activated immune cells allows to reinforce their anti-tumoral activity locally and specifically.

Accordingly, in a more specific aspect, the lipid, in particular the lipid moiety of the PEG-lipids includes those having a lengths of from about C16 to about C22 (C16, C17, C18, C19, C20, C21 or C22), preferably C16 to C20 (C16, C17, C18, C19 or C20), especially C18.

Such PEG lipid can be selected in the group consisting of 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), and PEG-dipalmitoyl phosphatidylethanolamine (PEG-DPPE), optionally in the group consisting of 1,2-distearoyl-sn-glycero-3-phosphoethanolamine-N-[amino(polyethylene glycol)](PEG-DSPE), PEG-disteryl glycerol (PEG-DSG), and PEG-dioleyl, PEG-distearyl. In a very particular aspect, the PEG-lipid is PEG-disteryl glycerol (PEG-DSG).

Optionally, the PEG may present a molecular weight within the range from 0.5 to 50 kD, more preferably from 1 to 20 kD. In some aspects, a PEG moiety, for example a mPEG-NH2, has a size of about 1000, 2000, 5000, 10,000, 15,000 or 20,000 Daltons. In a very particular aspect, the PEG has a size of about 2000. In a very particular aspect, the PEG-lipid is PEG 2000-DMG.

In a very particular aspect, the PEG has a size of between about 2000 Daltons and 5000 Daltons (i.e., PEG-2000 to PEG-5000). In some instances, the PEG is selected from the group consisting of PEG-2000, PEG-2500, PEG-3000, PEG-3500, PEG-4000, PEG-4500 and PEG-5000.

In one aspect, the PEG-lipid is PEG 2000-DMG. In another very particular aspect, the PEG-lipid is PEG 2000-DSG. In another very particular aspect, the PEG-lipid is ALC-0159.

In a particular aspect, the PEG has a size of about 2000. Alternatively, the PEG has a size of about 5000 Daltons.

In some instances, the PEG lipid is selected from the group consisting of DSPE-PEG-2000, DMG-PEG-2000, DSPE-PEG-5000, DMG-PEG-5000 or a mixture thereof.

In a very particular aspect, the PEG-lipid is PEG 5000-DMG. In another very particular aspect, the PEG-lipid is PEG 5000-DSG. In some aspects, the PEG lipid represents from about 1 mol % to about 100 mol %, from about 2 mol % to about 100 mol %, about 3 mol % to about 100 mol %, about 4 mol % to about 100 mol %, about 5 mol % to about 100 mol %, about 10 mol % to about 100 mol %, or about 15 mol % to about 100 mol % of the total lipids present in the lipid-based nanoparticle of the invention.

Optionally, the PEG lipid in the lipid-based nanoparticle is within the range from about 0.5 mol % to about 2 mol % of the total lipids present in the nanoparticle, for instance about 0.5, 0.6, 0.7, 0.8, 0.9, 1.0, 1.1, 1.2, 1.3, 1.4, 1.5, 1.6, 1.7, 1.8, 1.9 or 2.0 mol %, especially 1.5 mol %.

In addition, the inventors have surprisingly observed that the amount of PEG lipid has an impact of the biodistribution of the LNPs. It appears that a lower amount of PEG lipid may decrease the uptake of LNPs by capturing organs, especially liver but also spleen.

Accordingly, the PEG lipid in said lipid-based nanoparticle could be less than 1.5 mol % of the total lipids present in the nanoparticle, in particular, less than 1.4, 1.3, 1.2, 1.1, 1.0, 0.9, 0.8, 0.7, 0.6 or 0.5 mol % of the total lipids present in the nanoparticle.

In some aspects, the PEG lipid, preferably PEG-DMG or PEG DSPE, represents from about 0.5 mol % to about 5 mol % or from about 0.5 mol % to about 2.5 mol % of the total lipids present in the lipid-based nanoparticle of the invention.

Particularly, the PEG lipid, preferably PEG-DMG or PEG DSPE, represents from about 1 mol % to about 2 mol % of the total lipids present in the lipid-based nanoparticle of the invention.

Preferably, the PEG lipid, preferably PEG-DMG or PEG DSPE, represents about 1.5 mol % of the total lipids present in the lipid-based nanoparticle of the invention.

In some aspects, the lipid-based nanoparticle of the invention comprises one or more sterol.

The sterol can particularly be selected from the group consisting of cholesterol, fecosterol, sitosterol, ergosterol, campesterol, stigmasterol, brassicasterol, tomatidine, ursolic acid, alpha-tocopherol, and any mixtures thereof. Preferably, the sterol is cholesterol.

Sterol, more so cholesterol, enhances the stability of the nanoparticles by filling gaps between lipids, and aids fusion with the endosomal membrane during uptake into the cell.

In some aspects, the sterol represents from about 10 mol % to about 100 mol %, from about 20 mol % to about 100 mol %, about 30 mol % to about 100 mol %, about 40 mol % to about 100 mol %, about 50 mol % to about 100 mol %, about 60 mol % to about 100 mol %, or about 70 mol % to about 100 mol % of the total lipids present in the lipid-based nanoparticle of the invention.

In some aspects, the sterol, preferably cholesterol, represents from about 30 mol % to about 50 mol % or from about 35 mol % to about 45 mol % of the total lipids present in the lipid-based nanoparticle of the invention.

Particularly, the sterol, preferably cholesterol, represents from about 35 mol % to about 40 mol % of the total lipids present in the lipid-based nanoparticle of the invention. Such sterol being preferably cholesterol.

Particularly, the sterol, preferably cholesterol, represents about 38.5 mol % of the total lipids present in the lipid-based nanoparticle of the invention. Such sterol being preferably cholesterol.

In a preferred aspect, the lipid-based nanoparticle of the invention comprises an ionizable or cationic lipid, a helper lipid, a sterol and a PEG lipid.

In a preferred aspect, the lipid composition of the lipid-based nanoparticle of the invention consists of an ionizable or cationic lipid, a helper lipid, a sterol and a PEG lipid, these lipids being preferably as described above.

Preferably, in said lipid-based nanoparticle, the ionizable or cationic lipid is from about 10 mol % to about 70 mol % of the total lipids present in the nanoparticle, the helper lipid is from about 5 mol % to about 70 mol % of the total lipids present in the nanoparticle, the sterol is from about 10 mol % to about 70 mol % of the total lipids present in the nanoparticle, and the PEG lipid is from about 0.5 mol % to about 4 mol % of the total lipids present in the nanoparticle.

Particularly, the lipid-based nanoparticle comprises or consists of from about 35 mol % to about 55 mol % of a cationic or ionizable lipid, from about 5 mol % to about 20 mol % of a helper lipid, from about 30 mol % to about 60 mol % of a sterol, and from about 0.5 mol % to about 4 mol % of a PEG-lipid.

Preferably, the lipid-based nanoparticle comprises or consists of from about 45 mol % to about 55 mol % of a cationic or ionizable lipid, from about 5 mol % to about 15 mol % of a helper lipid from about 35 mol % to about 45 mol % of a sterol, and from about 0.5 mol % to about 2.5 mol % of a PEG-lipid.

In some aspects, a polymer may be included in and/or used to encapsulate or partially encapsulate the lipid-based nanoparticle according to the invention. The polymer may be biodegradable and/or biocompatible. The polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, the polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

In some aspects, the lipid based particle comprises a poloxamine and/or a poloxamer.

In some aspects, the lipid based particle comprises a plyethyleneimine, rotamine and/or polyaspartamide.

In some aspects, a surface altering agent may be disposed within a lipid-based nanoparticle of the invention and/or on the surface of the lipid-based nanoparticle (e.g., by coating, adsorption, covalent linkage, or other process). Surface altering agents include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin b4, dornase alfa, neltenexine, and erdosteine), and DNases (e.g., rhDNase).

In a very specific aspect, the lipid-based nanoparticle comprises a lipid mixture.

Preferably, the lipid mixture comprises or consists of an ionizable or cationic lipid, a helper lipid, a sterol and a PEG lipid, these lipids being preferably as described here above.

In a specific aspect, the lipid-based nanoparticle comprises [(4-hydroxybutyl)azanediyl]di(hexane-6,1-diyl) bis(2-hexyldecanoate) (ALC-0315) as ionizable or cationic lipid, 1,2-dioleoyl-sn-glycero-3-phosphoethanolamine (DOPE) as neutral lipid, cholesterol as sterol and one or more polyethylene glycol (PEG)-modified lipid(s).

In a more specific aspect, ALC-0315 is from about 35 mol % to about 55 mol % of the total lipids present in the LNP, DOPE is from about 5 mol % to about 20 mol % of the total lipids present in the LNP, cholesterol is from about 30 mol % to about 60 mol % of the total lipids present in the LNP, and said one or more polyethylene glycol (PEG)-modified lipid(s) is from about 0.5 mol % to about 4 mol % of the total lipids present in the LNP. Optionally, said one or more polyethylene glycol (PEG)-modified lipid(s) is from about 0.5 mol % to about 2 mol %, for instance about 0.5, 0.6, 0.7, 0.8, 0.9, 1.0, 1.1, 1.2, 1.3, 1.4, 1.5, 1.6, 1.7, 1.8, 1.9 or 2.0 mol %, especially 1.5 mol %. Optionally, said one or more polyethylene glycol (PEG)-modified lipid(s) is from about 0.5 mol % to about 1.5 mol % of the total lipids present in the LNP. Optionally, said one or more polyethylene glycol (PEG)-modified lipid(s) is from about 0.5 mol % to about 0.6, 0.7, 0.8, 0.9 or 1.0 mol % of the total lipids present in the LNP.

In some aspects, the lipid-based nanoparticle comprises [(4-hydroxybutyl)azanediyl]di(hexane-6,1-diyl) bis(2-hexyldecanoate) (ALC-0315) as ionizable or cationic lipid, 1,2-dioleoyl-sn-glycero-3-phosphoethanolamine (DOPE) as neutral lipid, cholesterol as sterol and 1-monomethoxypolyethyleneglycol-2,3-dimyristylglycerol with polyethylene glycol of average molecular weight 2000 (PEG2000-DMG) as PEG-modified lipid.

In a very specific aspect, ALC-0315 is from about 35 mol % to about 55 mol % of the total lipids present in the LNP, DOPE is from about 5 mol % to about 20 mol % of the total lipids present in the LNP, cholesterol is from about 30 mol % to about 60 mol % of the total lipids present in the LNP, and the PEG2000-DMG is from about 0.5 mol % to about 4 mol % of the total lipids present in the LNP.

Optionally, PEG2000-DMG is from about 0.5 mol % to about 2 mol % of the total lipids present in the LNP, for instance about 0.5, 0.6, 0.7, 0.8, 0.9, 1.0, 1.1, 1.2, 1.3, 1.4, 1.5, 1.6, 1.7, 1.8, 1.9 or 2.0 mol %, especially 1.5 mol %. Optionally, PEG2000-DMG is from about 0.5 mol % to about 1.5 mol % of the total lipids present in the LNP. Optionally, PEG2000-DMG is from about 0.5 mol % to about 0.6, 0.7, 0.8, 0.9 or 1.0 mol % of the total lipids present in the LNP, preferably about 0.5 mol %.

In another very specific aspect, the lipid-based nanoparticle comprises [(4-hydroxybutyl)azanediyl]di(hexane-6,1-diyl) bis(2-hexyldecanoate) (ALC-0315) as ionizable or cationic lipid, 1,2-dioleoyl-sn-glycero-3-phosphoethanolamine (DOPE) as neutral lipid, cholesterol as sterol and a disteryl glycerol (DSG) with polyethylene glycol of average molecular weight 2000 (PEG2000-DSG) as PEG-modified lipid.

In a very specific aspect, ALC-0315 is from about 35 mol % to about 55 mol % of the total lipids present in the LNP, DOPE is from about 5 mol % to about 20 mol % of the total lipids present in the LNP, cholesterol is from about 30 mol % to about 60 mol % of the total lipids present in the LNP, and the PEG2000-DSG is from about 0.5 mol % to about 4 mol % of the total lipids present in the LNP. Optionally, PEG2000-DSG is from about 0.5 mol % to about 2 mol % of the total lipids present in the LNP, for instance about 0.5, 0.6, 0.7, 0.8, 0.9, 1.0, 1.1, 1.2, 1.3, 1.4, 1.5, 1.6, 1.7, 1.8, 1.9 or 2.0 mol %, especially 1.5 mol %. Optionally, PEG2000-DSG is from about 0.5 mol % to about 1.5 mol % of the total lipids present in the LNP. Optionally, PEG2000-DSG is from about 0.5 mol % to about 0.6, 0.7, 0.8, 0.9 or 1.0 mol % of the total lipids present in the LNP, preferably about 0.5 mol %.

In a very specific aspect, the lipid composition of the LNP according to the invention comprises:
ALC-0315 from about 35 mol % to about 55 mol % of the total lipids present in the LNP,
DOPE from about 5 mol % to about 20 mol % of the total lipids present in the LNP,
Cholesterol from about 30 mol % to about 60 mol % of the total lipids present in the LNP,
and PEG 2000-DSG and/or PEG 2000-DMG, preferably PEG 2000-DSG, from about 0.5 mol % to about 4 mol % of the total lipids present in the LNP, preferably from about 0.5 mol % to about 1.5 or 2 mol % of the total lipids present in the LNP, optionally from about 0.5 mol % to about 1.0 mol % of the total lipids present in the LNP.

In some aspects, the lipid-based composition of the lipid-based nanoparticle comprises or consists of a lipid mixture selected from the group consisting of:
a) ALC-0315, DOPE, cholesterol and DMG-PEG,
b) ALC-0315, DDAB, cholesterol and DMG-PEG,
c) ALC-0315, POPE, cholesterol and DMG-PEG,
d) ALC-0315, DOPE, cholesterol and DSPE-PEG,
e) ALC-0315, DSPC, cholesterol and DMG-PEG,
f) ALC-0315, DSPC, cholesterol and ALC-0159;
g) SM-102, DSPC, cholesterol and DMG-PEG,
h) Dlin-MC3-DMA, DSPC, cholesterol and DMG-PEG,
i) ALC-0315, DOPE, cholesterol, DMG-PEG and DSPE-PEG,
j)SS-OP, DOPE, cholesterol and DMG-PEG;
k)SS-OP, DSPC, cholesterol and DSPE-PEG; and
l)SS-OP, DOPC, cholesterol and DMG-PEG.

In some aspects, the lipid-based composition comprises or consists of a lipid mixture selected from the group consisting of:
a)SS-OP, POPE, cholesterol and DMG-PEG 2000;
b)SS-OP, DEPC, cholesterol and DMG-PEG 2000;
c)SS-OP, DOPC, cholesterol and DMG-PEG 2000;
d)SS-OP, DOPC, cholesterol and DSG-PEG 5000 or 2000; and
e)SS-OP, DSPC, cholesterol and DSPE-PEG 2000.

Preferably, the lipid-based composition comprises or consists of the following lipid mixture SS-OP, DSPC, cholesterol and DSPE-PEG, preferably DSG-PEG 2000 or DSG-PEG 5000.

Alternatively, the lipid-based composition comprises or consists of a lipid mixture selected from the group consisting of:
a) ALC-0315, DOPE, cholesterol and DMG-PEG;
b) ALC-0315, DDAB, cholesterol and DMG-PEG;
c) ALC-0315, POPE, cholesterol and DMG-PEG;
d) ALC-0315, DOPE, cholesterol and DSPE-PEG;
e) ALC-0315, DSPC, cholesterol and DMG-PEG;
f) ALC-0315, DSPC, cholesterol and ALC-0159;
g) ALC-0315, DOPE, cholesterol, DMG-PEG and DSPE-PEG; Preferably, in such specific aspects, the PEG has a size of 2000 Daltons (i.e., PEG-2000).

Preferably, in such aspects, the lipid composition of the lipid-based nanoparticle comprises or consists of from about 35 mol % to about 55 mol % of a cationic or ionizable lipid, from about 5 mol % to about 20 mol % of a helper lipid, from about 30 mol % to about 60 mol % of a sterol, and from about 0.5 mol % to about 4 mol % of a PEG-lipid.

Preferably, in such aspects, the lipid composition of the lipid-based nanoparticle comprises or consists of from about 45 mol % to about 55 mol % of a cationic or ionizable lipid, from about 5 mol % to about 15 mol % of a helper lipid from about 35 mol % to about 45 mol % of a sterol, and from about 0.5 mol % to about 2.5 mol % of a PEG-lipid.

In a very specific aspect, the lipid composition of the LNP according to the invention comprises:
ALC-00315, SM-102, Dlin-MC3-DMA or SS-OP or any mixture thereof from about 45 mol % to about 55 mol 0%, preferably from about 48 mol % to about 52 mol %, more preferably of about 50 mol % of the total lipids present in the LNP,
DOPE, DDAB, DOPC, POPE or DSPC or any mixture thereof from about 5 mol % to about 15 mol %, preferably from about 8 mol % to about 12 mol %, more preferably of about 10 mol % of the total lipids present in the LNP,
Cholesterol from about 35 mol % to about 45 mol %, preferably from about 37 mol % to about 40 mol %, more preferably of about 38.5 mol % of the total lipids present in the LNP, and
PEG 2000-DSG, PEG 2000-DMG, PEG 5000-DSG, PEG 5000-DMG or ALC-0159 or any mixture thereof, from about 0.5 mol % to about 2.5 mol %, preferably from about 1 mol % to about 2 mol %, more preferably of about 1.5 mol % of the total lipids present in the LNP.

In a very specific aspect, the lipid-based composition comprises or consists of.
SS-OP from about 35 mol % to about 55 mol % of the total lipids present in the LNP, preferably from about 48 mol % to about 52 mol %, more preferably of about 50 mol % of the total lipids present in the LNP
DSPC from about 5 mol % to about 20 mol % of the total lipids present in the LNP, preferably from about 8 mol % to about 12 mol %, more preferably of about 10 mol % of the total lipids present in the LNP,
Cholesterol from about 35 mol % to about 45 mol %, preferably from about 37 mol % to about 40 mol %, more preferably of about 38.5 mol % of the total lipids present in the LNP, and
PEG 2000-DSPE from about 0.5 mol % to about 2.5 mol %, preferably from about 1 mol % to about 2 mol %, more preferably of about 1.5 mol % of the total lipids present in the LNP.

In a very specific aspect, the lipid-based composition comprises or consists of.
ALC-0315 from about 35 mol % to about 55 mol % of the total lipids present in the LNP, preferably from about 48 mol % to about 52 mol %, more preferably of about 50 mol % of the total lipids present in the LNP;
DOPE from about 5 mol % to about 20 mol % of the total lipids present in the LNP, preferably from about 8 mol % to about 12 mol %, more preferably of about 10 mol % of the total lipids present in the LNP,
Cholesterol from about 30 mol % to about 60 mol % of the total lipids present in the LNP, preferably from about 37 mol % to about 40 mol %, more preferably of about 38.5 mol % of the total lipids present in the LNP, and
and PEG 2000-DSG and/or PEG 2000-DMG, preferably PEG 2000-DSG, from about 0.5 mol % to about 4 mol % of the total lipids present in the LNP, preferably from about 0.5 mol % to about 1.5 or 2 mol % of the total lipids present in the LNP, optionally from about 0.5 mol % to about 1.5 mol % of the total lipids present in the LNP.

The lipid-based nanoparticle according to the invention may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, imaging, or for the conjugation of the antigen binding domain to the LNP.

The LNP of the invention may optionally comprise one or more coatings. For example, the LNP may be formulated in a capsule, film, or tablet having a coating. A capsule, film, or tablet including a LNP such as described herein may have any useful size, tensile strength, hardness, or density.

Lipid-based nanoparticles or a composition comprising LNPs may be characterized by a variety of methods. For example, microscopy (e.g., transmission electron microscopy or scanning electron microscopy) may be used to examine the morphology and size distribution of a LNP or of a composition comprising LNPs. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP or of a composition comprising LNPs, such as particle size, polydispersity index, and zeta potential.

Physiochemical properties of lipid-based nanoparticles may be altered in order to increase selectivity for particular bodily targets. For instance, particle sizes may be adjusted based on the fenestration sizes of different organs.

The mean size of the lipid-based nanoparticle of the invention may be between 10 of nm and 200 of nm, e.g., measured by dynamic light scattering (DLS). For example, the mean size may be from about 40 nm to about 200 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm or 200 nm. In some aspects, the mean size of a LNP is from about 50 nm to about 200 nm, from about 50 nm to about 190 nm, from about 50 nm to about 180 nm, from about 50 nm to about 170 nm, from about 50 nm to about 160 nm, from about 60 nm to about 200 nm, from about 60 nm to about 190 nm, from about 60 nm to about 180 nm, from about 60 nm to about 170 nm, from about 70 nm to about 200 nm, from about 70 nm to about 190 nm, from about 70 nm to about 180 nm, from about 80 nm to about 200 nm, from about 80 nm to about 190 nm, or from about 90 nm to about 200 nm. In some aspects, the mean size of a LNP is from about 50 nm to about 200 nm, from about 50 nm to about 190 nm, from about 50 nm to about 180 nm, from about 50 nm to about 170 nm, from about 50 nm to about 160 nm, from about 60 nm to about 190 nm, from about 60 nm to about 180 nm, from about 60 nm to about 170 nm, from about 60 nm to about 160 nm, from about 70 nm to about 180 nm, from about 70 nm to about 170 nm, from about 70 nm to about 160 nm, from about 80 nm to about 170 nm, from about 80 nm to about 160 nm, or from about 90 nm to about 160 nm. In certain aspects, the mean size of a LNP may be from about 70 nm to about 150 nm. In a particular aspect, the mean size is about 120 nm.

In other embodiments, the mean size of the lipid-based nanoparticle is about 150 nm.

In another aspect, the mean size of the lipid-based nanoparticle of the invention may be between 10 nm and 400 nm, e.g., measured by dynamic light scattering (DLS). For example, the mean size may be from about 40 nm to about 350 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 250 nm, 300 nm or 350 nm.

The zeta potential of a lipid-based nanoparticle may be used to indicate the electrokinetic potential of a composition comprising said lipid-based nanoparticle. For example, the zeta potential may describe the surface charge of a lipid-based nanoparticle. Lipid-based nanoparticles with relatively low charges, positive or negative, are generally desirable, as more highly charged species may interact undesirably with cells, tissues, and other elements in the body. In some aspects, the zeta potential of a lipid-based nanoparticle may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some aspects, the lipid-based nanoparticle comprises an imaging agent in particular for MRI (magnetic resonance imaging), PET (Positron Emission Tomography), SPECT (Single Photon Emission Computed Tomography), ultrasound, radiography, X-ray tomography and optical imaging (fluorescence, bioluminescence, diffusion . . . ). These imaging agents can make it possible to follow the position of the lipid-based nanoparticles (and therefore of the immune cell inhibiting protein) especially after their administration to a patient. Examples of imaging agents include but are not limited to paramagnetic gadolinium chelates, paramagnetic lanthanide chelate (DOTA, DO3A, DTPA, PCTA), especially with a membrane lipophilic part, and, iron gluconates, and iron sulfates or iron oxide comprised in the cavity of the lipid-based nanoparticle, or instance, magnetite (FesCU), maghemite (y-Fe203), wiistite (FeO), hematite (a-Fe203), or combinations thereof, for example such as described in WO2021194672 the disclosure thereof being incorporated herein by reference.

The lipid-based nanoparticle of the invention comprises i) an antigen-binding domain and ii) one or several mRNA molecule(s). Each of these two components are more readily described here below. Accordingly, any of the below aspects applies to all of the lipid-based nanoparticles disclosed here above.

### Antigen Binding Domains

The lipid-based nanoparticle according to the invention comprises an antigen binding domain capable of specific binding to a target expressed on activated immune cells surface.

The terms “specific binding”, “specifically binds to,” “specific for” or “selectively binds” a particular target or an epitope on a particular antigen mean that the antigen binding domain recognizes and binds a specific antigen or epitope, but does not substantially recognize or bind other molecules in a sample. For example, an antigen binding domain that specifically (or preferentially) binds to an antigen is an antigen binding domain that binds said antigen for example with greater affinity, avidity, more readily, and/or with greater duration than it binds to other/different antigens. Preferably, the term “specifically binds to” or “binds specifically” refers to the ability of an antigen receptor to bind to an antigen with an affinity of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or bind to a target with an affinity that is at least two-fold greater than its affinity for a nonspecific antigen. The affinity can be determined by various methods well known from one skilled in the art. These methods include, but are not limited to, Biacore Analysis, Blitz analysis and Scatchard plot.

In an aspect, the antigen-binding domain comprised in the lipid-based nanoparticle according to the invention has a KD value inferior or equal to 10⁻⁸ M, preferably inferior or equal to 10⁻⁹ M for the target expressed on activated immune cells, more preferably inferior or equal to 1·10⁻¹⁰ M, as may be determined by biosensor analysis, particularly by Biacore Analysis.

As used herein, the term “target” of the antigen binding domain refers to a carbohydrate, lipid, peptide, polypeptide, protein, antigen or epitope that is specifically recognized or bound by the antigen binding domain according to the invention and expressed on the external surface of activated immune cells. With regards to the expression of a target on the surface of immune cells, the term “expressed” refers to a target, such as carbohydrates, lipids, peptides, polypeptides, proteins, antigens or epitopes that are present or presented at the outer surface of an immune cell.

In one aspect, the target is specifically expressed by activated immune cells in a healthy subject or in a subject suffering from a disease, in particular such as an autoimmune disease or an inflammatory disease. This means that the target has a higher expression level in activated immune cells than in other cells or that the ratio of activated immune cells expressing the target by the total immune cells is higher than the ratio of other cells expressing the target by the total other cells. Preferably the expression level or ratio is higher by a factor 2, 5, 10, 20, 50 or 100.

“Immune cell” as used herein includes neutrophils, eosinophils, basophils, mast cells, monocytes, macrophages, dendritic cells, natural killer cells, and lymphocytes (B cells and T cells). It preferably refers to T cells, more specifically CD4+ T cells, CD8+ T cells, effector T cells and/or exhausted T cells.

By “activated immune cells” it is meant immune cells that are involved or have been activated during an immune response towards the presence of non-self cells such as pathogens or cancer cells, or toward self cells in the case of autoimmune diseases. Activated immune cells are particularly recruited in the localization wherein the inflammation, triggered by the presence of non-self cells, occurs. Particular markers of immune cells of activation that can be targeted by the antigen binding domain are particularly described here after.

Preferably, the activated immune cells are selected from the group consisting of activated T cells, activated B cells, activated myeloid cells including activated macrophages and activated dendritic cells. Preferably, the activated immune cell is an activated T cell.

In an embodiment, the target of the antigen binding domain is selected from the group consisting of PD-1, BCMA/TNFRSF17, BTLA, CD101/IGSF2, CD103, CD119, CD137/4-1BB/TNFRSF9, CD150, CD153, CD154, CD223, CD226, CD25, CD254, CD26, CD27, CD275, CD39/ENTPD1, CD40L, CD44, CD45RO, CD45RC, LGR6, CD69, GPR18, GPR35, FPR2, CD80, CD83, CD86, CD95, CMKLR1, CRTAM, CST7, CTLA4, CXCR3, CXCR4, CXCR5, CXCR6, FasL/TNFSF6, GITR/TNFRSF18, GPR32, TIM3/HAVCR2, ICOS, IL18R1/CXCR1/CD218a, ITGAE, LAG3, TRAILR, OX40L, LY108/SlamF6, NKG2D, OX40/TNFRSF4, PTPN22, RGS1, LOX1, SIGLEC 6, TACI/TNFRSF13B, TIGIT, CD163, CD206, LTBR/CD70, TNFSF14, SLAMF1, SLAMF7, NKG2A, KIR2DL2, CD96, CD112R, CD28H, IL2RB, TRAIL, CD48, CD53, CD164, CD138 (SDC1), CD38, CD39, FCRL4, CD30/TNFRSF8, CD78, TRAF1, TRAF2, TRAF3/CD40BP, TRAF3IP1, TRAF4, TRAF7, TRAP1, TNFR1/TNFRSF1A/CD120A, TRAP100/MED24, TNFR2/TNFRSF1811/CD120B, CDCR3/TNFRSF6B, TNFRSF12A/FN14/TWEAKR, BAFFR/TNFRSF13C/CD268, HVEM/TNFRSF14/CD270, GITR/TNFRSF8/CD357, RELT/TNFRSF19L, TNFRSF19/TROY, TNFRSF21/DR6, TNFRSF25/DR3/TNFRSF12, CD301, IL4R, CLEC-1A, CD21, CLEC-9A, CD180, CD59, CD54, CD71, CD35, CD218a, CD74, CD165, 4-1BBL/CD137L, ICOSL, CD160, CD127 and SIRPa.

In an embodiment, the target of the antigen binding domain is selected from the group consisting of BCMA/TNFRSF17, BTLA, CD101/IGSF2, CD103, CD119, CD137/4-1BB/TNFRSF9, CD150, CD153, CD154, CD223, CD226, CD25, CD254, CD26, CD27, CD275, CD39/ENTPD1, CD40L, CD44, CD45RO, CD45RC, LGR6, CD69, GPR18, GPR35, FPR2, CD80, CD83, CD86, CD95, CMKLR1, CRTAM, CST7, CTLA4, CXCR3, CXCR4, CXCR5, CXCR6, FasL/TNFSF6, GITR/TNFRSF18, GPR32, TIM3/HAVCR2, ICOS, IL18R1/CXCR1/CD218a, ITGAE, LAG3, TRAILR, OX40L, LY108/SlamF6, NKG2D, OX40/TNFRSF4, PDCD1, PTPN22, RGS1, LOX1, SIGLEC 6, TACI/TNFRSF13B, TIGIT, CD163, CD206, LTBR/CD70, TNFSF14, SLAMF1, SLAMF7, NKG2A, KIR2DL2, CD96, CD112R, CD28H, IL2RB, TRAIL, CD48, CD53, CD164, CD138 (SDC1), CD38, CD39, FCRL4, CD30/TNFRSF8, CD78, TRAF1, TRAF2, TRAF3/CD40BP, TRAF3IP1, TRAF4, TRAF7, TRAP1, TNFR1/TNFRSFIA/CD120A, TRAP100/MED24, TNFR2/TNFRSF1811/CD120B, CDCR3/TNFRSF6B, TNFRSF12A/FN14/TWEAKR, BAFFR/TNFRSF13C/CD268, HVEM/TNFRSF14/CD270, GITR/TNFRSF8/CD357, RELT/TNFRSF19L, TNFRSF19/TROY, TNFRSF21/DR6, TNFRSF25/DR3/TNFRSF12, CD301, IL4R, CLEC-1A, CD21, CLEC-9A, CD180, CD59, CD54, CD71, CD35, CD218a, CD74, CD165, 4-1BBL/CD137L, ICOSL and CD160. “T cell” or “T lymphocytes” as used herein includes CD4+ T cells, CD8+ T cells, T helper 1 type T cells, T helper 2 type T cells, T helper 17 type T cells, effector T cells, effector memory stem like T cells, Tumor Infiltrating Lymphocyte (TIL), anergic T cells and exhausted T cells. In a very particular aspect, the T cell is an effector T cell, an exhausted T cell, a Tumor Infiltrating Lymphocyte (TIL) or an effector memory stem like T cell. “Activated T cell” or “Activated T lymphocytes” are T cells activated by simultaneously receiving signal-1 from T-cell recognition of antigen via the T cell receptor and signal-2 from costimulatory molecule. Markers expressed by activated T cells include but are not limited to CD137/41BB/TNFRSF9, PD-1, CTLA4, FasL/TNFSF6, ITGAE and OX40/TNFRSF4. Exhausted T cells can be characterized by the presence of the marker TNFRSF9.

Preferably, the term “T cell” does not include regulatory T cells (Treg), inhibitory T cells and/or senescent T cells. Regulatory T cells can be characterized by the presence of the marker CD25.

“B cells” or “B lymphocytes” as used herein include but are not limited to B-1 B cells, follicular B cells, and marginal zone (MZ) B cells. “Activated B cells” or “Activated B lymphocytes” are B cells activated when the B cell binds to an antigen via its B cell Receptor. Activated B cells are particularly able to secrete immunoglobulins. Markers expressed by activated B cells include but are not limited to BCMA/TNFRSF17, CD150 and CD86.

“Neutrophils” are a type of white blood cells called granulocytes and are produced from stem cells in the bone marrow. They play an important role in innate immunity “Activated Neutrophils” are neutrophils activated upon their entry into inflammatory or infected tissue site, in response to pro-inflammatory stimuli in the tissue. Neutrophils activation is characterized by the release of granule proteins, acquisition of phagocytic capabilities, and production of NETs, all of which are designed to enhance the cells' pathogen-destruction capacity. Markers of activated neutrophils include but are not limited to CD11b, CD18, CD66b, CD177 and PRTN3.

“Eosinophils” are a variety of white blood cells and one of the immune system components responsible for combating multicellular parasites and certain infections in vertebrates. They are granulocytes that develop during hematopoiesis in the bone marrow before migrating into blood. “Activated Eosinophils” are eosinophils which were recruited from the blood into the tissues at sites of inflammation or infection, and received activation signal through cytokine mediation. Upon activation, eosinophils can release an array of inflammatory mediators. Markers of activated eosinophils include but are not limited to CD69, L-selectin, ICAM-1, CD44 and PSGL-1.

“Basophils” are a type of polymorphonuclear leukocyte characterized by having a nucleus with two or three lobes, and by the presence of cytoplasmic granules. “Activated Basophils” are activated by antigen crosslinking of FceRI receptor-bound IgE to undergo rapid degranulation and release their cellular contents. In addition, basophils can be activated without IgE crosslinking by inflammatory mediators such as complement factors C5a and C3a, MBP, PAF and chemokines. Markers of activated basophils include but are not limited to CD63, CD203c and CD164.

“Mast cells”, also known as mastocytes or labrocytes are resident cells of connective tissue that contains many granules rich in histamine and heparin. “Activated Mast cells” are mast cells stimulated by allergens through cross-linking with immunoglobulin E receptors (e.g., FcεRI), physical injury through pattern recognition receptors for damage-associated molecular patterns (DAMPs), microbial pathogens through pattern recognition receptors for pathogen-associated molecular patterns (PAMPs), and various compounds through their associated G-protein coupled or ligand-gated ion channels. Upon activation, they can selectively or quickly release mediators or inflammation-inducing components, such as histamine, heparin, cytokines, and growth factors. Markers of activated mast cells include but are not limited to CD63, CD203, c-Kit, IL-3Rα, and FcεRI.

“Macrophages” are a type of white blood cells that help eliminate foreign substances by engulfing foreign materials and initiating an immune response. As used herein, this term includes for example Adipose tissue macrophages, Monocytes, Kupffer cells, Sinus histiocytes, Alveolar macrophages (dust cells), Tissue macrophages (histiocytes) leading to giant cells, Microglia, Hofbauer cells, Intraglomerular mesangial cells, Osteoclasts, Langerhans cells, Epithelioid cells, Red pulp macrophages (sinusoidal lining cells), Peritoneal macrophages, LysoMac. “Activated Macrophages” are macrophages activated either by a priming signal through IFNg followed by encountering an appropriate stimulus, such as bacterial LPS, or by direct stimulation by IL4 and/or IL13. Activated macrophages are able to kill non-self cells through phagocytosis. Markers of activated macrophages include but are not limited to TNFRSF12A/FN14/TWEAKR.

“Dendritic cells” or “DCs” are antigen-presenting cells of the immune system. Their main function is to process antigen material and present it on the cell surface to the T cells of the immune system. As used herein, this term includes for example plasmacytoid dendritic cells (pDC) and myeloid dendritic cells (mDC). “Activated Dendritic cells” are dendritic cells directly activated by conserved pathogen molecules and indirectly by inflammatory mediators produced by other cell types that recognize such molecules. Markers of activated dendritic cells include but are not limited to TRAF4.

“Natural killer cell”, “NK cells” or “large granular lymphocytes (LGL)” are a type of cytotoxic lymphocyte critical to the innate immune system that contains small granules in their cytoplasm comprising proteins such as perforin and proteases known as granzymes. As used herein, this term includes natural killer cells, adaptive natural killer cells, memory natural killer cells and memory-like natural killer cells. “Activated Natural killer cell” are natural killer cells activated by a prevalence of activating receptor stimulation over inhibitory receptor stimulation. Markers of activated natural killer cells include but are not limited to NKG2A and TRAF3IP1.

The antigen binding domain of the invention particularly targets a marker of activation of an immune cell, in particular such as described here above.

The target expressed on activated immune cells can particularly be selected among the target described in Table D below.

TABLE D
Examples of targets of interest.

| | | UniProt |
|---|---|---|
| Name | Official name | reference |
| BCMA | B-cell maturation protein, Tumor necrosis factor receptor | Q02223 |
| | superfamily member 17 (CD antigen CD269) | |
| BTLA | B- and T-lymphocyte attenuator (B- and T-lymphocyte- | Q7Z6A9 |
| | associated protein) (CD antigen CD272) | |
| TACI | TNFRSF13B, Tumor necrosis factor receptor | O14836 |
| | superfamily member 13B, Transmembrane activator and | |
| | CAML interactor, CD267 | |
| CD101 | Immunoglobulin superfamily member 2, IgSF2 (Cell | Q93033 |
| | surface glycoprotein V7) (Glu-Trp-Ile EWI motif- | |
| | containing protein 101, EWI-101) (CD antigen CD101) | |
| CD103 | Integrin alpha-E, HML-1 antigen, Integrin alpha-IEL, | P38570 |
| | Mucosal lymphocyte 1 antigen | |
| CD119 | INGR1, IFNGR1, Interferon gamma receptor 1, IFN- | P15260 |
| | gamma receptor 1, IFN-gamma-R1, CDw119, Interferon | |
| | gamma receptor alpha-chain, IFN-gamma-R-alpha, | |
| | CD119 | |
| CD137 | Tumor necrosis factor receptor superfamily member 9 | Q07011 |
| | (4-1BB ligand receptor, CD137) | |
| CD150 | Signaling lymphocytic activation molecule, CDw150, | Q13291 |
| | IPO-3, SLAM family member 1, CD150 | |
| CD153 | Tumor necrosis factor ligand superfamily member | P32971 |
| | 8, CD30 ligand, CD30-L, CD153 | |
| CD154 | CD40 ligand, CD40-L, T-cell antigen Gp39, TNF-related | P29965 |
| | activation protein, TRAP, Tumor necrosis factor ligand | |
| | superfamily member 5, CD154 | |
| CD223 | Lymphocyte activation gene 3 protein, LAG-3, CD223 | P18627 |
| CD226 | CD226 antigen, DNAX accessory molecule 1 (DNAM-1) | Q15762 |
| CD25 | IL-2 receptor subunit alpha; IL-2-RA; IL-2R subunit | P01589 |
| | alpha; IL2-RA | |
| CD254 | Tumor necrosis factor ligand superfamily member | O14788 |
| | 11, Osteoclast differentiation factor, ODF, | |
| | Osteoprotegerin ligand, OPGL, Receptor activator of | |
| | nuclear factor kappa-B ligand, RANKL, TNF-related | |
| | activation-induced cytokine, TRANCE, CD254 | |
| CD26 | Dipeptidyl peptidase 4, 3.4.14.5, ADABP, Adenosine | P27487 |
| | deaminase complexing protein 2, ADCP-2, Dipeptidyl | |
| | peptidase IV, DPP IV, T-cell activation antigen CD26, | |
| | TP103, CD26 | |
| CD27 | CD27 antigen, CD27L receptor, T-cell activation antigen | P26842 |
| | CD27, T14, Tumor necrosis factor receptor superfamily | |
| | member 7, CD27 | |
| CD275 | ICOS ligand, B7 homolog 2, B7-H2, B7-like protein | O75144 |
| | G150, B7-related protein 1, B7RP-1, CD275 | |
| CD39 | Ectonucleoside triphosphate diphosphohydrolase-1 | P49961 |
| | (NTPDase 1, Ecto-apyrase, ATPDase 1, or Lymphoid | |
| | cell activation antigen) | |
| CD40L | CD40 ligand (T-cell antigen Gp39, TNF-related | P29965 |
| | activation protein, Tumor necrosis factor ligand | |
| | superfamily member 5, CD154) | |
| CD44 | CD44 antigen (Epican, Extracellular matrix receptor III, | P16070 |
| | GP90 lymphocyte homing/adhesion receptor, HUTCH-I, | |
| | Heparan sulfate proteoglycan, Hermes antigen, | |
| | Hyaluronate receptor, Phagocytic glycoprotein 1, | |
| | Phagocytic glycoprotein I) | |
| CD80 | T-lymphocyte activation antigen CD80, Activation B7-1 | P33681 |
| | antigen, BB1, CTLA-4 counter-receptor B7.1, B7, CD80 | |
| CD83 | CD83 antigen, hCD83, B-cell activation protein, Cell | Q01151 |
| | surface protein HB15, CD83 | |
| CD86 | T-lymphocyte activation antigen CD86, Activation B7-2 | P42081 |
| | antigen, B70, BU63, CTLA-4 counter-receptor B7.2, | |
| | FUN-1, CD86 | |
| CD95 | Tumor necrosis factor receptor superfamily member | P25445 |
| | 6, Apo-1 antigen, Apoptosis-mediating surface antigen | |
| | FAS, FASLG receptor, CD95 | |
| CMKLR1 | Chemerin-like receptor 1, Chemokine-like receptor 1, G- | Q99788 |
| | protein coupled receptor ChemR23, G-protein coupled | |
| | receptor DEZ | |
| CRTAM | Cytotoxic and regulatory T-cell molecule, Class-I MHC- | O95727 |
| | restricted T-cell-associated molecule, CD355 | |
| CST7 | Cystatin-F, Cystatin-7, Cystatin-like metastasis- | O76096 |
| | associated protein, CMAP, Leukocystatin | |
| CTLA-4 | Cytotoxic T-lymphocyte protein 4 (Cytotoxic T- | P16410 |
| | lymphocyte-associated antigen 4, CTLA-4) (CD antigen | |
| | CD152) | |
| CXCR3 | C-X-C chemokine receptor type 3 or CXC-R3 or CXCR- | P49682 |
| | 3 or CKR-L2 or G protein-coupled receptor 9 or | |
| | Interferon-inducible protein 10 receptor (IP-10 receptor) | |
| | or CD183 | |
| CXCR4 | C-X-C chemokine receptor type 4, CXC-R4, CXCR-4, | P61073 |
| | FB22, Fusin, HM89, LCR1, Leukocyte-derived seven | |
| | transmembrane domain receptor, LESTR, | |
| | Lipopolysaccharide-associated protein 3, LAP-3, LPS- | |
| | associated protein 3, NPYRL, Stromal cell-derived factor | |
| | 1 receptor, SDF-1 receptor, CD184 | |
| CXCR5 | C-X-C chemokine receptor type 5 or Burkitt lymphoma | P32302 |
| | receptor 1 or Monocyte-derived receptor 15 (MDR-15) | |
| | or CD185 | |
| CXCR6 | C-X-C chemokine receptor type 6, CXC-R6, CXCR-6, | O00574 |
| | CDw186, G-protein coupled receptor STRL33, G-protein | |
| | coupled receptor bonzo, CD186 | |
| FasL | Tumor necrosis factor ligand superfamily member | P48023 |
| | 6, Apoptosis antigen ligand, APTL, CD95 ligand, CD95- | |
| | L, Fas antigen ligand, Fas ligand, FasL, CD178 | |
| GITR | Tumor necrosis factor receptor superfamily member 18 | Q9Y5U5 |
| | (Activation-inducible TNFR family receptor, | |
| | Glucocorticoid-induced TNFR-related protein, CD357) | |
| GPR32 | G-protein coupled receptor 32 | O75388 |
| TIM-3 | Hepatitis A virus cellular receptor 2, HAVcr-2 (T-cell | Q8TDQ0 |
| | immunoglobulin and mucin domain-containing protein 3, | |
| | TIMD-3) (T-cell immunoglobulin mucin receptor 3, | |
| | TIM-3) (T-cell membrane protein 3) | |
| ICOS | Inducible T-cell costimulator (Activation-inducible | Q9Y6W8 |
| | lymphocyte immunomediatory molecule, CD278) | |
| IL18R1 | Interleukin-18 receptor 1, IL-18R-1, IL-18R1, 3.2.2.6, | Q13478 |
| | CD218 antigen-like family member A, CDw218a, IL1 | |
| | receptor-related protein, IL-1Rrp, ILIR-rp, Interleukin- | |
| | 18 receptor alpha, IL-18R-alpha, IL-18Ralpha, CD218a | |
| ITGAE | Integrin alpha-E, HML-1 antigen, Integrin alpha-IEL, | P38570 |
| | Mucosal lymphocyte 1 antigen, CD103 | |
| LAG-3 | Lymphocyte activation gene 3 protein, LAG-3, CD223 | P18627 |
| TRAILR1 | Tumor necrosis factor receptor superfamily member | O00220 |
| | 10A, Death receptor 4, TNF-related apoptosis-inducing | |
| | ligand receptor 1, TRAIL receptor 1, TRAIL-R1, CD261 | |
| TRAILR2 | Tumor necrosis factor receptor superfamily member | O14763 |
| | 10B, Death receptor 5, TNF-related apoptosis-inducing | |
| | ligand receptor 2, TRAIL receptor 2, TRAIL-R2, CD262 | |
| OX40L | Tumor necrosis factor ligand superfamily member 4, | P23510 |
| | Glycoprotein Gp34, OX40 ligand, OX40L, TAX | |
| | transcriptionally-activated glycoprotein 1, CD252 | |
| SlamF6 | SLAM family member 6, Activating NK receptor, NK- | Q96DU3 |
| | T-B-antigen, NTB-A, CD352 | |
| NKG2D | NKG2-D type II integral membrane protein, Killer cell | P26718 |
| | lectin-like receptor subfamily K member 1, NK cell | |
| | receptor D, NKG2-D-activating NK receptor, CD314 | |
| OX40 | Tumor necrosis factor receptor superfamily member 4 | P43489 |
| | (ACT35 antigen, AX transcriptionally-activated | |
| | glycoprotein 1 receptor) | |
| PD-1 | Programmed cell death protein 1 (CD279) | Q15116 |
| PTPN22 | Tyrosine-protein phosphatase non-receptor type | Q9Y2R2 |
| | 22, 3.1.3.48, Hematopoietic cell protein-tyrosine | |
| | phosphatase 70Z-PEP, Lymphoid phosphatase, LyP, | |
| | PEST-domain phosphatase, PEP | |
| RGS1 | Regulator of G-protein signaling 1, RGS1, B-cell | Q08116 |
| | activation protein BL34, Early response protein 1R20 | |
| LOX1 | Oxidized low-density lipoprotein receptor 1, Ox-LDL | P78380 |
| | receptor 1, C-type lectin domain family 8 member A, | |
| | Lectin-like oxidized LDL receptor 1, LOX-1, Lectin-like | |
| | oxLDL receptor 1, hLOX-1, Lectin-type oxidized LDL | |
| | receptor 1 | |
| SIGLEC 6 | Sialic acid-binding Ig-like lectin 6, Siglec-6, CD33 | O43699 |
| | antigen-like 1, CDw327, Obesity-binding protein 1, OB- | |
| | BP1, CD327 | |
| TACI/ | Tumor necrosis factor receptor superfamily member | O14836 |
| TNFRSF13B | 13B, Transmembrane activator and CAML interactor, | |
| | CD267 | |
| TIGIT | T-cell immunoreceptor with Ig and ITIM domains (V-set | Q495A1 |
| | and immunoglobulin domain-containing protein 9) (V- | |
| | set and transmembrane domain-containing protein 3) | |
| CD163 | Scavenger receptor cysteine-rich type 1 protein M130, | Q86VB7 |
| | Hemoglobin scavenger receptor, CD163 | |
| CD206 | Macrophage mannose receptor 1, MMR, C-type lectin | P22897 |
| | domain family 13 member D, C-type lectin domain | |
| | family 13 member D-like, Human mannose receptor, | |
| | hMR, Macrophage mannose receptor 1-like protein 1, | |
| | CD206 | |
| LTBR/CD70 | CD70 antigen, CD27 ligand, CD27-L, Tumor necrosis | P32970 |
| | factor ligand superfamily member 7, CD70 | |
| TNFSF14 | Tumor necrosis factor receptor superfamily member | Q92956 |
| | 14, Herpes virus entry mediator A, Herpesvirus entry | |
| | mediator A, HveA, Tumor necrosis factor receptor-like | |
| | 2, TR2, CD270 | |
| SLAMF1 | Signaling lymphocytic activation molecule, CDw150, | Q13291 |
| | IPO-3, SLAM family member 1, CD150 | |
| SLAMF7 | SLAM family member 7, CD2 subset 1, CD2-like | Q9NQ25 |
| | receptor-activating cytotoxic cells, CRACC, Membrane | |
| | protein FOAP-12, Novel Ly9, Protein 19A, CD319 | |
| NKG2A | NKG2-A/NKG2-B type II integral membrane protein, | P26715 |
| | CD159 antigen-like family member A, NK cell receptor | |
| | A, NKG2-A/B-activating NK receptor, CD159a | |
| KIR2DL2 | Killer cell immunoglobulin-like receptor 2DL2, CD158 | P10321 |
| | antigen-like family member B1, Natural killer-associated | |
| | transcript 6, NKAT-6, p58 natural killer cell receptor | |
| | clone CL-43, p58 NK receptor CL-43, CD158b1 | |
| CD96 | T-cell surface protein tactile, Cell surface antigen CD96, | P40200 |
| | T cell-activated increased late expression protein, CD96 | |
| CD112R | Transmembrane protein PVRIG, CD112 receptor, | Q6DKI7 |
| | CD112R, Poliovirus receptor-related immunoglobulin | |
| | domain-containing protein | |
| CD28H | Transmembrane and immunoglobulin domain-containing | Q96BF3 |
| | protein 2, CD28 homolog, Immunoglobulin and proline- | |
| | rich receptor 1, IGPR-1 | |
| IL2RB | Interleukin-2 receptor subunit beta, IL-2 receptor subunit | P14784 |
| | beta, IL-2R subunit beta, IL-2RB, High affinity IL-2 | |
| | receptor subunit beta, Interleukin-15 receptor subunit | |
| | beta, p70-75, p75, CD122 | |
| TRAIL | Tumor necrosis factor ligand superfamily member 10, | P50591 |
| | Apo-2 ligand, Apo-2L, TNF-related apoptosis-inducing | |
| | ligand, Protein TRAIL, CD253 | |
| CD48 | CD48 antigen, B-lymphocyte activation marker BLAST- | P09326 |
| | 1, BCM1 surface antigen, Leukocyte antigen MEM-102, | |
| | SLAM family member 2, SLAMF2, Signaling | |
| | lymphocytic activation molecule 2, TCT.1, CD48 | |
| CD53 | Leukocyte surface antigen CD53, Cell surface | P19397 |
| | glycoprotein CD53, Tetraspanin-25, Tspan-25, CD53 | |
| CD164 | Sialomucin core protein 24, MUC-24, Endolyn, Multi- | Q04900 |
| | glycosylated core protein 24, MGC-24, MGC-24v, | |
| | CD164 | |
| CD138 | Syndecan-1, SYND1, CD138 | P18827 |
| CD38 | ADP-ribosyl cyclase/cyclic ADP-ribose hydrolase 1, | P28907 |
| | 3.2.2.6, 2′-phospho-ADP-ribosyl cyclase, 2′-phospho- | |
| | ADP-ribosyl cyclase/2′-phospho-cyclic-ADP-ribose | |
| | transferase, 2.4.99.20, 2′-phospho-cyclic-ADP-ribose | |
| | transferase, ADP-ribosyl cyclase 1, ADPRC 1, Cyclic | |
| | ADP-ribose hydrolase 1, cADPr hydrolase 1, T10, CD38 | |
| CD39 | Ectonucleoside triphosphate diphosphohydrolase 1, | P49961 |
| | NTPDase 1, 3.6.1.5, Ecto-ATP diphosphohydrolase 1, | |
| | Ecto-ATPDase 1, Ecto-ATPase 1, Ecto-apyrase, | |
| | Lymphoid cell activation antigen, CD39 | |
| FCRL4 | Fc receptor-like protein 4, FcR-like protein 4, FcRL4, Fc | Q96PJ5 |
| | receptor homolog 4, FcRH4, IFGP family protein 2, | |
| | hIFGP2, Immune receptor translocation-associated | |
| | protein 1, CD307d | |
| CD30/TNFRSF8 | Tumor necrosis factor receptor superfamily member 8, | P28908 |
| | CD30L receptor, Ki-1 antigen, Lymphocyte activation | |
| | antigen CD30, CD30 | |
| TRAF1 | TNF receptor-associated factor 1, Epstein-Barr virus- | Q13077 |
| | induced protein 6 | |
| TRAF2 | TNF receptor-associated factor 2, 2.3.2.27, E3 ubiquitin- | Q12933 |
| | protein ligase TRAF2, RING-type E3 ubiquitin | |
| | transferase TRAF2, Tumor necrosis factor type 2 | |
| | receptor-associated protein 3 | |
| TRAF3/CD40BP | TNF receptor-associated factor 3, 2.3.2.27, CD40 | Q13114 |
| | receptor-associated factor 1, CRAF1, CD40-binding | |
| | protein, CD40BP, LMP1-associated protein 1, LAP1, | |
| | RING-type E3 ubiquitin transferase TRAF3 | |
| TRAF3IP1 | TRAF3-interacting protein 1, Interleukin-13 receptor | Q8TDR0 |
| | alpha 1-binding protein 1, Intraflagellar transport protein | |
| | 54 homolog, Microtubule-interacting protein associated | |
| | with TRAF3, MIP-T3 | |
| TRAF4 | TNF receptor-associated factor 4, 2.3.2.27, Cysteine-rich | Q9BUZ4 |
| | domain associated with RING and Traf domains protein | |
| | 1, Metastatic lymph node gene 62 protein, MLN 62, | |
| | RING finger protein 83 | |
| TRAF7 | E3 ubiquitin-protein ligase TRAF7, 2.3.2.27, RING | Q6Q0C0 |
| | finger and WD repeat-containing protein 1, RING finger | |
| | protein 119, RING-type E3 ubiquitin transferase TRAF7, | |
| | TNF receptor-associated factor 7 | |
| TRAP1 | Heat shock protein 75 kDa, mitochondrial, HSP 75, | Q12931 |
| | TNFR-associated protein 1, Tumor necrosis factor type 1 | |
| | receptor-associated protein, TRAP-1 | |
| TNFR1/ | Tumor necrosis factor receptor superfamily member 1A, | P19438 |
| TNFRSF1A/ | Tumor necrosis factor receptor 1, TNF-R1, Tumor | |
| CD120A | necrosis factor receptor type I, TNF-RI, TNFR-I, p55, | |
| | p60, CD120a | |
| TRAP100/ | Mediator of RNA polymerase II transcription subunit 24, | O75448 |
| MED24 | Activator-recruited cofactor 100 kDa component, | |
| | ARC100, Cofactor required for Sp1 transcriptional | |
| | activation subunit 4, CRSP complex subunit 4, Mediator | |
| | complex subunit 24, Thyroid hormone receptor- | |
| | associated protein 4, Thyroid hormone receptor- | |
| | associated protein complex 100 kDa component, | |
| | Trap100, hTRAP100, Vitamin D3 receptor-interacting | |
| | protein complex 100 kDa component, DRIP100 | |
| TNFR2/ | Tumor necrosis factor receptor superfamily member 1B, | P20333 |
| TNFRSF1811/ | Tumor necrosis factor receptor 2, TNF-R2, Tumor | |
| CD120B | necrosis factor receptor type II, TNF-RII, TNFR-II, p75, | |
| | p80 TNF-alpha receptor, CD120b, Etanercept | |
| CD45RO | Isotype of “receptor-type tyrosine-protein phosphatase C, | P08575 |
| | 3.1.3.48, Leukocyte common antigen, L-CA, T200, | (CD45) |
| | CD45” which lacks all three of the A, B, and C regions | |
| | of CD45 | |
| CD45RC | Isotype of “receptor-type tyrosine-protein phosphatase C, | P08575 |
| | 3.1.3.48, Leukocyte common antigen, L-CA, T200, | (CD45) |
| | CD45” which lacks C regions of CD45 | |
| CD69 | Early activation antigen CD69, Activation inducer | Q07108 |
| | molecule, AIM, BL-AC/P26, C-type lectin domain | |
| | family 2 member C, EA1, Early T-cell activation antigen | |
| | p60, GP32/28, Leukocyte surface antigen Leu-23, MLR- | |
| | 3, CD69 | |
| GPR18 | N-arachidonyl glycine receptor, NAGly receptor, G- | Q14330 |
| | protein coupled receptor 18 | |
| GPR35 | G-protein coupled receptor 35, Kynurenic acid receptor, | Q9HC97 |
| | KYNA receptor | |
| FPR2 | N-formyl peptide receptor 2, FMLP-related receptor I, | P25090 |
| | FMLP-R-I, Formyl peptide receptor-like 1, HM63, | |
| | Lipoxin A4 receptor, LXA4 receptor, RFP | |
| CDCR3/ | Tumor necrosis factor receptor superfamily member 6B, | O95407 |
| TNFRSF6B | Decoy receptor 3, DcR3, Decoy receptor for Fas ligand, | |
| | M68 | |
| TNFRSF12A/ | Tumor necrosis factor receptor superfamily member | Q9NP84 |
| FN14/TWEAKR | 12A, Fibroblast growth factor-inducible immediate-early | |
| | response protein 14, FGF-inducible 14, Tweak-receptor, | |
| | TweakR, CD266 | |
| BAFFR/ | Tumor necrosis factor receptor superfamily member | Q96RJ3 |
| TNFRSF13C/ | 13C, B-cell-activating factor receptor, BAFF receptor, | |
| CD268 | BAFF-R, BLyS receptor 3, CD268 | |
| HVEM/ | Tumor necrosis factor receptor superfamily member 14, | Q92956 |
| TNFRSF14/ | Herpes virus entry mediator A, Herpesvirus entry | |
| CD270 | mediator A, HveA, Tumor necrosis factor receptor-like | |
| | 2, TR2, CD270 | |
| GITR/ | Tumor necrosis factor receptor superfamily member 8, | P28908 |
| TNFRSF8/ | CD30L receptor, Ki-1 antigen, Lymphocyte activation | |
| CD357 | antigen CD30, CD30 | |
| RELT/ | Tumor necrosis factor receptor superfamily member 19L, | Q969Z4 |
| TNFRSF19L | Receptor expressed in lymphoid tissues | |
| TNFRSF19/ | Tumor necrosis factor receptor superfamily member 19, | Q9NS68 |
| TROY | TRADE, Toxicity and JNK inducer | |
| TNFRSF21/ | Tumor necrosis factor receptor superfamily member 21, | O75509 |
| DR6 | Death receptor 6, CD358 | |
| TNFRSF25/ | Tumor necrosis factor receptor superfamily member 25, | Q93038 |
| DR3/ | Apo-3, Apoptosis-inducing receptor AIR, Apoptosis- | |
| TNFRSF12 | mediating receptor DR3, Apoptosis-mediating receptor | |
| | TRAMP, Death receptor 3, Lymphocyte-associated | |
| | receptor of death, LARD, Protein WSL, Protein WSL-1 | |
| CD301 | C-type lectin domain family 10 member A, C-type lectin | Q8IUN9 |
| | superfamily member 14, Macrophage lectin 2, CD301 | |
| IL4R | Interleukin-4 receptor subunit alpha, IL-4 receptor | P24394 |
| | subunit alpha, IL-4R subunit alpha, IL-4R-alpha, IL- | |
| | 4RA, CD124 | |
| CLEC-1A | C-type lectin domain family 1 member A, C-type lectin- | Q8NC01 |
| | like receptor 1, CLEC-1 | |
| CD21 | Complement receptor type 2, Cr2, Complement C3d | P20023 |
| | receptor, Epstein-Barr virus receptor, EBV receptor, | |
| | CD21 | |
| CLEC-9A | C-type lectin domain family 9 member A, CD370 | Q6UXN8 |
| CD180 | CD180 antigen, Lymphocyte antigen 64, Radioprotective | Q99467 |
| | 105 kDa protein, CD180 | |
| CD59 | CD59 glycoprotein, 1F5 antigen, 20 kDa homologous | P13987 |
| | restriction factor, HRF-20, HRF20, MAC-inhibitory | |
| | protein, MAC-IP, MEM43 antigen, Membrane attack | |
| | complex inhibition factor, MACIF, Membrane inhibitor | |
| | of reactive lysis, MIRL, Protectin, CD59 | |
| CD54 | Intercellular adhesion molecule 1, ICAM-1, Major group | P05362 |
| | rhinovirus receptor, CD54 | |
| CD71 | Transferrin receptor protein 1, TR, TfR, TfR1, Trfr, T9, | P02786 |
| | p90, CD71 | |
| CD35 | Complement receptor type 1, C3b/C4b receptor, CD35 | P17927 |
| CD218a | Interleukin-18 receptor 1, IL-18R-1, IL-18R1, 3.2.2.6, | Q13478 |
| | CD218 antigen-like family member A, CDw218a, IL1 | |
| | receptor-related protein, IL-1Rrp, ILIR-rp, Interleukin- | |
| | 18 receptor alpha, IL-18R-alpha, IL-18Ralpha, CD218a | |
| CD74 | HLA class II histocompatibility antigen gamma chain, | P04233 |
| | HLA-DR antigens-associated invariant chain, Ia antigen- | |
| | associated invariant chain, Ii, CD74 | |
| 4-1BBL/ | Tumor necrosis factor ligand superfamily member 9, 4- | P41273 |
| CD137L | 1BB ligand, 4-1BBL | |
| ICOSL | ICOS ligand, B7 homolog 2, B7-H2, B7-like protein | O75144 |
| | G150, B7-related protein 1, B7RP-1, CD275 | |
| CD160 | CD160 antigen, Natural killer cell receptor BY55, | O95971 |
| | CD160 | |
| SIRPa | Signal-regulatory protein alpha, Tyrosine-protein | P78324 |
| | phosphatase non-receptor type substrate 1 | |
| CD127 | Interleukin-7 receptor subunit alpha, IL7R | P16871 |

Optionally, the target expressed on activated immune cells surface is in addition specifically expressed on activated immune cells surface.

By “specifically expressed” on activated immune cells surface is meant that the target of the antigen binding domain is expressed at the outer surface of immune cells, but is not substantially expressed by other cell types, for example such as tumour cells. It particularly means that the expression of the target is higher in immune cells than in other cells (i.e., non-immune cells).

The target specifically expressed on activated immune cells surface is selected from the group consisting of BCMA/TNFRSF17, BTLA, CD101/IGSF2, CD103, CD119, CD137/4-1BB/TNFRSF9, CD150, CD153, CD154, CD223, CD226, CD25, CD254, CD26, CD27, CD275, CD30, CD39/ENTPD1, CD40L, CD44, CD45RO, CD45RC, LGR6, CD69, GPR18, GPR35, FPR2, CD80, CD83, CD86, CD95, CMKLR1, CRTAM, CST7, CTLA4, CXCR3, CXCR4, CXCR5, CXCR6, FasL/TNFSF6, GITR/TNFRSF18, GPR32, TIM3/HAVCR2, ICOS, IL18R1/CXCR1/CD218a, ITGAE, LAG3, TRAILR, OX40L, LY108/SlamF6, NKG2D, OX40/TNFRSF4, PDCD1, PTPN22, RGS1, LOX1, SIGLEC 6, TACI/TNFRSF13B, TIGIT, CD163, CD206, LTBR/CD70, TNFSF14, SLAMF1, SLAMF7, NKG2A, KIR2DL2, CD96, CD112R, CD28H, IL2RB, TRAIL, CD48, CD53, CD164, CD138 (SDC1), CD38, CD39, FCRL4, CD30/TNFRSF8, CD78, TRAF1, TRAF2, TRAF3/CD40BP, TRAF3IP1, TRAF4, TRAF7, TRAP1, TNFR1/TNFRSF1A/CD120A, TRAP100/MED24, TNFR2/TNFRSF1811/CD120B, CDCR3/TNFRSF6B, TNFRSF12A/FN14/TWEAKR, BAFFR/TNFRSF13C/CD268, HVEM/TNFRSF14/CD270, GITR/TNFRSF8/CD357, RELT/TNFRSF19L, TNFRSF19/TROY, TNFRSF21/DR6, TNFRSF25/DR3/TNFRSF12, CD301, IL4R, CLEC-1A, CD21, CLEC-9A, CD180, CD59, CD54, CD71, CD35, CD218a, CD74, CD165, 4-1BBL/CD137L, ICOSL and CD160.

For instance, the target expressed on activated B cells surface is selected from the group consisting of BCMA/TNFRSF17, CD150, CD86, OX40L, LOX1, TACI/TNFRSF13B, CD138 (SDC1), FCRL4, CD78, FRAF3/CD40BP, TRAP1, BAFFR/TNFRSF13C/CD268, CD21, CLEC-9A, CD180, CD59, CD54, CD71, CD35, CD218a, CD74, CD165.

For instance, the target expressed on activated myeloid cells surface is selected from the group consisting of CD163, CD206, SIGLEC 6, TRAF1, TRAF4, TRAF7, TRAP100/MED24, TNFRSF12A/FN14/TWEAKR, CD301, TL4R, CLEC-1A.

For instance, the target expressed on activated natural killer cells surface is selected from the group consisting of CST7, CXCR4, NKG2A, TRAF3IP1, CMKLR1.

Preferably, the target specifically expressed on activated immune cells surface is not an antigen of the TCR pathway (interaction between antigen presenting cells and T cells).

For instance, the target expressed on activated T cells surface is selected from the group consisting of CD101/IGSF2, CD103, CD119, CD137/4-1BB/TNFRSF9, CD154, CD183, CD25, CD254, CD26, CD275, CD40L, CD44, CD45RO, CD45RC, LGR6, CD69, GPR18, CD80, CD95, CTLA4, CXCR3, CXCR6, FasL/TNFSF6, GITR/TNFRSF18, GPR32, ICOS, TL18R1/CXCR1/CD218a, ITGAE, LY108/SlamF6, OX40/TNFRSF4, RGS1, LTBR/CD70, TNFSF14, CD112R, CD28H, CD164, TRAF2, CDCR3/TNFRSF6B, GITR/TNFRSF8/CD357, RELT/TNFRSF19L, TNFRSF19/TROY, TNFRSF21/DR6, TNFRSF25/DR3/TNFRSF12, ICOSL, CD160.

Preferably, the target expressed on activated immune cells surface is selected from the group consisting of PD-1, CD127, SIRPa and CLEC-1A.

In a preferred aspect, the target is PD-1.

In a particular aspect, the immune cell is a T cell so that the lipid-based nanoparticle according to the invention comprises an antigen-binding domain capable of specifically binding to a target expressed on activated T cells surface. Preferably, the target expressed on activated T cells surface is selected from the group consisting of CD137/41BB/TNFRSF9, PD-1, CRTAM, CD39, CXCR5, CD70, CTLA-4, TIM-3/HAVCR2, ITGAE, LAG-3, OX40/TNFRSF4,and TIGIT.

Preferably, the target specifically expressed on activated immune cells surface is not a pan-T-cell marker, i.e., a marker that is expressed on multiple sub-types of T cells. In one aspect, the pan-T antigen is CD2, CD3, CD5 or CD7. Then, when the lipid-based nanoparticle is aimed at targeting immune cells such as T cells, the target of the antigen binding domain is not CD2, CD3, CD5 and/or CD7.

In a particular aspect, the activated T cell is an effector memory stem like T cell and the target is a factor expressed on the surface of effector memory stem like T cells, preferably specifically expressed on the surface of effector memory stem like T cells. Preferably, the target expressed on the surface of effector memory stem like T cells is selected from the group consisting of CXCR5, SLAMF7 and CRTAM.

In a particular aspect, the activated T cell is a Tumour Infiltrating lymphocytes (TILs) and the target is a factor expressed on the surface of Tumour Infiltrating lymphocytes, preferably specifically expressed on the surface of TILs. Preferably, the target expressed on the surface of TILs is selected from the group consisting of CD101, CD137 (Tnfrsf9/4-1BBL), CRTAM, CST7, CTLA4, CXCR3, FAS, IL18R1/CXCR1/CD218A, LAG-3 PTPN22, RGS1, TNFSF14 and PD1.

In a particular aspect, the activated T cell is a cytotoxic T cell and the target is a factor expressed on the surface of cytotoxic T cell, preferably specifically expressed on the surface of cytotoxic T cells. Preferably, the target expressed on the surface of cytotoxic T cells is selected from the group consisting of CD25, CD38, CD69, PD1, TIM-3, LAG-3 and TIGIT.

In an aspect, the antigen binding domain has an antagonist activity on the target when the target is a receptor having an inhibitory effect on the cell.

The term “antagonist” as used herein, refers to a substance that blocks or reduces the activity or functionality of another substance. Particularly, this term refers to a binding domain that binds to a cellular receptor (e.g., PD-1) as a reference substance (e.g., PD-L1 and/or PD-L2), preventing it from producing all or part of its usual biological effects (e.g., the creation of an immune suppressive microenvironment). The antagonist activity may be assessed by competitive ELISA.

In an alternative aspect, the antigen binding domain has an agonist activity on the target when the target is a receptor having an inhibitor effect on the cell.

The term “agonist” as used herein, refers to a substance that activates or increases the activity or functionality of another substance. Particularly, this term refers to a binding domain that binds to a cellular receptor as a reference substance, causing it to produce all or part of its usual biological effects (e.g., the creation of an immune suppressive microenvironment).

In an alternative aspect, the antigen binding domain has no activity on the target.

In an aspect, the antigen binding domain does not interfere with the binding between its target and its natural ligand. In other words, the antigen binding domain according to the invention is not an agonist and/or not an antagonist of the interaction between its target and its natural ligand. The absence of such an agonist and/or antagonist capability may be assessed with methods usually known by the person skilled in the art.

In a preferred aspect, the antigen binding domain of the lipid-based nanoparticle does not compete with the natural ligand for the binding to the target specifically expressed on activated immune cells. The absence of competition between the antigen binding domain of the invention and the natural ligand of the target specifically expressed on the activated immune cells may be determined when, in presence of the antigen binding domain of the invention, the binding of the natural ligand to the target specifically expressed on the activated immune cells is at least 50%, more preferably at least 80%, still more preferably at least 90% and most preferably similar, to the binding of the natural ligand to the target specifically expressed on the activated immune cells, under the same experimental conditions but without the presence of the antigen binding domain of the invention.

In an aspect, the antigen binding domain is an antibody, a fragment thereof or a derived thereof. Preferably, the antigen binding domain is preferably derived from a format selected from the group consisting of IgA, IgM, IgE, IgD and IgG, or a variant thereof.

The terms “derive from” and “derived from” as used herein refers to a compound having a structure derived from the structure of a parent compound or protein and whose structure is sufficiently similar to those disclosed herein and based upon that similarity, would be expected by one skilled in the art to exhibit the same or similar properties, activities and utilities as the claimed compounds.

The antigen binding domain is preferably a monoclonal antibody, preferably a human, humanized, chimeric or recombinant antibody or antigen binding fragment thereof.

In some aspects, the antigen binding domain is a monoclonal antibody or an antigen binding fragment thereof.

In some aspects, the antigen binding domain is a monoclonal antibody or an antigen binding fragment thereof. The term “monoclonal antibody” as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity. In an aspect, the antibodies of the disclosure are monoclonal antibodies.

In some aspects, the antigen binding domain is a recombinant antibody or an antigen binding fragment thereof. As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies.

In some aspects, the antigen binding domain is a chimeric antibody or an antigen binding fragment thereof. As used herein, a “chimeric antibody” refers to an antibody in which the sequence of the variable domain derived from the germline of a mammalian species, such as a mouse, have been grafted onto the sequence of the constant domain derived from the germline of another mammalian species, such as a human. Chimeric antibodies generally comprise constant domains from human and variable domains from another mammalian species, reducing the risk of a reaction to foreign antibodies from a non-human animal when they are used in therapeutic treatments.

In some aspects, the antigen binding domain is a humanized antibody or an antigen binding fragment thereof. As used herein “humanized antibody” refers to an antibody in which CDR sequences derived from the germline of another mammalian species, such as mouse, have been grafted onto human framework sequences”. A “humanized form” of an antibody, e.g., a non-human antibody, also refers to an antibody that has undergone humanization. A humanized antibody is generally a human immunoglobulin (recipient antibody) in which residues from one or more CDRs are replaced by residues from at least one CDR of a non-human antibody (donor antibody) while maintaining the desired specificity, affinity, and capacity of the original antibody. Additional framework region modifications may be made within the human framework sequences. Preferably humanized antibody has a T20 humanness score greater than 80%, 85% or 90%. “Humanness” of an antibody can for example be measured using the T20 score analyzer to quantify the humanness of the variable region of antibodies as described in Gao S H, Huang K, Tu H, Adler A S. BMC Biotechnology. 2013: 13:55 or via a web-based tool to calculate the T20 score of antibody sequences using the T20 Cutoff Human Databases: http://abAnalyzer.lakepharma.com.

In another aspect, the antigen binding domain according to the present disclosure is a modified antibody or an antigen binding fragment thereof. As used herein, a “modified antibody” corresponds to a molecule comprising an antibody or an antigen-binding fragment thereof, wherein said antibody or functional fragment thereof is associated with a functionally different molecule. A modified antibody of the invention may be either a fusion chimeric protein or a conjugate resulting from any suitable form of attachment including covalent attachment, grafting, chemical bonding or coupling with a chemical or biological group or with a molecule, such as a PEG polymer or another protective group or molecule suitable for protection against proteases cleavage in vivo, for improvement of stability and/or half-life of the antibody or functional fragment. PEGylation of the antibody or functional fragments thereof is a particular interesting embodiment as it improves the delivery conditions of the active substance to the host, especially for a therapeutic application. PEGylation can be site specific to prevent interference with the recognition sites of the antibodies or functional fragments and can be performed with high molecular weight PEG. PEGylation can be achieved through free cysteine residues present in the sequence of the antibody or functional fragment or through added free Cysteine residues in the amino sequence of the antibody or functional fragment. In some aspects, the antigen binding domain comprises a hydrophobic domain. The hydrophobic domain may comprise or consist of one or more alpha-helical regions. The hydrophobic region is particularly configured to interact with the hydrophobic lipid of the lipid-based nanoparticle of the invention.

In some aspects, the antigen binding domain comprises or is modified to comprise a group such as a thiol group, capable of reacting with a group carried by a lipid of the nanoparticle, such as a PEG-maleimide, so as to conjugate the antigen binding domain to a lipid of the lipid-based nanoparticle. The suitable reactive groups and the conjugation chemistry is well-known be the person skilled in the art. For instance, the conjugation can be carried out by click chemistry. Alternatively the conjugation can be carried out by an enzyme.

In an aspect, the antigen binding domain is conjugated to a lipid of the lipid-based nanoparticle, for instance through a maleimide moiety which is conjugated, attached or linked to a PEG-derivative. Preferably, the antigen binding domain may comprise a thiol group capable of reacting with the PEG-maleimide to conjugate the antigen binding domain to the lipid-based nanoparticle. Other methods known to the person skilled in the art may be used to conjugate the antigen binding domain to a lipid of the lipid-based nanoparticle, optionally through a linker, such as disclosed in Kedmi, Ranit et al. “A modular platform for targeted RNAi therapeutics.” Nature nanotechnology vol. 13, 3 (2018): 214-219. doi:10.1038/s41565-017-0043-5, the disclosure thereof being incorporated herein by reference.

In a particular aspect, the antigen binding domain is not covalently bound to any of the lipids of the LNP or does not comprise any modification for coupling or grafting the antigen binding domain to a lipid. In particular, the antigen binding domain does not comprise a lipophilic moiety or a grafting moiety such as free cysteine(s) or a thiol group.

In a particular aspect, the LNP does not include any antigen binding domain specific for an antigen present on the antigen binding domain capable of specifically binding to a target expressed on activated immune cells surface, in particular an antigen binding domain directed against the Fc domain of an antibody.

In a particular aspect, the antigen binding domain capable of specifically binding to a target expressed on activated immune cells surface is not bound to the LNP by a type of interaction “antigen-antibody”. More specifically, the antigen binding domain is not bound by an antigen binding domain specific for an antigen present on the antigen binding domain capable of specifically binding to a target expressed on activated immune cells surface, in particular an antigen binding domain directed against the Fc domain of an antibody.

Preferably, the lipid-based nanoparticle does not comprise a secondary antibody that allows the attachment of the antigen binding domain to the lipid-based nanoparticle (e.g., such as an anti-Fc antibody or antigen binding fragment or derivative thereof).

Particularly, the lipid-based nanoparticle does not comprise an antibody or any fragment or derivative thereof that is lipidated or that is covalently bound to a peptide or motif that is lipidated. Preferably, the lipid-based nanoparticle does not comprise a lipidated secondary antibody that allows the attachment of the antigen binding domain to the lipid-based nanoparticle.

In some aspects, the lipid-based nanoparticle does not comprise a moiety comprising a lipidation peptide or motif.

In some aspects, the antigen binding domain does not comprise or is not covalently bound to an anchoring moiety comprising a lipidation peptide or motif.

As used herein, the term “anchoring moiety” or “anchoring molecule” or “anchoring entity” refers to a component that anchors or attaches the antigen binding domain into the lipid-based nanoparticle. Preferably, the anchoring moiety is a protein.

As used herein, the terms “lipidation peptide or motif” refers to a specific sequence pattern in proteins or proteic entity (such as antibodies or fragment thereof) that is associated with the attachment or anchoring of lipid moieties.

In the context of the invention, an entity that comprises a lipidation peptide, motif or pattern is an entity (e.g., an antigen binding domain, scFv or antibody) that will be anchored or attachment to lipid molecules, in particular to lipids of the lipid-based nanoparticle.

Lipidation peptide or motif can involve different types of lipid modifications, including: cysteine prenylation (e.g., the attachment of hydrophobic isoprene polymers such as farnesyl or geranylgeranyl to cysteine residues of proteins), N-terminal Glycine Myristoylation, Cysteine Palmitoylation, Serine and Lysine Fatty Acylation (e.g., the addition of fatty acyl groups to serine and lysine residues of proteins), Palmitoylation, GPI-Anchor Addition, or peptides that derive from part of an inner membrane bacterial lipoprotein. One common example of a lipidation motif is the CAAX box, which serves as a recognition motif for isoprenylation.

In some aspects, the lipid based nanoparticle does not comprise a bacterial anchor polypeptide, a lipoprotein, such as a bacterial lipoprotein, or a recombinant membrane-anchored lipoprotein. Preferably, the antigen binding domain does not comprise or is not covalently bound to a bacterial anchor polypeptide, a lipoprotein, such as a bacterial lipoprotein, or a recombinant membrane-anchored lipoprotein.

Preferably, the lipid based nanoparticle of the invention does not comprise a NipA lipoprotein or any fragment thereof. Preferably, the antigen binding domain does not comprise or is not covalently bound to a NipA lipoprotein or any fragment thereof.

Preferably, the lipid based nanoparticle or antigen binding domain does not comprise a moiety comprising a lipidation peptide or motif that comprises or consists of the amino acid sequence: CDNSSS (SEQ ID NO: 41) or CDQSSS (SEQ ID NO: 42).

In an aspect, the antigen-binding domain to be comprised in the lipid-based nanoparticle according to the invention has a binding activity which is similar to the same antigen-binding domain in a free form. As used herein, a “free form antigen binding domain” refers to an antigen-binding domain which is not linked, grafted or conjugated to an LNP. In a preferred embodiment, the antigen-binding domain to be comprised in the lipid-based nanoparticle according to the invention has a binding activity equal to about 70% of the binding activity of the free form antigen-binding domain, even preferably to about 75%, even more preferably to about 80%.

In one another aspect, the antigen binding domain according to the present disclosure is an antigen-binding antibody mimetic. As used herein the term “antigen-binding antibody mimetic” refers to artificial proteins, peptides and any chemical compounds with the capacity to bind antigens mimicking that of antibodies. Such mimetics comprise affitins and anticalins as well as aptamers (peptide aptamers and oligonucleotide aptamers).

Alternatively, the antigen binding domain according to the present disclosure is a targeting peptide. Preferably, the peptide is not covalently linked to a lipid or to a lipidation motif. For example, the peptide is the RGD peptide such as described in Qin J, Xue L, Gong N, Zhang H, Shepherd S J, Haley R M, Swingle K L, Mitchell M J. RGD peptide-based lipids for targeted mRNA delivery and gene editing applications. RSC Adv. 2022 Sep. 7; 12(39):25397-25404. doi: 10.1039/d2ra02771b. PMID: 36199352; PMCID: PMC9450108.

In one aspect, the antigen binding domain is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods.

The antigen binding domain of the lipid-based nanoparticle can be of any format known in the art.

In a preferred aspect, the antigen binding domain is an antibody, a fragment or a derivative thereof such as a Fab, a F(ab)2, a Fab′, a F(ab′)2, a Fd, a Fv, a crossMAb or a single-chain variable fragment (scFV) a VHH or a single-chain Fab fragment.

Examples of antigen binding fragments encompassed typically include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain, or any fusion proteins comprising such antigen-binding fragments. Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody. These antibody fragments are obtained using conventional techniques known to those of skill in the art, and the fragments are screened for utility in the same manner as are intact antibodies.

As used herein, the term “CrossMAb” refers to antigen binding domains with an inversion of CL and CH1 domains, in particular in one binding arm of antibodies. Thus, such binding domain comprises a VH domain linked to a CL domain and a VL domain linked to a CH1 domain. Such format reduces the byproduct formation caused by a mismatch of a light chain of a first binding domain that specifically binds to a first antigen with the wrong heavy chain of a second biding domain that specifically binds to a second antigen (when compared to approaches without such CL-CH1 domain exchanges). CrossMAb are for example described in WO 2009/080253 and Schaefer, W. et al, PNAS, 108 (2011) 11187-1191, the disclosure of which being incorporated herein by reference.

In some instances, the antigen binding domain is an antibody or comprises or consists of a Fab, a Fv, a Fab′, a scFV or a VHH covalently linked to a Fc domain, preferably an IgG Fc domain such as described herein.

In some instances, the antigen binding domain is an aptamer or a short peptide sequence such as RGD peptide.

Aptamers are short ribonucleic acid (RNA) or deoxyribonucleic acid (DNA) sequences generated in vitro to bind with high affinity and specificity to a given target.

In a preferred aspect, the antigen binding domain is an antibody, a fragment or a derivative thereof such as a F(ab′)2, a Fab, a Fab′, a F(ab)2, Fd, Fv, a crossMAb, a single-chain variable fragment (scFV), a VHH or a single chain Fab fragment that is specific to PD-1, CTLA-4, BTLA, TIGIT, CD160, LAG-3 or TIM-3. Numerous antibodies directed against PD-1, TIM-3, CTLA-4, LAG-3, BTLA, TIGIT and CD160 have already been described in the art.

As used herein, the terms “Programmed Death 1”, “Programmed Cell Death 1”, “PD-1”, “PDCD1”, “PD-1 antigen”, “human PD-1”, “hPD-1” and “hPD-1” are used interchangeably and refer to the Programmed Death-1 receptor, also known as CD279, and include variants and isoforms of human PD-1, and analogs having at least one common epitope with PD-1.

PD-1 is a key regulator of the threshold of immune response and peripheral immune tolerance. It is expressed on activated T cells, B cells, monocytes, and dendritic cells and binds to its ligands PD-L1 and PD-L2. Human PD-1 is encoded by the PDCD1 gene. As an example, the amino acid sequence of a human PD-1 is disclosed under GenBank accession number NP_005009. PD-1 has four splice variants expressed on human Peripheral blood mononuclear cells (PBMC). Accordingly, PD-1 proteins include full-length PD-1, as well as alternative splice variants of PD-1, such as PD-iAex2, PD-iAex3, PD-iAex2,3 and PD-iAex2,3,4. Unless specified otherwise, the terms include any variant and, isoform of human PD-1 that are naturally expressed by PBMC, or that are expressed by cells transfected with a PD-1 gene.

Several anti-PD-1 are already clinically approved, and others are still in clinical developments. For instance, the anti-PD-1 antibody can be selected from the group consisting of Pembrolizumab (also known as Keytruda lambrolizumab, MK-3475), Nivolumab (Opdivo, MDX-1106, BMS-936558, ONO-4538), OSE-279 (see WO2020/127366), Pidilizumab (CT-011), Cemiplimab (Libtayo), Camrelizumab, AUNP12, AMP-224, AGEN-2034, BGB-A317 (Tisleizumab), PDR001 (spartalizumab), MK-3477, SCH-900475, PF-06801591, JNJ-63723283, Genolimzumab (CBT-501), LZM-009, BCD-100, SHR-1201, BAT-1306, AK-103 (HX-008), MEDI-0680 (also known as AMP-514), JS001 (see Si-Yang Liu et al., J. Hematol. Oncol.10:136 (2017)), BI-754091, CBT-501, INCSHRI210 (also known as SHR-1210), TSR-042 (also known as ANBO11), GLS-010 (also known as WBP3055), AM-0001 (Armo), STI-1110 (see WO 2014/194302), AGEN2034 (see WO 2017/040790), MGA012 (see WO 2017/19846), or IBI308 (see WO 2017/024465, WO 2017/025016, WO 2017/132825, and WO 2017/133540, the disclosure thereof being incorporated herein by reference), monoclonal antibodies 5C4, 17D8, 2D3, 4H1, 4A11, 7D3, and 5F4, described in WO 2006/121168, the disclosure thereof being incorporated herein by reference.

Antibodies directed against TIM-3 are also known such as Sym023, TSR-022, MBG453, LY3321367, INCAGN02390, BGTB-A425, LY3321367. In some aspects, the TFM-3 antibody is as disclosed in International Patent Application Publication Nos.

WO2013006490, WO2016/161270, WO 2018/085469, or WO 2018/129553, WO 2011/155607, U.S. Pat. No. 8,552,156, EP 2581113 and U.S 2014/044728, the disclosure thereof being incorporated herein by reference.

Antibodies directed against CTLA-4 are also known such as Ipilimumab, Tremelimumab, MK-1308, AGEN-1884, XmAb20717 (Xencor), MEDI5752 (AstraZeneca). Anti-CTLA-4 antibodies are also disclosed in WO18025178, WO19179388, WO19179391, WO19174603, WO19148444, WO19120232, WO19056281, WO19023482, WO18209701, WO18165895, WO18160536, WO18156250, WO18106862, WO18106864, WO18068182, WO18035710, WO18025178, WO17194265, WO17106372, WO17084078, WO17087588, WO16196237, WO16130898, WO16015675, WO12120125, WO09100140 and WO07008463, the disclosure thereof being incorporated herein by reference.

Antibodies directed against LAG-3 are also known such as BMS-986016, IMP701 or MGD012. Anti-LAG-3 antibodies are also disclosed in WO2008132601, EP2320940, WO19152574, the disclosure thereof being incorporated herein by reference.

Antibodies directed against BTLA are also known in the art such as hu Mab8D5, hu Mab8A3, hu Mab21H6, hu Mab19A7, or hu Mab4C7. The antibody TAB004 against BTLA are currently under clinical trial in subjects with advanced malignancies. Anti-BTLA antibodies are also disclosed in WO08076560, WO10106051 (e.g., BTLA8.2), WO11014438 (e.g., 4C7), WO17096017 and WO17144668 (e.g., 629.3), the disclosure thereof being incorporated herein by reference.

Antibodies directed against TIGIT are also known in the art, such as BMS-986207 or AB154, BMS-986207 CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8 as disclosed in WO19232484. Anti-TIGIT antibodies are also disclosed in WO16028656, WO16106302, WO16191643, WO17030823, WO17037707, WO17053748, WO17152088, WO18033798, WO18102536, WO18102746, WO18160704, WO18200430, WO18204363, WO19023504, WO19062832, WO19129221, WO19129261, WO19137548, WO19152574, WO19154415, WO19168382 and WO19215728, the disclosure thereof being incorporated herein by reference.

In some aspects, the target is CD127 or IL-7R and the antigen binding domain is specific to CD127, preferably human CD127. Preferably, the antigen binding domain is an antagonist of CD127.

As used herein, the term “IL-7R” refers to any form of IL-7R and variants thereof that retain at least part of the activity of IL-7R. One exemplary human IL-7R is found as Uniprot Accession Number P16871. Antagonist IL-7R antibodies encompass antibodies that block, antagonize, suppress or reduce (to any degree including significantly) IL-7R biological activity, including downstream pathways mediated by IL-7R signaling, such interaction with IL-7 and/or elicitation of a cellular response to IL-7.

Antibodies directed against CD127 or IL7-R are also known in the art, such as GSK2618960, RN168, AbD11590, MAB306-100, R34.34, A019D5, eBioRDR5, 40131, 1A12, M21, 47H4, HIL-7R-M21, eBioYL8, RDR5. Anti-CD127 antibodies are also disclosed in WO14102430, WO20077190, WO04000238, WO11104687, WO16059512 and WO17062748, the disclosure thereof being incorporated herein by reference.

In some aspects, the target is SIRPa and the antigen binding domain is specific to SIRPa, preferably human SIRPa. Preferably, the antigen binding domain is an antagonist of SIRPa.

As used herein, the terms “Signal-regulatory protein alpha”, “SIRPα” and “SIRPa” refers to a receptor-type transmembrane glycoprotein that is mammalian Immunoglobulin-like cell surface receptor for CD47. The term “anti-SIRP” refer to an antibody of the disclosure which is intended for use as a therapeutic or diagnostic agent, and specifically binds to SIRPa, in particular to a human SIRPa, to one or both of two common variants identified, SIRPaV1 and SIRPaV2. For example, the human SIRPa amino acid sequence is about 504 amino acids and has a Genbank accession number of NP_001035111.1, NP_001035112.1, NP_001317657.1, or NP_542970.1.

Antibodies directed against SIRPa are also known in the art, such as OSE-172, CC-95251, BI 765063, HPA054437, Magrolimab, TTI-621, TTI-622 and Evorpacept (ALX148). Anti-SIRPa antibodies are also disclosed in WO17178653, WO19073080, WO22254379, WO20102422, WO23202672, WO21222746, WO18008470, WO16205042, WO22121980, WO22110922, WO19226973, WO22254379, WO23020459 and WO18107058 the disclosure thereof being incorporated herein by reference.

In some aspects, the antigen binding domain comprises or consists of an anti-SIRPa antibody such as disclosed in WO19073080. Particularly, the antigen binding domain is an anti-SIRPa domain, comprising or consisting of a VH domain comprising or consisting of a sequence as set forth in SEQ ID NO: 39 and a VL domain comprising or consisting of a sequence as set forth in SEQ ID NO: 40. Preferably, said antigen binding domain further comprises or is covalently linked to a Fc domain, preferably an IgG Fc domain such as described herein.

In some aspects, the antigen binding domain is an anti-SIRPa antibody, comprising or consisting of: an heavy chain comprising or consisting of a sequence as set forth in SEQ ID NO: 51 and a light chain comprising or consisting of a sequence as set forth in SEQ ID NO: 52.

In some aspects, the target is CLEC-1A and the antigen binding domain is specific to CLEC-1A, preferably human CLEC-1A. Preferably, the antigen binding domain is an antagonist of CLEC-1A.

As used herein, the term “CLEC-1A” relates to a C-type lectin-like receptor-1 A from a mammal species, preferably a human CLEC-1A. A reference sequence of the human CLEC-1A corresponds to the sequence associated to the Accession number Q8NC01 Uniprot. As used herein, the term “CLEC-1 antagonist” has its general meaning in the art and refers to any compound, such as an antibody or a fragment thereof, that blocks, suppresses, or reduces the biological activity of CLEC-1. In particular, the CLEC-1 antagonist inhibits the interactions between the CLEC-1 and at least one of its ligands.

Antibodies directed against CLEC-1A are also known in the art, such as MAB1704, ABIN526589, AF1704 and ABIN526590.

In some aspects, the target is PD-1 and the antigen binding domain is specific to PD-1. Preferably, the antigen binding domain is an antagonist of PD-1. Even more preferably, the anti-PD-1 antibody is Pembrolizumab (also known as Keytruda lambrolizumab, MK-3475), Nivolumab (Opdivo, MDX-1106, BMS-936558, ONO-4538) or OSE-279 (such as described in WO2020/127366, the disclosure thereof being incorporated herein by reference).

Accordingly, in some aspects, the invention concerns a t-LNP, comprising an anti-PD-1 antigen-binding domain such as disclosed herein, capable of specifically binding to PD-1 expressed on activated T cells surface and one or several mRNA molecule(s) encoding an activity-inhibiting protein of said activated T cells.

Preferably, the antigen binding domain comprised in the lipid-based nanoparticle according to the invention is an anti-PD-1 antibody such as described above an or antigen binding fragment thereof, preferably a human, humanized or chimeric anti-PD-1 antibody or antigen binding fragment thereof. Particularly, the antigen binding domain is a F(ab′)2, a Fab, a crossMAb or a scFv that is specific to PD-1. Preferably, said antigen binding domain further comprises or is covalently linked to a Fc domain, preferably an IgG Fc domain such as described herein.

In a very specific aspect of the present disclosure, the antigen binding domain targets PD-1 and is derived from the antibody disclosed in WO2020/127366, the disclosure thereof being incorporated herein by reference in its entirety.

Then, in an aspect, the antigen binding domain is an anti-PD-1 antigen-binding domain comprising:
(i) a heavy chain variable domain comprising HCDR1, HCDR2 and HCDR3, and
(ii) a light chain variable domain comprising LCDR1, LCDR2 and LCDR3,
wherein:
   the heavy chain CDR1 (HCDR1) comprises or consists of an amino acid sequence of SEQ ID NO: 1, optionally with one, two or three modification(s) selected from substitution(s), addition(s), deletion(s) and any combination thereof, in particular at any position but position 3 of SEQ ID NO: 1;
   the heavy chain CDR2 (HCDR2) comprises or consists of an amino acid sequence of SEQ ID NO: 2, optionally with one, two or three modification(s) selected from substitution(s), addition(s), deletion(s) and any combination thereof, in particular at any position but positions 13, 14 and 16 of SEQ ID NO: 2;
   the heavy chain CDR3 (HCDR3) comprises or consists of an amino acid sequence of SEQ ID NO: 3; optionally with one, two or three modification(s) selected from substitution(s), addition(s), deletion(s) and any combination thereof, in particular at any position but positions 2, 3, 7 and 8 of SEQ ID NO: 3;
   the light chain CDR1 (LCDR1) comprises or consists of an amino acid sequence of SEQ ID NO: 4, optionally with one, two or three modification(s) selected from substitution(s), addition(s), deletion(s) and any combination thereof, in particular at any position but positions 5, 6, 10, 11 and 16 of SEQ ID NO: 4;
   the light chain CDR2 (LCDR2) comprises or consists of an amino acid sequence of SEQ ID NO: 5, optionally with one, two or three modification(s) selected from substitution(s), addition(s), deletion(s) and any combination thereof; and
   the light chain CDR3 (LCDR3) comprises or consists of an amino acid sequence of SEQ ID NO: 6, optionally with one, two or three modification(s) selected from substitution(s), addition(s), deletion(s) and any combination thereof at any position but positions 1, 4 and 6 of SEQ ID NO: 6.

In another aspect, the anti-PD-1 antigen-binding domain comprises or consists essentially of: (i) a heavy chain variable region (VH) comprising a CDR1 of SEQ ID NO: 1, a CDR2 of SEQ ID NO: 2 and a CDR3 of SEQ ID NO: 3; and (ii) a light chain variable region (VL) comprising a CDR1 of SEQ ID NO: 4, a CDR2 of SEQ ID NO: 5 and a CDR3 of SEQ ID NO: 6.

In one embodiment, the anti-PD1 antibody or antigen binding fragment according to the invention comprises framework regions, in particular heavy chain variable region framework regions (HFR) HFR1, HFR2, HFR3 and HFR4 and light chain variable region framework regions (LFR) LFR1, LFR2, LFR3 and LFR4.

Preferably, the anti-PD-1 antigen-binding domain comprises or consists essentially of:
(i) a heavy chain variable region (VH) comprising a HFR1 of SEQ ID NO: 7, optionally with one, two or three modification(s) selected from substitution(s), addition(s), deletion(s) and any combination thereof, a HCDR1 of SEQ ID NO: 1, a HFR2 of SEQ ID NO: 8, optionally with one, two or three modification(s) selected from substitution(s), addition(s), deletion(s) and any combination thereof, a HCDR2 of SEQ ID NO: 2, a HFR3 of SEQ ID NO: 9, optionally with one, two or three modification(s) selected from substitution(s), addition(s), deletion(s) and any combination thereof, a HCDR3 of SEQ ID NO: 3; and a HFR4 of SEQ ID NO: 10, optionally with one, two or three modification(s) selected from substitution(s), addition(s), deletion(s) and any combination thereof, and
(ii) a light chain variable region (VL) comprising a LFR1 of SEQ ID NO: 11, optionally with one, two or three modification(s) selected from substitution(s), addition(s), deletion(s) and any combination thereof, a LCDR1 of SEQ ID NO: 4, a LFR2 of SEQ ID NO: 12, optionally with one, two or three modification(s) selected from substitution(s), addition(s), deletion(s) and any combination thereof, a LCDR2 of SEQ ID NO: 5, a LFR3 of SEQ ID NO: 13, optionally with one, two or three modification(s) selected from substitution(s), addition(s), deletion(s) and any combination thereof, a LCDR3 of SEQ ID NO: 6 and a LFR4 of SEQ ID NO: 14, optionally with one, two or three modification(s) selected from substitution(s), addition(s), deletion(s) and any combination thereof.

In an aspect, the anti-PD-1 antigen-binding domain comprises or consists essentially of:
(a) a heavy chain variable region (VH) comprising or consisting of an amino acid sequence SEQ ID NO: 15, optionally with one, two or three modification(s) selected from substitution(s), addition(s), deletion(s) and any combination thereof, in particular at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 15;
(b) a light chain variable region (VL) comprising or consisting of an amino acid sequence of SEQ ID NO: 16, optionally with one, two or three modification(s) selected from substitution(s), addition(s), deletion(s) and any combination thereof, in particular at any position but positions 3, 4, 7, 14, 17, 18, 28, 29, 33, 34, 39, 42, 44, 50, 81, 88, 94, 97, 99 and 105 of SEQ ID NO: 16.

In another aspect, the anti-PD-1 antigen-binding domain comprises or consists essentially of:
(a) a heavy chain variable region (VH) comprising or consisting of an amino acid sequence of SEQ ID NO: 15; optionally with one, two or three modification(s) selected from substitution(s), addition(s), deletion(s) and any combination thereof outside of the CDRs (i.e., in the framework region only);
(b) a light chain variable region (VL) comprising or consisting of an amino acid sequence of SEQ ID NO: 16 optionally with one, two or three modification(s) selected from substitution(s), addition(s), deletion(s) and any combination thereof outside of the CDRs (i.e., in the framework region only).

Preferably, the anti-PD-1 antigen-binding domain comprises or consists essentially of:
(a) a heavy chain variable region (VH) comprising or consisting of an amino acid sequence of SEQ ID NO: 15 and (b) a light chain variable region (VL) comprising or consisting of an amino acid sequence of SEQ ID NO: 16.

In an aspect, the anti-PD-1 antigen-binding domain comprises VH, VL, CH1 and a CL domains, so that the antigen binding domain is a Fab.

In such aspect, the heavy chain constant domain (CH1) comprises or consists essentially of SEQ ID NO: 17, optionally with one, two or three modification(s) selected from substitution(s), addition(s), deletion(s) and any combination thereof.

Preferably, the light chain constant domain (CL) comprises or consists essentially of SEQ ID NO: 18 optionally with one, two or three modification(s) selected from substitution(s), addition(s), deletion(s) and any combination thereof.

In an embodiment, the anti-PD-1 antigen-binding domain is a Fab or a Fab′, a Fab or a F(ab′)2 and comprises i) a VH domain and a CH1 domain, said VH and CH1 domains having the amino acid sequence as set forth in SEQ ID Nos: 15 and 17, respectively, optionally with one, two or three modification(s) selected from substitution(s), addition(s), deletion(s) and any combination thereof, and ii) a VL domain and a CL domain, said domains having the amino acid sequence as set forth in SEQ ID Nos: 16 and 18, respectively, optionally with one, two or three modification(s) selected from substitution(s), addition(s), deletion(s) and any combination thereof.

Preferably, the antigen binding domain is an anti-PD-1 Fab or F(ab′)2, comprising or consisting of i) a chain comprising or consisting of a VH domain and a CH1 domain, said VH and CH1 domains having the amino acid sequence as set forth in SEQ ID Nos: 15 and 17 respectively and ii) a chain comprising or consisting of VL and CL domains, said domains having the amino acid sequence as set forth in SEQ ID Nos: 16 and 18, respectively.

In an aspect, the antigen binding domain is an anti-PD-1 Fab or F(ab′)2, comprising or consisting of i) a chain comprising or consisting of an amino acid sequence as set forth in SEQ ID NO: 19 optionally with one, two or three modification(s) selected from substitution(s), addition(s), deletion(s) and any combination thereof and of ii) a chain comprising or consisting of an amino acid sequence as set forth in SEQ ID NO: 20 optionally with one, two or three modification(s) selected from substitution(s), addition(s), deletion(s) and any combination thereof.

In an aspect, the antigen binding domain comprises an anti-PD-1 CrossMAb comprising or consisting of i) a chain comprising or consisting of VH and CL domains, said domains having the amino acid sequence as set forth in SEQ ID NOs: 15 and 18, respectively, optionally with one, two or three modification(s) selected from substitution(s), addition(s), deletion(s) and any combination thereof, ii) a chain comprising or consisting of VL and CH1 domains, said domains having the amino acid sequence as set forth in SEQ ID NOs: 16 and 17, respectively, optionally with one, two or three modification(s) selected from substitution(s), addition(s), deletion(s) and any combination thereof.

Preferably, the antigen binding domain comprises an anti-PD-1 CrossMAb, comprising or consisting of i) a chain comprising or consisting of a VH domain and a CL domain, said domains having the amino acid sequence as set forth in SEQ ID NOs: 15 and 18, respectively, and ii) a chain comprising or consisting of VL and CH1 domains, said domains having the amino acid sequence as set forth in SEQ ID NOs: 16 and 17, respectively.

In an aspect, the antigen binding domain is an anti-PD-1 CrossMAb, comprising or consisting of i) a chain comprising or consisting of an amino acid sequence as set forth in SEQ ID NO: 21 optionally with one, two or three modification(s) selected from substitution(s), addition(s), deletion(s) and any combination thereof and of ii) a chain comprising or consisting of an amino acid sequence as set forth in SEQ ID NO: 22 optionally with one, two or three modification(s) selected from substitution(s), addition(s), deletion(s) and any combination thereof.

In some aspects, when the antigen binding domain is described as having one, two or three modification(s) selected from substitution(s), addition(s), deletion(s) and any combination thereof, said modifications are outside of the CDRs.

Alternatively, the antigen binding domain is an anti-PD-1 antigen-binding domain comprising:
(i) a heavy chain variable domain comprising HCDR1, HCDR2 and HCDR3, and
(ii) a light chain variable domain comprising LCDR1, LCDR2 and LCDR3,
wherein
HCDR1 comprises or consists of an amino acid sequence of SEQ ID NO: 23,
HCDR2 comprises or consists of an amino acid sequence of SEQ ID NO: 24,
HCDR3 comprises or consists of an amino acid sequence of SEQ ID NO: 25,
LCDR1 comprises or consists of an amino acid sequence of SEQ ID NO: 26,
LCDR2 comprises or consists of an amino acid sequence of SEQ ID NO: 27, and
LCDR3 comprises or consists of an amino acid sequence of SEQ ID NO: 28,

Preferably, the antigen binding domain is an anti-PD-1 antigen-binding domain comprising:
(a) a heavy chain variable region (VH) comprising or consisting of an amino acid sequence of SEQ ID NO: 29;
(b) a light chain variable region (VL) comprising or consisting of an amino acid sequence of SEQ ID NO: 30.
(i) a heavy chain variable domain comprising HCDR1, HCDR2 and HCDR3, and
(ii) a light chain variable domain comprising LCDR1, LCDR2 and LCDR3,
wherein
HCDR1 comprises or consists of an amino acid sequence of SEQ ID NO: 31,
HCDR2 comprises or consists of an amino acid sequence of SEQ ID NO: 32,
HCDR3 comprises or consists of an amino acid sequence of SEQ ID NO: 33,
LCDR1 comprises or consists of an amino acid sequence of SEQ ID NO: 34,
LCDR2 comprises or consists of an amino acid sequence of SEQ ID NO: 35, and
LCDR3 comprises or consists of an amino acid sequence of SEQ ID NO: 36,

Preferably, the antigen binding domain is an anti-PD-1 antigen-binding domain comprising:
(a) a heavy chain variable region (VH) comprising or consisting of an amino acid sequence of SEQ ID NO: 37;
(b) a light chain variable region (VL) comprising or consisting of an amino acid sequence of SEQ ID NO: 38.

In some aspects, the antigen binding domain is an anti-PD-1 antibody, comprising or consisting of:
an heavy chain comprising or consisting of a sequence as set forth in SEQ ID NO: 45 and a light chain comprising or consisting of a sequence as set forth in SEQ ID NO: 46;
an heavy chain comprising or consisting of a sequence as set forth in SEQ ID NO: 47 and a light chain comprising or consisting of a sequence as set forth in SEQ ID NO: 48; or
an heavy chain comprising or consisting of a sequence as set forth in SEQ ID NO: 49 and a light chain comprising or consisting of a sequence as set forth in SEQ ID NO: 50.

Preferably, the antigen binding domain is an antagonist anti-PD-1 antibody comprising or consisting of an heavy chain comprising or consisting of a sequence as set forth in SEQ ID NO: 45 and a light chain comprising or consisting of a sequence as set forth in SEQ ID NO: 46.

In a particular aspect of the disclosure, the variable regions of the antigen binding domain as described above may be associated with antibody constant regions, in particular from IgA, IgM, IgE, IgD or IgG such as IgG1, IgG2, IgG3, IgG4, preferably IgG1, IgG2, or IgG4.

Preferably, the antigen binding domain comprises an IgG Fc region, preferably an IgG1, IgG2, or IgG4 Fc region. Preferably, the Fc domain includes all or a portion of a hinge region. The hinge region can be derived from an immunoglobulin heavy chain, e.g., IgG1, IgG2, IgG3, IgG4, or other classes. Preferably, the hinge region is derived from a human IgG1, IgG2, IgG3 or IgG4. Preferably, the hinge region is derived from a human or humanized IgG1, IgG2 or IgG4.

Preferably, the antigen binding domain comprises or is covalently linked to a Fc domain, preferably an IgG Fc domain. Particularly, the Fc region is derived from human or humanized IgG1, IgG2 or IgG4. Preferably, the antigen binding domain comprises a Fc domain and a hinge region derived from a human or humanized IgG1, IgG2 or IgG4.

Preferably, the antigen binding domain comprises or is covalently linked to a Fc domain, preferably an IgG Fc domain. Particularly, the Fc region is derived from human or humanized IgG1, IgG2 or IgG4. Preferably, the antigen binding domain comprises a Fc domain and a hinge region derived from a human or humanized IgG1, IgG2 or IgG4.

Preferably, the antigen binding domain comprises a Fc domain, preferably an IgG Fc domain. For example, this means that the antigen binding domain is an antibody that comprises an IgG Fc domain and/or that the antigen binding domain comprises an antigen binding fragment of an antibody (such as a Fab or scFv) that is covalently linked to an IgG Fc domain.

As used herein, the term “IgG Fc region” or “IgG Fc domain” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The constant regions may be further mutated or modified, by methods known in the art, for modifying their binding capability towards Fc receptor.

Typically a Fc domain comprises two heavy chain constant domain, known as CH2 and CH3 domains. Optionally, the Fc domain envisioned herein also comprises a hinge region.

In one embodiment, the antigen binding domain comprises a truncated Fc region or a fragment of the Fc region. In one embodiment, the Fc region includes a CH2 domain. In another embodiment, the Fc region includes CH2 and CH3 domains or includes hinge-CH2-CH3. Alternatively, the Fc domain can include all or a portion of the hinge region, a CH2 domain and/or a CH3 domain.

Antibodies or antigen-binding fragments thereof with amino acid sequences having at least 90%, for example, at least 95%, 96%, 97%, 98%, or 99% identity to any one of the above defined amino acid sequences are also part of the present disclosure.

In some aspects, the amino acid differences are conservative substitutions, i.e., substitutions of one amino acid with another having similar chemical or physical properties (size, charge or polarity), which substitution generally does not adversely affect the biochemical, biophysical and/or biological properties of the antibody or antigen-binding fragment thereof.

In particular, the substitution does not disrupt the interaction of the antibody or antigen-binding fragment thereof with its target. Said conservative substitution(s) are advantageously chosen within one of the following five groups: Group 1-small aliphatic, non-polar or slightly polar residues (A, S, T, P, G); Group 2-polar, negatively charged residues and their amides (D, N, E, Q); Group 3-polar, positively charged residues (H, R, K); Group 4-large aliphatic, nonpolar residues (M, L, I, V, C); and Group 5-large, aromatic residues (F, Y, W).

The skilled person is able to determine the location of the various regions/domains of antibodies by reference to the standard definitions in this respect set forth, including a reference numbering system, a reference to the numbering system of KABAT or by application of the IMGT algorithm. In this respect, for the definition of the sequences of the invention, it is noted that the delimitation of the regions/domains may vary from one reference system to another. Accordingly, the regions/domains as defined in the present invention encompass sequences showing variations in length or localization of the concerned sequences within the full-length sequence of the variable domains of the antibodies, of approximately +/−10%.

In an aspect, the lipid-based nanoparticle comprises an additional antigen binding domain which binds to a target specifically expressed on activated immune cell surface (i.e., in addition to the first antigen binding domain). In this aspect, the lipid-based nanoparticle particularly comprises a first antigen binding domain which binds to a first target specifically expressed on activated immune cell surface and a second antigen binding domain which binds to a second target specifically expressed on activated immune cell surface.

Particularly, the additional or second antigen binding domain is i) not covalently bound to any of the lipids of the lipid-based nanoparticle, ii) does not comprise any modification for coupling or grafting the antigen binding domain to a lipid and/or iii) is not covalently bound to a lipidation peptide or motif, in particular a lipidation peptide or motif such as described herein.

Preferably, the additional or second antigen binding domain is not a secondary antibody, typically a secondary antibody that allows the attachment of the first antigen binding domain to the lipid-based nanoparticle (e.g., such as an anti-Fc antibody or antigen binding fragment or derivative thereof).

Preferably, the additional or second antigen binding domain is not a secondary antibody comprising a lipidation peptide or motif such as described herein.

In a preferred aspect, the first antigen binding domain and the additional (second) antigen binding domain bind to targets expressed on the surface of the same activated immune cells.

Said targets can be the same or different. For example, when the immune cell is a T cell, the first antigen binding domain binds for example to PD-1, and the second antigen binding domain binds to an antigen selected from the group consisting of CD137/41BB/TNFRSF9, CRTAM, CTLA4, FasL/TNFSF6, TIM-3/HAVCR2, ITGAE, LAG-3, OX40/TNFRSF4, SIRPg and TIGIT. Alternatively, when the immune cell is a T cell, both the first and second antigen binding domains may bind to PD-1. Preferably, the first and second antigen binding domains are not competing antibodies for PD-1, which means that the first and second antigen binding domains recognize a different (non-overlapping) epitope of PD-1.

In some embodiments, each of the first and second targets specifically expressed on activated immune cell surface (i.e., said targets being recognized by the first and second antigen binding domains, respectively) are selected from the group comprising CD4, CD8, BCMA/TNFRSF17, BTLA, CD101/IGSF2, CD103, CD119, CD137/4-1BB/TNFRSF9, CD150, CD153, CD154, CD223, CD226, CD25, CD254, CD26, CD27, CD275, CD39/ENTPD1, CD40L, CD44, CD45RO, CD45RC, LGR6, CD69, GPR18, GPR35, FPR2, CD80, CD83, CD86, CD95, CMKLR1, CRTAM, CST7, CTLA4, CXCR3, CXCR4, CXCR5, CXCR6, FasL/TNFSF6, GITR/TNFRSF18, GPR32, TIM3/HAVCR2, ICOS, IL18R1/CXCR1/CD218a, ITGAE, LAG3, TRAILR, OX40L, LY108/SlamF6, NKG2D, OX40/TNFRSF4, PDCD1, PTPN22, RGS1, LOX1, SIGLEC 6, TACI/TNFRSF13B, TIGIT, CD163, CD206, LTBR/CD70, TNFSF14, SLAMF1, SLAMF7, NKG2A, KIR2DL2, CD96, CD112R, CD28H, IL2RB, TRAIL, CD48, CD53, CD164, CD138 (SDC1), CD38, CD39, FCRL4, CD30/TNFRSF8, CD78, TRAF1, TRAF2, TRAF3/CD40BP, TRAF3IP1, TRAF4, TRAF7, TRAP1, TNFR1/TNFRSF1A/CD120A, TRAP100/MED24, TNFR2/TNFRSF1811/CD120B, CDCR3/TNFRSF6B, TNFRSF12A/FN14/TWEAKR, BAFFR/TNFRSF13C/CD268, HVEM/TNFRSF14/CD270, GITR/TNFRSF8/CD357, RELT/TNFRSF19L, TNFRSF19/TROY, TNFRSF21/DR6, TNFRSF25/DR3/TNFRSF12, CD301, IL4R, CLEC-1A, CD21, CLEC-9A, CD180, CD59, CD54, CD71, CD35, CD218a, CD74, CD165, 4-1BBL/CD137L, ICOSL and CD160.

Adding the additional antigen binding domain enhances the specificity of the lipid-based nanoparticle for a specific activated immune cell population, based on their nature and/or localization. Enhancing the already high specificity of the lipid-based nanoparticle of the invention limits or completely removes the risk of off-target or toxicity in healthy tissue and/or organs.

In a particular aspect, the first antigen binding domain and the second antigen binding domain are derived from Fab, Fab′, F(ab′)2, Fv, single chain (scFv), CrossMAb or nanobody (VHH), preferably a F(ab′)2, a Fab, a CrossMAb or a scFV and the first and second antigen binding domains may have the same or different formats. For example, the first antigen binding domain and the second antigen binding domain can be both Fab or scFV.

Alternatively, the first antigen binding domain and the second antigen binding domain are not both scFv. Preferably, the first antigen binding domain and the second antigen binding antigen binding domain comprise or are covalently linked to a Fc domain, preferably an IgG Fc domain such as described herein.

The first antigen binding domain and the second antigen binding domain may have the same or different formats. For example, the first antigen binding domain and the second antigen binding domain can be both antibodies, Fab or scFV. Alternatively, the first antigen binding domain and the second antigen binding domain are not both scFv.

In some aspects, the lipid-based nanoparticle comprises 1, 2, 3, 4, 5, 6, 7, 8, 9 or 10 different antigen binding domains. Such antigen binding domains can be two distinct antigen biding domains that recognize the same target (e.g., such as pembrolizumab and nivolumab that both binds to PD-1) or two distinct antigen biding domains that recognize two different target (e.g., such as PD-1 and CTLA-4).

In some embodiments, in addition to the antigen binding domain, additional targeting molecules may be used in the lipid-based nanoparticle of the invention. For example, modified sugars or modified lipids can be used in combination with the antigen binding domain of the invention.

mRNAs

The lipid-based nanoparticle of the invention comprises a mRNA molecule encoding an immune cell activity inhibiting protein.

Preferably, the lipid-based nanoparticle of the invention comprises one or more isolated mRNA molecule(s).

The lipid-based nanoparticle of the invention particularly includes an mRNA encoding a polypeptide of interest capable of being translated by the immune cell to produce the polypeptide of interest.

In a particular aspect, the lipid-based nanoparticle comprises a mRNA polynucleotide or a set of mRNA polynucleotides. The technology of mRNA polynucleotide is now well-known by the person skilled in the art, as illustrated in WO21159130, the disclosure thereof being incorporated herein by reference.

The amount of a mRNA molecule in a lipid-based nanoparticle may depend on the size, composition, desired target and/or application, or other properties of the lipid-based nanoparticle. For example, the amount of mRNA useful in the lipid-based nanoparticle may also depend on the size, sequence, and other characteristics of the mRNA. The relative amounts of a mRNA molecule and other elements (e.g., lipids) in a lipid-based nanoparticle may also vary. In some aspects, the wt/wt ratio of the lipid component to a mRNA molecule in a lipid-based nanoparticle may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1,30:1,35:1, 40:1, 45:1, 50:1, and 60:1. For example, the wt/wt ratio of the lipid component to a mRNA molecule may be from about 10:1 to about 40:1. In certain aspects, the wt/wt ratio is about 20:1. The amount of a mRNA molecule in a lipid-based nanoparticle may, for example, be measured using absorption spectroscopy (e.g., ultraviolet-visible spectroscopy).

Alternatively, the amount of lipid and mRNA may be selected to provide a specific N:P ratio. The N:P ratio of the composition refers to the molar ratio of nitrogen atoms in one or more lipids to the number of phosphate groups in an mRNA. In general, a lower N:P ratio is preferred. The one or more mRNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain aspects, the N:P ratio may be from about 2:1 to about 8:1. In other aspects, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1. Preferably, the N:P ratio is between about 5:1 and about 7:1. Preferably, the N:P ratio is about 6:1.

The efficiency of encapsulation of mRNA molecules describes the amount of mRNA encapsulated or otherwise associated with a LNP after preparation, relative to the initial amount provided. The encapsulation efficiency is preferably desirably high (e.g., close to 100%). The encapsulation efficiency may be measured, for example, by comparing the amount of mRNA in a solution containing the lipid-based nanoparticle before and after breaking up the lipid-based nanoparticle with one or more organic solvents or detergents.

Fluorescence may be used to measure the amount of free mRNA in a solution. For the lipid-based nanoparticles described herein, the encapsulation efficiency may be of at least 50%, for example 50%, 55%, 60%, 65%, 70%, 75%, 80%, 85%, 90%, 91%, 92%, 93%, 94%, 95%, 96%, 97%, 98%, 99%, or 100%. In some aspects, the encapsulation efficiency is of at least 70%. In certain aspects, the encapsulation efficiency is of at least 80%, preferably 90%.

The mRNA molecule of the invention particularly comprises structural elements that allows its encapsulation into the lipid-based nanoparticle and/or its expression into the targeted immune cell.

Preferably, the mRNA molecule contains stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Preferably, mRNA polynucleotides comprise at least one base modification and at least one 5′-terminal cap. mRNA envisioned herein typically include a region of linked nucleosides encoding a polypeptide of interest (e.g., a coding region), a first flanking region located at the 5′-terminus of the region of linked nucleosides (e.g., a 5′-UTR), a second flanking region located at the 3′-terminus of the region of linked nucleosides (e.g., a 3′-UTR), at least one 5′-cap region, and a 3′-stabilizing region.

In some aspects, the mRNA of the invention includes a poly-A region or a Kozak sequence (e.g., in the 5′-UTR). In some cases, the mRNA of the invention comprises one or more intronic nucleotide sequences capable of being excised from the polynucleotide.

In some aspects, the mRNA of the invention includes a flanking region, a 5′-cap structure, a chain terminating nucleotide, a stem loop, a poly-A sequence and/or a polyadenylation signal.

In some aspects, the mRNA of the invention comprises a flanking region. A 5′-UTR or a 3′-UTR may be provided as a flanking region to the mRNA of the invention. A 5′-UTR may be homologous or heterologous to the coding region of the mRNA. Multiple 5′-UTRs or 3′-UTRs may be included in the flanking region and may be of the same or of different sequences. Any portion of the flanking regions, including none, may be codon optimized and any may independently contain one or more different structural or chemical alterations, before and/or after codon optimization.

To alter one or more properties of an mRNA, 5′-UTRs or 3′-UTRs which are heterologous to the coding region of an mRNA may be engineered. The mRNA may then be administered to cells, tissue or organisms and outcomes such as protein level, localization, and/or half-life may be measured to evaluate the beneficial effects the heterologous 5′-UTR and/or 3′-UTR may have on the mRNA. Variants of the 5′-UTRs and/or 3′-UTRs may be utilized wherein one or more nucleotides are added or removed to the termini, including A, T, C or G. 5′-UTRs and/or 3′-UTR may also be codon-optimized, or altered in any manner described herein.

In some aspects, the mRNA comprises an Internal ribosome entry site (IRES) or a Kozak sequence in the 5′-UTR region. The Kozak consensus sequence (Kozak consensus or Kozak sequence) is a nucleic acid motif that functions as the protein translation initiation site. An internal ribosome entry site (IRES) is an RNA element that allows for translation initiation in a cap-independent manner.

In some aspects, the mRNA of the invention comprises a 5′-capping region or structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns during mRNA splicing. Endogenous polynucleotide molecules may be 5′-end capped generating a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′-end of the polynucleotide may optionally also be 2′-O-methylated. 5′-decapping through hydrolysis and cleavage of the guanylate cap structure may target a polynucleotide molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional alternative guanosine nucleotides may be used such as a-methyl-phosphonate and seleno-phosphate nucleotides. Additional alterations include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of an mRNA molecule.

mRNAs may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, recombinant Vaccinia Virus Capping Enzyme or a Faustovirus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the Capl structure. This cap results in a higher translational-competency, cellular stability, and a reduced activation of cellular pro-inflammatory cytokines, as compared, e.g., to other 5′-cap analog structures known in the art. Other exemplary cap structures include 7mG(5′)ppp(5′)N,pN2p (Cap 0), 7mG(5′)ppp(5′)NlmpNp (Cap 1), 7mG(5′)-ppp(5′)NlmpN2mp (Cap 2), and m(7)Gpppm(3)(6,6,2′)Apm(2′)Apm(2′)Cpm(2)(3,2′)Up (Cap 4).

Because the mRNAs may be capped post-transcriptionally, and because this process is more efficient, nearly 100% of the mRNA may be capped. This is in contrast to 80% when a cap analog is linked to a polynucleotide in the course of an in vitro transcription reaction. 5′-terminal caps may include endogenous caps or cap analogs. A 5′-terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5′-cap. A modification on the 5′-cap may increase the stability of polynucleotide, increase the half-life of the polynucleotide, and could increase the polynucleotide translational efficiency. The modified 5′-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme or a Faustovirus Capping Enzyme to generate the “Cap 0” structure: m7G(5′)ppp(5′)G (New England BioLabs, Ipswich, MA). Cap 1 structure may be generated using both Vaccinia Vims Capping Enzyme or a Faustovirus Capping Enzyme and a 2′-O-methyl-transferase to generate: m7G(5′)ppp(5′)G-2-O-methyl. Cap 2 structure may be generated from the Cap 1 structure followed by the 2′-O-methylation of the 5-antepenultimate nucleotide using a 2′-Omethyl-transferase. Cap 3 structure may be generated from the Cap 2 structure followed by the 2′-O-methylation of the 5′-preantepenultimate nucleotide using a 2′-O-methyl-transferase. Enzymes may be derived from a recombinant source. Alternatively, other caps may be used, such as CleanCap structures (Trilink). Cleancap is a trinucleotide with a 5′-m7G joined by a 5′-5′ triphosphate linkage to an AG sequence.

The mRNA of the invention may particularly comprise a 5′-cap analog. Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide. For example, the Anti-Reverse Cap Analog (ARCA) cap contains two guanosines linked by a 5′-5′-triphosphate group, wherein one guanosine contains an N7-methyl group as well as a 3′-O-methyl group (i.e., N7, 3′—O-dimethyl-guanosine-5′-triphosphate-5′-guanosine, m7G-3′mppp-G, which may equivalently be designated 3′O-Me-m7G(5′)ppp(5′)G). The 3′-O atom of the other, unaltered, guanosine becomes linked to the 5-terminal nucleotide of the capped polynucleotide (e.g., an mRNA). The N7- and 3′-O-methylated guanosine provides the terminal moiety of the capped polynucleotide (e.g., mRNA). Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

The 5′-cap of the mRNA may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference. Alternatively, a cap analog may be a N7-(4-chlorophenoxy-ethyl) substituted dinucleotide cap analog known in the art and/or described herein. Non-limiting examples of N7-(4-chlorophenoxy-ethyl) substituted dinucleotide cap analogs include a N7-(4-chlorophenoxy-ethyl)-G(5)ppp(5′)G and a N7-(4-chlorophenoxy-ethyl)-m3′-OG(5)ppp(5′)G cap analog (see, e.g., the various cap analogs and the methods of synthesizing cap analogs described in Kore et al. Bioorganic & Medicinal Chemistry 20132: 4570-4574; the cap structures of which are herein incorporated by reference). In other instances, a cap analog useful in the polynucleotides of the present disclosure is a 4-chloro/bromophenoxy-ethyl analog.

In some aspects, the mRNA of the invention includes a stem loop such as, but not limited to, a histone stem loop. The histone stem loop may be before and/or after the poly-A region. The mRNA including the histone stem loop and a poly-A region sequence may include a chain terminating nucleoside described herein. In other instances, the mRNA includes a histone stem loop and a 5′-cap structure such as described herein and/or known in the art. In some cases, the conserved stem loop region may include a miR sequence. As a non-limiting example, the stem loop region may include the seed sequence of a miR sequence. For example, the stem loop region may include a miR-122 seed sequence.

Preferably, the stem loop is a nucleotide sequence of about 25 or about 26 nucleotides in length. The histone stem loop may be located 3′-relative to the coding region (e.g., at the 3′-terminus of the coding region). As a non-limiting example, the stem loop may be located at the 3′-end of a mRNA described herein. In some cases, an mRNA includes more than one stem loop (e.g., two stem loops). A stem loop may be located in a second terminal region of a polynucleotide. As a non-limiting example, the stem loop may be located within an untranslated region (e.g., 3′-UTR) in a second terminal region. In some cases, a mRNA which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide.

In other cases, the mRNA, which includes the histone stem loop is stabilized by an alteration to the 3′-region of the polynucleotide that can prevent and/or inhibit the addition of oligo(U). In yet other cases, the mRNA, which includes the histone stem loop is stabilized by the addition of an oligonucleotide that terminates in a 3′-deoxynucleoside, 2′, 3′-dideoxynucleoside 3′-O-methylnucleosides, 3′-O-ethylnucleosides, 3′-arabinosides, and other alternative nucleosides known in the art and/or described herein.

The mRNA may particularly include at least one histone stem-loop and a poly-A region or polyadenylation signal.

In some aspects, the mRNA comprised in the lipid-based nanoparticle of the invention includes a poly-A sequence and/or polyadenylation signal. A poly-A sequence may be comprised entirely or mostly of adenine nucleotides or analogs or derivatives thereof. A poly-A sequence may be a tail located adjacent to a 3′-untranslated region of a nucleic acid. During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the mRNAs of the present disclosure. Generally, the length of a poly-A region of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides. In some instances, the poly-A region may be 80 nucleotides, 120 nucleotides, 160 nucleotides in length on an mRNA molecule described herein. In other instances, the poly-A region is of 20, 40, 80, 100, 120, 140 or 160 nucleotides in length on an mRNA molecule described herein. In some cases, the poly-A region is designed relative to the length of the overall mRNA. This design may be based on the length of the coding region, the length of a particular feature or region of the mRNA or based on the length of the ultimate product expressed from the mRNA. When relative to any feature of the mRNA (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the mRNA to which it belongs. In this context, the poly-A region may be 10, 20, 30, 40, 50, 60, 70, 80, or 90% or more of the total length of the construct or the total length of the construct minus the poly-A region.

In some instances, the mRNA includes a poly-A-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant mRNA may be assayed for stability, protein production and other parameters including half-life at various time points. It has been discovered that the poly-A-G quartet results in protein production equivalent to at least 75% of that seen using a poly-A region of 120 nucleotides alone. The mRNA with a poly-A-G Quartet may further include a 5′-cap structure. In some cases, the 3′-stabilizing region which may be used to stabilize mRNA includes a poly-A region or poly-A-G Quartet. In other cases, the 3′-stabilizing region which may be used with the present disclosure include a chain termination nucleoside such as 3′-deoxyadenosine (cordycepin), 3′-deoxyuridine, 3′-deoxycytosine, 3′-deoxyguanosine, 3′-deoxythymine, 2′,3′-dideoxynucleosides, such as 2′, 3′-dideoxyadenosine, 2′, 3′-dideoxyuridine, 2′, 3′-dideoxycytosine, 2′, 3′-dideoxyguanosine, 2′, 3′-dideoxythymine, a 2′-deoxynucleoside, or an O-methylnucleoside. In other cases, mRNA which includes a poly-A region or a poly-A-G Quartet may be stabilized by an alteration to the 3′-region of the polynucleotide that can prevent and/or inhibit the addition of oligo(U). In yet other instances, mRNA which includes a poly-A region or a poly-A-G Quartet may be stabilized by the addition of an oligonucleotide that terminates in a 3′-deoxynucleoside, 2′,3′-dideoxynucleoside 3-O-methylnucleosides, 3′-O-ethylnucleosides, 3′-arabinosides, and other alternative nucleosides known in the art and/or described herein.

In certain cases, engineered binding sites and/or the conjugation of mRNA for poly-A binding protein is used to enhance expression. The engineered binding sites may be sensor sequences which can operate as binding sites for ligands of the local microenvironment of the mRNA. As a non-limiting example, the mRNA may include at least one engineered binding site to alter the binding affinity of poly-A binding protein (PABP) and analogs thereof. Additionally, multiple distinct mRNA molecules may be linked together to the PABP (poly-A binding protein) through the 3′-end using alternative nucleotides at the 3′-terminus of the poly-A region. While not wishing to be bound by theory, the poly-A region recruits PABP which in turn can interact with translation initiation complex and thus may be useful for protein synthesis.

In some aspects, the mRNA molecule of the invention may include one or more naturally occurring components, including any of the canonical nucleotides A (adenosine), G (guanosine), C (cytosine), U (uridine), or T (thymidine). Any one of the regions of a nucleic acid may include one or more alternative components (e.g., an alternative nucleoside) which impart useful properties including increased stability and/or the lack of a substantial induction of the innate immune response of a cell into which the polynucleotide is introduced. For example, a modRNA may exhibit reduced degradation in a cell into which the modRNA is introduced, relative to a corresponding unaltered mRNA. These alternative species may enhance the efficiency of protein production, intracellular retention of the polynucleotides, and/or viability of contacted cells, as well as possess reduced immunogenicity. Non-limiting examples of such non-naturally occurring modified nucleotides and nucleosides can be found, inter alia, in published patent application Nos. WO2013052523; WO2014093924; WO2015051173; WO2015051169; WO2015089511; WO2015196130; WO2015196118; WO2015196128; or WO2017153936 all of which are incorporated by reference herein.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus. The mRNA may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

The mRNA may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some aspects, the nucleobase is an alternative uracil. Exemplary nucleobases and nucleosides having an alternative uracil include pseudouridine (ψ), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (mU), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5s2U), 5-aminomethyl-2-thio-uracil (nmVu), 5-methylaminomethyl-uracil (mnm5U), 5-methylaminomethyl-2-thio-uracil (mnmVu), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnmVu), 5-propynyl-uracil, 1-propynyl-pseudouracil, 5-taurinomethyl-uracil (xm5U), 1-taurinomethyl-pseudouridine, 5-taurinomethyl-2-thio-uracil(xm5s2U), 1-taurinomethyl-4-thio-pseudouridine, 5-methyl-uracil (m5U, i.e., having the nucleobase deoxythymine), 1-methyl-pseudouridine (mlW), 5-methyl-2-thio-uracil (m5s2U), 1-methyl-4-thio-pseudouridine (m xψ), 4-thio-1-methyl-pseudouridine, 3-methyl-pseudouridine (m3ψ), 2-thio-1-methyl-pseudouridine, 1-methyl-1-deaza-pseudouridine, 2-thiol-methyl-1-deaza-pseudouridine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, Nl-methyl-pseudouridine, 3-(3-amino-3-carboxypropyl)uracil (acp U), 1-methyl-3-(3-amino-3-carboxypropyl)pseudouridine (acp ψ), 5-(isopentenylaminomethyl)uracil (inm5U), 5-(isopentenylaminomethyl)-2-thio-uracil (inm5s2U), 5,2′-O-dimethyl-uridine (m5Um), 2-thio-2′-O-methyl-uridine (s2Um), 5-methoxycarbonylmethyl-2′-O-methyl-uridine (mem Urn), 5-carbamoylmethyl-2′-O-methyl-uridine (ncm5Um), 5-carboxymethylaminomethyl-2′-O-methyl-uridine (cmnm5Um), 3,2′-O-dimethyl-uridine (mUrn), and 5-(isopentenylaminomethyl)-2′-O-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In some aspects, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-1-pseudoisocytidine, 4-thio-1-methyl-1-deaza-pseudoisocytidine, 1-methyl-1-deaza-pseudoisocytidine, zebularine, 5-aza-zebularine, 5-ethyl-1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some aspects, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methy 1-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl) adenine (io6A), 2-methylthio-N6-(cis-hydroxyisopentenyl) adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (ml Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some aspects, the nucleobase is an alternative guanine. Exemplary nucleobases and nucleosides having an alternative guanine include inosine (I), 1-methyl-inosine (mil), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQI), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (mIGm), N2,7-dimethyl-2′-O-methyl-guanosine (m2,7Gm), 2′-O-methyl-inosine (Im), 1,2′-O-dimethyl-inosine (mllm), 1-thio-guanine, and O-6-methyl-guanine. The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5-triazine. When the nucleotides are depicted using the shorthand A, G, C, T or U, each letter refers to the representative base and/or derivatives thereof, e.g., A includes adenine or adenine analogs, e.g., 7-deaza adenine).

Optionally, the mRNA can be a circular RNA, in particular a circular mRNA, especially as described in WO2014/186334 and WO2022/261490.

### Immune Cell Inhibiting Protein

The mRNA molecule as described here above encodes an immune cell inhibiting protein. Accordingly, the lipid-based nanoparticle of the invention comprises one or more different mRNA molecule encoding an immune cell inhibiting protein.

The term “immune cell inhibiting protein” refers to a protein that decreases, suppresses or dampens the activity of immune cells, in particular in a subject. These proteins typically play a role in modulating the immune response to prevent excessive inflammation or inappropriate immune reactions. Such protein can typically act by inhibiting the proliferation, activation, or function of various types of immune cells, such as T cells, B cells, natural killer cells, or antigen-presenting cells. Immune cell inhibiting proteins are known to be important for maintaining immune homeostasis and preventing autoimmunity.

In particular, the lipid-based nanoparticle may comprise 1, 2, 3, 4, 5, 6, 7, 8, 9 or 10 mRNA molecule(s) encoding 1, 2, 3, 4, 5, 6, 7 8, 9 or 10 different immune cell inhibiting protein(s), respectively. In a particular aspect, the lipid-based nanoparticle comprises mRNA molecules encoding 2 different immune cell inhibiting proteins such as disclosed herein. The immune cell inhibiting protein can be selected from a single or from different cell types. For example, the mRNA molecules may encode different T cells inhibiting proteins. Alternatively, the mRNA molecules may encode different immune cell inhibiting protein, for example for a T cell inhibiting protein and for another immune cell inhibiting protein such as a Natural killer inhibiting protein.

The immune cell inhibiting protein encoded by a mRNA molecule included in the lipid-based nanoparticle of the invention may particularly be selected based on the type of immune cell targeted by the antigen binding domain comprised in the lipid-based nanoparticle. In particular, when the immune cell targeted is an activated T-cell (i.e., so that the lipid-based nanoparticle comprises an antigen binding domain that binds to a target expressed on activated T cells such as PD-1), the mRNA encodes an activated T cell inhibiting protein.

Additionally, the immune cell inhibiting protein encoded by a mRNA molecule included in the lipid-based nanoparticle of the invention may particularly be selected based on the desired effect. For example, the immune cell inhibiting protein may be selected for a particular indication, condition, disease, or disorder.

In particular, the immune cell inhibiting protein exhibits an effect on activated immune cells selected from the group consisting of:
Inducing or increasing the exhaustion of immune cells
Inhibiting or decreasing the proliferation or renewal of immune cells
Inducing or increasing the apoptosis or cell death of immune cells
Increasing the production or concentration of mitochondrial enzymes and/or transporters of immune cells
Increasing the production or concentration of transcription factors of immune cells
Inducing or increasing hypoxia of the microenvironment or tissue
Increasing the production or concentration of metabolism enzymes of immune cells
Inducing or increasing the production of anti-inflammatory signalling molecule of immune cells
Inhibiting or decreasing the production of cytotoxic compounds by immune cells
Inducing or increasing the internalization and exocytosis of immune cells
Increasing the production or concentration of chaperone protein of immune cells
Inducing or increasing the production of cytoskeleton regulation protein of immune cells
Inhibiting or decreasing the degradation of protein through immuno-proteasome and ubiquitination, as well as the antigen presentation of immune cells
Inducing or increasing the migration and/or motility of immune cells
Inducing or increasing the inflammation resolution of the tissue
Inducing or increasing the active membrane transport of immune cells
Increasing the production or concentration of carrier protein of immune cells
Modifying the epigenetic of immune cells
Increasing the production or concentration of tRNA of immune cells,
Inducing or increasing the production and/or secretion of checkpoint inhibitors,
Inducing a phenotype switch of the immune cell, from a pro-inflammatory to an anti-inflammatory immune cell type,
Inhibiting a compound, protein or molecule that inhibits the exhaustion of immune cells, the apoptosis of immune cells, the production or concentration of mitochondrial enzymes and/or transporters of immune cells, the production or concentration of transcription factors of immune cells, the production or concentration of metabolism enzymes of immune cells, the hypoxia of the microenvironment or tissue, the efficiency of signalling pathways of immune cells, the secretion of immune cells, the internalization of endosome of immune cells, the production or concentration of chaperone protein of immune cells, the production of cytoskeleton regulation protein of immune cells, the migration and/or motility of immune cells, the active membrane transport of immune cells, the production or concentration of carrier protein of immune cells or the production or concentration of tRNA of immune cells, the production and/or secretion of checkpoint inhibitors, or the production of inflammation resolution factors; or that induces or increases the proliferation of immune cells, the production of cytotoxic compounds by immune cells or the degradation of protein through immuno-proteasome and ubiquitination, as well as the antigen presentation of immune cells.

In particular, the immune cell inhibiting protein inducing or increasing the exhaustion of immune cells is selected from the group comprising or consisting of TIM3, ENTPD1, LAG3, PD-1 and TIGIT.

In particular, the immune cell inhibiting protein inhibiting or decreasing the proliferation or renewal of immune cells is selected from the group comprising or consisting of FOXO1, MLH1, MSH2, MSH6, APC and CDKN2A.

In particular, the immune cell inhibiting protein inducing or increasing the apoptosis or cell death of immune cells is selected from the group comprising or consisting of CASPASE, FAS, TRAIL, Bax, Bak, Bok, Bad, Bid et Bim, FADD, FASL, TRAILR, TNFR, ATG5, LC3, GABARAP, GATE16, ATG 5/7/10/12, NOXA, PUMA and P53.

In particular, the immune cell inhibiting protein increasing the production or concentration of mitochondrial enzymes and/or transporters of immune cells is from the SLC25 family.

In particular, the immune cell inhibiting protein increasing the production or concentration of transcription factors of immune cells is selected from the group comprising or consisting of FOXP3, TOX, EOMES, BCL6 and BACH2. In particular, the immune cell inhibiting protein increasing the production or concentration of transcription factors of immune cells is selected from the group comprising or consisting of FOXP3, TOX, EOMES, BCL6, BACH2 SOCS, RIPK1 and any member of the STAT family.

In particular, the immune cell inhibiting protein inducing or increasing hypoxia of the microenvironment or tissue is selected from the group comprising or consisting of HIFa, PKCq and VHL.

In particular, the immune cell inhibiting protein increasing the production or concentration of metabolism enzymes of immune cells is selected from the group comprising or consisting of IDO-1, IDO-2, ARG1 and TDO.

In particular, the immune cell inhibiting protein inducing or increasing the production of anti-inflammatory signaling molecule of immune cells is selected from the group comprising or consisting of mTOR/DAPTOR/RAPTOR, SHP and SMAD.

In particular, the immune cell inhibiting protein inhibiting or decreasing the production of cytotoxic compounds by immune cells is selected from the group comprising or consisting of SHIP-1, SHP-1/2, PTEN, PTP1B, IKAROS, EGR2/3, CREM and P27 (KIP1).

In particular, the immune cell inhibiting protein inducing or increasing the internalization and exocytosis of immune cells is selected from the group comprising or consisting of CD107a, RAC1, AP2, RB7, M6P and MPR.

In particular, the immune cell inhibiting protein increasing the production or concentration of chaperone protein of immune cells is selected from the group comprising or consisting of BBS10, BBS12, TCP1 and HSP.

In particular, the immune cell inhibiting protein inducing or increasing the production of cytoskeleton regulation protein, the migration and/or motility of immune cells is selected from the group comprising or consisting of APC, Laminin, Actin, Vimentin, DEF1, Dynein and Kinesin.

In particular, the immune cell inhibiting protein inhibiting or decreasing the degradation of protein through immune-proteasome and ubiquitination, as well as the antigen presentation of immune cells is selected from the group comprising or consisting of TAPASIN, LMP7, Erp57 and Cbl-b.

In particular, the immune cell inhibiting protein inducing or increasing the inflammation resolution of the tissue is selected from the group comprising or consisting of CHEMR23, GPR37, GPR32, GPR18, FPR2 and GPR35.

In particular, the immune cell inhibiting protein modifying the epigenetic of immune cells is selected from the group comprising or consisting of tDAC, HoT, SIRT1, DOT1L, PRDME, PRMT1, PRMT3, PRMT5, PRMT6, SETD7, SETD8, SMYD2, SMYD3 and SUV420H2.

These proteins are detailed in Table E here below. In some aspects, the mRNA molecule encodes for a protein selected in Table E.

TABLE E

| | | Uniprot |
|---|---|---|
| Name | Official name | reference |
| TIM3 | Hepatitis A virus cellular receptor 2, HAVcr-2, T-cell | Q8TDQ0 |
| | immunoglobulin and mucin domain-containing protein 3, | |
| | TIMD-3, T-cell immunoglobulin mucin receptor 3, TIM-3, T- | |
| | cell membrane protein 3, CD366 | |
| ENTPD1 | Ectonucleoside triphosphate diphosphohydrolase 1, NTPDase | P49961 |
| | 1, 3.6.1.5, Ecto-ATP diphosphohydrolase 1, Ecto-ATPDase 1, | |
| | Ecto-ATPase 1, Ecto-apyrase, Lymphoid cell activation | |
| | antigen, CD39 | |
| LAG3 | Lymphocyte activation gene 3 protein, LAG-3, CD223 | P18627 |
| PD-1 | Programmed cell death protein 1, Protein PD-1, hPD-1, | Q15116 |
| | CD279 | |
| TIGIT | T-cell immunoreceptor with Ig and ITIM domains, V-set and | Q495A1 |
| | immunoglobulin domain-containing protein 9, V-set and | |
| | transmembrane domain-containing protein 3 | |
| FOXO1 | Forkhead box protein O1, Forkhead box protein O1A, | Q12778 |
| | Forkhead in rhabdomyosarcoma | |
| MLH1 | DNA mismatch repair protein Mlh1, MutL protein homolog 1 | P40692 |
| MSH2 | DNA mismatch repair protein Msh2, hMSH2, MutS protein | P43246 |
| | homolog 2 | |
| MSH6 | DNA mismatch repair protein Msh6, hMSH6, G/T mismatch- | P52701 |
| | binding protein, GTBP, GTMBP, MutS protein homolog 6, | |
| | MutS-alpha 160 kDa subunit, p160 | |
| APC | Adenomatous polyposis coli protein, Protein APC, mAPC | Q61315 |
| CDKN2A | Tumor suppressor ARF, Alternative reading frame, ARF, | Q8N726 |
| | Cyclin-dependent kinase inhibitor 2A, p14ARF | |
| FAS | Fatty acid synthase, 2.3.1.85, Type I fatty acid synthase, | P49327 |
| | [Acyl-carrier-protein] S-acetyltransferase, 2.3.1.38, [Acyl- | |
| | carrier-protein] S-malonyltransferase, 2.3.1.39, 3-oxoacyl- | |
| | [acyl-carrier-protein] synthase, 2.3.1.41, 3-oxoacyl-[acyl- | |
| | carrier-protein] reductase, 1.1.1.100, 3-hydroxyacyl-[acyl- | |
| | carrier-protein] dehydratase, 4.2.1.59, Enoyl-[acyl-carrier- | |
| | protein] reductase, 1.3.1.39, Acyl-[acyl-carrier-protein] | |
| | hydrolase, 3.1.2.14 | |
| TRAIL | Tumor necrosis factor ligand superfamily member 10, Apo-2 | P50591 |
| | ligand, Apo-2L, TNF-related apoptosis-inducing ligand, | |
| | Protein TRAIL, CD253 | |
| Bax | Apoptosis regulator BAX, Bcl-2-like protein 4, Bcl2-L-4 | Q07812 |
| Bak | Bcl-2 homologous antagonist/killer, Apoptosis regulator | Q16611 |
| | BAK, Bcl-2-like protein 7, Bcl2-L-7 | |
| Bok | Bcl-2-related ovarian killer protein, hBOK, Bcl-2-like protein | Q9UMX3 |
| | 9, Bcl2-L-9 | |
| Bad | Bcl2-associated agonist of cell death, BAD, Bcl-2-binding | Q92934 |
| | component 6, Bcl-2-like protein 8, Bcl2-L-8, Bcl-xL/Bcl-2- | |
| | associated death promoter, Bcl2 antagonist of cell death | |
| Bid | BH3-interacting domain death agonist, p22 BID, BID | P55957 |
| Bim | Bcl-2-like protein 11, Bcl2-L-11, Bcl2-interacting mediator of | 043521 |
| | cell death | |
| FADD | FAS-associated death domain protein, FAS-associating death | Q13158 |
| | domain-containing protein, Growth-inhibiting gene 3 protein, | |
| | Mediator of receptor induced toxicity | |
| FASL | Tumor necrosis factor ligand superfamily member 6, | P48023 |
| | Apoptosis antigen ligand, APTL, CD95 ligand, CD95-L, Fas | |
| | antigen ligand, Fas ligand, FasL, CD178 | |
| TRAILR1 | Tumor necrosis factor receptor superfamily member 10A, | O00220 |
| | Death receptor 4, TNF-related apoptosis-inducing ligand | |
| | receptor 1, TRAIL receptor 1, TRAIL-R1, CD261 | |
| TRAILR2 | Tumor necrosis factor receptor superfamily member 10B, | O14763 |
| | Death receptor 5, TNF-related apoptosis-inducing ligand | |
| | receptor 2, TRAIL receptor 2, TRAIL-R2, CD262 | |
| ATG5 | Autophagy protein 5, APG5-like, Apoptosis-specific protein | Q9H1Y0 |
| LC3A | Microtubule-associated proteins 1A/1B light chain 3A, | Q9H492 |
| | Autophagy-related protein LC3 A, Autophagy-related | |
| | ubiquitin-like modifier LC3 A, MAP1 light chain 3-like | |
| | protein 1, MAP1A/MAP1B light chain 3 A, MAP1A/MAP1B | |
| | LC3 A, Microtubule-associated protein 1 light chain 3 alpha | |
| LC3B | Microtubule-associated proteins 1A/1B light chain 3B, | Q9GZQ8 |
| | Autophagy-related protein LC3 B, Autophagy-related | |
| | ubiquitin-like modifier LC3 B, MAP1 light chain 3-like | |
| | protein 2, MAP1A/MAP1B light chain 3 B, MAP1A/MAP1B | |
| | LC3 B, Microtubule-associated protein 1 light chain 3 beta | |
| LC3C | Microtubule-associated proteins 1A/1B light chain 3C, | Q9BXW4 |
| | Autophagy-related protein LC3 C, Autophagy-related | |
| | ubiquitin-like modifier LC3 C, MAP1 light chain 3-like | |
| | protein 3, MAP1A/MAP1B light chain 3 C, MAP1A/MAP1B | |
| | LC3 C, Microtubule-associated protein 1 light chain 3 gamma | |
| GABARAP | Gamma-aminobutyric acid receptor-associated protein, | O95166 |
| | GABA(A) receptor-associated protein, MM46 | |
| GATE16 | Gamma-aminobutyric acid receptor-associated protein-like 2, | P60520 |
| | GABA(A) receptor-associated protein-like 2, Ganglioside | |
| | expression factor 2, GEF-2, General protein transport factor | |
| | p16, Golgi-associated ATPase enhancer of 16 kDa, GATE-16, | |
| | MAP1 light chain 3-related protein | |
| ATG 5 | Autophagy protein 5, APG5-like, Apoptosis-specific protein | Q9H1Y0 |
| ATG 7 | Ubiquitin-like modifier-activating enzyme ATG7, ATG12- | O95352 |
| | activating enzyme E1 ATG7, Autophagy-related protein 7, | |
| | APG7-like, hAGP7, Ubiquitin-activating enzyme E1-like | |
| | protein | |
| ATG 10 | Ubiquitin-like-conjugating enzyme ATG10, 2.3.2.-, | Q9H0Y0 |
| | Autophagy-related protein 10, APG10-like | |
| ATG 12 | Ubiquitin-like protein ATG12, Autophagy-related protein 12, | 094817 |
| | APG12-like | |
| NOXA | Phorbol-12-myristate-13-acetate-induced protein 1, PMA- | Q13794 |
| | induced protein 1, Immediate-early-response protein APR, | |
| | Protein Noxa | |
| PUMA | Bcl-2-binding component 3, isoforms 1/2, JFY-1, p53 up- | Q9BXH1 |
| | regulated modulator of apoptosis | Q96PG8 |
| | Bcl-2-binding component 3, isoforms 3/4, JFY-1, p53 up- | |
| | regulated modulator of apoptosis | |
| P53 | Cellular tumor antigen p53, Antigen NY-CO-13, | P04637 |
| | Phosphoprotein p53, Tumor suppressor p53 | |
| FOXP3 | Forkhead box protein P3, Scurfin | Q9BZS1 |
| TOX | Thymocyte selection-associated high mobility group box | O94900 |
| | protein TOX, Thymus high mobility group box protein TOX | |
| EOMES | Eomesodermin homolog, T-box brain protein 2, T-brain-2, | O95936 |
| | TBR-2 | |
| BCL6 | B-cell lymphoma 6 protein, BCL-6, B-cell lymphoma 5 | P41182 |
| | protein, BCL-5, Protein LAZ-3, Zinc finger and BTB domain- | |
| | containing protein 27, Zinc finger protein 51 | |
| BACH2 | Transcription regulator protein BACH2, BTB and CNC | Q9BYV9 |
| | homolog 2 | |
| HIF1a | Hypoxia-inducible factor 1-alpha, HIF-1-alpha, HIF1-alpha, | Q16665 |
| | ARNT-interacting protein, Basic-helix-loop-helix-PAS | |
| | protein MOP1, Class E basic helix-loop-helix protein 78, | |
| | bHLHe78, Member of PAS protein 1, PAS domain-containing | |
| | protein 8 | |
| HIF3a | Hypoxia-inducible factor 3-alpha, HIF-3-alpha, HIF3-alpha, | Q9Y2N7 |
| | Basic-helix-loop-helix-PAS protein MOP7, Class E basic | |
| | helix-loop-helix protein 17, bHLHe17, HIF3-alpha-1, | |
| | Inhibitory PAS domain protein, IPAS, Member of PAS protein | |
| | 7, PAS domain-containing protein 7 | |
| PKCq | Protein kinase C theta type, 2.7.11.13, nPKC-theta | Q04759 |
| VHL | von Hippel-Lindau disease tumor suppressor, Protein G7, | P40337 |
| | pVHL | |
| IDO-1 | Indoleamine 2,3-dioxygenase 1, IDO-1, 1.13.11.52, | P14902 |
| | Indoleamine-pyrrole 2,3-dioxygenase | |
| IDO-2 | Indoleamine 2,3-dioxygenase 2, IDO-2, 1.13.11.-, | Q6ZQW0 |
| | Indoleamine 2,3-dioxygenase-like protein 1, Indoleamine- | |
| | pyrrole 2,3-dioxygenase-like protein 1 | |
| ARG1 | Arginase-1, 3.5.3.1, Liver-type arginase, Type I arginase | P05089 |
| TDO | Tryptophan 2,3-dioxygenase, TDO, 1.13.11.11, Tryptamin | P48775 |
| | 2,3-dioxygenase, Tryptophan oxygenase, TO, TRPO, | |
| | Tryptophan pyrrolase, Tryptophanase | |
| mTOR/DAP | Serine/threonine-protein kinase mTOR, 2.7.11.1, FK506- | P42345 |
| TOR | binding protein 12-rapamycin complex-associated protein 1, | |
| | FKBP12-rapamycin complex-associated protein, Mammalian | |
| | target of rapamycin, mTOR, Mechanistic target of rapamycin, | |
| | Rapamycin and FKBP12 target 1, Rapamycin target protein 1 | |
| RAPTOR | Regulatory-associated protein of mTOR, Raptor, p150 target | Q8N122 |
| | of rapamycin (TOR)-scaffold protein | |
| SHP | Nuclear receptor subfamily 0 group B member 2, Orphan | Q15466 |
| | nuclear receptor SHP, Small heterodimer partner | |
| SHIP-1 | Phosphatidylinositol 3,4,5-trisphosphate 5-phosphatase 1, | Q92835 |
| | 3.1.3.86, Inositol polyphosphate-5-phosphatase D, 3.1.3.56, | |
| | Inositol polyphosphate-5-phosphatase of 145 kDa, SIP-145, | |
| | Phosphatidylinositol 4,5-bisphosphate 5-phosphatase, | |
| | 3.1.3.36, SH2 domain-containing inositol 5'-phosphatase 1, | |
| | SH2 domain-containing inositol phosphatase 1, SHIP-1, | |
| | p150Ship, hp51CN | |
| SHP-1 | Tyrosine-protein phosphatase non-receptor type 6, 3.1.3.48, | P29350 |
| | Hematopoietic cell protein-tyrosine phosphatase, Protein- | |
| | tyrosine phosphatase 1C, PTP-1C, Protein-tyrosine | |
| | phosphatase SHP-1, SH-PTP1 | |
| SHP-2 | Tyrosine-protein phosphatase non-receptor type 11, 3.1.3.48, | Q06124 |
| | Protein-tyrosine phosphatase 1D, PTP-1D, Protein-tyrosine | |
| | phosphatase 2C, PTP-2C, SH-PTP2, SHP-2, Shp2, SH-PTP3 | |
| PTEN | Phosphatidylinositol 3,4,5-trisphosphate 3-phosphatase and | P60484 |
| | dual-specificity protein phosphatase PTEN, 3.1.3.16, 3.1.3.48, | |
| | 3.1.3.67, Mutated in multiple advanced cancers 1, Phosphatase | |
| | and tensin homolog | |
| PTP1B | Tyrosine-protein phosphatase non-receptor type 1, 3.1.3.48, | P18031 |
| | Protein-tyrosine phosphatase 1B, PTP-1B | |
| IKAROS | DNA-binding protein Ikaros, Ikaros family zinc finger protein | Q13422 |
| | 1, Lymphoid transcription factor LyF-1 | |
| EGR2 | E3 SUMO-protein ligase EGR2, 2.3.2.-, AT591, E3 SUMO- | P11161 |
| | protein transferase ERG2, Early growth response protein 2, | |
| | EGR-2, Zinc finger protein Krox-20 | |
| EGR3 | Early growth response protein 3, EGR-3, Zinc finger protein | Q06889 |
| | pilot | |
| CREM | CAMP-responsive element modulator, Inducible cAMP early | Q03060 |
| | repressor, ICER | |
| P27 (KIP1) | Cyclin-dependent kinase inhibitor 1B, Cyclin-dependent | P46527 |
| | kinase inhibitor p27, p27Kip1 | |
| TRAIL | Tumor necrosis factor ligand superfamily member 10, Apo-2 | P50591 |
| | ligand, Apo-2L, TNF-related apoptosis-inducing ligand, | |
| | Protein TRAIL, CD253 | |
| CD107a | Lysosome-associated membrane glycoprotein 1, LAMP-1, | P11279 |
| | Lysosome-associated membrane protein 1, CD107 antigen- | |
| | like family member A, CD107a | |
| RAC1 | Ras-related C3 botulinum toxin substrate 1, 3.6.5.2, Cell | P63000 |
| | migration-inducing gene 5 protein, Ras-like protein TC25, | |
| | p21-Rac1 | |
| CDC42 | Cell division control protein 42 homolog, 3.6.5.2, G25K GTP- | P60953 |
| | binding protein | |
| AP2A1 | AP-2 complex subunit alpha-1, 100 kDa coated vesicle protein | O95782 |
| | A, Adaptor protein complex AP-2 subunit alpha-1, Adaptor- | |
| | related protein complex 2 subunit alpha-1, Alpha-adaptin A, | |
| | Alpha1-adaptin, Clathrin assembly protein complex 2 alpha-A | |
| | large chain, Plasma membrane adaptor HA2/AP2 adaptin | |
| | alpha A subunit | |
| AP2A2 | AP-2 complex subunit alpha-2, 100 kDa coated vesicle protein | O94973 |
| | C, Adaptor protein complex AP-2 subunit alpha-2, Adaptor- | |
| | related protein complex 2 subunit alpha-2, Alpha-adaptin C, | |
| | Alpha2-adaptin, Clathrin assembly protein complex 2 alpha-C | |
| | large chain, Huntingtin yeast partner J, Huntingtin-interacting | |
| | protein 9, HIP-9, Huntingtin-interacting protein J, Plasma | |
| | membrane adaptor HA2/AP2 adaptin alpha C subunit | |
| AP2B1 | AP-2 complex subunit beta, AP105B, Adaptor protein | P63010 |
| | complex AP-2 subunit beta, Adaptor-related protein complex | |
| | 2 subunit beta, Beta-2-adaptin, Beta-adaptin, Clathrin | |
| | assembly protein complex 2 beta large chain, Plasma | |
| | membrane adaptor HA2/AP2 adaptin beta subunit | |
| AP2M1 | AP-2 complex subunit mu, AP-2 mu chain, Adaptin-mu2, | Q96CW1 |
| | Adaptor protein complex AP-2 subunit mu, Adaptor-related | |
| | protein complex 2 subunit mu, Clathrin assembly protein | |
| | complex 2 mu medium chain, Clathrin coat assembly protein | |
| | AP50, Clathrin coat-associated protein AP50, HA2 50 kDa | |
| | subunit, Plasma membrane adaptor AP-2 50 kDa protein | |
| AP2S1 | AP-2 complex subunit sigma, Adaptor protein complex AP-2 | P53680 |
| | subunit sigma, Adaptor-related protein complex 2 subunit | |
| | sigma, Clathrin assembly protein 2 sigma small chain, Clathrin | |
| | coat assembly protein AP17, Clathrin coat-associated protein | |
| | AP17, HA2 17 kDa subunit, Plasma membrane adaptor AP-2 | |
| | 17 kDa protein, Sigma2-adaptin | |
| M6P | Cation-independent mannose-6-phosphate receptor, CI Man- | P11717 |
| | 6-P receptor, CI-MPR, M6PR, 300 kDa mannose 6-phosphate | |
| | receptor, MPR 300, Insulin-like growth factor 2 receptor, | |
| | Insulin-like growth factor II receptor, IGF-II receptor, | |
| | M6P/IGF2 receptor, M6P/IGF2R, CD222 | |
| BBS10 | Bardet-Biedl syndrome 10 protein | Q8TAM1 |
| BBS12 | Bardet-Biedl syndrome 12 protein | Q6ZW61 |
| TCP1 | T-complex protein 1 subunit alpha, TCP-1-alpha, CCT-alpha | P17987 |
| Vimentin | Vimentin | P08670 |
| DEF1 | Neutrophil defensin 1, Defensin, alpha 1, HNP-1, HP-1, HP1 | P59665 |
| TAPASIN | Tapasin, TPN, TPSN, NGS-17, TAP-associated protein, TAP- | O15533 |
| | binding protein | |
| LMP7 | Proteasome subunit beta type-8, 3.4.25.1, Low molecular mass | P28062 |
| | protein 7, Macropain subunit C13, Multicatalytic | |
| | endopeptidase complex subunit C13, Proteasome component | |
| | C13, Proteasome subunit beta-5i, Really interesting new gene | |
| | 10 protein | |
| ERp57 | Protein disulfide-isomerase A3, 5.3.4.1, 58 kDa glucose- | P30101 |
| | regulated protein, 58 kDa microsomal protein, p58, Disulfide | |
| | isomerase ER-60, Endoplasmic reticulum resident protein 57, | |
| | ER protein 57, ERp57, Endoplasmic reticulum resident protein | |
| | 60, ER protein 60, ERp60 | |
| Cbl-b | E3 ubiquitin-protein ligase CBL-B, 2.3.2.27, Casitas B- | Q13191 |
| | lineage lymphoma proto-oncogene b, RING finger protein 56, | |
| | RING-type E3 ubiquitin transferase CBL-B, SH3-binding | |
| | protein CBL-B, Signal transduction protein CBL-B | |
| CHEMR23 | Chemerin-like receptor 1, Chemokine-like receptor 1, G- | Q99788 |
| | protein coupled receptor ChemR23, G-protein coupled | |
| | receptor DEZ | |
| GPR37 | Prosaposin receptor GPR37, Endothelin B receptor-like | O15354 |
| | protein 1, ETBR-LP-1, G-protein coupled receptor 37, Parkin- | |
| | associated endothelin receptor-like receptor, PAELR | |
| GPR32 | Probable G-protein coupled receptor 32 | O75388 |
| GPR18 | N-arachidonyl glycine receptor, NAGly receptor, G-protein | Q14330 |
| | coupled receptor 18 | |
| FPR2 | N-formyl peptide receptor 2, FMLP-related receptor I, FMLP- | P25090 |
| | R-I, Formyl peptide receptor-like 1, HM63, Lipoxin A4 | |
| | receptor, LXA4 receptor, RFP | |
| GPR35 | G-protein coupled receptor 35, Kynurenic acid receptor, | Q9HC97 |
| | KYNA receptor | |
| HMT | Histone methyltransferase | CAS |
| | | no.9055- |
| | | 08-7 |
| SIRT1 | NAD-dependent protein deacetylase sirtuin-1, hSIRT1, | Q96EB6 |
| | 2.3.1.286, NAD-dependent protein deacylase sirtuin-1, 2.3.1.-, | |
| | Regulatory protein SIR2 homolog 1, SIR2-like protein 1, | |
| | hSIR2 | |
| DOTIL | Histone-lysine N-methyltransferase, H3 lysine-79 specific, | Q8TEK3 |
| | 2.1.1.360, DOT1-like protein, Histone H3-K79 | |
| | methyltransferase, H3-K79-HMTase, Lysine N- | |
| | methyltransferase 4 | |
| PRMT1 | Protein arginine N-methyltransferase 1, 2.1.1.319, Histone- | Q99873 |
| | arginine N-methyltransferase PRMT1, Interferon receptor 1- | |
| | bound protein 4 | |
| PRMT3 | Protein arginine N-methyltransferase 3, 2.1.1.319, | O60678 |
| | Heterogeneous nuclear ribonucleoprotein methyltransferase- | |
| | like protein 3 | |
| PRMT5 | Protein arginine N-methyltransferase 5, PRMT5, 2.1.1.320, 72 | O14744 |
| | kDa ICln-binding protein, Histone-arginine N- | |
| | methyltransferase PRMT5, Jak-binding protein 1, Shk1 | |
| | kinase-binding protein 1 homolog, SKB1 homolog, SKB1Hs | |
| PRMT6 | Protein arginine N-methyltransferase 6, 2.1.1.319, | Q96LA8 |
| | Heterogeneous nuclear ribonucleoprotein methyltransferase- | |
| | like protein 6, Histone-arginine N-methyltransferase PRMT6 | |
| SETD7 | Histone-lysine N-methyltransferase SETD7, 2.1.1.364, | Q8WTS6 |
| | Histone H3-K4 methyltransferase SETD7, H3-K4-HMTase | |
| | SETD7, Lysine N-methyltransferase 7, SET domain- | |
| | containing protein 7, SET7/9 | |
| SETD8 | [histone H4]-lysine(20) N-methyltransferase | E3VVS3 |
| SMYD2 | N-lysine methyltransferase SMYD2, 2.1.1.-, HSKM-B, | Q9NRG4 |
| | Histone methyltransferase SMYD2, 2.1.1.354, Lysine N- | |
| | methyltransferase 3C, SET and MYND domain-containing | |
| | protein 2 | |
| SMYD3 | Histone-lysine N-methyltransferase SMYD3, 2.1.1.354, SET | Q9H7B4 |
| | and MYND domain-containing protein 3, Zinc finger MYND | |
| | domain-containing protein 1 | |
| SUV420H2 | Histone-lysine N-methyltransferase KMT5C, Lysine N- | Q86Y97 |
| | methyltransferase 5C, Lysine-specific methyltransferase 5C, | |
| | Suppressor of variegation 4-20 homolog 2, Su(var)4-20 | |
| | homolog 2, Suv4-20h2, [histone H4]-N-methyl-L-lysine20 N- | |
| | methyltransferase KMT5B, 2.1.1.362, [histone H4]-lysine20 | |
| | N-methyltransferase KMT5B, 2.1.1.361 | |
| IL-12α | Interleukin-12 subunit alpha, IL-12A, Cytotoxic lymphocyte | P29459 |
| | maturation factor 35 kDa subunit, CLMF p35, IL-12 subunit | |
| | p35, NK cell stimulatory factor chain 1, NKSF1 | |
| IL-27β | Interleukin-27 subunit beta, IL-27 subunit beta, IL-27B, | Q14213 |
| | Epstein-Barr virus-induced gene 3 protein, EBV-induced gene | |
| | 3 protein | |
| IL37 | Interleukin-37, IL-37, FIL1 zeta, IL-1X, Interleukin-1 family | Q9NZH6 |
| | member 7, IL-1F7, Interleukin-1 homolog 4, IL-1H, IL-1H4, | |
| | Interleukin-1 zeta, IL-1 zeta, Interleukin-1-related protein, IL- | |
| | 1RP1 | |
| IL38 | Interleukin-1 family member 10, IL-1F10, Family of | Q8WWZ1 |
| | interleukin 1-theta, FILI theta, Interleukin-1 HY2, IL-1HY2, | |
| | Interleukin-1 theta, IL-1 theta, Interleukin-38, IL-38 | |
| SOCS1 | Suppressor of cytokine signaling 1, SOCS-1, JAK-binding | O15524 |
| | protein, JAB, STAT-induced STAT inhibitor 1, SSI-1, Tec- | |
| | interacting protein 3, TIP-3 | |
| SOCS2 | Suppressor of cytokine signaling 2, SOCS-2, Cytokine- | O14508 |
| | inducible SH2 protein 2, CIS-2, STAT-induced STAT | |
| | inhibitor 2, SSI-2 | |
| SOCS3 | Suppressor of cytokine signaling 3, SOCS-3, Cytokine- | O14543 |
| | inducible SH2 protein 3, CIS-3, STAT-induced STAT | |
| | inhibitor 3, SSI-3 | |
| SOCS4 | Suppressor of cytokine signaling 4, SOCS-4, Suppressor of | Q8WXH5 |
| | cytokine signaling 7, SOCS-7 | |
| SOCS5 | Suppressor of cytokine signaling 5, SOCS-5, Cytokine- | O75159 |
| | inducible SH2 protein 6, CIS-6, Cytokine-inducible SH2- | |
| | containing protein 5 | |
| SOCS6 | Suppressor of cytokine signaling 6, SOCS-6, Cytokine- | O14544 |
| | inducible SH2 protein 4, CIS-4, Suppressor of cytokine | |
| | signaling 4, SOCS-4 | |
| SOCS7 | Suppressor of cytokine signaling 7, SOCS-7, Nck, Ash and | O14512 |
| | phospholipase C gamma-binding protein, Nck-associated | |
| | protein 4, NAP-4 | |
| RIPK1 | Receptor-interacting serine/threonine-protein kinase 1, | Q13546 |
| | 2.7.11.1, Cell death protein RIP, Receptor-interacting protein | |
| | 1, RIP-1 | |
| STAT1 | Signal transducer and activator of transcription 1-alpha/beta, | P42224 |
| | Transcription factor ISGF-3 components p91/p84 | |
| STAT2 | Signal transducer and activator of transcription 2, p113 | P52630 |
| STAT3 | Signal transducer and activator of transcription 3, Acute-phase | P40763 |
| | response factor | |
| STAT4 | Signal transducer and activator of transcription 4 | Q14765 |
| STAT5A | Signal transducer and activator of transcription 5A | P42229 |
| STAT5B | Signal transducer and activator of transcription 5B | P51692 |
| STAT6 | Signal transducer and activator of transcription 6, IL-4 Stat | P42226 |

The one or several mRNA molecule(s) encode(s) for a molecule selected from the group consisting of:
a) a protein selected from the group consisting of TIM3, ENTPD1, LAG3, PD-1, PD-L1, TIGIT, FOXO1, MLH1, MSH2, MSH6, APC, CDKN2A, CASPASE, FAS, TRAIL, Bax, Bak, Bok, Bad, Bid et Bim, BIM, BIM-S, FADD, FASL, TRAILR, TNFR, ATG5, LC3, GABARAP, GATE16, ATG 5/7/10/12, NOXA, PUMA, P53, FOXP3, TOX, EOMES, BCL6 and BACH2, HIFa, PKCq and VHL, IDO-1, IDO-2, ARG1; TDO, mTOR/DAPTOR/RAPTOR, SHP, SMAD, SHIP-1, SHP-1/2, PTEN, PTP1B, IKAROS, EGR2/3, CREM, P27 (KIP1), CD107a, RAC1, AP2, RB7, M6P, MPR, BBS10, BBS12, TCP1, HSP, APC, Laminin, Actin, Vimentin, DEF1, Dynein, Kinesin, TAPASIN, LMP7, Erp57, Cbl-b, CHEMR23, GPR37, GPR32, GPR18, FPR2, GPR35, HDAC, HMT, SIRT1, DOT1L, PRDME, PRMT1, PRMT3, PRMT5, PRMT6, SETD7, SETD8, SMYD2, SMYD3 or SUV420H2, preferably selected from the group consisting of TIM3, LAG3, PD-L1, TIGIT, FAS, TRAIL, PUMA, BIM-S, FOXP3, TOX, IDO-1, IDO-2, ARG1, CHEMR23 and FPR2, and any combination thereof,
b) a cytokine, preferably selected from the group consisting of IFNG, IFNa, IL2, IL13, IL4 and IL-10, IL35 (dimeric protein comprising IL-12a and IL-270 chains), IL37, IL38 and any combination thereof,
c) a cytokine receptor, preferably selected from the group consisting of IL-1R, IL-4R, IL-6R, IL-8R, IL-10R, IL-11R, IL-17R, IL-18R, IL-21R, IL-23R, IL-35R, and any combination thereof, and
d) a chemokine receptor, preferably selected from the group consisting of CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6 and XCR1, and any combination thereof.

In some aspects, the one or several mRNA molecule(s) encode(s) for TIM3, ENTPD1, LAG3, PD-1, PD-L1, TIGIT, FOXO1, MLH1, MSH2, MSH6, APC, CDKN2A, CASPASE, FAS, TRAIL, Bax, Bak, Bok, Bad, Bid et Bim, BIM, BIM-S, FADD, FASL, TRAILR, TNFR, ATG5, LC3, GABARAP, GATE16, ATG 5/7/10/12, NOXA, PUMA, P53, FOXP3, TOX, EOMES, BCL6 and BACH2, HIFa, PKCq and VHL, IDO-1, IDO-2, ARG1; TDO, mTOR/DAPTOR/RAPTOR, SHP, SMAD, SHIP-1, SHP-1/2, PTEN, PTP1B, IKAROS, EGR2/3, CREM, P27 (KIP1), CD107a, RAC1, AP2, RB7, M6P, MPR, BBS10, BBS12, TCP1, HSP, APC, Laminin, Actin, Vimentin, DEF1, Dynein, Kinesin, TAPASIN, LMP7, Erp57, Cbl-b, CHEMR23, GPR37, GPR32, GPR18, FPR2, GPR35, HDAC, HMT, SIRT1, DOT1L, PRDME, PRMT1, PRMT3, PRMT5, PRMT6, SETD7, SETD8, SMYD2, SMYD3 or SUV420H2, or any combination thereof.

Preferably, the one or several mRNA molecule(s) encode(s) for a protein selected from the group consisting of TIM3, LAG3, PD-L1, TIGIT, FAS, TRAIL, PUMA, FOXP3, TOX, IDO-1, IDO-2, ARG1, CHEMR23, FPR2 or BIM-S or any combination thereof.

In some aspects, the mRNA molecule encodes for BIM-S and/or PUMA.

Preferably, the mRNA molecule encoding for PUMA comprises or consists of a nucleic acid sequence as set forth in SEQ ID NO: 43, or of a nucleic acid sequence having at least 80%,85%, 90%, 95% or 97% sequence identity thereto.

In some aspects, the mRNA molecule encodes for a BIM protein. As used herein, the term “BIM protein” encompasses the three Bim isoforms (i.e., Bim-EL, Bim-L and Bim-S, e.g., such as described in Sionov R. Vogt, Vlahopoulos S. A., Granot Z. Regulation of Bim in Health and Disease. Oncotarget. 2015; 6: 23058-23134.) that are generated by alternative splicing. Preferably, the mRNA molecule encoding for BIM-S comprises or consists of a nucleic acid sequence as set forth in SEQ ID NO: 44 or of a nucleic acid sequence having at least 80%, 85%, 90%, 95% or 97% sequence identity thereto.

The effect of an immune cell inhibiting protein on the activated immune cells may be determined when, in presence of the immune cell inhibiting protein of the invention, activated immune cells exhibits a lower activity compared to activated immune cells under the same experimental conditions but without the presence of the immune cell inhibiting protein. Said sample may be an immune cell culture, from a sample of at least one healthy patient or from a sample of at least one patient in need of a treatment as detailed hereunder. Immune cell activity can be measured by any method known to the person skilled in the art. In particular, the inhibition of immune cell activity may be measured by comparing the immune cell activity of a population of immune cells obtained from a sample, without the immune cell inhibiting protein to be assessed, to the immune cell activity of a population of immune cells obtained from a sample and treated with the immune cell inhibiting protein to be assessed.

In an aspect, the immune cell inhibiting protein is an intracellular protein having an intracellular effect on the activated immune cell or a transmembrane protein, preferably an intracellular protein.

Use of intracellular proteins, in combination with the targeting of a specific subset of immune cells, results in a very specific and efficient inhibition of said immune cells. Potent intracellular proteins may be used, with an elevated activation and/or proliferation of immune cells in a specific environment.

As used herein, a “protein that have an intracellular effect on the activated immune cell” or an “intracellular protein” refers to a protein which is produced/expressed inside a cell and which does not enter the extracellular medium, either alone or in a vesicle, nor that is expressed in the cellular membrane. The intracellular protein is thus contained in the boundaries of the cellular membrane, and acts in one of the cellular compartments (e.g. cytosol, endoplasmic reticulum, mitochondria, nucleus, etc.,). Such protein can be present in any of the cellular compartments, such as nucleus, intercisternal space, organelles or cytosol. Accordingly, the protein can be a cytoplasmic protein, a nuclear protein or a mitochondrial protein, or an intercisternal protein, preferably nuclear or cytoplasmic protein.

Optionally, the immune cell intracellular protein can be an enzyme, an intracellular signalling protein or a transcription factor, preferably a transcription factor.

In a particular aspect, the immune cell inhibiting protein is a transcription factor. As used herein, a “transcription factor” refers to a DNA-binding protein that regulates the transcription of a gene. Preferably, the transcription factor is selected from the group consisting of SOCS family member, STAT family member, and RIPK1, and a variant thereof having at least 80% of identity with the wildtype protein or having 1 to 10 modifications selected from the group consisting of addition, deletion, substitution and combinations thereof.

In a specific aspect, the immune cell inhibiting protein is not a chimeric antigen receptor (CAR). In an aspect, the immune cell activating protein is not a CAR, a T cell receptor (TcR) (i.e., including TCR alpha, TCR beta, CD3 and CD247) or a B cell receptor (BcR).

In some aspects, the immune cell compound or molecule is a secreted protein.

As used herein, “secreted compound” or “secreted protein” or “secreted molecule” refers to a compound which exits the cell into the extracellular medium, is addressed to the membrane of the immune cell or is directed to an adjacent cell.

In an aspect, the secreted compound or molecule does not act on the immune cell which secreted it. In an aspect, the secreted compound or molecule acts on an additional cell as well as on the immune cell which secreted it.

In some aspects, the one or more mRNA molecule(s) does not encode a cytokine and/or a chemokine. In a specific aspect, the immune cell inhibiting protein is not a cytokine and/or a chemokine.

In a particular aspect, the transmembrane protein is selected from the group comprising CCR4, CCR10, CXCR3, CCR10, CCR5, CCR2, CX3CR1, CCR7, CXCR4, CXCR3, CXCR5, CCR7, CXCR5.

Alternatively, the immune cell inhibiting protein is an intracellular signalling protein. For example, such intracellular signalling molecules can be D3-phosphoinositides and derivatives of phosphatidylinositol such as phosphorylated at the D-3 position of the inositol ring and encompasses the compounds phosphatidylinositol-(3)-monophosphate (PtdIns(3)P), phosphatidylinositol(3,4)-bisphosphate (PtdIns(3,4)P2), and phosphatidylinositol(3,4,5)-trisphosphate (PtdIns(3,4,5)P3). Therefore, the intracellular signalling protein can be those involved in the synthesis of these molecules.

In a specific aspect, the immune cell inhibiting protein is not a chimeric antigen receptor (CAR). Preferably, the immune cell inhibiting protein is not a CAR, a T cell receptor (TcR) (i.e., including TCR alpha, TCR beta, CD3 and CD247) or a B cell receptor (BcR).

In some aspects, the one or more mRNA molecule(s) does not encode a cytokine and/or a chemokine.

In a particular aspect, the immune cell inhibiting protein is a cytokine receptor. Preferably, the cytokine receptor is selected from the group consisting of IL10R, TGFR, TNFR, ILR1A, GCSFR and IL4R.

In a particular aspect, the immune cell inhibiting protein is a chemokine receptor. Preferably, the chemokine receptor is selected from the group consisting of CXCR3, CXCR5, CXCR4, CXCR1, CXCR2, CXCR6, CCR1, CCR2, CCR3, CCR4, CCR5, CCR6, CCR7, CCR8, CCR9, CCR10, CCR11, CX3CR1 and XCR1.

In a particular aspect, the immune cell inhibiting protein is a lectin receptor. Preferably, the lectin receptor is selected from the group consisting of CD72, DCIR, MICL and CLEC-1.

In a particular aspect, the immune cell inhibiting protein is an engineered surface receptor or anchored membrane cytokine. Preferably, the engineered surface receptor or anchored membrane cytokine is selected from the group consisting of Membrane engineered cytokine (TGFB, IL13, IL4, IL-10), Dominant negative TLR, Dominant negative receptor (IL-6R, TNFR, IL17R, IL23R, IL35R, IL21R, IFNa R).

In a particular aspect, the immune cell inhibiting protein is a coinhibitory receptor or ligand.

Preferably, the coinhibitory receptor or ligand is selected from the group consisting of BTLA, CTLA4, B7H1, B7H3, B7H4, PD1H, LAIR1, TIM1, TIM3, TIM4, 2B4, PD-1, PDL1, PDL2, galectin9, CD48, HVEM, B7DC, CD155, CD113, CD112, ILT2, ILT3, NKG2A, LILRB1, IRp60, KIR, CD22, CD5, CD66a, PIR-B, KIR2DL1, KIR2DL2/3, ILT4, KIR2DL5, KIR3DL1, KIR3DL2, KIR3DL3, TIGIT, LAG3 and A2AR.

These proteins are detailed in Table F here below. In some aspects, the mRNa molecule encodes for a protein selected in Table F.

TABLE F

| | | Uniprot |
|---|---|---|
| Name | Official name | reference |
| TGFR1 | TGF-beta receptor type-1, TGFR-1, 2.7.11.30, Activin A | P36897 |
| | receptor type II-like protein kinase of 53 kD, Activin | |
| | receptor-like kinase 5, ALK-5, ALK5, Serine/threonine- | |
| | protein kinase receptor R4, SKR4, TGF-beta type I receptor, | |
| | Transforming growth factor-beta receptor type I, TGF-beta | |
| | receptor type I, TbetaR-I | |
| TGFR2 | TGF-beta receptor type-2, TGFR-2, 2.7.11.30, TGF-beta | P37173 |
| | type II receptor, Transforming growth factor-beta receptor | |
| | type II, TGF-beta receptor type II, TbetaR-II | |
| GCSFR | Granulocyte colony-stimulating factor receptor, G-CSF | Q99062 |
| | receptor, G-CSF-R, CD114 | |
| IL13 | Interleukin-13, IL-13 | P35225 |
| IL4 | Interleukin-4, IL-4, B-cell stimulatory factor 1, BSF-1, | P05112 |
| | Binetrakin, Lymphocyte stimulatory factor 1, Pitrakinra | |
| IL10 | Interleukin-10, IL-10, Cytokine synthesis inhibitory factor, | P22301 |
| | CSIF | |
| IL17R | Interleukin-17 receptor A, IL-17 receptor A, IL-17RA, | Q96F46 |
| | CDw217, CD217 | |
| IFNa R | Interferon alpha/beta receptor 1, IFN-R-1, IFN-alpha/beta | P17181 |
| | receptor 1, Cytokine receptor class-II member 1, Cytokine | |
| | receptor family 2 member 1, CRF2-1, Type I interferon | |
| | receptor 1 | |
| IL21R | Interleukin-21 receptor, IL-21 receptor, IL-21R, Novel | Q9HBE5 |
| | interleukin receptor, CD360 | |
| IL11R | Interleukin-11 receptor subunit alpha, IL-11 receptor | Q14626 |
| | subunit alpha, IL-11R subunit alpha, IL-11R-alpha, IL- | |
| | 11RA | |
| IL23R | Interleukin-23 receptor, IL-23 receptor, IL-23R | Q5VWK5 |
| IL10R | Interleukin-10 receptor subunit alpha, IL-10 receptor | Q13651 |
| | subunit alpha, IL-10R subunit alpha, IL-10RA, CDw210a, | |
| | Interleukin-10 receptor subunit 1, IL-10R subunit 1, IL- | |
| | 10R1, CD210 | |
| ILIR | Interleukin-1 receptor type 1, IL-1R-1, IL-1RT-1, IL-1RT1, | P14778 |
| | 3.2.2.6, CD121 antigen-like family member A, Interleukin- | |
| | 1 receptor alpha, IL-1R-alpha, Interleukin-1 receptor type I, | |
| | p80, CD121a | |
| CXCR3 | C-X-C chemokine receptor type 3, CXC-R3, CXCR-3, | P49682 |
| | CKR-L2, G protein-coupled receptor 9, Interferon-inducible | |
| | protein 10 receptor, IP-10 receptor, CD183 | |
| CXCR5 | C-X-C chemokine receptor type 5, CXC-R5, CXCR-5, | P32302 |
| | Burkitt lymphoma receptor 1, Monocyte-derived receptor | |
| | 15, MDR-15, CD185 | |
| CXCR4 | C-X-C chemokine receptor type 4, CXC-R4, CXCR-4, | P61073 |
| | FB22, Fusin, HM89, LCR1, Leukocyte-derived seven | |
| | transmembrane domain receptor, LESTR, | |
| | Lipopolysaccharide-associated protein 3, LAP-3, LPS- | |
| | associated protein 3, NPYRL, Stromal cell-derived factor 1 | |
| | receptor, SDF-1 receptor, CD184 | |
| CXCR1 | C-X-C chemokine receptor type 1, CXC-R1, CXCR-1, | P25024 |
| | CDw128a, High affinity interleukin-8 receptor A, IL-8R A, | |
| | IL-8 receptor type 1, CD181 | |
| CXCR2 | C-X-C chemokine receptor type 2, CXC-R2, CXCR-2, | P25025 |
| | CDw128b, GRO/MGSA receptor, High affinity interleukin- | |
| | 8 receptor B, IL-8R B, IL-8 receptor type 2, CD182 | |
| CXCR6 | C-X-C chemokine receptor type 6, CXC-R6, CXCR-6, | O00574 |
| | CDw186, G-protein coupled receptor STRL33, G-protein | |
| | coupled receptor bonzo, CD186 | |
| CCR1 | C-C chemokine receptor type 1, C-C CKR-1, CC-CKR-1, | P32246 |
| | CCR-1, CCR1, HM145, LD78 receptor, Macrophage | |
| | inflammatory protein 1-alpha receptor, MIP-1alpha-R, | |
| | RANTES-R, CD191 | |
| CCR2 | C-C chemokine receptor type 2, C-C CKR-2, CC-CKR-2, | P41597 |
| | CCR-2, CCR2, Monocyte chemoattractant protein 1 | |
| | receptor, MCP-1-R, CD192 | |
| CCR3 | C-C chemokine receptor type 3, C C CKR3, C-C CKR-3, | P51677 |
| | CC-CKR-3, CCR-3, CCR3, CKR 3, CKR3, Eosinophil | |
| | eotaxin receptor, CD193 | |
| CCR4 | C-C chemokine receptor type 4, C-C CKR-4, CC-CKR-4, | P51679 |
| | CCR-4, CCR4, K5-5, CD194 | |
| CCR5 | C-C chemokine receptor type 5, C-C CKR-5, CC-CKR-5, | P51681 |
| | CCR-5, CCR5, CHEMR13, HIV-1 fusion coreceptor, | |
| | CD195 | |
| CCR6 | C-C chemokine receptor type 6, C-C CKR-6, CC-CKR-6, | P51684 |
| | CCR-6, Chemokine receptor-like 3, CKR-L3, DRY6, G- | |
| | protein coupled receptor 29, GPR-CY4, GPRCY4, LARC | |
| | receptor, CD196 | |
| CCR7 | C-C chemokine receptor type 7, C-C CKR-7, CC-CKR-7, | P32248 |
| | CCR-7, BLR2, CDw197, Epstein-Barr virus-induced G- | |
| | protein coupled receptor 1, EBI1, EBV-induced G-protein | |
| | coupled receptor 1, MIP-3 beta receptor, CD197 | |
| CCR8 | C-C chemokine receptor type 8, C-C CKR-8, CC-CKR-8, | P51685 |
| | CCR-8, CC chemokine receptor CHEMR1, CMKBRL2, | |
| | Chemokine receptor-like 1, CKR-L1, GPR-CY6, GPRCY6, | |
| | TER1, CDw198 | |
| CCR9 | C-C chemokine receptor type 9, C-C CKR-9, CC-CKR-9, | P51686 |
| | CCR-9, G-protein coupled receptor 28, GPR-9-6, CDw199 | |
| CCR10 | C-C chemokine receptor type 10, C-C CKR-10, CC-CKR- | P46092 |
| | 10, CCR-10, G-protein coupled receptor 2 | |
| CCR11 | C-C chemokine receptor-like 2, Chemokine receptor | O00421 |
| | CCR11, Chemokine receptor X, Putative MCP-1 chemokine | |
| | receptor | |
| CX3CR1 | CX3C chemokine receptor 1, C-X3-C CKR-1, CX3CR1, | P49238 |
| | Beta chemokine receptor-like 1, CMK-BRL-1, CMK- | |
| | BRL1, Fractalkine receptor, G-protein coupled receptor 13, | |
| | V28 | |
| XCR1 | Chemokine XC receptor 1, G-protein coupled receptor 5, | P46094 |
| | Lymphotactin receptor, XC chemokine receptor 1 | |
| CD72 | B-cell differentiation antigen CD72, Lyb-2, CD72 | P21854 |
| DCIR | C-type lectin domain family 4 member A, C-type lectin | Q9UMR7 |
| | DDB27, C-type lectin superfamily member 6, Dendritic cell | |
| | immunoreceptor, Lectin-like immunoreceptor, CD367 | |
| MICL | C-type lectin domain family 12 member A, C-type lectin- | Q5QGZ9 |
| | like molecule 1, CLL-1, Dendritic cell-associated lectin 2, | |
| | DCAL-2, Myeloid inhibitory C-type lectin-like receptor, | |
| | MICL, CD371 | |
| TIM1 | Protein timeless homolog, hTIM | Q9UNS1 |
| TIM4 | T-cell immunoglobulin and mucin domain-containing | Q96H15 |
| | protein 4, TIMD-4, T-cell immunoglobulin mucin receptor | |
| | 4, TIM-4, T-cell membrane protein 4 | |
| galectin9 | Galectin-9, Gal-9, Ecalectin, Tumor antigen HOM-HD-21 | O00182 |
| ILT2 | Leukocyte immunoglobulin-like receptor subfamily B | Q8NHL6 |
| | member 1, LIR-1, Leukocyte immunoglobulin-like receptor | |
| | 1, CD85 antigen-like family member J, Immunoglobulin- | |
| | like transcript 2, ILT-2, Monocyte/macrophage | |
| | immunoglobulin-like receptor 7, MIR-7, CD85j | |
| ILT3 | Leukocyte immunoglobulin-like receptor subfamily B | Q8NHJ6 |
| | member 4, B4, CD85 antigen-like family member K, | |
| | Immunoglobulin-like transcript 3, ILT-3, Leukocyte | |
| | immunoglobulin-like receptor 5, LIR-5, Monocyte | |
| | inhibitory receptor HM18, CD85k | |
| CD5 | T-cell surface glycoprotein CD5, Lymphocyte antigen | P06127 |
| | T1/Leu-1, CD5 | |
| KIR2DL1 | Killer cell immunoglobulin-like receptor 2DL1, CD158 | P43626 |
| | antigen-like family member A, Natural killer-associated | |
| | transcript 1, NKAT-1, p58 natural killer cell receptor clones | |
| | CL-42/47.11, p58 NK receptor CL-42/47.11, p58.1 MHC | |
| | class-I-specific NK receptor, CD158a | |
| KIR2DL3 | Killer cell immunoglobulin-like receptor 2DL3, CD158 | P43628 |
| | antigen-like family member B2, KIR-023GB, Killer | |
| | inhibitory receptor cl 2-3, NKAT2a, NKAT2b, Natural | |
| | killer-associated transcript 2, NKAT-2, p58 natural killer | |
| | cell receptor clone CL-6, p58 NK receptor CL-6, p58.2 | |
| | MHC class-I-specific NK receptor, CD158b2 | |
| KIR2DL5A | Killer cell immunoglobulin-like receptor 2DL5A, CD158f1 | Q8N109 |
| KIR2DL5B | Killer cell immunoglobulin-like receptor 2DL5B, CD158 | Q8NHK3 |
| | antigen-like family member F2, Killer cell immunoglobulin- | |
| | like receptor 2DLX, CD158f2 | |
| KIR3DL1 | Killer cell immunoglobulin-like receptor 3DL1, CD158 | P43629 |
| | antigen-like family member E, HLA-BW4-specific | |
| | inhibitory NK cell receptor, Natural killer-associated | |
| | transcript 3, NKAT-3, p70 natural killer cell receptor clones | |
| | CL-2/CL-11, p70 NK receptor CL-2/CL-11, CD158e | |
| KIR3DL2 | Killer cell immunoglobulin-like receptor 3DL2, CD158 | P43630 |
| | antigen-like family member K, Natural killer-associated | |
| | transcript 4, NKAT-4, p70 natural killer cell receptor clone | |
| | CL-5, p70 NK receptor CL-5, CD158k | |
| KIR3DL3 | Killer cell immunoglobulin-like receptor 3DL3, CD158 | Q8N743 |
| | antigen-like family member Z, Killer cell inhibitory receptor | |
| | 1, CD158z | |
| A2AR | Adenosine receptor A2a | P29274 |
| B7H3 | CD276 antigen, 4Ig-B7-H3, B7 homolog 3, B7-H3, | Q5ZPR3 |
| | Costimulatory molecule, CD276 | |
| B7H4 | V-set domain-containing T-cell activation inhibitor 1, B7 | Q7Z7D3 |
| | homolog 4, B7-H4, B7h.5, Immune costimulatory protein | |
| | B7-H4, Protein B7S1, T-cell costimulatory molecule B7x | |
| PD1H | V-type immunoglobulin domain-containing suppressor of | Q9H7M9 |
| | T-cell activation, Platelet receptor Gi24, Stress-induced | |
| | secreted protein-1, Sisp-1, V-set domain-containing | |
| | immunoregulatory receptor, V-set immunoregulatory | |
| | receptor | |
| LAIR1 | Leukocyte-associated immunoglobulin-like receptor 1, | Q6GTX8 |
| | LAIR-1, hLAIR1, CD305 | |
| PDL1 | Programmed cell death 1 ligand 1, PD-L1, PDCD1 ligand 1, | Q9NZQ7 |
| | Programmed death ligand 1, hPD-L1, B7 homolog 1, B7- | |
| | H1, CD274 | |
| PDL2 | Programmed cell death 1 ligand 2, PD-1 ligand 2, PD-L2, | Q9BQ51 |
| | PDCD1 ligand 2, Programmed death ligand 2, Butyrophilin | |
| | B7-DC, B7-DC, CD273 | |
| CD155 | Poliovirus receptor, Nectin-like protein 5, NECL-5, CD155 | P15151 |
| CD113 | Nectin-3, CDw113, Nectin cell adhesion molecule 3, | Q9NQS3 |
| | Poliovirus receptor-related protein 3, CD113 | |
| CD112 | Nectin-2, Herpes virus entry mediator B, Herpesvirus entry | Q92692 |
| | mediator B, HveB, Nectin cell adhesion molecule 2, | |
| | Poliovirus receptor-related protein 2, CD112 | |
| IRp60 | CMRF35-like molecule 8, CLM-8, CD300 antigen-like | Q9UGN4 |
| | family member A, CMRF-35-H9, CMRF35-H9, CMRF35- | |
| | H, IRC1/IRC2, Immunoglobulin superfamily member 12, | |
| | IgSF12, Inhibitory receptor protein 60, IRp60, NK | |
| | inhibitory receptor, CD300a | |
| KIR | GTP-binding protein GEM, GTP-binding mitogen-induced | P55040 |
| | T-cell protein, RAS-like protein KIR | |
| CD66a | Carcinoembryonic antigen-related cell adhesion molecule 1, | P13688 |
| | Biliary glycoprotein 1, BGP-1, CD66a | |
| PIR-B | Paired immunoglobulin-like type 2 receptor beta, Activating | Q9UKJ0 |
| | receptor PILR-beta, Cell surface receptor FDFACT | |

In a particular aspect, the one or several mRNA molecule(s) encode(s) for CCR4, CCR10, CXCR3, CCR5, CCR2, CX3CR1, CCR7, CXCR4, CXCR5, IL10R, TGFR, TNFR, ILR1A, GCSFR, IL4R, CXCR1, CXCR2, CXCR6, CCR1, CCR3, CCR6, CCR8, CCR9, CCR11, XCR1, CD72, DCIR, MICL, CLEC-1, TGFB, IL13, IL4, IL-10, IL-6R, IL17R, IL23R, TL35R, IL21R, IFNa R, BTLA, CTLA4, B7H1, B7H3, B7H4, PD1H, LAIR1, TIM1, TIM3, TIM4, 2B4, PD-1, PDL1, PDL2, galectin9, CD48, HVEM, B7DC, CD155, CD113, CD112, ILT2, ILT3, NKG2A, LILRB1, IRp60, KIR, CD22, CD5, CD66a, PIR-B, KIR2DL1, KIR2DL2/3, ILT4, KIR2DL5, KIR3DL1, KIR3DL2, KIR3DL3, TIGIT, LAG3 or A2AR, or any combination thereof.

In a particular aspect, the one or several mRNA molecule(s) encode(s) for CCR4, CCR10, CXCR3, CCR5, CCR2, CX3CR1, CCR7, CXCR4, CXCR5, IL10R, TGFR, TNFR, ILR1A, GCSFR, IL4R, CXCR1, CXCR2, CXCR6, CCR1, CCR3, CCR6, CCR8, CCR9, CCR11, XCR1, CD72, DCIR, MICL, CLEC-1, TGFB, IL13, IL4, IL-10, IL35, IL37, IL38 IL-6R, IL17R, IL23R, IL35R, IL21R, IFNa R, BTLA, CTLA4, B7H1, B7H3, B7H4, PD1H, LAIR1, TIM1, TIM3, TIM4, 2B4, PD-1, PDL1, PDL2, galectin9, CD48, HVEM, B7DC, CD155, CD113, CD112, ILT2, ILT3, NKG2A, LILRB1, IRp60, KIR, CD22, CD5, CD66a, PIR-B, KIR2DL1, KIR2DL2/3, ILT4, KIR2DL5, KIR3DL1, KIR3DL2, KIR3DL3, TIGIT, LAG3 or A2AR, or any combination thereof.

In a particular aspect, the immune cell inhibiting protein is a protein inhibiting a compound, protein or molecule that inhibits the exhaustion of immune cells, the apoptosis of immune cells, the production or concentration of mitochondrial enzymes and/or transporters of immune cells, the production or concentration of transcription factors of immune cells, the production or concentration of metabolism enzymes of immune cells, the hypoxia of the microenvironment or tissue, the efficiency of signalling pathways of immune cells, the secretion of immune cells, the internalization of endosome of immune cells, the production or concentration of chaperone protein of immune cells, the production of cytoskeleton regulation protein of immune cells, the migration and/or motility of immune cells, the active membrane transport of immune cells, the production or concentration of carrier protein of immune cells or the production or concentration of tRNA of immune cells, the production and/or secretion of checkpoint inhibitors, or the production of inflammation resolution factors; or that induces or increases the proliferation of immune cells, the production of cytotoxic compounds by immune cells or the degradation of protein through immuno-proteasome and ubiquitination, as well as the antigen presentation of immune cells.

In some aspects, the invention concerns a lipid-based nanoparticle comprising an antigen-binding domain capable of specifically binding to a target expressed on activated T cells surface and one or several mRNA molecule(s) encoding an activity-inhibiting protein of said T cells.

Preferably, the invention concerns a lipid-based nanoparticle comprising an anti-PD-1 antigen-binding domain and one or several mRNA molecule(s) encoding an activity-inhibiting protein of said T cells.

Even more preferably, the invention concerns a lipid-based nanoparticle comprising an anti-PD-1 antigen-binding domain and one or several mRNA molecule(s) encoding T cells transcription factors or T cell factors inhibiting growth.

Additionally, the lipid-based nanoparticle of the invention may comprise several mRNA molecules encoding different immune cell activity inhibiting proteins. In this aspect, the lipid-based nanoparticle comprises a first mRNA molecule encoding a T cell activity inhibiting protein and a second mRNA molecule encoding a second immune cell activity inhibiting protein. The second immune cell activity inhibiting protein may be another T cell activity inhibiting protein (i.e., different form the first T cell activity inhibiting component) or can be an activity inhibiting component of another type of immune cell, such as a NK or a macrophage.

In an aspect, the lipid-based nanoparticle comprises at least two mRNA molecule(s), wherein one of said at least two mRNA molecules encodes a transmembrane protein that is a receptor and another of said at least two mRNA molecules encodes a secreted protein that is a ligand of said receptor. For example, the lipid-based nanoparticle comprises a mRNA encoding an interleukin receptor (e.g., GLFI-R) and a mRNA encoding the associated interleukin (e.g., TL-10). This allows the inhibition at the level of the same/single immune cell (i.e., cis-activation). Other examples are the followings:
TGFB and TGFBR,
TNF and TNFR,
TL13 and IL13R,
GCF and GCFR,
TL4 and TL4R,
CD72 and CD 100,
CD22 and α2-6Sia,
Siglec-10 and α2-3Sia,
LTLRB and BILA

Receptors and ligands are detailed in Table G hereunder. In some aspects, the mRNa molecule encodes for a protein selected in Table G.

TABLE G
Receptors and associated ligands

| | | Uniprot |
|---|---|---|
| Name | Official name | reference |
| TGFB1 | Transforming growth factor beta-1 proprotein | P01137 |
| TGFB2 | Transforming growth factor beta-2 proprotein, Cetermin, | P61812 |
| | Glioblastoma-derived T-cell suppressor factor, G-TSF | |
| TGFB3 | Transforming growth factor beta-3 proprotein | P10600 |
| TGFBR1 | TGF-beta receptor type-1, TGFR-1, 2.7.11.30, Activin A | P36897 |
| | receptor type II-like protein kinase of 53 kD, Activin receptor- | |
| | like kinase 5, ALK-5, ALK5, Serine/threonine-protein kinase | |
| | receptor R4, SKR4, TGF-beta type I receptor, Transforming | |
| | growth factor-beta receptor type I, TGF-beta receptor type I, | |
| | TbetaR-I | |
| TGFBR2 | TGF-beta receptor type-2, TGFR-2, 2.7.11.30, TGF-beta type II | P37173 |
| | receptor, Transforming growth factor-beta receptor type II, | |
| | TGF-beta receptor type II, TbetaR-II | |
| TGFBR3 | Transforming growth factor beta receptor type 3, TGF-beta | Q03167 |
| | receptor type 3, TGFR-3, Betaglycan, Transforming growth | |
| | factor beta receptor III, TGF-beta receptor type III | |
| TNF | Tumor necrosis factor, Cachectin, TNF-alpha, Tumor necrosis | P01375 |
| | factor ligand superfamily member 2, TNF-a | |
| TNFR1A | Tumor necrosis factor receptor superfamily member 1A, Tumor | P19438 |
| | necrosis factor receptor 1, TNF-R1, Tumor necrosis factor | |
| | receptor type I, TNF-RI, TNFR-I, p55, p60, CD120a | |
| TNFR1B | Tumor necrosis factor receptor superfamily member 1B, Tumor | P20333 |
| | necrosis factor receptor 2, TNF-R2, Tumor necrosis factor | |
| | receptor type II, TNF-RII, TNFR-II, p75, p80 TNF-alpha | |
| | receptor, CD120b, Etanercept | |
| IL13R1 | Interleukin-13 receptor subunit alpha-1, IL-13 receptor subunit | P78552 |
| | alpha-1, IL-13R subunit alpha-1, IL-13R-alpha-1, IL-13RA1, | |
| | Cancer/testis antigen 19, CT19, CD213a1 | |
| IL13R2 | Interleukin-13 receptor subunit alpha-2, IL-13 receptor subunit | Q14627 |
| | alpha-2, IL-13R subunit alpha-2, IL-13R-alpha-2, IL-13RA2, | |
| | Interleukin-13-binding protein, CD213a2 | |
| GCF | Intron Large complex component GCFC2, GC-rich sequence | P16383 |
| | DNA-binding factor, GC-rich sequence DNA-binding factor 2, | |
| | Transcription factor 9, TCF-9 | |
| GCFR | Epidermal growth factor receptor, 2.7.10.1, Proto-oncogene c- | P00533 |
| | ErbB-1, Receptor tyrosine-protein kinase erbB-1 | |
| IL4R | Interleukin-4 receptor subunit alpha, IL-4 receptor subunit alpha, | P24394 |
| | IL-4R subunit alpha, IL-4R-alpha, IL-4RA, CD124 | |
| CD100 | Semaphorin-4D, A8, BB18, GR3, CD100 | Q92854 |
| CD22 | B-cell receptor CD22, B-lymphocyte cell adhesion molecule, | P20273 |
| | BL-CAM, Sialic acid-binding Ig-like lectin 2, Siglec-2, T-cell | |
| | surface antigen Leu-14, CD22 | |
| Siglec-10 | Sialic acid-binding Ig-like lectin 10, Siglec-10, Siglec-like | Q96LC7 |
| | protein 2 | |
| LILRB | Leukocyte immunoglobulin-like receptor subfamily B member | Q8NHL6, |
| | 1, LIR-1, Leukocyte immunoglobulin-like receptor 1, CD85 | Q8N423, |
| | antigen-like family member J, Immunoglobulin-like transcript 2, | O75022, |
| | ILT-2, Monocyte/macrophage immunoglobulin-like receptor 7, | Q8NHJ6, |
| | MIR-7, CD85j; Leukocyte immunoglobulin-like receptor | O75023 |
| | subfamily B member 2, LIR-2, Leukocyte immunoglobulin-like | |
| | receptor 2, CD85 antigen-like family member D, | |
| | Immunoglobulin-like transcript 4, ILT-4, | |
| | Monocyte/macrophage immunoglobulin-like receptor 10, MIR- | |
| | 10, CD85d; Leukocyte immunoglobulin-like receptor subfamily | |
| | B member 3, LIR-3, Leukocyte immunoglobulin-like receptor 3, | |
| | CD85 antigen-like family member A, Immunoglobulin-like | |
| | transcript 5, ILT-5, Monocyte inhibitory receptor HL9, CD85a; | |
| | Leukocyte immunoglobulin-like receptor subfamily B member | |
| | 4, B4, CD85 antigen-like family member K, Immunoglobulin- | |
| | like transcript 3, ILT-3, Leukocyte immunoglobulin-like | |
| | receptor 5, LIR-5, Monocyte inhibitory receptor HM18, CD85k; | |
| | Leukocyte immunoglobulin-like receptor subfamily B member | |
| | 5, CD85 antigen-like family member C, Leukocyte | |
| | immunoglobulin-like receptor 8, LIR-8, CD85c | |
| HLA-A | HLA class I histocompatibility antigen, A alpha chain, Human | P04439 |
| | leukocyte antigen A, HLA-A | |
| HLA-B | HLA class I histocompatibility antigen, B alpha chain, Human | P01889 |
| | leukocyte antigen B, HLA-B | |
| HLA-C | HLA class I histocompatibility antigen, C alpha chain, HLA-C, | P10321 |
| | HLA-Cw, Human leukocyte antigen C | |

In some aspects, the mRNa molecule encodes for a protein selected in Table E, F and G.

Additionally, α2-3Sia and α2-6Sia refers to sialic acids linked together, either with an α2-3 linkage or an α2-6 linkage, respectively. Linkage of sialic acids residues is known in the art, see for example FIGS. 23 and 24 of Cao, H., Chen, X. (2012). General Consideration on Sialic Acid Chemistry. In: Chevolot, Y. (eds) Carbohydrate Microarrays. Methods in Molecular Biology, vol 808. Humana Press. https://doi.org/10.1007/978-1-61779-373-8_3.

In some embodiments, the lipid-based nanoparticle comprises one or more mRNA molecule selected from the group consisting of:
a mRNA encoding for BIM, preferably a mRNA molecule comprising a nucleic acid sequence such as described in SEQ ID NO: 43 or having at least 80%, 90%, 95% or 99% sequence identity thereto;
a mRNA encoding for PUMA, preferably a mRNA molecule comprising a nucleic acid sequence such as described in SEQ ID NO: 44 or having at least 80%, 90%, 95% or 99% sequence identity thereto.

In some aspects, the invention particularly concerns a lipid-based nanoparticle comprising:
a) a lipid based composition comprising:
   ALC-00315, SM-102, Dlin-MC3-DMA or SS-OP or any mixture thereof from about 45 mol % to about 55 mol 0%, preferably from about 48 mol % to about 52 mol %, more preferably of about 50 mol % of the total lipids present in the LNP,
   DOPE, DDAB, DOPC, POPE or DSPC or any mixture thereof from about 5 mol % to about 15 mol %, preferably from about 8 mol % to about 12 mol %, more preferably of about 10 mol % of the total lipids present in the LNP,
   Cholesterol from about 35 mol % to about 45 mol %, preferably from about 37 mol % to about 40 mol %, more preferably of about 38.5 mol % of the total lipids present in the LNP, and
   PEG 2000-DSG, PEG 2000-DMG, PEG 5000-DSG, PEG 5000-DMG or ALC-0159 or any mixture thereof, from about 0.5 mol % to about 2.5 mol %, preferably from about 1 mol % to about 2 mol %, more preferably of about 1.5 mol % of the total lipids present in the LNP;
b) an antigen binding domain that specifically binds to human PD-1 or human CLEC-1;
c) one or more mRNA molecule that encodes for:
   a protein selected from the group consisting of TIM3, ENTPD1, LAG3, PD-1, PD-L1, TIGIT, FOXO1, MLH1, MSH2, MSH6, APC, CDKN2A, CASPASE, FAS, TRAIL, Bax, Bak, Bok, Bad, Bid et Bim, BIM, BIM-S, FADD, FASL, TRAILR, TNFR, ATG5, LC3, GABARAP, GATE16, ATG 5/7/10/12, NOXA, PUMA, P53, FOXP3, TOX, EOMES, BCL6 and BACH2, HIFa, PKCq and VHL, IDO-1, IDO-2, ARG1; TDO, mTOR/DAPTOR/RAPTOR, SUP, SMAD, SHIP-1, SHP-1/2, PTEN, PTP1B, IKAROS, EGR2/3, CREM, P27 (KIP1), CD107a, RAC1, AP2, RB7, M6P, MPR, BBS10, BBS12, TCP1, HSP, APC, Laminin, Actin, Vimentin, DEF1, Dynein, Kinesin, TAPASIN, LMP7, Erp57, Cbl-b, CHEMR23, GPR37, GPR32, GPR18, FPR2, GPR35, HDAC, HMT, SIRT1, DOT1L, PRDME, PRMT1, PRMT3, PRMT5, PRMT6, SETD7, SETD8, SMYD2, SMYD3 or SUV420H2, preferably selected from the group consisting of TIM3, LAG3, PD-L1, TIGIT, FAS, TRAIL, PUMA, BIM-S, FOXP3, TOX, IDO-1, IDO-2, ARG1, CHEMR23 and FPR2, and any combination thereof,
   a cytokine, preferably selected from the group consisting of IFNG, IFNa, IL2, IL13, IL4 and IL-10, and any combination thereof,
a cytokine receptor, preferably selected from the group consisting of IL-1R, IL-4R, IL-6R, IL-8R, IL-10R, IL-11R, IL-17R, IL-18R, IL-21R, IL-23R, IL-35R, and any combination thereof, and
   a chemokine receptor, preferably selected from the group consisting of CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6 and XCR1, and any combination thereof.

In some aspects, the invention particularly concerns a lipid-based nanoparticle comprising:
a) a lipid based composition comprising:
ALC-0315, DOPE, cholesterol and DMG-PEG,
ALC-0315, DDAB, cholesterol and DMG-PEG,
ALC-0315, POPE, cholesterol and DMG-PEG,
ALC-0315, DOPE, cholesterol and DSPE-PEG,
ALC-0315, DSPC, cholesterol and DMG-PEG,
ALC-0315, DSPC, cholesterol and ALC-0159;
SM-102, DSPC, cholesterol and DMG-PEG,
Dlin-MC3-DMA, DSPC, cholesterol and DMG-PEG,
ALC-0315, DOPE, cholesterol, DMG-PEG and DSPE-PEG,
SS-OP, DOPE, cholesterol and DMG-PEG;
SS-OP, DSPC, cholesterol and DSPE-PEG; and
SS-OP, DOPC, cholesterol and DMG-PEG;
in particular according to the lipid percentages described herein;
b) an antigen binding domain that specifically binds to human PD-1;
c) one or more mRNA molecule that encodes for a protein selected from the group consisting of FOXO1, Fas, TRATL, PUMA, NOXA, BIM, LAG3, CTLA4, PDL1, FOXP3, TOX, IDO, ARG1 IL35+IL35R, TGFB+TGFBR, TIM3, TIGIT, ChemR23, FPR2, and any combination thereof, preferably PUMA or BIMS.

### Pharmaceutical Compositions

The present invention also relates to a pharmaceutical composition comprising a lipid-based nanoparticle as described hereabove, preferably as the active ingredient or compound and optionally a pharmaceutically acceptable carrier or excipient.

As used herein, a “pharmaceutical composition” refers to a preparation of one or more of the active agents, such as comprising a LNP according to the invention, with optional other chemical components such as physiologically suitable carriers and excipients. The purpose of a pharmaceutical composition is to facilitate administration of the active agent to an organism. Compositions of the present invention can be in a form suitable for any conventional route of administration or use. In one aspect, a “composition” typically intends a combination of the active agent, e.g., compound or composition, and a naturally-occurring or non-naturally-occurring carrier, inert or active, such as an adjuvant, diluent, binder, stabilizer, buffers, preservative or the like and include pharmaceutically acceptable carriers. An “acceptable vehicle” or “acceptable carrier” as referred to herein, is any known compound or combination of compounds that are known to those skilled in the art to be useful in formulating pharmaceutical compositions.

The pharmaceutical compositions can be sterilized and, if desired, mixed with auxiliary agents such as pharmaceutically acceptable carriers, excipients, salts, antioxidant and/or stabilizers which do not deleteriously interact with the lipid-based nanoparticle of the invention and does not impart any undesired toxicological effects.

Particularly, the pharmaceutical composition according to the invention can be formulated for any conventional route of administration including a topical, enteral, oral, parenteral, intranasal, intravenous, intramuscular, subcutaneous or intraocular administration and the like. To facilitate administration, the lipid-based nanoparticle as described herein can particularly be made into a pharmaceutical composition for in vivo administration. The means of making such a composition have been described in the art (see, for instance, Remington: The Science and Practice of Pharmacy, Lippincott Williams & Wilkins, 21^{st} edition (2005)). In yet another embodiment, the pharmaceutical composition is administered intranodally or intratumorally.

The pharmaceutical composition may be prepared by mixing a lipid-based nanoparticle having the desired degree of purity with optional pharmaceutically acceptable carriers, excipients, antioxidant, and/or stabilizers in the form of lyophilized formulations or aqueous solutions. Such suitable carriers, excipients, antioxidants, and/or stabilizers are well known in the art and have been for example described in Remington's Pharmaceutical Sciences 16^{th} edition, Osol, A. Ed. (1980).

Preferably, the pharmaceutical composition comprising the lipid-based nanoparticle is relatively homogenous. A polydispersity index may be used to indicate the homogeneity of the composition, e.g., the particle size distribution of the lipid-based nanoparticles comprised in the composition. A small (e.g., less than 0.3) polydispersity index generally indicates a narrow particle size distribution. Preferably, the pharmaceutical composition has a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of the pharmaceutical composition is from about 0.10 to about 0.25.

Pharmaceutical compositions according to the invention may be formulated to release the active ingredients (e.g. the lipid-based nanoparticle of the invention) substantially immediately upon administration or at any predetermined time or time period after administration. The pharmaceutical composition in some aspects can employ time-released, delayed release, and sustained release delivery systems such that the delivery of the composition occurs prior to, and with sufficient time to cause, sensitization of the site to be treated. Means known in the art can be used to prevent or minimize release and absorption of the composition until it reaches the target tissue or organ, or to ensure timed-release of the composition. Such systems can avoid repeated administrations of the composition, thereby increasing convenience to the subject and the physician.

It will be understood by one skilled in the art that the formulations of the invention may be isotonic with human blood that is the formulations of the invention have essentially the same osmotic pressure as human blood. Such isotonic formulations generally have an osmotic pressure from about 250 mOSm to about 350 mOSm. Isotonicity can be measured by, for example, a vapor pressure or ice-freezing type osmometer.

Pharmaceutical composition typically must be sterile and stable under the conditions of manufacture and storage. Prevention of presence of microorganisms may be ensured both by sterilization procedures (for example by microfiltration), and/or by the inclusion of various antibacterial and antifungal agents.

In some embodiments, the pharmaceutical composition includes one or more pharmaceutically acceptable excipients or accessory ingredients such as, but not limited to, one or more solvents, dispersion media, diluents, dispersion aids, suspension aids, surface active agents, buffering agents and/or preservatives.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium and/or combinations thereof.

Examples of preservatives may include, but are not limited to, antioxidants, chelating agents, free radical scavengers, antimicrobial preservatives, antifungal preservatives, alcohol preservatives and/or acidic preservatives.

Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite and/or sodium sulfite.

Examples of chelating agents include, but are not limited to, ethylenediaminetetraacetic acid (EDTA), citric acid monohydrate, disodium edetate, dipotassium edetate, edetic acid, fumaric acid, malic acid, phosphoric acid, sodium edetate, tartaric acid and/or trisodium edetate.

Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate and/or sorbic acid.

Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™ and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution and/or ethyl alcohol.

In some embodiments, the pharmaceutical composition including a lipid-based nanoparticle according to the invention further includes a salt, such as a chloride salt. In some embodiments, the pharmaceutical composition including a lipid-based nanoparticle further includes a sugar such as a disaccharide. In some embodiments, the pharmaceutical composition further includes a sugar but not a salt, such as a chloride salt. In some embodiments, a pharmaceutical composition further includes one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

Formulations comprising amphiphilic polymers and lipid-based nanoparticles may be formulated in whole or in part as pharmaceutical compositions. Pharmaceutical compositions may particularly include one or more amphiphilic polymers and one or more lipid-based nanoparticles. For example, a pharmaceutical composition may include one or more amphiphilic polymers and one or more lipid-based nanoparticles including one or more different mRNA therapeutics and/or prophylactics. In addition, excipients and accessory ingredients may be used in said pharmaceutical composition, except insofar as any conventional excipient or accessory ingredient may be incompatible with one or more components of the lipid-based nanoparticle(s) or amphiphilic polymer(s).

In some embodiments, the pharmaceutical composition comprises between 0.1% and 15% (wt/vol) of one or more amphiphilic polymers (e.g., 0.5%, 1%, 2.5%, 5%, 10%, or 12.5% w/vol).

Relative amounts of the lipid-based nanoparticles, pharmaceutically acceptable excipients and/or any additional ingredients in a pharmaceutical composition in accordance with the present disclosure may vary, depending upon the identity, size, and/or condition of the subject treated and further depending upon the route by which the composition is to be administered. In some embodiments, the pharmaceutical composition comprises between 0.1% and 100% (wt/wt) of one or more lipid-based nanoparticles such as disclosed herein. The amount of Lipid-based nanoparticles which can be combined with a carrier material to produce a single dosage form will generally be that amount of the lipid-based nanoparticles which produces a therapeutic effect.

In some embodiments, one or more excipients or accessory ingredients may make up greater than 50% of the total mass or volume of the pharmaceutical composition including a lipid-based nanoparticle such as disclosed herein. For example, the one or more excipients or accessory ingredients may make up 50%, 60%, 70%, 80%, 90%, or more of a pharmaceutical composition. In some embodiments, a pharmaceutically acceptable excipient is at least 95%, at least 96%, at least 97%, at least 98%, at least 99%, or 100% pure. In some embodiments, an excipient is approved for use in humans and/or for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

In a particular embodiment, the pharmaceutical composition comprises 2, 3, 4, 5, 6, 7, 8, 9 or 10 different lipid-based nanoparticles comprising an antigen binding domain capable of specifically binding to a target expressed on activated immune cells surface and comprising one or several mRNA molecule(s) encoding an immune cell activity inhibiting component. By “different lipid-based nanoparticles” it is meant lipid-based nanoparticles that differ from i) the lipid composition of the nanoparticle, ii) the antigen binding domain (e.g., different sequences and/or target) and/or iii) the mRNA molecule(s).

Preferably, the pharmaceutical composition comprises at least two different lipid-based nanoparticles comprising an antigen binding domain capable of specifically binding to a target expressed on activated immune cells surface and comprising one or several mRNA molecule(s) encoding an immune cell activity inhibiting component.

Preferably, the pharmaceutical composition comprises at least two different lipid-based nanoparticles comprising an antigen binding domain capable of specifically binding to a target expressed on activated immune cells surface and comprising one or several mRNA molecule(s) encoding an immune cell activity inhibiting component, wherein the antigen binding domain is an antibody or an antigen binding fragment thereof and preferably comprises a Fc domain, preferably an IgG Fc domain.

In some particular embodiments, the pharmaceutical composition comprises at least two different lipid-based nanoparticles as described above, wherein none of the lipid-based nanoparticles comprises one or more antigen binding domain(s) that i) is/are not covalently bound to any of the lipids of the lipid-based nanoparticle, ii) does/do not comprise any modification for coupling or grafting the antigen binding domain to a lipid and/or iii) is/are not covalently bound to a lipidation peptide or motif.

Then, the pharmaceutical composition according to the invention may comprise:
A first lipid-based nanoparticle comprising a first antigen binding domain capable of specifically binding to a first target expressed on activated immune cells surface and one or several mRNA molecule(s) encoding an immune cell activity inhibiting component; and
A second lipid-based nanoparticle comprising a second antigen binding domain capable of specifically binding to a second (different) target expressed on activated immune cells surface and one or several mRNA molecule(s) encoding an immune cell activity inhibiting component.

Preferably, the one or several mRNA molecule(s) encoding an immune cell activity inhibiting component may be the same or different in the first and second lipid-based nanoparticles.

In a preferred aspect, the antigen binding domain of the lipid-based nanoparticle and the antigen binding domain of the additional lipid-based nanoparticle are capable of specifically binding to the same target (e.g., PD-1) while each having mRNA molecule(s) encoding for a different immune cell activity inhibiting component, respectively.

Preferably, the antigen binding domain of the first lipid-based nanoparticle and the antigen binding domain of the additional (second) lipid-based nanoparticle are capable of specifically binding to a different/non overlapping epitope of the same target (e.g., different epitopes of PD-1).

Alternatively, the antigen binding domain of the first lipid-based nanoparticle and the antigen binding domain of the additional (second) lipid-based nanoparticle are capable of specifically binding to the same epitope of the target (e.g., the same epitope on PD-1).

### Therapeutic Uses

The lipid-based nanoparticle and the pharmaceutical composition comprising such as defined above have numerous in vitro and in vivo utilities and applications. Particularly, the lipid-based nanoparticles and the pharmaceutical compositions provided herein may be used in therapeutic methods and/or for therapeutic purposes.

The present invention relates to a lipid-based nanoparticle or a pharmaceutical composition comprising such for use as a medicament or vaccine and/or for use in the treatment of a disorder or disease, such as an autoimmune disease or an inflammatory disease.

The present invention relates to the use of one or more lipid-based nanoparticle(s) according to the invention or of the pharmaceutical composition according to the invention, for the manufacture of a medicament for the treatment of an autoimmune disease or an inflammatory disease.

The present invention relates to a method for treating an autoimmune disease or an inflammatory disease in a subject, wherein the method comprises administering to said subject one or more lipid-based nanoparticle(s) according to the invention or the pharmaceutical composition according to the invention.

It also relates to the use of a lipid-based nanoparticle or a pharmaceutical composition comprising such for treating a disease or disorder, such as an autoimmune disease or an inflammatory disease, in a subject. It also concerns the use a lipid-based nanoparticle or pharmaceutical composition as disclosed herein in the manufacture of a medicament for treating a disease or disorder, such as an autoimmune disease or an inflammatory disease, in a subject.

Finally, it relates to a method for treating a disease or a disorder in a subject, such as an autoimmune disease or an inflammatory disease, in a subject comprising administering to said subject a therapeutically effective amount of a pharmaceutical composition or a lipid-based nanoparticle such as disclosed herein.

In one embodiment, the invention relates to a method of treatment of a disease and/or disorder selected from the group consisting of a disease wherein the resolution of inflammation is delayed or disrupted, and/or a disease selected from the group of inflammatory diseases, autoimmune diseases in a subject in need thereof, comprising administering to said subject an effective amount of the lipid-based nanoparticle or pharmaceutical composition as defined above. Examples of such diseases and disorders are more particularly described hereafter.

Particularly, the invention concerns a treatment method that comprises: (a) identifying a patient in need of treatment; and (b) administering to the patient a therapeutically effective amount of the lipid-based nanoparticle or pharmaceutical composition described herein.

“An effective amount” or a “therapeutic effective amount” as used herein refers to the amount of active agent (i.e., the lipid-based nanoparticle disclosed herein) required to confer therapeutic effect on the subject, either alone or in combination with one or more other active agents, e.g., the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art.

### Auto Immune Disease

The lipid-based nanoparticle or pharmaceutical compositions of the invention can be used for the treatment of patients affected by an autoimmune diseases such as diabetes, NASH, in particular type I diabetes, psoriasis, lupus, rheumatoid arthritis, multiple sclerosis, Sjögren's syndrome, celiac disease, vasculitis, myasthenia gravis; infection diseases such as sepsis, severe viral indications with severe inflammatory conditions, such as coronavirus (e.g. COVID-19), peritonitis; degenerative diseases; wound healing disorders, dry eye syndrome; cancer diseases, in particular solid and liquid cancers, metastatic cancers, in particular carcinoma, in particular mammary carcinoma or colon carcinoma, or colorectal cancer or lung cancer or mesothelioma, or myeloid cancer, in particular leukaemia.

Fibrosis (notably lung and hepatic fibrosis) and ANCA (Anti neutrophil Cytoplasmic Autoantibodies) pathologies (vasculitis) are in particular concerned.

### Inflammatory Disease

The lipid-based nanoparticle or pharmaceutical compositions of the invention can be used for the treatment of patients affected by a disease wherein the resolution of inflammation is delayed or disrupted, and/or a disease selected from the group of inflammatory diseases, in particular acute inflammatory diseases, chronic inflammatory diseases such as chronic inflammatory pulmonary diseases (e.g., asthma), keratoconjunctivitis, periodontal disease, eczema, inflammatory bowel disease, in particular Crohn's disease or colitis, in particular ulcerative colitis or spontaneous colitis, cystic fibrosis, cutaneous inflammation.

### Combined Therapies

In some embodiment, the lipid-based nanoparticle or the pharmaceutical composition according to the invention may be used in combination with another therapeutic agent or therapy, in particular for the treatment of autoimmune diseases or autoimmune diseases.

The present invention also relates to a method for treating a disease, such as an autoimmune diseases or a disease wherein the resolution of inflammation is delayed or disrupted, and/or an inflammatory diseases, in a subject, comprising administering to said subject a therapeutically effective amount of the lipid-based nanoparticle or the pharmaceutical composition described herein and a therapeutically effective amount of an additional or second therapeutic agent or therapy.

Specific examples of additional or second therapeutic agents are provided in WO2021/069709.

Accordingly, also provided herein are combined therapies with any of the lipid-based nanoparticle or pharmaceutical composition comprising such, as described herein and a suitable second agent, for the treatment of a disease or disorder.

In an aspect, the lipid-based nanoparticle and the second agent can be present in a unique pharmaceutical composition. Alternatively, the terms “combination therapy” or “combined therapy”, as used herein, embrace administration of these two agents (e.g., a lipid-based nanoparticle as described herein and an additional or second suitable therapeutic agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route. The agents can be administered by the same route or by different routes. For example, a first agent (e.g., a lipid-based nanoparticle) can be administered intramuscularly, and an additional therapeutic agent (e.g., an anti-inflammatory agent, or an immune modulator) can be administered intravenously. Alternatively, an agent of the combination selected may be administered by intravenous injection while the other agents of the combination may be administered intramuscularly.

In an aspect, the additional therapeutic agent can be selected in the non-exhaustive list comprising alkylating agents, angiogenesis inhibitors, antibodies, antimetabolites, antimitotic, antiproliferative, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, leukaemia viral oncogene homolog (ErbB2) receptor inhibitors, growth factor inhibitors, heat shock protein (HSP)-90 inhibitors, histone deacetylase (HDAC) inhibitors, hormonal therapies, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids, plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

Combination therapy could also rely on the combination of the administration of the lipid-based nanoparticle according to the invention or the pharmaceutical composition comprising such with surgery.

Subject, regimen and administration A subject in need of a treatment may be a human having, at risk for, or suspected of having a disease such as an autoimmune disease or an inflammatory disease. Such a patient can be identified by routine medical examination.

As used herein, the term “subject”, “host”, “individual,” or “patient” refers to human, including adult and child. The subject to treat may particularly be a human, particularly a human at the prenatal stage, a new-born, a child, an infant, an adolescent or an adult, in particular an adult of at least 30 years old, 40 years old, preferably an adult of at least 50 years old, still more preferably an adult of at least 60 years old, even more preferably an adult of at least 70 years old.

The form of the pharmaceutical compositions, the route of administration and the dose of administration can be adjusted by the man skilled in the art according to the type and severity of the infection, and to the patient, in particular its age, weight, size, sex, and/or general physical condition. The compositions of the present invention may be administered in a number of ways depending upon whether local or systemic treatment is desired.

Conventional methods, known to those of ordinary skill in the art of medicine, can be used to administer the lipid-based nanoparticle, the pharmaceutical composition or the combined therapy disclosed herein to a subject, depending upon the type of diseases to be treated or the site of the disease e.g., administered orally, parenterally, enterally, by inhalation spray, topically, rectally, nasally, buccally, vaginally or via an implanted reservoir. Preferably, the lipid-based nanoparticle, the pharmaceutical composition or the combined therapy is administered via subcutaneous, intra-cutaneous, intravenous, intramuscular, intra-articular, intra-arterial, intra-synovial, intra-tumoral, intra-sternal, intra-thecal, intra-lesion, and intracranial injection or infusion techniques. Preferably, the lipid-based nanoparticle, the pharmaceutical composition or the combined therapy is administered intravenously.

Alternatively, the lipid-based nanoparticle, the pharmaceutical composition or the combined therapy is administered intratumorally.

In some embodiments, the subject has already received at least one line of treatment, preferably several lines of treatment, prior to the administration of the lipid-based nanoparticle, the pharmaceutical composition or the combined therapy of the invention.

### Methods to Inhibit Immune Cells

The invention also relates to a method of inhibiting immune cell activity, comprising the step of contacting immune cells with a lipid-based nanoparticle or a pharmaceutical composition according to the invention. Such methods aim to inhibit the immune potential of T cells and provide powerful tools to reduce the immune response, in particular for the treatment of a disease such as an autoimmune disease or an inflammatory disease.

In a particular aspect, the lipid-based nanoparticle disclosed herein can be administered to a subject, e.g., in vivo, to reduce immunity or to reduce the immune response, preferably in order to treat a disorder and/or disease. Accordingly, in one aspect, the invention provides a method of inhibiting an immune response in a subject comprising administering to the subject a lipid-based nanoparticle or pharmaceutical composition of the invention such that the immune response in the subject is inhibited. The lipid-based nanoparticle or pharmaceutical composition is preferably used to inhibit immune responses such as immune cell activation in a subject in need of a treatment.

In a particular embodiment, the lipid-based nanoparticle or pharmaceutical composition according to the invention is used to increase T cells exhaustion or to deactivate activated T cells.

The invention particularly provides a method of inhibiting an immune response in a subject, comprising administering to the subject a therapeutic effective amount of any of the lipid-based nanoparticles or pharmaceutical compositions comprising such described herein, such that an immune response in the subject is decreased. In a particular embodiment, the lipid-based nanoparticle or pharmaceutical composition can be used to inhibit the cytotoxic T-lymphocyte (CTL) response, to increase T cells exhaustion or to deactivate activated T cells.

In some embodiments, the method is an ex vivo or an in vitro method.

In such embodiments, the lipid-based nanoparticle or pharmaceutical composition according to the invention may be contacted with immune cells taken from a biological sample of a subject, such as a blood sample.

Accordingly, the method for inhibiting immune cell activation may comprise a first step of providing a biological sample from a subject.

In some embodiments, the method of inhibiting immune cells activation comprises a step of purification/isolation of immune cells from a biological sample, in particular from a blood sample. General techniques for purifying immune cells by cell sorting or antibody and complement treatment are routinely performed and are well-known to those skilled in the art (see for example Leo, O, Sachs, DH, Samelson, L, et al. J. Immunol. 137:3874 (1986)).

In some embodiments, the method of immune cells inhibition comprises a step of immune cell culture, expansion or amplification in particular in an immune cell culture medium. Culture of immune cells are well described in the art (see for example Samuelson, L E, Germain, R N, Schwartz, R H, PNAS(USA) 80:6972 (1983)). Immune cells may particularly be cultured/amplified prior to their contact with the lipid-based nanoparticle or pharmaceutical composition of the invention.

As used herein, the term “immune cells culture medium” or “immune cells culture media” refers to a medium that enables immune cell growth and/or survival and includes all conventional media used in the field appropriate for immune cell culture, in particular an aqueous or liquid medium. The basic medium used for immune cell culture is preferably a cell culture minimum medium (CCMM), and generally includes a carbon source, a nitrogen source, and a trace element component. The medium for immune cell culture may also be selected from the group consisting of DMEM (Dulbecco's Modified Eagle's Medium®), MEM (Minimal Essential Medium Fix), BME (Basal Animal Medium National Eagle®), RPMI1640 ®, F −10 MS, F −12 MS, (Minimal Essential Medium Fix), GMEM SCF (Glaggo's Animal Essential Medium Fix), Iscove's Modified Deldulbecco's Medium, Lonza X-VIVO15™ medium, Corning 88-581-CM DC CIK cell serum-free medium and Lonza X-VIVO15™ phenol-free red medium.

Preferably, the lipid-based nanoparticles according to the invention are comprised in or added to the immune cell culture medium and to contact with immune cells in such medium.

In some embodiments, the method further comprises a step of measuring the activation of immune cells, in particular after the step of contacting the lipid-based nanoparticles or pharmaceutical compositions of the invention with the immune cells.

In some embodiments, the method further comprises a step of measuring the inhibition of immune cells, in particular after the step of contacting the lipid-based nanoparticles or pharmaceutical compositions of the invention with the immune cells.

Immune cell activity can be measured by any method known to the person skilled in the art. Activities which may be monitored to indicate degree of activation or inhibition include, but are not limited to, cell proliferation, cytokine secretion, target cell killing, helper cell function (inducing other cell types to activity), cell migration, cell differentiation, or secretion of other cell products such as hormones or proteins.

In some embodiment, the immune cell activity is monitored by assessing the presence or amount of the immune cell inhibiting component encoded by the mRNA comprised in the lipid-based nanoparticle among the immune cell population and/or in the immune cell culture medium.

In some embodiments, the activated immune cells are administered to a patient to inhibit his or her immune system, in particular to treat various forms of autoimmune diseases or inflammatory diseases. Accordingly, the method of inhibiting immune cells activation may comprise:
a. Providing a biological sample from a subject, in particular a blood sample;
b. Isolating immune cells from the biological sample;
c. Culturing/amplifying the isolated immune cells, preferably in an immune cell culture medium;
d. Contacting the amplified immune cells with the lipid-based nanoparticles or pharmaceutical compositions of the invention, preferably in the immune cell culture medium;
e. Optionally, assessing the activity of immune cells.

Such method may further comprise a step f. of selecting the immune cells that are inhibited.

Such method may further comprise a step f. or g of administering the activated immune cells that are inhibited into the subject, in particular for inhibiting his immune response or for the treatment of a disease such as autoimmune diseases or inflammatory diseases.

### Kits

Any of the lipid-based nanoparticle or compositions described herein may be included in a kit provided by the present invention. The present disclosure particularly provides kits for use in treating diseases or disorders (e.g., autoimmune disease and/or inflammatory disease) or for inhibiting immune cells activation.

In the context of the present invention, the term “kit” means two or more components (one of which corresponding to the lipid-based nanoparticle) packaged in a container, recipient or otherwise. A kit can hence be described as a set of products and/or utensils that are sufficient to achieve a certain goal, which can be marketed as a single unit. The kits of this invention are in suitable packaging.

The kit may include, in suitable container means, the pharmaceutical composition or lipid-based nanoparticle of the present invention.

The components comprised in the kit according to the invention may particularly be formulated into a syringe compatible composition.

In some embodiments, in particular when the kit is for use in the treatment of a disease, the kit further includes an additional agent for treating autoimmune diseases or inflammatory diseases, and the additional agent may be combined with the pharmaceutical composition and/or lipid-based nanoparticle of the present invention, or other components of the kit of the present invention or may be provided separately in the kit. Particularly, the kit described herein may include one or more additional therapeutic agents such as those described in the “Combined Therapy” described hereabove. The kit(s) may be tailored to a particular autoimmune disease for an individual and comprise respective second autoimmune disease therapies for the individual as described hereabove.

When the kit is for use in the method for inhibiting immune cell activation such as disclosed herein, the kit may further comprise an immune cell culture medium. For instance, the lipid-based nanoparticles according to the invention may be comprised in such immune cell culture medium.

The instructions related to the use of the lipid-based nanoparticle or pharmaceutical composition described herein generally include information as to dosage, dosing schedule, route of administration for the intended treatment, means for reconstituting the lipid-based nanoparticle and/or means for diluting the lipid-based nanoparticle of the invention.

Instructions supplied in the kits of the invention are typically written instructions on a label or package insert (e.g., a paper sheet included in the kit in the form of a leaflet or instruction manual).

All the references cited in this description are incorporated by reference in the present application. Others features and advantages of the invention will become clearer in the following figures and examples which are given for purposes of illustration and not by way of limitation.

### BRIEF DESCRIPTION OF THE DRAWINGS

FIG. 1—Flow cytometry analyses of U937 (WT vs transduced), Jurkat (WT vs transduced) and HPB-ALL. Flow cytometry was used to measure the percentage of PD-1+ cells. Dashed line: cells were stained with a PE-Cy7 labelled control isotype mouse IgG (#557646 batch: 8155598, BD Biosciences). Dark grey: cells were stained with a PE-Cy7 labelled anti-human PD-1 antibody (#561272 batch: 1319137, BD Biosciences). Dead cells were stained and excluded from analysis with LIVE/DEAD™ Fixable Yellow Dead Cell Stain Kit (#L34968A batch: 2438368, Life Technologies).

FIG. 2—Addition of the anti-PD-1 mAb OSE-279 before the formation of LNPs allows for the preparation of targeted-LNPs with improved transfection potency in PD-1+ cells. Different modalities for mAb OSE-279 addition during the LNPs production process were tested. Transfection efficiencies of LNPs encapsulating Fluc-mRNA were assessed by comparing OSE-279 addition modalities #1A, #1B and #1C in WT and PD-1+U937 cells (FIG. 2A), and in WT and PD-1+ Jurkat cells (FIG. 2B). Non-targeted-LNPs (nt-LNPs=LNPs produced without addition of mAb) were used as negative control.

FIG. 3—Increasing the dose of mAb in targeted-LNPs induces improved transfection. Targeted-LNPs were prepared using increasing amounts of mAb OSE-279 (1, 10 and 50 μL) added during LNPs preparation. Transfection efficiencies of t-LNPs encapsulating Fluc-mRNA were assessed in WT and PD-1+U937 cells (FIG. 3A), in WT and PD-1+ Jurkat cells (FIG. 3B) and in HPB-ALL cells (FIG. 3C). Non-targeted-LNPs were used as negative control.

FIG. 4—Different ratios of lipids can be used for the preparation of efficient targeted-LNPs. OSE-279 t-LNPs were prepared with varying the relative ratios of lipids constituting the LNPs (ratio 1 and ratio 2, as described in Table 2). Comparative transfection efficiency of targeted-LNPs encapsulating Fluc-mRNA were performed in WT and PD-1+U937 cells (FIG. 4A), in WT and PD-1+ Jurkat cells (FIG. 4B) and in HPB-ALL cells (FIG. 4C). Non-targeted-LNPs and targeted-LNPs prepared with a control isotype mAb were used as negative control.

FIG. 5—Blocking the PD-1 receptors on PD-1+ cells reduced the potency of targeted-LNPs for improved transfection. Targeted-LNPs were prepared using increasing amounts of mAb OSE-279 (1, 10 and 50 μL) added in the course of LNPs preparation. Pre-incubation experiments were performed with the addition of mAb OSE-279—in a concentration allowing for the full occupancy of the cells PD-1 receptors—prior to the addition of targeted-LNPs. Transfection efficiency of LNPs encapsulating Fluc-mRNA were compared in the presence or the absence of the pre-incubation step in WT and PD-1+U937 cells (FIG. 5A), in WT and PD-1+ Jurkat cells (FIG. 5B) and in HPB-ALL cells (FIG. 5C). Non-targeted-LNPs were used as negative control.

FIG. 6—Targeted-LNPs display efficient binding to PD-1 receptors. ELISA binding assays were performed to characterize the binding efficiency to PD-1 receptors of OSE-279 targeted-LNPs compared to OSE-279 mAb “alone” (meaning mAb not complexed in an LNP).

FIG. 7—The nature of the anti-PD-1 mAb used in targeted-LNPs can be modified while maintaining improved transfection in PD-1 positive cells. Targeted-LNPs were prepared using either Pembrolizumab, Nivolumab or OSE-279 anti-PD-1 mAbs. Transfection efficiency of LNPs encapsulating Fluc-mRNA were compared for Pembrolizumab t-LNPs, Nivolumab t-LNPs and OSE-279 t-LNPs (in the presence or the absence of the anti-PD-1 pre-incubation step), in WT and PD-1+U937 cells (FIG. 7A), in WT and PD-1+ Jurkat cells (FIG. 7B) and in HPB-ALL cells (FIG. 7C). Non-targeted-LNPs and targeted-LNPs prepared with a control isotype mAb were used as negative control.

FIG. 8—Targeted-LNPs are able to more efficiently transfect activated T-cells expressing PD-1 compared to non-targeted-LNPs. Activated T-cells (isolated from PBMC and stimulated via agonistic CD3/CD28 mAbs) were analysed by flow cytometry to determine the percentage of PD-1 positive cells (FIG. 8A). Dashed line: cells were stained with a PE-Cy7 labelled control isotype mouse IgG (#557646 batch: 8155598, BD Biosciences). Dark grey: cells were stained with a PE-Cy7 labelled anti-human PD-1 antibody (#561272 batch: 1319137, BD Biosciences). Dead cells were stained and excluded from analysis with LIVE/DEAD™ Fixable Yellow Dead Cell Stain Kit (#L34968A batch: 2438368, Life Technologies). The transfection efficiency of OSE-279 targeted-LNPs on activated T-cells was compared to non-targeted-LNPs under different experimental conditions: 60 000/100 000/300 000/500 000 cells and 25/35/50 μL of LNPs added into the wells (FIG. 8B and FIG. 8C).

FIG. 9—The receptors targeted by the t-LNPs can be modified by varying the mAb introduced in the t-LNPs. U937 cells were transduced to express both PD-1 and target 2 receptors, while HPB-ALL cells naturally express PD-1 and target 2. Flow cytometry was used to measure the percentage of PD-1+ and cells expressing target 2 (FIG. 9A). Dashed line: cells were stained with a PE-Cy7 labelled control isotype mouse IgG (#557646 batch: 8155598, BD Biosciences). Dark grey: cells were stained with a PE-Cy7 labelled anti-human PD-1 antibody (#561272 batch: 1319137, BD Biosciences) to measure PD-1+ cells or with PE-Cy7 labelled anti-human CD-127 antibody (#351320 batch: B251081, BioLegend) to measure cells expressing target 2. Dead cells were stained and excluded from analysis with LIVE/DEAD™ Fixable Yellow Dead Cell Stain Kit (#L34968A batch: 2438368, Life Technologies). Targeted-LNPs were prepared using either an anti-target 2 mAb or an anti-PD-1 mAb and their relative transfection efficiency was assessed in U937 (FIG. 9B-C) and in HPB-ALL (FIG. 9D-E) cells expressing both target 2 and PD-1 receptors. Non-targeted-LNPs and targeted-LNPs prepared with a control isotype mAb were used as negative control. Target 2 is CD127.

FIG. 10—Improved apoptosis is observed in PD-1 expressing cells transfected with targeted-LNPs encapsulating mRNA encoding for BIM-S or PUMA proteins. mRNA encoding for apoptosis-inducing proteins BIM-S(FIGS. 10A, C and E) and PUMA (FIGS. 10B, D and F) were produced internally using IVT transcription and respectively encapsulated in targeted-LNP obtained with OSE-279 mAb. Transfection experiments were conducted in PD-1 expressing cell lines: U937 (FIG. 10A-B), Jurkat (FIG. 10C-D) and HPB ALL (FIG. 10E-F). Transfection experiments were compared in the presence or the absence of the pre-incubation step with an anti-PD-1 mAb. 48 h after treatment, cell viability was quantified using a flow cytometer CytoFLEX (Beckman Coulter). Non-targeted-LNPs and targeted-LNPs prepared with a control isotype mAb were used as negative control for targeting experiments.

FIG. 11—LNPs targeted with an anti-CD127 antagonist mAb are able to target CD-127 expressing cells. U937 and Jurkat cells were transduced to express CD-127 receptors. Flow cytometry was used to measure the percentage of CD127+ cells (FIG. 11A). Dashed line: cells were stained with a PE-Cy7 labelled control isotype mouse IgG1 (#557646 batch: 8155598, BD Biosciences). Dark grey: cells were stained with a PE-Cy7 labelled anti-human CD-127 antibody (#351320 batch: B251081, BioLegend) to measure CD-127 positive cells. Dead cells were stained and excluded from analysis with LIVE/DEAD™ Fixable Yellow Dead Cell Stain Kit (#L34968Abatch: 2438368, Life Technologies). Transfection efficiency of anti-human CD-127 targeted-LNPs was assessed in U937 (FIG. 11B) and in HPB-ALL (FIG. 11C) cells expressing CD-127 receptors. Non-targeted-LNPs and targeted-LNPs prepared with a control isotype mAb were used as negative control.

FIG. 12—LNPs targeted with an anti-CLEC antagonist mAb are able to target CLEC-1 expressing cells. U937 and THP-1 cells were transduced to express CLEC-1 receptors. Flow cytometry was used to measure the percentage of CLEC-1 positive cells (FIG. 12A). Dashed line: cells were stained with a purified control isotype human IgG1 (OSE Immunotherapeutics) at 10 μg/mL. Dark grey: cells were stained with a purified anti-human CLEC-1 antibody (OSE Immunotherapeutics) at 10 μg/mL to measure CLEC-1 positive cells. A PE-labelled anti-human IgG antibody (clone: QA19A42 #366904 batch: B359783, BioLegend) was used for detection of primary purified antibodies. Dead cells were stained and excluded from analysis with LIVE/DEAD™ Fixable Yellow Dead Cell Stain Kit (#L34968Abatch: 2438368, Life Technologies) and human Fc Receptor were saturated with Human FcBlock (#564220 batch: 2122225, BD Biosciences). Transfection efficiency of anti-CLEC targeted-LNPs was assessed in U937 (FIG. 12B) and in THP-1 (FIG. 12C) cells expressing CLEC-1 receptors. Non-targeted-LNPs and targeted-LNPs prepared with a control isotype mAb were used as negative control.

FIG. 13—LNPs targeted with an anti-SIRPα antagonist mAb are able to target SIRPα expressing cells. U937 cells were transduced to express SIRPα receptors. Flow cytometry was used to measure the percentage of SIRPα positive cells (FIG. 13A). Dashed line: cells were stained with a purified control isotype human IgG4m (MOTA hIgG4m #PI08898, EVITRIA) at 10 μg/mL. Dark grey: cells were stained with a purified anti-human SIRPα antibody at 10 μg/mL to measure SIRPα positive cells. A PE-labelled anti-human IgG antibody (clone: QA19A42 #366904 batch: B359783, BioLegend) was used for detection of primary purified antibodies. Dead cells were stained and excluded from analysis with LIVE/DEAD™ Fixable Yellow Dead Cell Stain Kit (#L34968A batch: 2622316, Life Technologies) and human Fc Receptor were saturated with Human FcBlock (#564220, 2122225, BD Biosciences). Transfection efficiency of anti-human SIRPα targeted-LNPs was assessed in U937 cells (FIG. 13B) cells expressing SIRPα receptors. Non-targeted-LNPs and targeted-LNPs prepared with a control isotype mAb were used as negative control.

FIG. 14—LNPs targeted with both an anti-PD-1 and an anti-CD-127 antagonist mAb are able to target PD-1 and CD-127 expressing cells. U937 cells were transduced to express both PD-1 and CD-127 receptors. Flow cytometry was used to measure the percentage of PD-1 and CD127 positive cells (FIG. 14A). Dashed line: cells were stained with a PE-Cy7 labelled control isotype mouse IgG1 (#557646 batch: 8155598, BD Biosciences). Dark grey: cells were stained with a PE-Cy7 labelled anti-human PD-1 antibody (#561272 batch: 1319137, BD Biosciences) to measure PD-1 positive cells or with PE-Cy7 labelled anti-human CD-127 antibody (#351320 batch: B251081, BioLegend) to measure CD-127 positive cells. Dead cells were stained and excluded from analysis with LIVE/DEAD™ Fixable Yellow Dead Cell Stain Kit (#L34968A batch: 2438368, Life Technologies). Transfection efficiency of dual (anti-CD172+OSE-279) targeted-LNPs was assessed in U937 cells (FIG. 14B) cells expressing both PD-1 and CD-127 receptors. Non-targeted-LNPs and targeted-LNPs prepared with a control isotype mAb were used as negative control.

FIG. 15—Different ratios of lipids can be used for the preparation of efficient targeted-LNPs. OSE-279 targeted-LNPs were prepared while varying the relative ratios of DOPE and Cholesterol constituting the LNPs. Comparative transfection efficiency of targeted-LNPs were performed in PD-1 positive cells: U937 cells (FIG. 15A), and in HPB-ALL cells (FIG. 15B). Non-targeted-LNPs and targeted-LNPs prepared with a control isotype mAb were used as negative control.

FIG. 16—Different nature of helper lipids can be used for the preparation of efficient targeted-LNPs. OSE-279 targeted-LNPs were prepared while varying the nature of the helper lipid constituting the LNPs (phospholipids DOPE and POPE or cationic lipid DDAB). Comparative transfection efficiency of targeted-LNPs were performed in U937 cells PD-1 positive cells. Non-targeted-LNPs and targeted-LNPs prepared with a control isotype mAb were used as negative control.

FIG. 17—Different lipid length of the PEG-lipid can be used for the preparation of efficient targeted-LNPs. OSE-279 targeted-LNPs were prepared while varying the length of the lipid tail of the PEG-lipid constituting the LNPs (C14-long DMG-PEG2000 and C18-long DSG-PEG2000). Comparative transfection efficiency of targeted-LNPs were performed in PD-1 positive cells: U937 cells (FIG. 17A) and Jurkat cells (FIG. 17B). Non-targeted-LNPs and targeted-LNPs prepared with a control isotype mAb were used as negative control.

FIG. 18—Different lipidic composition can be used for the preparation of efficient targeted-LNPs. OSE-279 targeted-LNPs were prepared using the same lipid constituents as several FDA and EMA-approved mRNA-based LNPs (Cominarty®, SpikeVax® and Onpattro®). Comparative transfection efficiency of non-targeted vs targeted-LNPs were performed in PD-1 positive cells: U937 cells (FIG. 18A-D), Jurkat cells (FIG. 18E-H) and HPB-ALL (FIG. 18I-K).

FIG. 19—Comparison of transfection efficiency and binding of two types of OSE-279 targeted LNPs: LNPs comprising OSE-279 mAbs grafted to lipids or LNPs comprising OSE-279 mAbs that are not grafted to lipids (free OSE-279). Experiments comparing LNPs comprising free OSE279 with LNP obtained with a classical method used for grafting mAbs on LNP surface (via Thiol-Michael addition) were conducted. Firstly, different experimental conditions were tested to optimize the process of Thiol-Maleimide reaction by modifying the amount of DSPE-PEG-Maleimide in the LNP (FIG. 19A-C) and the amount of OSE-279 mAb added to the reaction (FIG. 19D-F). With the optimized reaction conditions, the transfection capacities of OSE-279 targeted LNP prepared either via the Thiol-Maleimide route or via an alternative method were then compared Jurkat (FIG. 19G) and HPB-ALL (FIG. 19H) cells. Binding studies of the two types of targeted LNPs were also assessed (FIG. 19I). FIG. 19J illustrates a process of synthesis of targeted LNPs of the prior art, whereas FIG. 19K presents a process of synthesis of targeted LNPs according to an alternative method.

FIG. 20—Different formats of OSE-279 mAbs can be used to target PD-1 positive cells with targeted LNPs. OSE-279 targeted-LNPs were prepared with various formats of anti-PD-1 mAb OSE-279 (IgG vs Monovalent IgG formats) and their transfection efficiency assessed in different cell lines expressing the PD-1 receptors: U937 (FIG. 20A), Jurkat (FIG. 20B) and HPB-ALL (FIG. 20C). Non-targeted-LNPs and targeted-LNPs prepared with a control isotype mAb were used as negative control.

FIG. 21—Different formats of OSE-279 mAbs can be used to target PD-1 positive cells with targeted LNPs. OSE-279 targeted-LNPs were prepared with various formats of anti-PD-1 mAb OSE-279 (Monovalent IgG vs ScFv-Fc formats) and their transfection efficiency assessed in different cell lines expressing the PD-1 receptors: U937 (FIG. 21A), Jurkat (FIG. 21B) and HPB-ALL (FIG. 21C). Non-targeted-LNPs and targeted-LNPs prepared with a control isotype mAb were used as negative control.

### EXAMPLES

### Materials

Lipids: ALC-0315 (890900), DDAB (890810), DSPC (850365), DMG-PEG-2000 (880151), DSG-PEG2000 (880152), DSPE-PEG2000 (880120) were purchased from Avanti Lipids (8909000), Cholesterol (1046720001) and POPE (01991) were purchased from Merck, DOPE was purchased from Corden Pharma (LP-R4-069), ALC-0159 (BP-25711) and DSPE-PEG2000-Maleimide (BP-23307) were purchased from Broadpharm, Coatsome SS-OP was purchased from NOF Corporation, MC3-DLin-DMA (N-1282) was purchased from Echelon Biosciences, SM-102 (LP001-2) was purchased from ABP Biosciences.

mRNA: CleanCap Fluc mRNA (5moU) was purchased from Trilink Biotechnologies (L-7202)
the mRNA molecule encoding for PUMA comprises SEQ ID NO: 43;
the mRNA encoding for BIM-S comprises SEQ ID NO: 44.

### Cell Culture:

Cells were cultured in T75 flasks using RPMI 1640 medium (Gibco) supplemented with 10% heat inactivated fetal bovine serum (FBS) (Jurkat and U937) or 20% FBS (HPB-ALL), 100 U/mL of penicillin, 0.1 mg/mL of streptomycin, and 2 mM of L-Glutamine. The cells were maintained at 37 °C in a humidified atmosphere containing 5% C02.

The Jurkat and U937 cell lines stably expressing human PD-1 were obtained by lentiviral transduction. Briefly, an optimized cDNA sequence (Genscript) of human PD-1 Uniprot (Q15116) was inserted in the BamHI/SbfI sites of the lentiviral T54-pHRSIN plasmid. The construct was transfected along with packaging plasmids psPAX2 (Trono Lab) and pLTRG (Reiser Lab) into HEK cells to produce lentiviral particles. Wild-type U937 and Jurkat cells were transduced by the lentiviral particles, cultured for two weeks before being sorted for PD-1 expression.

### T-Cells Preparation:

Human Peripheral Blood Mononuclear Cells (PBMCs) were isolated from whole blood of healthy volunteers by density gradient centrifugation. Then, untouched T-cells were purified from PBMCs by depletion of non-T-cells using the human Pan T-Cell Isolation Kit (#130-096-535, Miltenyi), an LS Column (#130-042-401, Miltenyi), and a QuadroMACS™ Separator (#130-090-976, Miltenyi).

For activation, purified T-cells were then plated in a 6-well plate previously coated with agonistic anti-human CD3 (clone: OKT3) and CD28 (clone: CD28.2) monoclonal antibodies at 3 μg/mL in PBS for 2 hours at 37 °C, 5% C02. T cells were incubated overnight at 37 °C, 5% CO₂ before use.

### Flow Cytometry:

Flow cytometry was used to measure the percentage of PD-1+ and cells expressing target 2. A Cytoflex flow cytometer (Beckman) was used for reading and analysis were performed with FlowJo software. Target 2 is CD127.

### Monoclonal Antibodies:

Anti-human PD-1 OSE-279 was produced by LFB Biomanufacturing—BMG167 batch: 21M00297 at 50 mg/mL.

The OSE-279 antibody comprises a heavy and light chain having an amino-acid sequence as disclosed in WO2020/127366.

OSE-279 particularly comprises a VH sequence as set forth in SEQ ID NO: 15 and a VL sequence as set forth in SEQ ID NO: 16. The OSE-279 antibody particularly comprises a heavy chain sequence as set forth in SEQ ID NO: 45 and a light chain sequence as set forth in SEQ ID NO: 46.

Anti-human PD-1 Pembrolizumab—batch: OFR229207LA at 25 mg/mL. Pembrolizumab typically comprises a VH sequence as set forth in SEQ ID NO: 37 and a VL sequence as set forth in SEQ ID NO: 38. Pembrolizumab particularly comprises a heavy chain sequence as set forth in SEQ ID NO: 47 and a light chain sequence as set forth in SEQ ID NO: 48.

Anti-human PD-1 Nivolumab—batch: AXY5480 at 10 mg/mL. Nivolumab typically comprises a VH sequence as set forth in SEQ ID NO: 29 and a light chain sequence sequence as set forth in SEQ ID NO: 30. Nivolumab particularly comprises a heavy chain sequence as set forth in SEQ ID NO: 49 and a light chain sequence as set forth in SEQ ID NO: 50.

Anti-human target 2 (CD127) was produced by OSE Immunotherapeutics—batch #140521 at 1.6 mg/mL.

Anti-human CLEC was produced by OSE Immunotherapeutics.

Anti-human SIRPa was produced by LFB Biomanufacturing—batch: LCP21-CP04

The anti-SIRPa antibody particularly comprises a VH sequence as set forth in SEQ ID NO: 39 and a VL sequence as set forth in SEQ ID NO: 40. The anti-SIRPa antibody particularly comprises a heavy chain sequence as set forth in SEQ ID NO: 51 and a light chain sequence as set forth in SEQ ID NO: 52.

Control isotype was produced by EVITRIA at 7.4 mg/mL.

All mAbs used for the preparation of t-LNPs were diluted to 0.95 mg/mL in PBS.

In all examples but Example 21, the mAbs are not covalently bound to any of the lipids of the lipid-based nanoparticle or do not comprise any modification for coupling or grafting the antigen binding domain to a lipid.

Example 21 features mAbs that are covalently bound to the lipids of the lipid-based nanoparticle, and mAbs that are not covalently bound to any of the lipids of the lipid-based nanoparticle or do not comprise any modification for coupling or grafting the antigen binding domain to a lipid.

mRNA

mRNA encoding for BIM-S and PUMA proteins were produced internally using classical in-vitro transcription procedures (using CleanCap reagent and 1-methyl-pseudouridine as modified NTP).

In particular, the mRNA sequences are the following:
the mRNA molecule encoding for PUMA is as described in SEQ ID NO: 43
the mRNA encoding for BIM-S is as described in SEQ ID NO: 44.

### In Vitro Cellular Uptake

LNPs were 4-fold diluted into RPMI medium supplemented with 10% FBS, 10% heat inactivated fetal bovine serum (FBS), 100 U/mL of penicillin, 0.1 mg/mL of streptomycin, and 2 mM of L-Glutamine. 2.5 μL, 10 μL or 25 μL of the obtained solution were added on cells previously prepared in a final volume of 60 μL per well in their culture medium: 0.1 M cells/well (U937 PD1+ and Jurkat PD1+) or 0.2 M cells/well (HPB-ALL) in a 96-well transparent flat bottom culture plate.

In anti-human PD-1 mAb pre-incubation conditions, 5 μL of mAb were added on the cells for a final concentration of 500 μg/mL (saturating conditions) and incubated at room temperature for 30 minutes before treatment with LNPs.

48 h post-treatment, cells were harvested in a 96-well conical bottom plate and cell viability was evaluated by cell death staining for 30 minutes at 4 °C with LIVE/DEAD™ Fixable Yellow Dead Cell Stain Kit (#L34959, Invitrogen) following manufacturer's recommendations. Cell death was quantified by using a flow cytometer CytoFLEX (Beckman Coulter) and analysed with FlowJo software. Fluorescent threshold was obtained by staining on control conditions (untreated cells and cells treated with LNP containing non-coding mRNA).

### Methods

Preparation of Lipid-Based Nanoparticles (Non-Targeted-LNPs) Entrapping mRNA LNPs were prepared using microfluidic mixing. Briefly, lipids were dissolved in ethanol with the following molar ratios ALC-0315:38%/DOPE:12%/Cholesterol:48%/DMG-PEG-2000:2% (examples 1-3) or ALC-0315:50%/DOPE:10%/Cholesterol:38.5%/DMG-PEG-2000:1.5% (examples 4-9), at a total lipid concentration of 9.1 mM (unless otherwise specified). Fluc-mRNA (1 mg/mL) was diluted with 25 mM acetate buffer (pH 4.3) to a final concentration of 0.083 mg/mL. The selected experimental conditions allow for a N/P ratio of 6.

The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was immediately dialyzed against PBS 1M (pH 7.4) for at least 3.5 h using 3.5 kDa MWCO dialysis cassettes (Pur-A-Lyzer, Sigma Aldrich). After dialysis, the LNPs were stored in a final volume of 1 mL in PBS at 4 °C.

Preparation of Targeted-LNPs (t-LNPs) Entrapping mRNA

Targeted-LNPs (t-LNPs) were prepared as described previously, with one additional step consisting of the addition of a monoclonal antibody (mAb) used as a targeting agent.

The addition of the mAb (0.95 μg/μL in PBS) during the LNP preparation process was performed before the microfluidic mixing step (while maintaining the final mRNA and lipid concentrations constant), by introducing the mAb either in the mRNA solution (modality #1A) or in the lipid solution (modality #1B), as detailed below:

### Modality #1A:

Channel 1=mRNA+mAb in acetate buffer pH 4.3
Channel 2=lipid mix in EtOH

### Modality #1B:

Channel 1=mRNA in acetate buffer pH 4.3
Channel 2=mAb+lipid mix in EtOH

### LNP Characterization

The mean hydrodynamic diameter of the mRNA-LNPs was measured by dynamic light scattering using a Malvern NanoZS (Malvern Instruments, UK).

mRNA encapsulation efficiency was determined by mRNA accessibility to Ribogreen using the Quant-iT Ribogreen RNA assay (Thermo Fisher).

### In Vitro Cellular Uptake

LNPs were 5-fold diluted into PBS 1× and 25 μL were added on cells previously prepared in a final volume of 100 μL per well in their culture medium.

The luciferase assay (ONE-Glo™ Luciferase Assay System, Promega) was performed 48 h post-treatment in a 96-wells white cell culture plate and TECAN Spark plate reader were used to quantify the luminescence.

In mAb pre-incubation experiments, 5 μL of mAb were added on the cells for a final concentration of 100 μg/mL (saturating conditions) and incubated at room temperature for 30 minutes before treatment with LNPs.

### ELISA Binding Assays

For activity ELISA assay, recombinant hPD-1 (Sino Biologicals, Beijing, China; reference 10377-H08H) was immobilized on plastic at 1 μg/mL in carbonate buffer (pH 9.2) and t-LNPs or OSE-279 mAb were added to measure binding. After incubation and washing, peroxidase-labeled donkey anti-human IgG (Jackson Immunoresearch; USA; reference 709-035-149) was added and revealed by conventional methods.

### Example 1: Transduced U937 and Jurkat Cells, and HPB-ALL Cells Stably Express PD-1

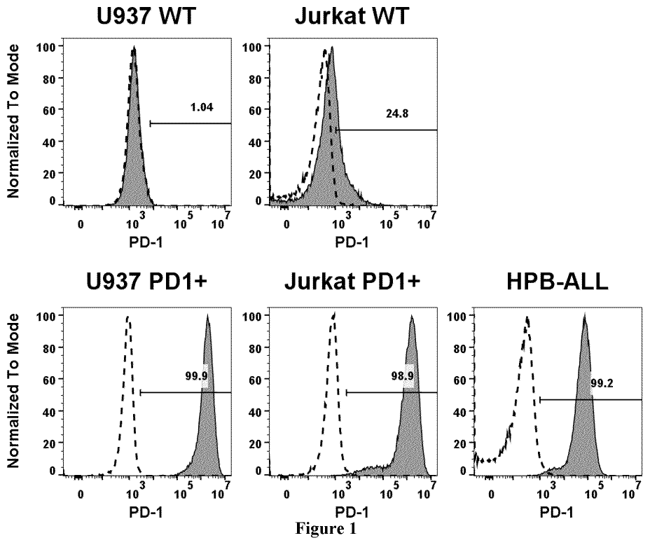

Flow cytometry was used to measure the percentage of PD-1+ cells. Analyses confirmed that all 3 cell types effectively express the PD-1 receptors (FIG. 1). Wild type U937 cells do not express PD-1 receptors, while wild type Jurkat cells express PD-1 receptors at a low level.

### Example 2: Addition of Anti-PD-1 Monoclonal Antibody OSE-279 During the Preparation of LNPs Allows for Improved Transfection in PD-1 Positive Cells Compared to WT Cells and to LNPs without Targeting Agent

The ability of anti-PD-1 mAb OSE-279 to be incorporated into the core of the LNPs for effective targeting of PD-1 expressing cells was evaluated by adding OSE-279 (10 μL) during the process of LNP formation.

Results show that OSE-279 can be added before the microfluidic mixing step via 2 different modalities to produce targeted-LNPs able to transfect PD-1 expressing cells with improved efficacy compared to WT cells.

The following configurations for the microfluidic equipment were assessed:

### Modality #1A:

Channel 1=mRNA (50 μL, 1 mg/mL in sodium citrate)+mAb (15 μL, 0.95 mg/mL in PBS)+acetate buffer pH 4.3 (535 μL, 25 mM)
Channel 2=lipid mix (200 μL, 9.1 mM in EtOH)

### Modality #1B:

Channel 1=mRNA (50 μL, 1 mg/mL in sodium citrate)+acetate buffer pH 4.3 (550 μL, 25 mM)
Channel 2=lipid mix (185 μL, 9.8 mM in EtOH)+mAb (15 μL, 0.95 mg/mL in PBS)

Transfection data for targeted-LNPS (t-LNPs) are presented below as fold-increase compared to non-transfected-LNPs (nt-LNPs).

Data obtained indicate that targeted-LNPs can be produced by adding OSE-279 mAb either in the mRNA solution (mixing modality #IA) or in the lipid solution (mixing modality #1B).

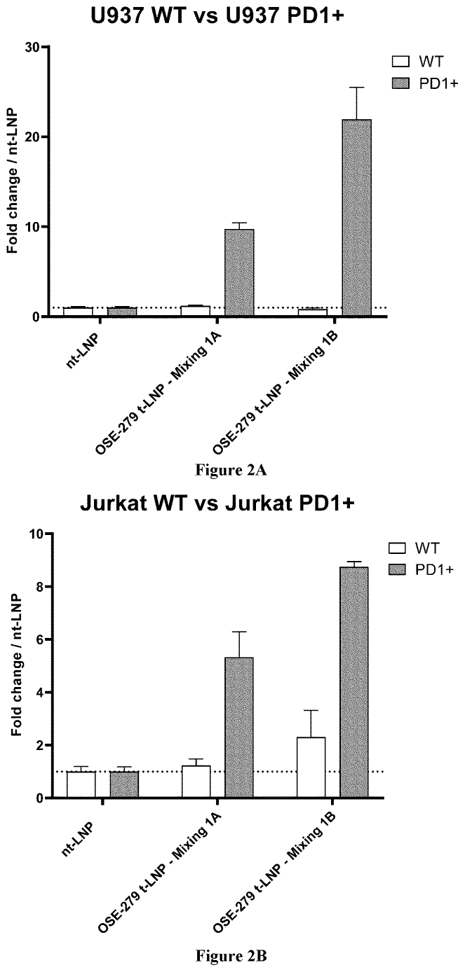

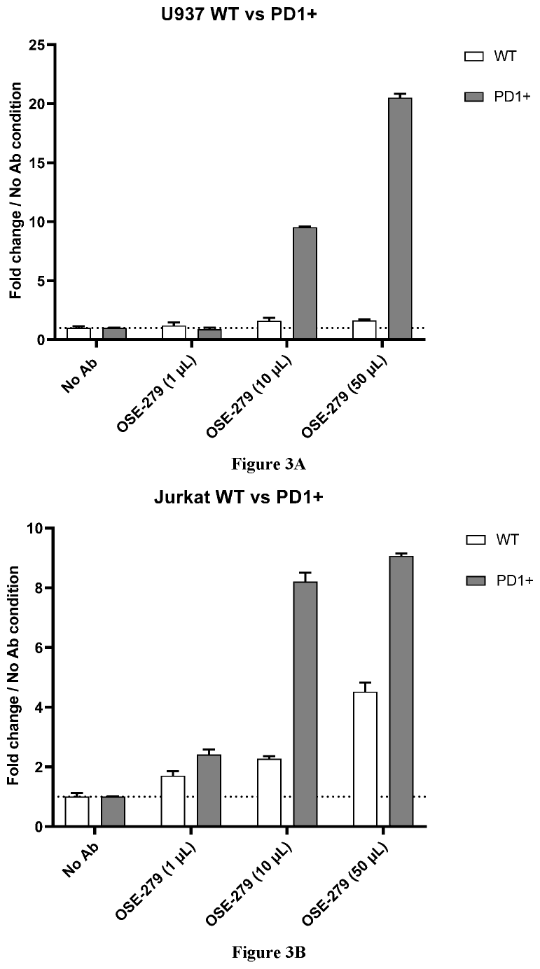

In both cases, targeted-LNPs displayed improved transfection efficacy in PD-1-expressing U937 (FIG. 2A) and Jurkat (FIG. 2B) cells, compared to WT cells and compared to non-targeted-LNPs.

### Example 3: Preparation and Characterization of Non-Targeted LNPs and Targeted-LNPs Encapsulating Fluc-mRNA

The non-targeted-LNPs and targeted-LNPs were prepared as detailed above using microfluidic mixing system (Inside Tx).

For non-targeted-LNPS (no addition of mAb OSE-279), this method resulted in particles with Z-Ave=132 nm (number-weighed diameter=84 nm) and PDI=0.153 and with good encapsulation (94% EF).

Addition of increasing amounts of OSE-279 monoclonal antibody (0.95 μg/μL, using mixing modality #1A) for the preparation of targeted-LNPs resulted in the production of particles characterized by i) larger diameters, ii) increased polydispersity index (PDI) and iii) reduced encapsulation efficiency compared to non-targeted-LNPs, as evidenced by DLS measurements and by Ribogreen assay (Table 1). As shown in Example 3 and following, this was not detrimental to the efficacity of the LNPs of the invention.

TABLE 1
Characterization of t-LNPs.

| Dose of OSE-279 in | | | Encapsulation |
|---|---|---|---|
| the LNPs (μL) | Z-Ave (nm) | PDI | Efficiency (%) |
| 0 | 132 | 0.153 | 94% |
| 10 | 159 | 0.169 | 83% |
| 15 | 160 | 0.187 | 77% |
| 25 | 208 | 0.247 | 66% |
| 50 | 251 | 0.285 | 52% |

### Example 4: Targeted-LNPs Display Improved Transfection Efficacy in PD-1 Positive Cells in a Dose-Dependent Manner

The mixing modality #1A was used in the following examples.

To confirm the potency of targeted-LNPs to preferentially deliver mRNA in cells expressing the PD-1 receptors, 3 different targeted-LNPs were prepared with increasing amount of OSE-279 mAb (1, 10 and 50 μL).

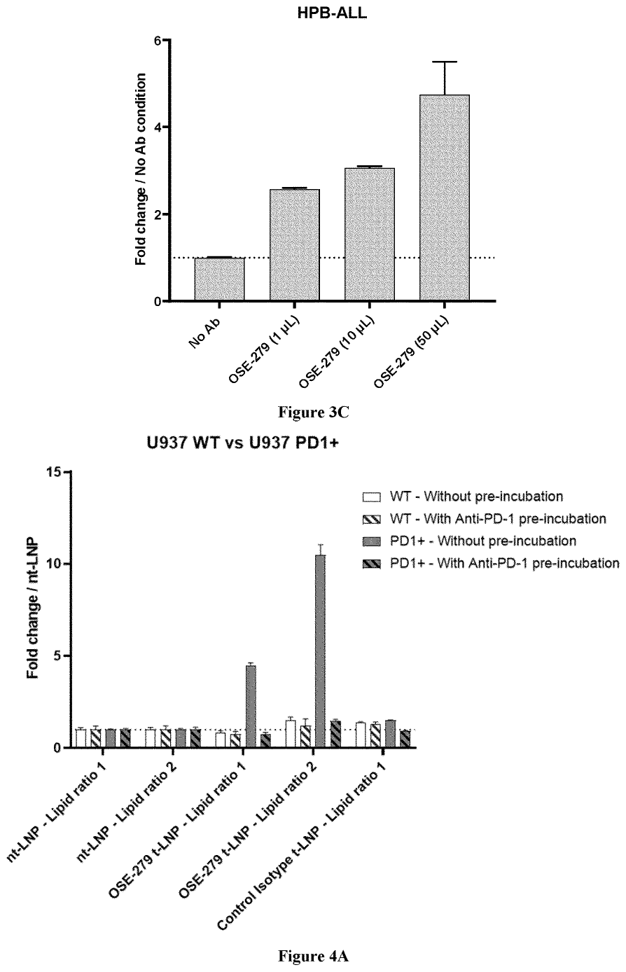

Non-targeted-LNPs and targeted-LNPs were then compared for their transfection potency, on different cell lines expressing or not-expressing PD-1: U937 WT vs U937 PD-1+ (FIG. 3A), Jurkat WT vs Jurkat PD-1+ (FIG. 3B) and HPB-ALL (cell line naturally expressing PD-1, FIG. 3C).

Results indicate that improved transfection compared to non-targeted-LNPs was observed in conditions combining PD-1-expressing cells and targeted-LNPs, in a dose-dependent manner (with the exception of the t-LNP with 50 μL of OSE-279 in WT Jurkat cells, where an increase in transfection was observed compared to nt-LNP).

### Example 5: OSE-279 Targeted-LNPs Prepared Using Different Lipid Ratios are Efficient in Preferentially Transfecting PD-1 Positive Cells

The influence of the ratio between the different lipidic constituents of the LNPs was assessed by preparing OSE-279 targeted-LNP with the lipid ratios described in Table 2.

TABLE 2
Variation of the ratio of lipids used in the LNPs

| Lipid component | Ratio 1 (mol %) | Ratio 2 (mol %) |
|---|---|---|
| ALC-0315 | 38 | 50 |
| DOPE | 12 | 10 |
| Cholesterol | 48 | 38.5 |
| DMG-PEG-2000 | 2 | 1.5 |

Results show that targeted-LNPs prepared with 10 μL of OSE-279 (mixing modality #1A) were able to preferably transfect PD-1 positive cells, compared i) to WT cells, ii) to non-targeted-LNPs and iii) to targeted-LNPs prepared with a control isotype mAb.

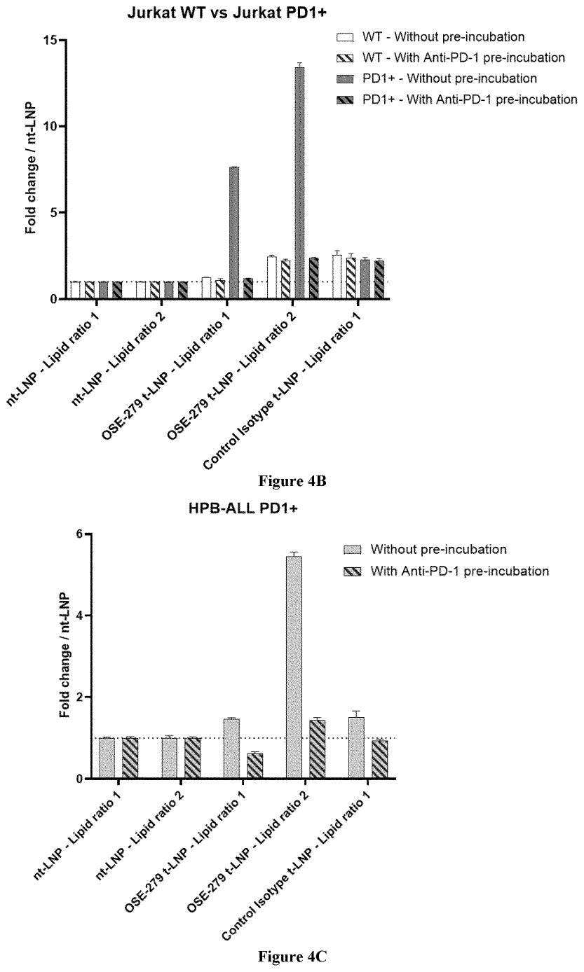

Both lipid ratios 1 and 2 allow to generate targeted-LNPs capable of inducing improved transfection on PD-1 expressing cells U937 (FIG. 4A), Jurkat (FIG. 4B) and HPB-ALL (FIG. 4C).

### Example 6: Incubation of PD-1 Positive Cells with the PD-1 Inhibitor OSE-279 Before the Transfection Reduced the Efficacy of Targeted-LNPs, Confirming a PD-1-Mediated Endocytosis Pathway for Targeted-LNPs

The lipid ratio 2 was used in the following examples.

To confirm that the improved transfection efficacy on PD-1 expressing cells was linked to the binding of the targeted-LNPs with the PD-1 receptors expressed on the cell surface, experiments were conducted where cells were pre-incubated 30 min before transfection with a saturating concentration of the anti-PD-1 mAb OSE-279 (100 μg/mL), with the objective of blocking the PD-1 receptors and therefore suppressing any possible binding with targeted-LNPs.

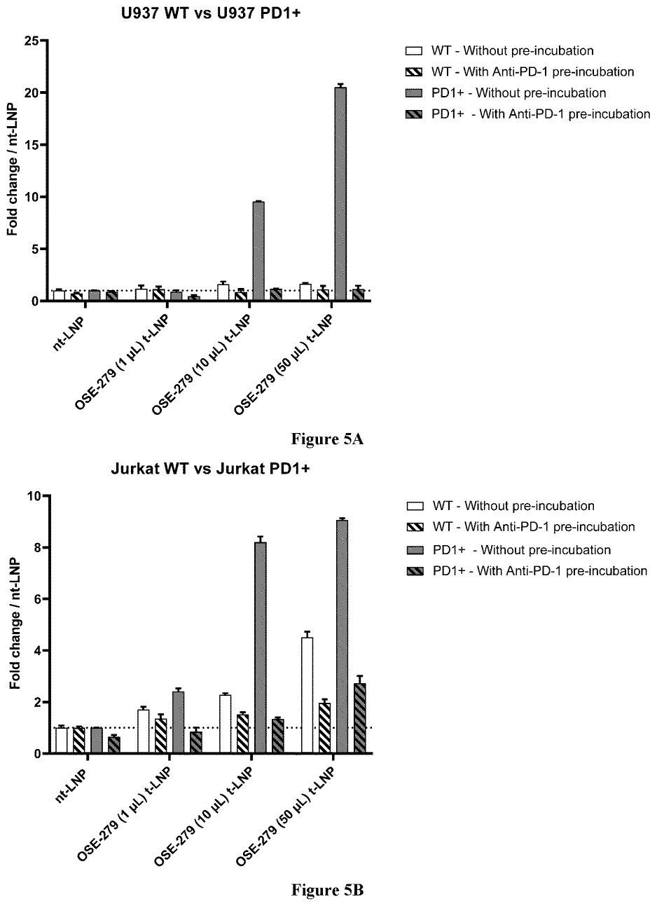

The results obtained in the pre-incubation experiments show that the transfection efficacy of targeted-LNPs in PD-1 expressing cells was significantly reduced to the level of non-targeted-LNPs (FIG. 5A for U937, FIG. 5B for Jurkat, FIG. 5C for HPB-ALL). Moreover, the pre-incubation step had no impact on U937 PD-1 negative WT cells (FIG. 5A) and a minor effect on PD-1 low WT Jurkat cells (FIG. 5B).

These results confirmed that the improved transfection observed on PD-1 expressing cells with targeted-LNPs (compared to WT cells and to nt-LNPs conditions) is correlated to the effective binding of the targeted-LNPs with the PD-1 receptors expressed on the cells. This binding allows for the targeted-LNP to be in close contact with the cells, leading to LNP endocytosis and subsequent expression of the protein encoded by the mRNA encapsulated in the LNP.

### Example 7: ELISA Binding Assays Confirmed that the Anti-PD-1 mAb OSE-279 in the Targeted-LNPs Efficiently Binds to its Target PD-1

To assess the functionality of mAb OSE-279 incorporation on the targeted-LNPs, ELISA binding assays were performed to compare the binding of mAb OSE-279 with OSE-279 targeted-LNPs.

Dose-response analyses were compared for the following groups:
OSE-279,
OSE-279 targeted-LNPs,
Isotype control targeted-LNPs.

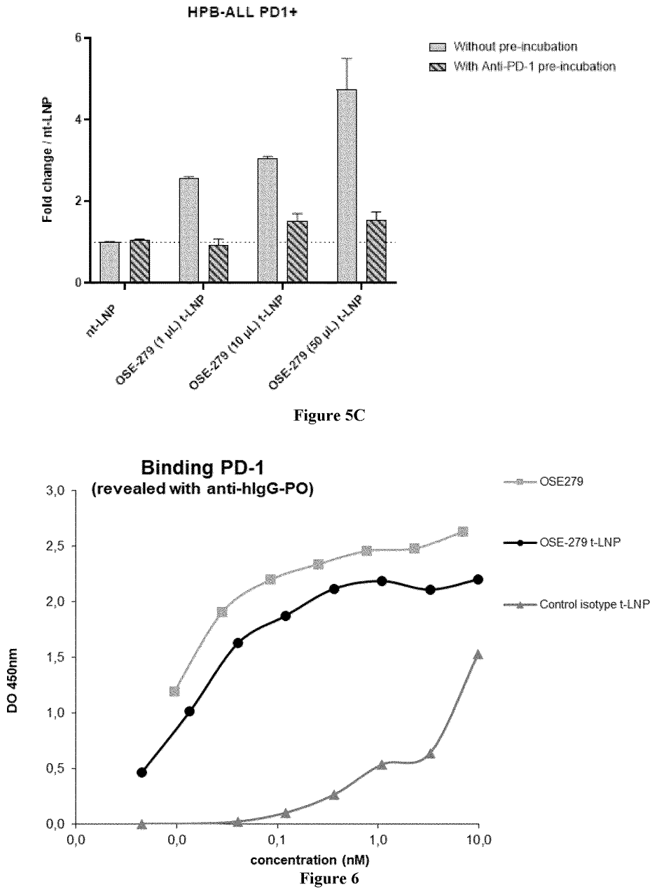

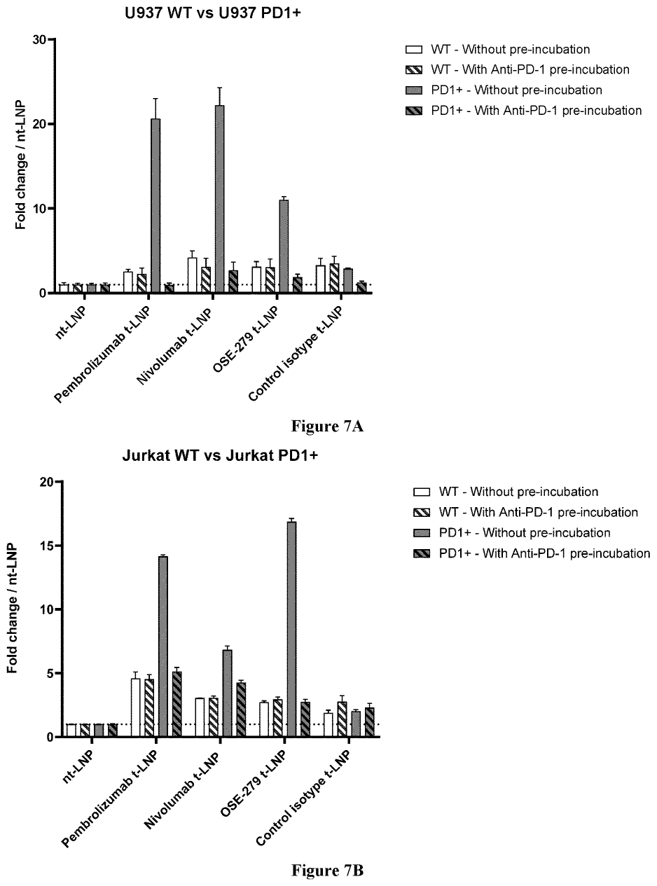

Results showed an effective binding of the targeted-LNPs to the PD-1 receptor (FIG. 6); this binding was almost identical to that of the mAb OSE-279 “alone”. More precisely, data indicated that 80% of the total mAb initially introduced during the preparation of targeted-LNPs were able to efficiently bind to their targets PD-1 when complexed in the targeted-LNPs.

### Example 8: Different Anti-PD-1 mAbs can be Used to Prepare Targeted-LNPs with Improved Transfection on PD-1 Positive Cells

To address the possibility of using various anti-PD-1 mAbs to prepare targeted-LNPs, Pembrolizumab, Nivolumab and OSE-279 were used to produce targeted-LNPs (addition of 10 μL of mAb during the formation of the t-LNP).

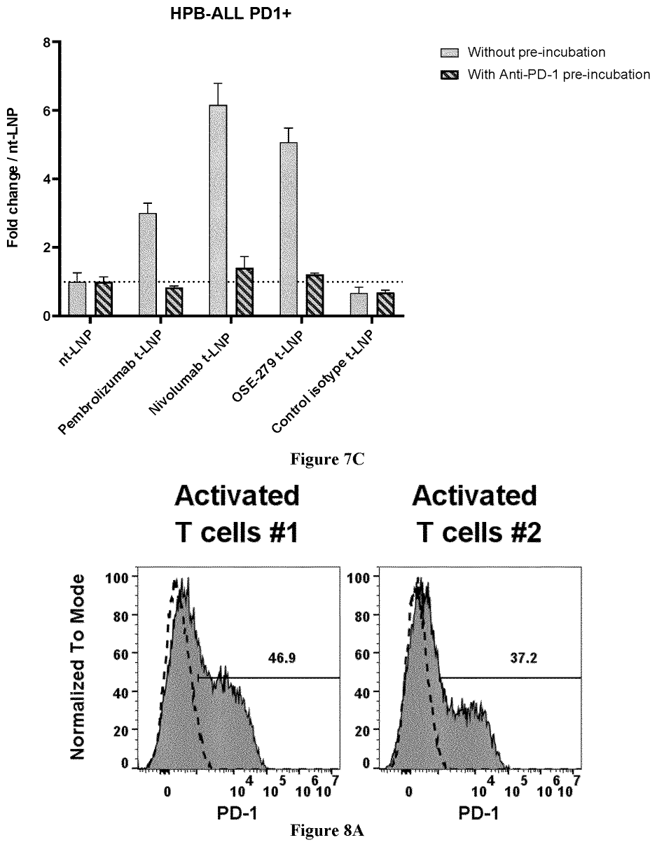

Results show that targeted-LNPs prepared from Pembrolizumab, Nivolumab or OSE-279 all display improved transfection in PD-1 expressing-cells compared to WT-cells and to non-targeted-LNPs (FIG. 7A for U937, FIG. 7B for Jurkat, FIG. 7C for HPB-ALL).

As a complement, pre-incubation experiments were conducted using the same mAb than the one used for preparing the targeted-LNPs (i.e., Pembrolizumab was used to saturate the PD-1 receptors in the Pembrolizumab t-LNP pre-incubation). In these experiments, as observed previously, the pre-incubation step suppressed the beneficial effect of targeted-LNPs on the transfection of PD-1 expressing cells.

Control experiments with targeted-LNPs prepared with control isotype mAb did not show any differences i) between WT or PD-1+ cell types and ii) between experiments performed with or without pre-incubation with saturating concentration of anti-PD-1 mAbs.

### Example 9: Targeted-LNPs Display Improved Transfection on Hard-to-Transfect Activated T Cells Expressing PD-1 Receptor, in a Dose-Dependent Manner, Compared to Non-Targeted-LNPs

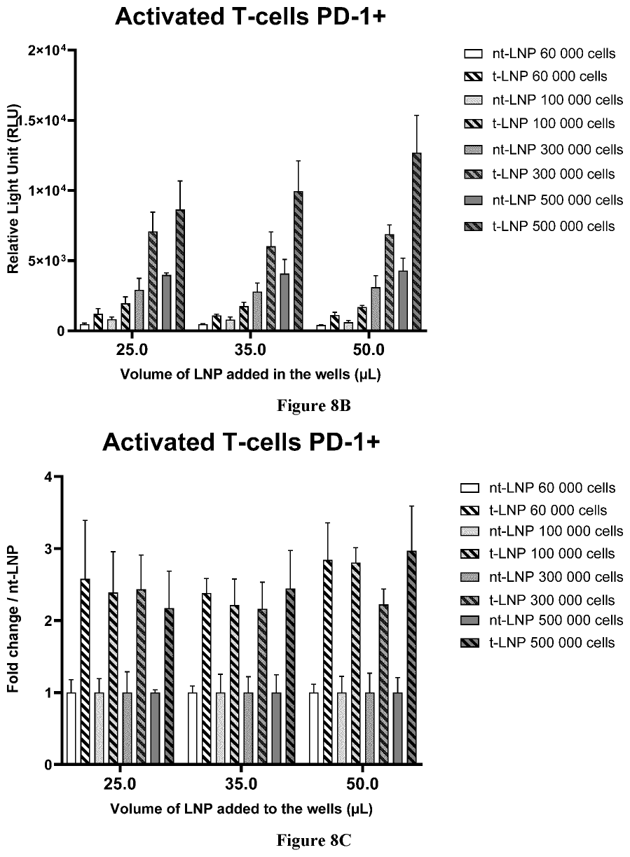

T cells were isolated from human PBMCs and activated via CD3/CD28 stimulation to induce expression of PD-1 receptor. Flow cytometry was used to measure the percentage of PD-1+ and cells expressing target 2 (CD127). A Cytoflex flow cytometer (Beckman) was used for reading and analysis were performed with FlowJo software. Analysis show that activated T cells express PD-1 receptors (46.9% for donor 1, 37.2% for donor 2, FIG. 8A).

The ability of OSE-279 targeted-LNPs to transfect activated T cells was evaluated compared to non-targeted-LNPs, either in Relative Light Units (FIG. 8B) or illustrated in fold changes (FIG. 8C). Different experimental conditions were tested for the transfection, with varying amounts of cells (60 000 to 500 000 cells) and varying concentrations of LNP added to the wells (25 to 50 μL/well). In all tested conditions, OSE-279 targeted LNPs display improved transfection compared to non-targeted-LNPs (2-3 fold increase).

### Example 10: Targeted-LNPs are Able to Target Different Cell Receptors Depending on the Nature of the mAb Used in their Preparation, for an Improved Transfection in the Cells Expressing these Receptors

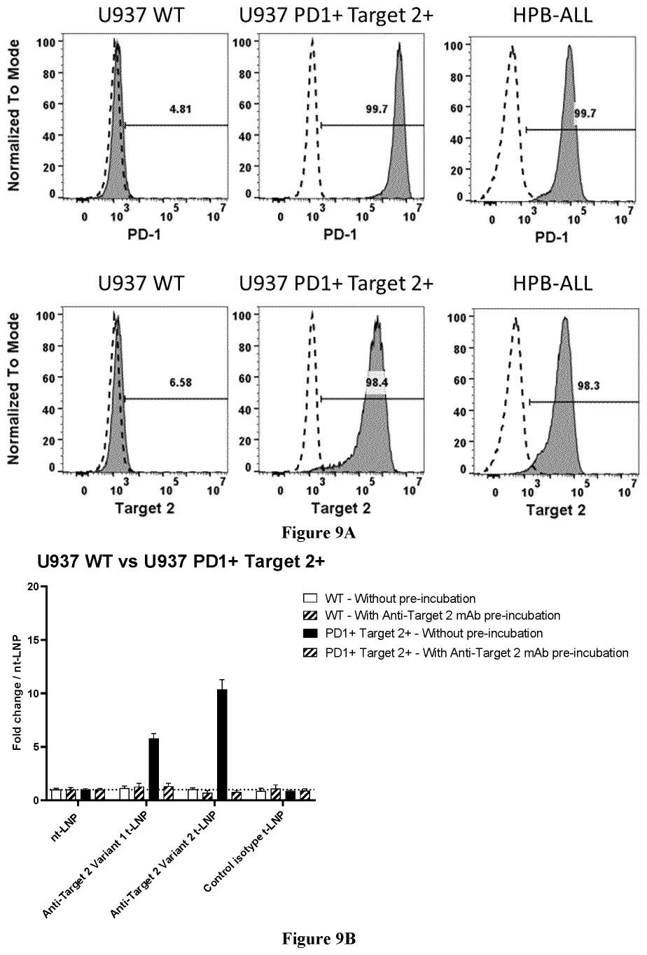

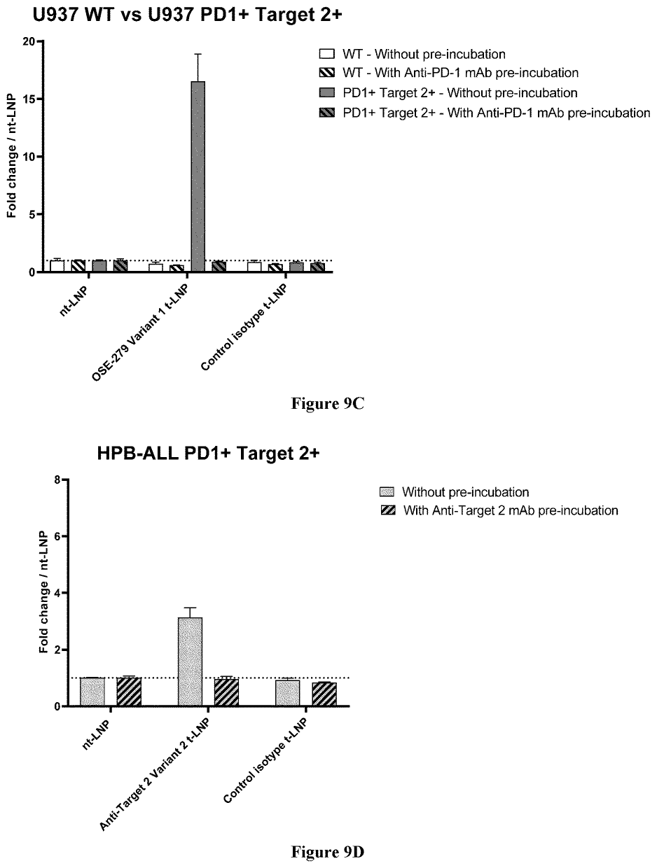

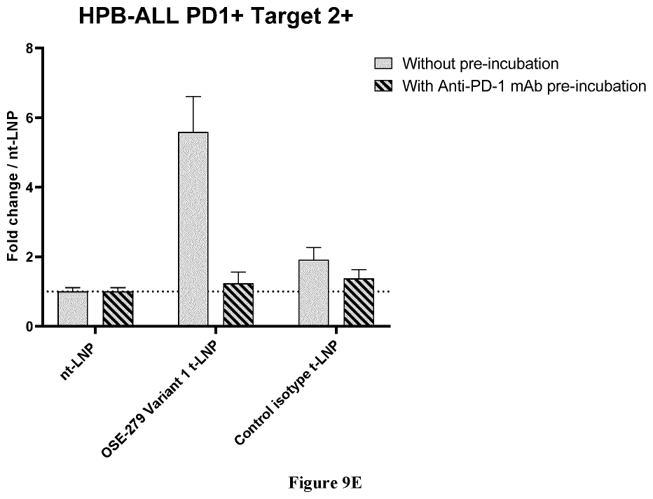

To further broaden the possible applications of targeted-LNPs, their ability to efficiently target and transfect cells expressing various receptors was investigated. In this context, a model of U937 cells transduced to express both PD-1 and target 2 receptors was used. Flow cytometry was used to measure the percentage of PD-1+ and cells expressing target 2 (FIG. 9A). Target 2 is CD127.

Targeted-LNPs were prepared using 15 μL of mAb, being either an anti-target 2 antagonist mAb or an anti-PD-1 mAb (OSE-279, as described previously). Results show that targeted-LNPs were able to induce an improved transfection in cells expressing the target 2 and the PD-1 receptors (U937 cells in FIG. 9A-B and HPB-ALL cells in FIG. 9C-D):
Either via target 2-targeting, by using targeted-LNPs carrying an anti-target 2 mAb (FIGS. 9B and 9D),
Or via PD-1 targeting, by using targeted-LNPs carrying an anti-PD-1 mAb (FIGS. 9C and 9E).

As observed in the previous experiments, the improvements in transfection potency were suppressed when a pre-incubation step with a saturating concentration of either an anti-target 2 or an anti-PD-1 mAbs was performed before the transfection protocols, confirming that the binding of the targeted-LNP with its receptor is a prerequisite for an improved transfection.

### Example 11: Anti-PD-1 Targeted-LNPs Encapsulating mRNA Encoding for Apoptosis-Inducing Proteins Allow for Higher Cell Death in PD-1 Expressing Cells, Compared to Non-Targeted-LNPs

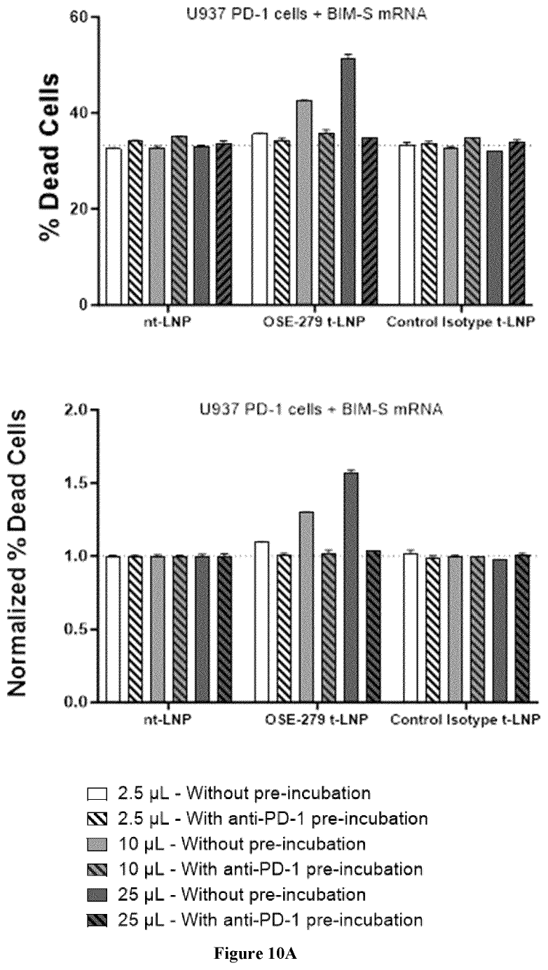

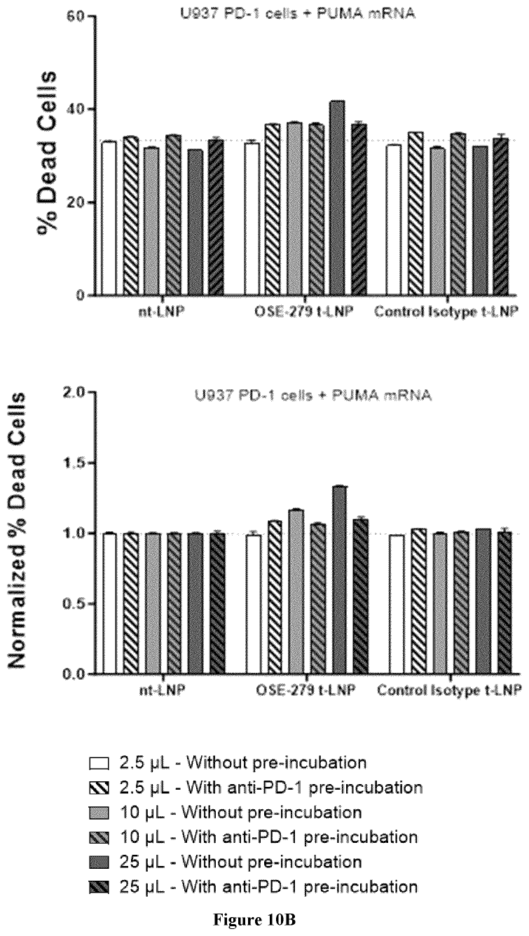

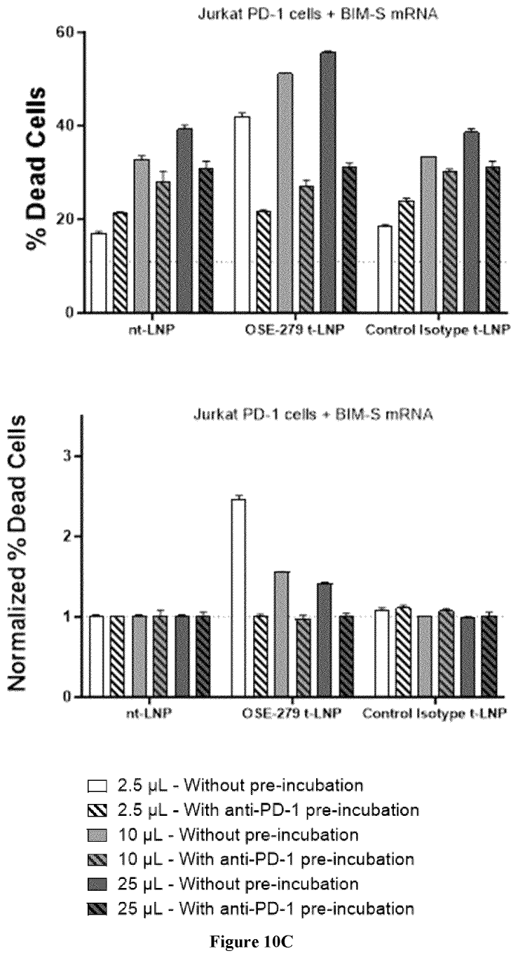

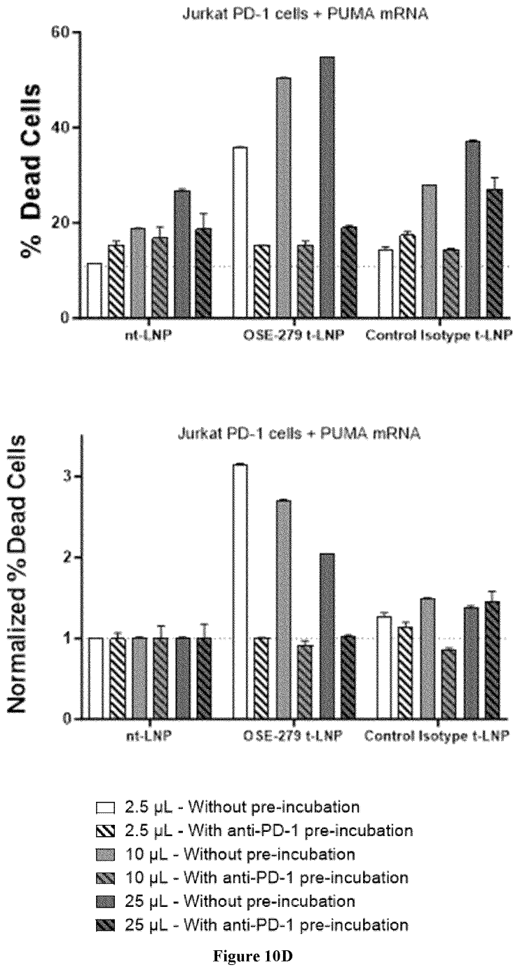

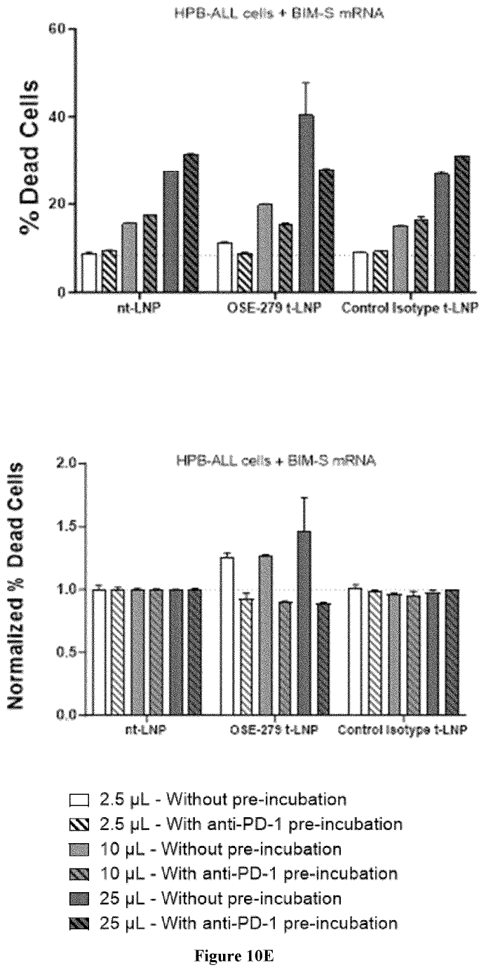

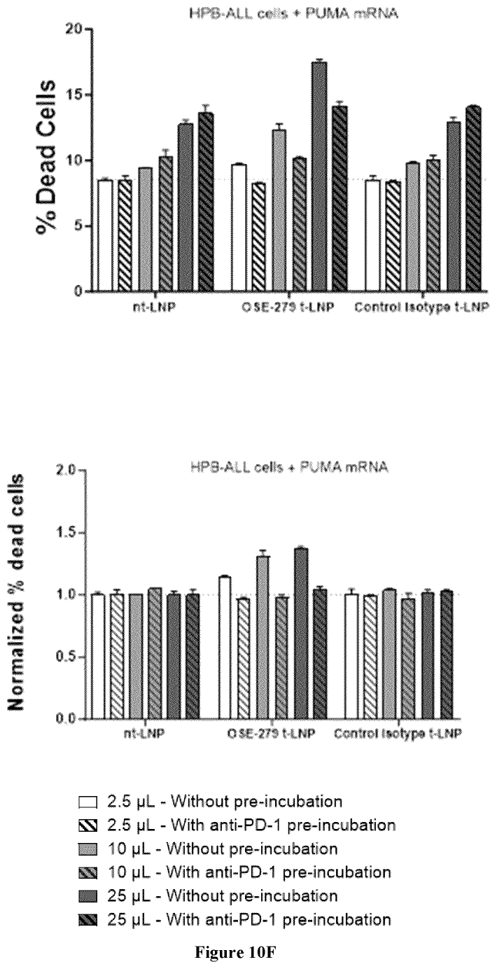

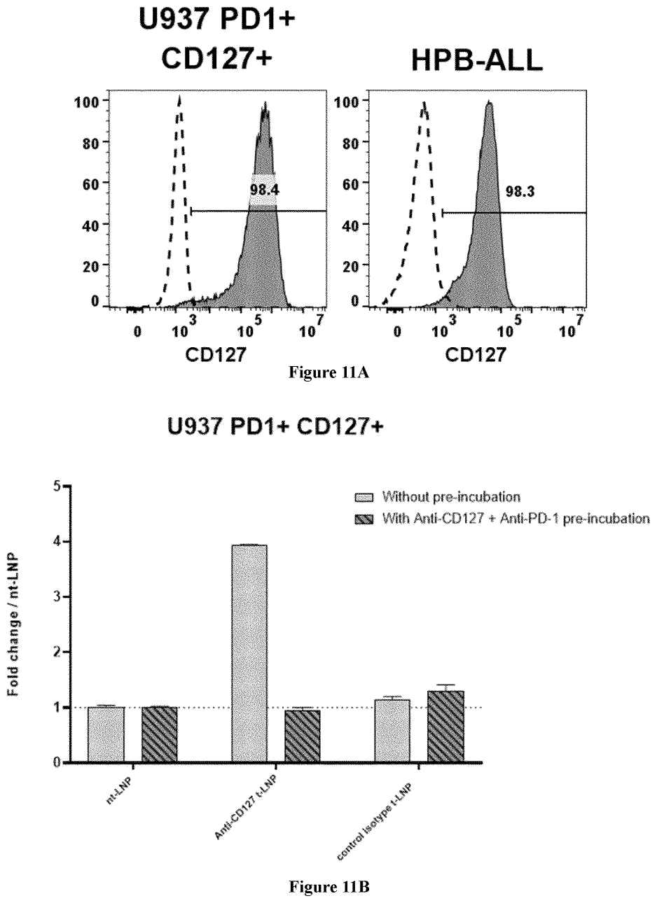

Targeted LNPs were tested for their potency to induce directed apoptosis in PD-1 expressing cells. In this purpose, mRNAs encoding for apoptosis-inducing proteins, namely BIM-S or PUMA, were encapsulated in targeted-LNPs prepared using OSE-279 as anti-PD-1 monoclonal antibody. In-vitro transfection was assessed in 3 different cell types expressing the PD-1 receptor: U937 (FIGS. 10A and 10B), Jurkat (FIGS. 10C and 10D) and HPB-ALL (FIGS. 10E and 10F) with encapsulated mRNA encoding either for the BIM-S protein (FIGS. 10A, 10C and 10E) or the PUMA protein (FIGS. 10B, 10D and 10F). Non-targeted LNPs and targeted LNPs with a control isotype mAb were used as negative controls.

Data are presented in percentage of cell death (top figure—dashed line representing the basal cell death in the absence of LNP treatment) and in percentage of cell death normalized versus the non-targeted LNP conditions (bottom figure—dashed line representing the normalized percentage for non-targeted conditions).

Results show that t-LNPs are able to induce higher cell death in PD-1 expressing cells compared to negative controls, in all 3 cell types tested. Moreover, as observed in the previous experiments, the improvements in potency were suppressed when the pre-incubation step with a saturating concentration of an anti-PD-1 mAb was performed before the transfection protocols (as evidenced by the comparison between empty bars and crossed bars in FIG. 10), confirming that the binding of the targeted-LNP with the cell receptor is a prerequisite for the observed improved biological effect.

### Example 12: Targeted-LNPs are Able to Target Different Cell Receptors Depending on the Nature of the mAb Used in their Preparation, for an Improved Transfection in the Cells Expressing these Receptors: Example of CD-127 Expressing Cells Targeted with Anti-CD-127 Targeted LNPs

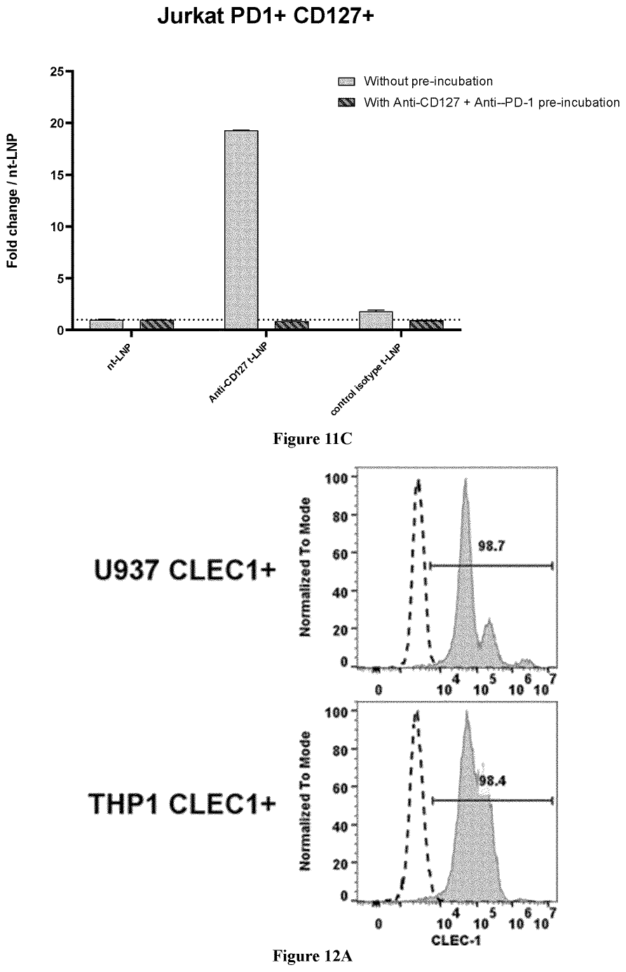

To further broaden the possible applications of targeted LNPs, their ability to efficiently target and transfect cells expressing various receptors was investigated. In this context, a model of U937 and Jurkat cells transduced to express CD-127 receptors was used. Flow cytometry was used to measure the percentage of CD-127 positive cells (FIG. 11A).

### Preparation of LNP

LNPs were prepared using microfluidic mixing. Briefly, lipids were dissolved in ethanol with the following molar ratios ALC-0315:50%/DOPE:10%/Cholesterol:38.5%/DMG-PEG-2000:1.5%, at a total lipid concentration of 4.55 mM. FLuc-mRNA (25 μL, 1 mg/mL) was diluted with 25 mM acetate buffer (pH 4.3) to a final concentration of 0.041 mg/mL. For the preparation of targeted-LNPs, the anti-human CD-127 mAb (0.95 μg/μL in PBS, 1.3×10⁻⁶ μmol mAb/μg RNA) was added to the mRNA solution prior to microfluidic mixing. The selected experimental conditions allow for a N/P ratio of 6.

The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 3
Targeting agent and characteristics of LNP produced and tested

| Ref | Targeting agent | Z-Ave | PDI | % EE |
|---|---|---|---|---|
| 3121 | No Ab | 108 | 0.125 | 83% |
| 3123 | anti-human CD-127 | 137 | 0.181 | 76% |
| 3127 | Control isotype | 136 | 0.232 | 77% |

### In Vitro Transfection

LNPs were then diluted to 3.9 ng/μL with PBS 1× and added on cells previously prepared in a final volume of 60 μL per well in their culture medium (addition of 25 μL LNP/well corresponding to 97 ng RNA/well for U937 cells and 2.5 μL LNP/well corresponding to 9.7 ng RNA/well for Jurkat cells).

The luciferase assay (ONE-Glo™ Luciferase Assay System, Promega) was performed 48 h post-treatment in a 96-wells white cell culture plate and TECAN Spark plate reader were used to quantify the luminescence.

In mAb pre-incubation experiments, 5 μL of each anti-CD-127 and OSE-279 mAb (9 mg/mL) were added on the cells for a final concentration of 500 μg/mL (saturating conditions for both CD-127 and PD-1 receptors) and incubated at room temperature for 30 minutes before treatment with LNPs.

Results show that anti-CD-127-targeted LNPs were able to induce an improved transfection in cells expressing the CD-127 receptors (U937 cells in FIG. 11B and Jurkat cells in FIG. 11C).

As observed in the previous experiments, the improvements in transfection potency were suppressed when a pre-incubation step with a saturating concentration of an anti-CD-127 mAbs was performed before the transfection protocols, confirming that the binding of the targeted-LNP with its receptor is a prerequisite for an improved transfection.

### Example 13: Targeted-LNPs are Able to Target Different Cell Receptors Depending on the Nature of the mAb Used in their Preparation, for an Improved Transfection in the Cells Expressing these Receptors: Example of CLEC-1 Expressing Cells Targeted with Anti-CLEC-1 Targeted LNPs

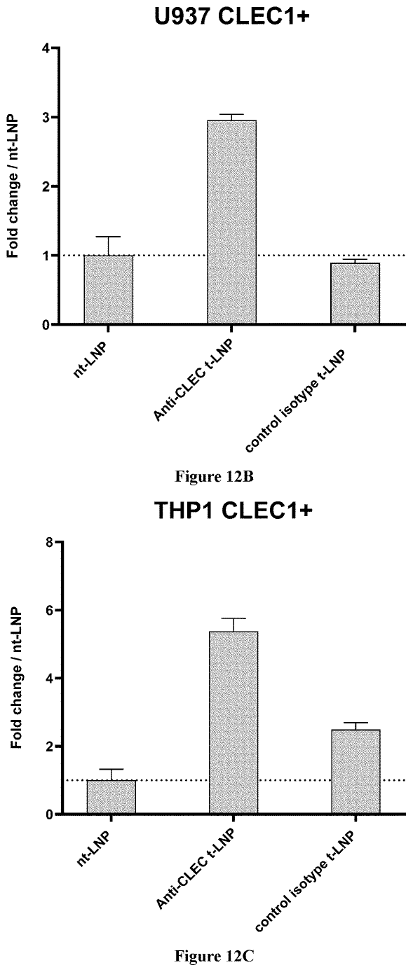

To further broaden the possible applications of targeted LNPs, their ability to efficiently target and transfect cells expressing various receptors was investigated. In this context, a model of U937 and Jurkat cells transduced to express CLEC-1 receptors was used. Flow cytometry was used to measure the percentage of CLEC-1 positive cells (FIG. 12A).

### Preparation of LNP

LNPs were prepared using microfluidic mixing. Briefly, lipids were dissolved in ethanol with the following molar ratios ALC-0315:50%/DOPE:10%/Cholesterol:38.5%/DMG-PEG-2000:1.5%, at a total lipid concentration of 9.1 mM. FLuc-mRNA (50 μL, 1 mg/mL) was diluted with 25 mM acetate buffer (pH 4.3) to a final concentration of 0.083 mg/mL. For the preparation of targeted-LNPs, the anti-CLEC1 mAb (0.95 μg/μL in PBS, 1.3×10⁻⁶ μmol mAb/μg RNA) was added to the mRNA solution prior to microfluidic mixing. The selected experimental conditions allow for a N/P ratio of 6.

The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 4
Targeting agents and characteristics of LNP produced and tested

| Ref | Targeting agent | Z-Ave | PDI | % EE |
|---|---|---|---|---|
| 377 | No Ab | 126 | 0.139 | 92% |
| 380 | Anti-CLEC1 | 145 | 0.203 | 79% |
| 381 | Control isotype | 158 | 0.185 | 75% |

### In Vitro Transfection

LNPs were diluted to 5 ng/μL with PBS 1× and added (5 μL of LNP/well corresponding to 25 ng RNA for U937 cells and 2.5 μL of LNP/well corresponding to 12.5 ng RNA for THP1 cells) on cells previously prepared in a final volume of 60 μL per well in their culture medium and pre-treated with 5 μL of pure serum albumin bovine (SAB pre-treatment 15 min before LNP addition).

The luciferase assay (ONE-Glo™ Luciferase Assay System, Promega) was performed 48 h post-treatment in a 96-wells white cell culture plate and TECAN Spark plate reader were used to quantify the luminescence.

Results show that anti-CLEC1-targeted LNPs were able to induce an improved transfection in cells expressing the CLEC-1 receptors (U937 cells in FIG. 12B and THP-1 cells in FIG. 12C).

### Example 14: Targeted-LNPs are Able to Target Different Cell Receptors Depending on the Nature of the mAb Used in their Preparation, for an Improved Transfection in the Cells Expressing these Receptors: Example of SIRPα Expressing Cells Targeted with Anti-SIRPα Targeted LNPs

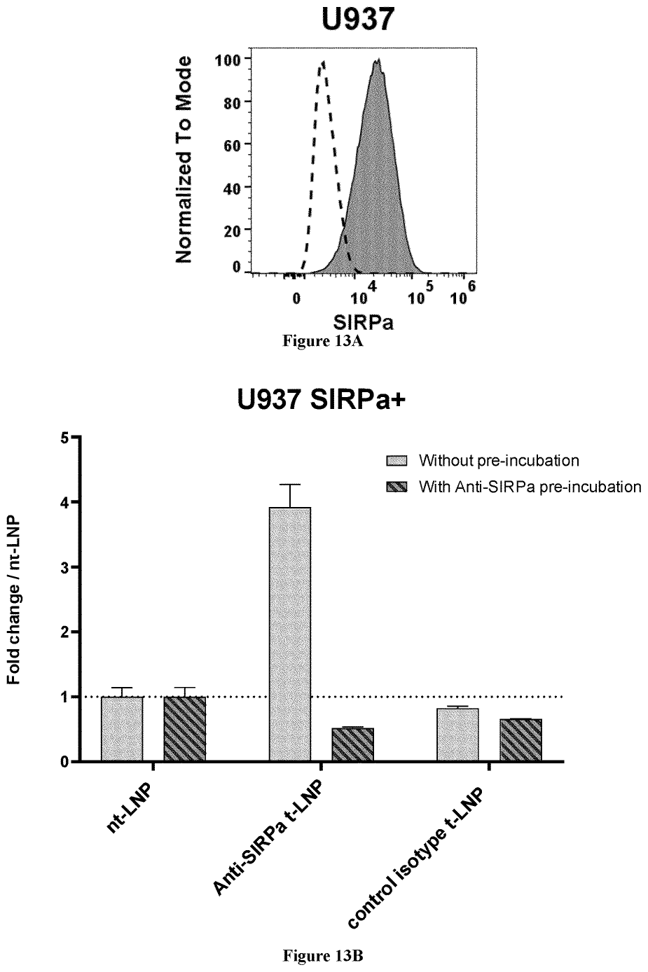

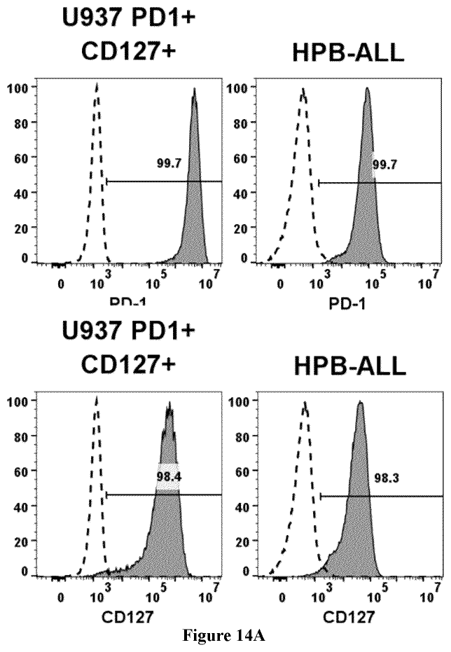

To further broaden the possible applications of targeted LNPs, their ability to efficiently target and transfect cells expressing various receptors was investigated. In this context, a model of U937 cells transduced to express SIRPα receptors was used. Flow cytometry was used to measure the percentage of SIRPα positive cells (FIG. 13A).

### Preparation of LNP

LNPs were prepared using microfluidic mixing. Briefly, lipids were dissolved in ethanol with the following molar ratios ALC-0315:50%/DOPE:10%/Cholesterol:38.5%/DMG-PEG-2000:1.5%, at a total lipid concentration of 9.1 mM. FLuc-mRNA (50 μL, 1 mg/mL) was diluted with 25 mM acetate buffer (pH 4.3) to a final concentration of 0.083 mg/mL. For the preparation of targeted-LNPs, the anti-human SIRPα mAb (0.95 μg/μL in PBS, 1.3×10⁻⁶ μmol mAb/μg RNA) was added to the mRNA solution prior to microfluidic mixing. The selected experimental conditions allow for a N/P ratio of 6.

The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 5
Targeting agent and characteristics of LNP produced and tested

| Ref | Conditions | Z-Ave | PDI | % EE |
|---|---|---|---|---|
| 3152 | No Ab | 148 | 0.061 | 87% |
| 3183 | Anti-human SIRPα | 191 | 0.192 | 82% |
| | antibody | | | |
| 3186 | Control isotype | 195 | 0.163 | 78% |

### In Vitro Transfection

LNPs were diluted to 1.2 ng/μL with PBS 1× and added on cells previously prepared in a final volume of 60 μL per well in their culture medium (addition of 1 μL LNP/well corresponding to 1.2 ng RNA/well).

The luciferase assay (ONE-Glo™ Luciferase Assay System, Promega) was performed 24 h post-treatment in a 96-wells white cell culture plate and TECAN Spark plate reader were used to quantify the luminescence.

In mAb pre-incubation experiments, 5 μL of each mAb (9 mg/mL) were added on the cells for a final concentration of 500 μg/mL (saturating conditions) and incubated at room temperature for 30 minutes before treatment with LNPs.

Results show that anti-human SIRPα targeted LNPs were able to induce an improved transfection in cells expressing the SIRPα receptors (U937 cells in FIG. 13B).

As observed in the previous experiments, the improvements in transfection potency were suppressed when a pre-incubation step with a saturating concentration of an anti-SIRPα mAbs was performed before the transfection protocols, confirming that the binding of the targeted-LNP with its receptor is a prerequisite for an improved transfection.

### Example 15: Targeted-LNPs are Able to Target Different Cell Receptors Depending on the Nature of the mAb Used in their Preparation, for an Improved Transfection in the Cells Expressing these Receptors: Example of PD-1 and CD-127 Expressing Cells Targeted with Anti-PD-1 (OSE-279) and Anti-CD-127 Dual-Targeted LNPs

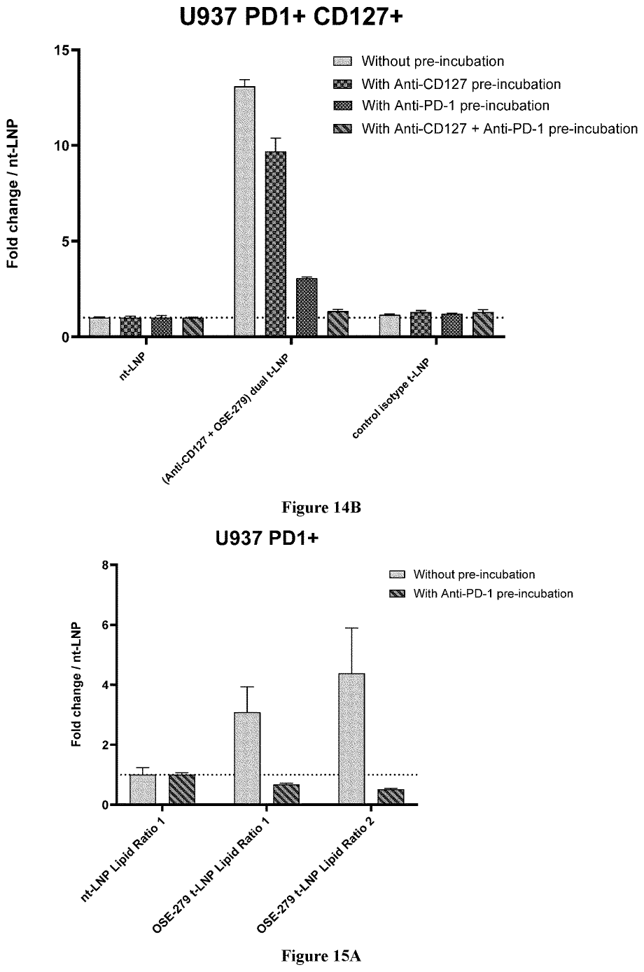

To further broaden the possible applications of targeted LNPs, their ability to efficiently target and transfect cells expressing various receptors was investigated. In this context, a model of U937 cells transduced to express both PD-1 and CD-127 receptors was used. Flow cytometry was used to measure the percentage of both PD-1 and CD-127 positive cells (FIG. 14A).

### Preparation of LNP

LNPs were prepared using microfluidic mixing. Briefly, lipids were dissolved in ethanol with the following molar ratios ALC-0315:50%/DOPE:10%/Cholesterol:38.5%/DMG-PEG-2000:1.5%, at a total lipid concentration of 9.1 mM. FLuc-mRNA (50 μL, 1 mg/mL) was diluted with 25 mM acetate buffer (pH 4.3) to a final concentration of 0.083 mg/mL. For the preparation of targeted-LNPs, the 2 mAbs anti-CD-127 and OSE-279 (0.95 μg/μL in PBS, each 0.65×10⁻⁶ μmol mAb/μg RNA) were successively added in equimolar amount to the mRNA solution prior to microfluidic mixing. The selected experimental conditions allow for a N/P ratio of 6.

The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 6
Targeting agent and characteristics of LNP produced and tested

| Ref | Targeting agent | Z-Ave | PDI | % EE |
|---|---|---|---|---|
| 3121 | No Ab | 108 | 0.125 | 83% |
| 3125 | anti-CD-127 + OSE-279 | 115 | 0.201 | 79% |
| 3127 | Control isotype | 136 | 0.232 | 77% |

### In Vitro Transfection

LNPs were diluted to 3.9 ng/μL with PBS 1× and added on cells previously prepared in a final volume of 60 μL per well in their culture medium (addition of 25 μL LNP/well corresponding to 97 ng RNA/well).

The luciferase assay (ONE-Glo™ Luciferase Assay System, Promega) was performed 48 h post-treatment in a 96-wells white cell culture plate and TECAN Spark plate reader were used to quantify the luminescence.

In mAb pre-incubation experiments, 5 μL of each mAb (9 mg/mL) were added on the cells for a final concentration of 500 μg/mL (saturating conditions) and incubated at room temperature for 30 minutes before treatment with LNPs.

Results show that (anti-human CD-127 antibody+OSE-279) dual-targeted LNPs were able to induce an improved transfection in cells expressing the PD-1 and CD-127 receptors (U937 cells in FIG. 14B).

As observed in the previous experiments, the improvements in transfection potency were suppressed when a pre-incubation step with a saturating concentration of an both anti-PD-1 and anti-CD-127 mAbs was performed before the transfection protocols, confirming that the binding of the targeted-LNP with its receptor is a prerequisite for an improved transfection.

Of note, the targeting efficacy was still observed when only a single mAb was used for the pre-incubation step (either anti-PD-1 alone or anti-CD-127 alone), showing that both types of receptors were involved in the improvement of transfection observed with the dual-targeted LNPs.

### Example 16: OSE-279 Targeted-LNPs Prepared Using 2 Different DOPE/Cholesterol Ratios are Both Efficient in Preferentially Transfecting PD-1 Positive Cells

The influence of the ratio between the phospholipid DOPE and Cholesterol in the LNPs was assessed by preparing OSE-279 targeted-LNP with the lipid ratios described below.

### Preparation of LNP

LNPs were prepared using microfluidic mixing. Briefly, lipids were dissolved in ethanol with the following molar ratios, at a total lipid concentration of 9.1 mM:
Ratio 1: ALC-0315:50%/DOPE:10%/Cholesterol:38.5%/DMG-PEG-2000:1.5 0,
Ratio 2: ALC-0315:50%/DOPE:38.5%/Cholesterol:10%/DMG-PEG-2000:1.5%.

FLuc-mRNA (50 μL, 1 mg/mL) was diluted with 25 mM acetate buffer (pH 4.3) to a final concentration of 0.083 mg/mL. For the preparation of targeted-LNPs, the mAb (0.95 μg/μL in PBS, 1.9×10⁻⁶ μmol mAb/μg RNA) was added to the mRNA solution prior to microfluidic mixing. The selected experimental conditions allow for a N/P ratio of 6.

The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 7
Targeting agent and characteristics of LNP produced and tested

| Ref | Targeting agent | Lipid ratio | Z-Ave | PDI | % EE |
|---|---|---|---|---|---|
| 350 | No Ab | 1 | 140 | 0.139 | 83% |
| 351 | OSE-279 | 1 | 183 | 0.219 | 60% |
| 352 | OSE-279 | 2 | 214 | 0.230 | 62% |

### In Vitro Transfection

LNPs were diluted to 5.0 ng/μL with PBS 1× and added on cells previously prepared in a final volume of 60 μL per well in their culture medium (addition of 25 μL LNP/well corresponding to 125 ng RNA/well).

The luciferase assay (ONE-Glo™ Luciferase Assay System, Promega) was performed 48 h post-treatment in a 96-wells white cell culture plate and TECAN Spark plate reader were used to quantify the luminescence.

In mAb pre-incubation experiments, 5 μL of mAb (9 mg/mL) were added on the cells for a final concentration of 500 μg/mL (saturating conditions) and incubated at room temperature for 30 minutes before treatment with LNPs.

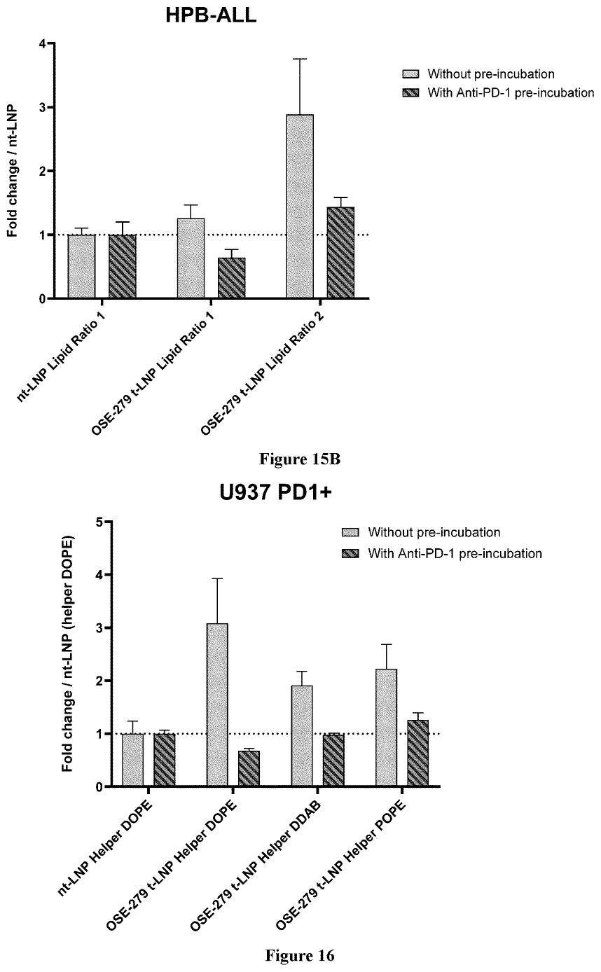

Results show that, independently of the DOPE/Cholesterol ratio used, OSE-279 targeted LNPs were able to induce an improved transfection in cells expressing the PD-1 receptors (U937 cells in FIG. 15A, and HPB-ALL cells in FIG. 15B).

As observed in the previous experiments, the improvements in transfection potency were suppressed when a pre-incubation step with a saturating concentration of an anti-PD-1 mAbs was performed before the transfection protocols, confirming that the binding of the targeted-LNP with its receptor is a prerequisite for an improved transfection.

### Example 17: OSE-279 Targeted-LNPs Prepared Using 3 Different Helper Lipids are Efficient in Preferentially Transfecting PD-1 Positive Cells

The influence of the nature of the helper lipid in the LNPs was assessed by preparing OSE-279 targeted-LNP with either DOPE, POPE or DDAB as helper lipid.

### Preparation of LNP

LNPs were prepared using microfluidic mixing. Briefly, lipids were dissolved in ethanol with the following molar ratios ALC-0315: 50%/DOPE: 10%/Cholesterol: 38.5%/DMG-PEG-2000: 1.5% (Ratio 1) or ALC-0315: 50%/DOPE or DDAB or POPE: 38.5%/Cholesterol: 10%/DMG-PEG-2000: 1.5% (Ratio 2), at a total lipid concentration of 9.1 mM. FLuc-mRNA (50 μL, 1 mg/mL) was diluted with 25 mM acetate buffer (pH 4.3) to a final concentration of 0.083 mg/mL. For the preparation of targeted-LNPs, the mAb (0.95 μg/μL in PBS, 1.9×10⁻⁶ μmol mAb/μg RNA) was added to the mRNA solution prior to microfluidic mixing. The selected experimental conditions allow for a N/P ratio of 6.

The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 8
Targeting agent and characteristics of LNP produced and tested

| Ref | Targeting agent | Helper lipid | Z-Ave | PDI | % EE |
|---|---|---|---|---|---|
| 350 | No Ab | DOPE | 140 | 0.139 | 83% |
| 351 | OSE-279 | DOPE | 183 | 0.219 | 60% |
| 353 | OSE-279 | DDAB | 229 | 0.213 | 59% |
| 355 | OSE-279 | POPE | 167 | 0.206 | 50% |

### In Vitro Transfection

LNPs were diluted to 5.0 ng/μL with PBS 1× and added on cells previously prepared in a final volume of 60 μL per well in their culture medium (addition of 25 μL LNP/well corresponding to 125 ng RNA/well).

The luciferase assay (ONE-Glo™ Luciferase Assay System, Promega) was performed 48 h post-treatment in a 96-wells white cell culture plate and TECAN Spark plate reader were used to quantify the luminescence.

In mAb pre-incubation experiments, 5 μL of each mAb (9 mg/mL) were added on the cells for a final concentration of 500 μg/mL (saturating conditions) and incubated at room temperature for 30 minutes before treatment with LNPs.

Results show that, irrespective of the nature of the helper lipid, OSE-279 targeted LNPs were able to induce an improved transfection in U937 cells expressing the PD-1 receptors (FIG. 16). As observed in the previous experiments, the improvements in transfection potency were suppressed when a pre-incubation step with a saturating concentration of an anti-PD-1 mAbs was performed before the transfection protocols.

### Example 18: OSE-279 Targeted-LNPs Prepared Using 2 Different PEG-Lipid are Efficient in Preferentially Transfecting PD-1 Positive Cells

The influence of the chain-length of the PEG-lipid in the LNPs was assessed by preparing OSE-279 targeted-LNP with either DMG-PEG2000 or DSG-PEG2000 as PEG lipid.

### LNP Preparation

LNPs were prepared using microfluidic mixing. Briefly, lipids were dissolved in ethanol with the following molar ratios ALC-0315: 50%/DOPE: 10%/Cholesterol: 38.5%/DMG-PEG2000 or DSG-PEG2000: 1.5%, at a total lipid concentration of 9.1 mM. FLuc-mRNA (25 μL, 1 mg/mL) was diluted with 25 mM acetate buffer (pH 4.3) to a final concentration of 0.083 mg/mL. For the preparation of targeted-LNPs, the mAb (0.95 μg/μL in PBS, 1.3×10⁻⁶ μmol mAb/μg RNA) was added to the mRNA solution prior to microfluidic mixing. The selected experimental conditions allow for a N/P ratio of 6.

The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 9
Targeting agent and characteristics of LNP produced and tested

| Ref | Targeting agent | PEG-lipid | Z-Ave | PDI | % EE |
|---|---|---|---|---|---|
| 3173 | No Ab | DMG- | 148 | 0.105 | 92% |
| | | PEG2000 | | | |
| 3176 | OSE-279 | DMG- | 184 | 0.148 | 85% |
| | | PEG2000 | | | |
| 3178 | No Ab | DSG- | 165 | 0.103 | 92% |
| | | PEG2000 | | | |
| 3179 | OSE-279 | DSG- | 117 | 0.241 | 74% |
| | | PEG2000 | | | |

### In Vitro Transfection

LNPs were diluted to 1.7 ng/μL with PBS 1× and added on cells previously prepared in a final volume of 60 μL per well in their culture medium (addition of 25 μL LNP/well corresponding to 41.7 ng RNA/well for U937 cells and addition of 10 μL LNP/well corresponding to 16.7 ng RNA/well for Jurkat cells).

The luciferase assay (ONE-Glo™ Luciferase Assay System, Promega) was performed 48 h post-treatment in a 96-wells white cell culture plate and TECAN Spark plate reader were used to quantify the luminescence.

In mAb pre-incubation experiments, 5 μL of each mAb (9 mg/mL) were added on the cells for a final concentration of 500 μg/mL (saturating conditions) and incubated at room temperature for 30 minutes before treatment with LNPs.

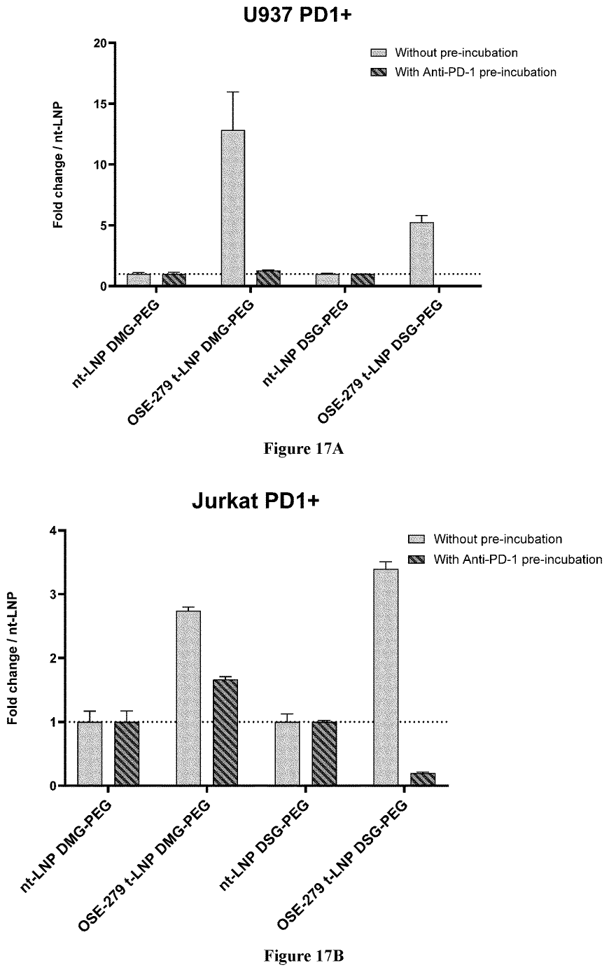

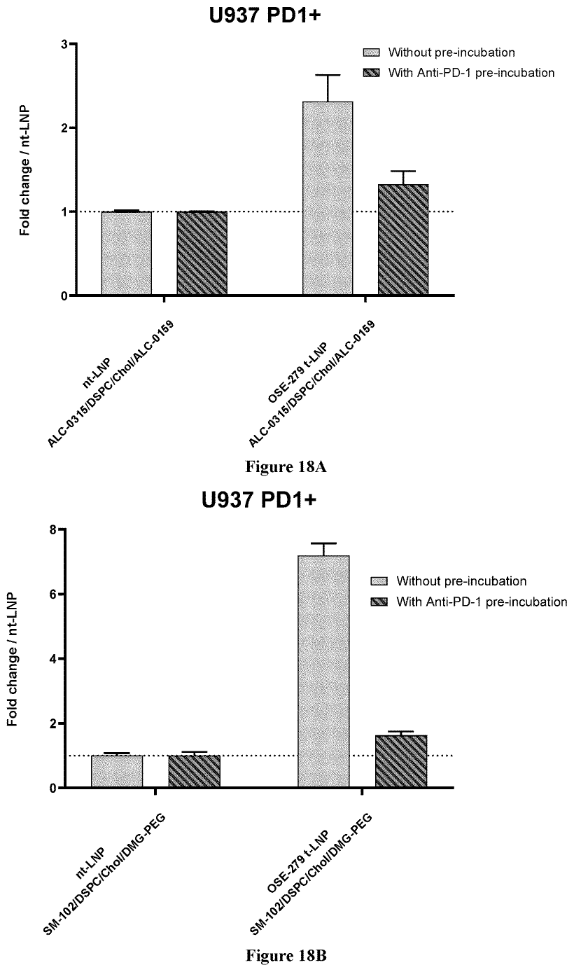

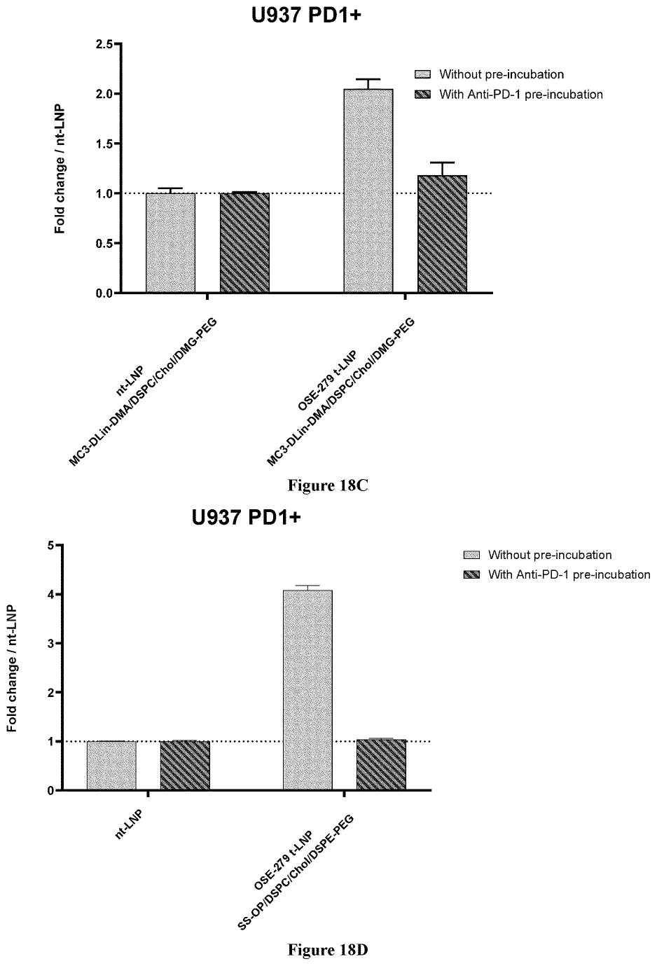

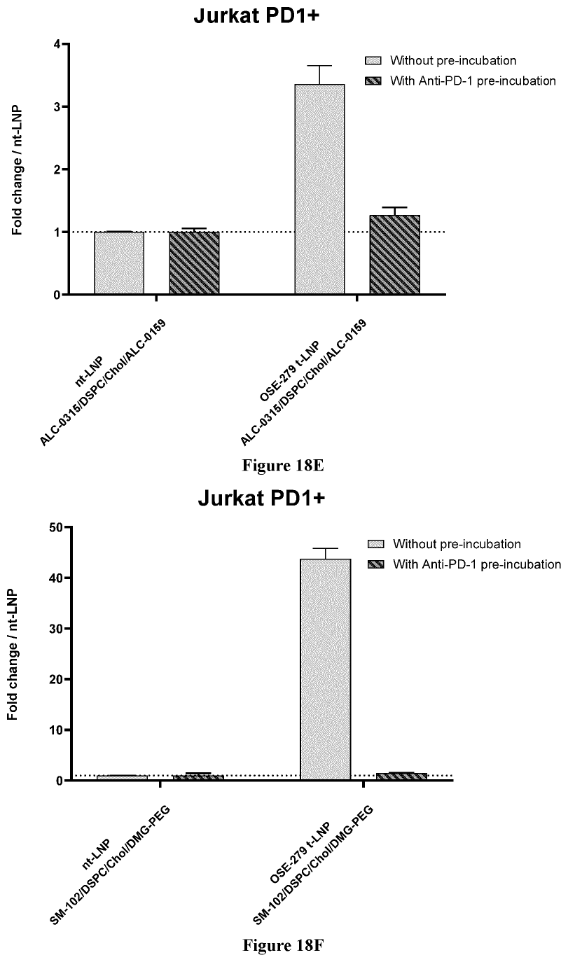

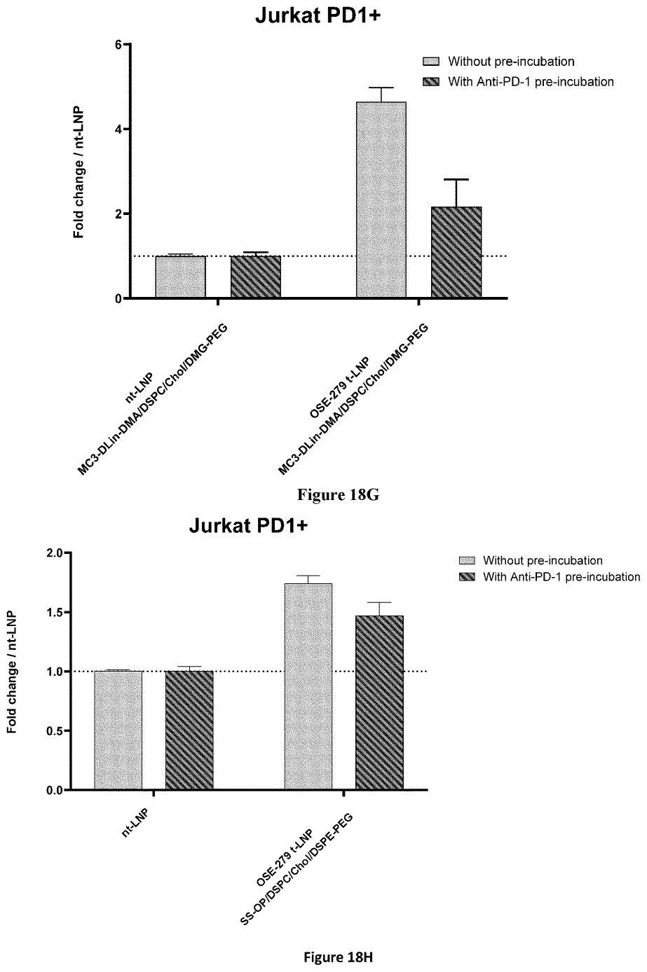

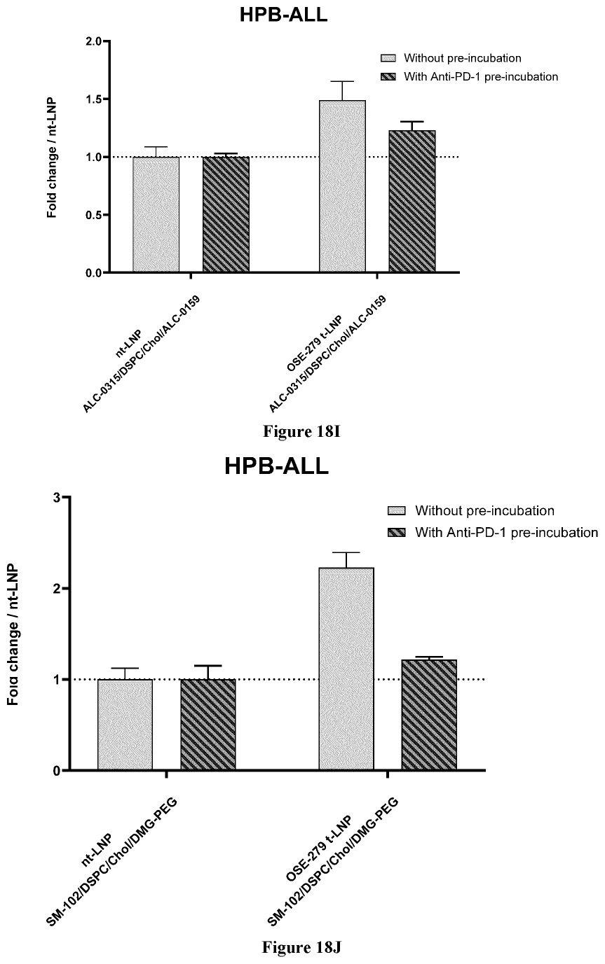

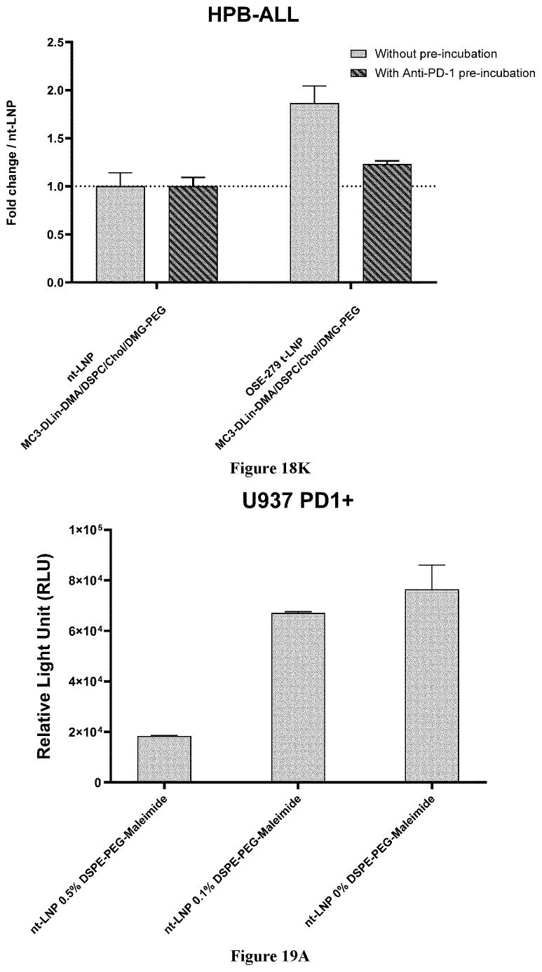

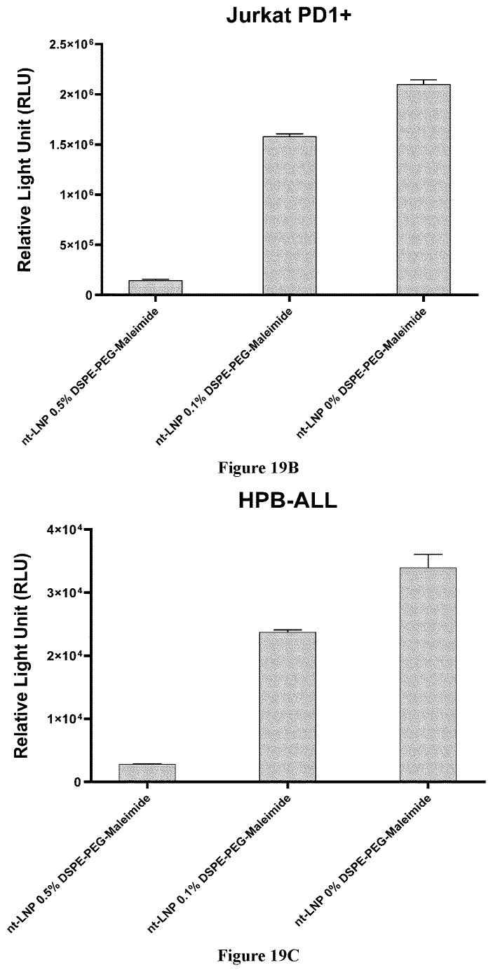

Results show that the chain-length of the PEG-lipid does not impact on the targeting capacity of targeted-LNPs, as targeted-LNPs prepared with either DSG-PEG or DMG-PEG were able to induce an improved transfection in cells expressing the PD-1 receptors (U937 cells in FIG. 17A, Jurkat cells in FIG. 17B and HPB-ALL cells in FIG. 17C). As observed in the previous experiments, the improvements in transfection potency were suppressed when a pre-incubation step with a saturating concentration of an anti-PD-1 mAbs was performed before the transfection protocols.

### Example 19: OSE-279 Targeted-LNPs Prepared Using Various Lipid Formulations are Efficient in Preferentially Transfecting PD-1 Positive Cells

The influence of the lipidic composition of the LNPs was assessed by preparing OSE-279 targeted-LNP with the lipids used in the Cominarty®, SpikeVax® and Onpattro® formulations, respectively, or with a formulation using Coatsome® SS-OP as ionizable lipid.

### Preparation of LNP

LNPs were prepared using microfluidic mixing. Briefly, lipids were dissolved in ethanol with the following molar ratios, at a total lipid concentration of 9.1 mM:
Formulation A1: ALC-0315:46.3%/DSPC:9.4%/Cholesterol:42.7%/ALC-0159:1.6%,
Formulation A2: SM-102:50%/DSPC:10%/Cholesterol:38.5%/DMG-PEG-2000:1.5%,
Formulation A3: DLin-MC3-DMA:50%/DSPC:10%/Cholesterol:38.5%/DMG-PEG-2000:1.5%,
Formulation A4: SS-OP:50%/DSPC:10%/Cholesterol:38.5%/DSPE-PEG-2000:1.5%.

FLuc-mRNA (25 μL, 1 mg/mL) was diluted with 25 mM acetate buffer (pH 4.3) to a final concentration of 0.083 mg/mL. For the preparation of targeted-LNPs, the mAb (0.95 μg/μL in PBS, 1.3×10⁻⁶ μmol mAb/μg RNA) was added to the mRNA solution prior to microfluidic mixing.

The selected experimental conditions allow for a N/P ratio of 6.

The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 10
Formulation, targeting agent and
characteristics of LNP produced and tested

| Ref | Formulation | Targeting agent | Z-Ave | PDI | % EE |
|---|---|---|---|---|---|
| 3298 | A1 | No Ab | 130 | 0.171 | 80% |
| 3299 | A1 | OSE-279 | 133 | 0.240 | 72% |
| 3300 | A2 | No Ab | 137 | 0.089 | 79% |
| 3301 | A2 | OSE-279 | 151 | 0.196 | 50% |
| 3302 | A3 | No Ab | 126 | 0.117 | 76% |
| 3303 | A3 | OSE-279 | 150 | 0.221 | 70% |
| 3358 | A4 | No Ab | 95 | 0.079 | 94% |
| 3359 | A4 | OSE-279 | 121 | 0.224 | 88% |

### In Vitro Transfection

LNPs were diluted to 4 ng/μL with PBS 1× and added on cells previously prepared in a final volume of 60 μL per well in their culture medium (addition of 25 μL LNP/well corresponding to 100 ng RNA/well for U937 cells and addition of 10 μL LNP/well corresponding to 40 ng RNA/well for Jurkat and HPB-ALL cells).

The luciferase assay (ONE-Glo™ Luciferase Assay System, Promega) was performed 48 h post-treatment in a 96-wells white cell culture plate and TECAN Spark plate reader were used to quantify the luminescence.

In mAb pre-incubation experiments, 5 μL of each mAb (9 mg/mL) were added on the cells for a final concentration of 500 μg/mL (saturating conditions) and incubated at room temperature for 30 minutes before treatment with LNPs.

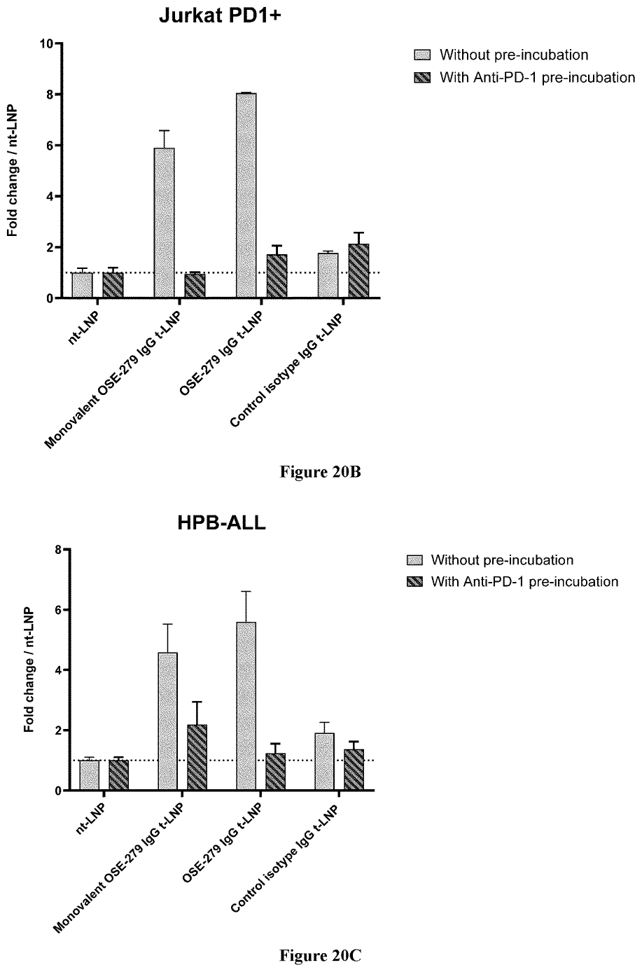

Results show that the nature of the lipids used to formulate mRNA does not influence the capacity of OSE-279 targeted-LNPs to induce improved transfection in PD-1 expressing cells. Indeed, improved transfection was observed with targeted-LNPs, compared to non-targeted LNPs and control isotype targeted-LNPS, for the 4 formulations tested (A1, A2, A3 and A4) in the 3 cell types tested (U937, Jurkat and HPB-ALL, FIGS. 20A-H). Moreover, as observed in the previous experiments, the improvements in transfection potency were suppressed when a pre-incubation step with a saturating concentration of an anti-PD-1 mAbs was performed before the transfection protocols.

### Example 20: OSE-279 Targeted-LNPs Comprising OSE-279 mAbs Grafted to Lipids and OSE-279 Targeted-LNPs Comprising OSE-279 mAbs that are not Grafted to Lipids (Free OSE-279)Display Differences in Terms of Targeted Transfection and Yields of Production

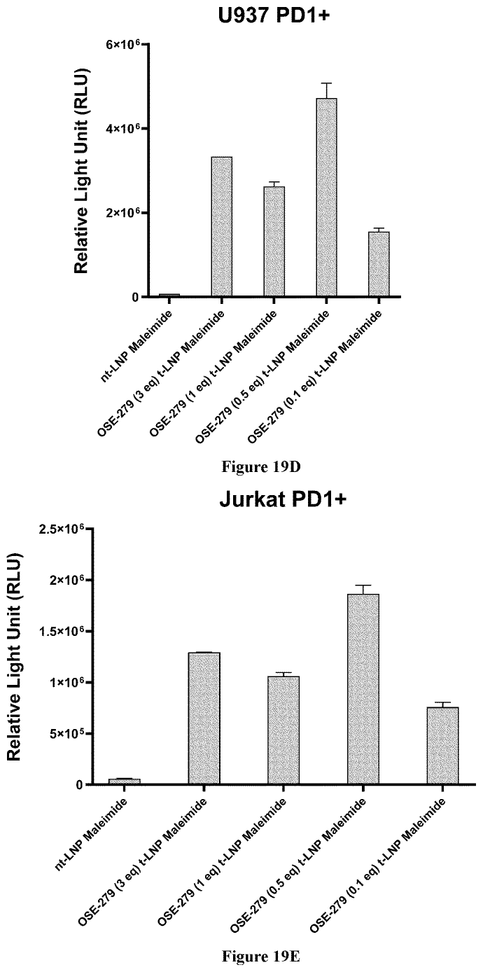

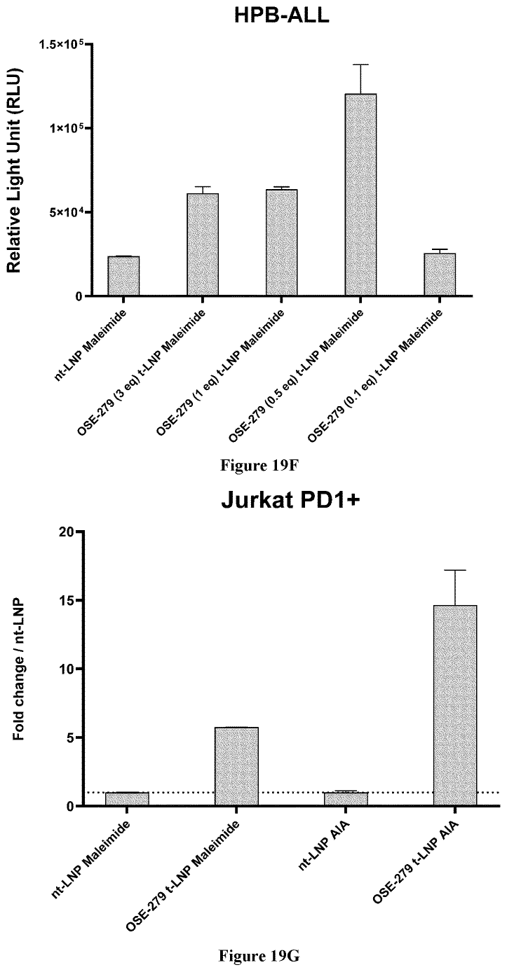

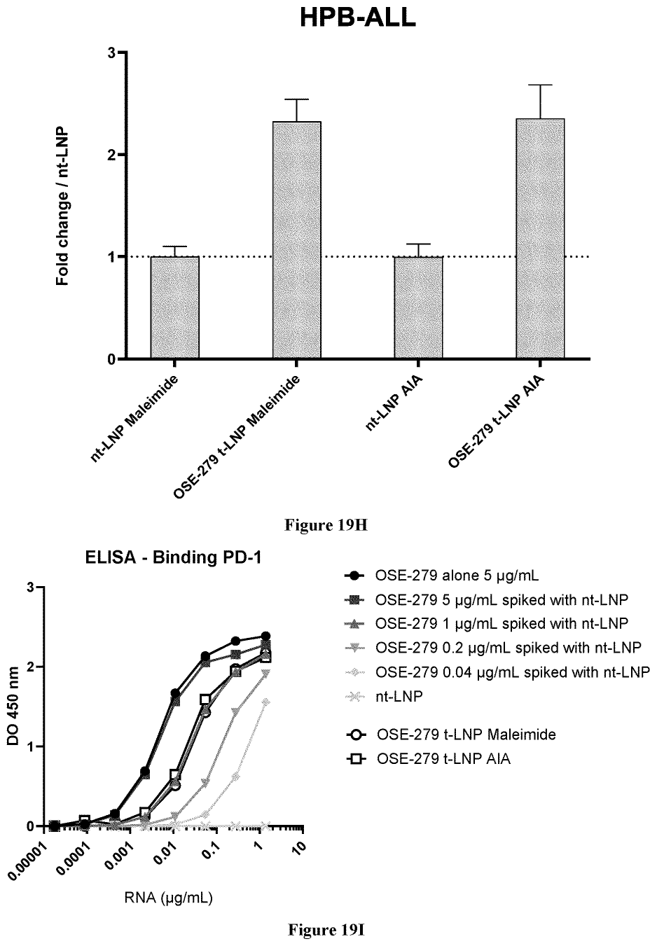

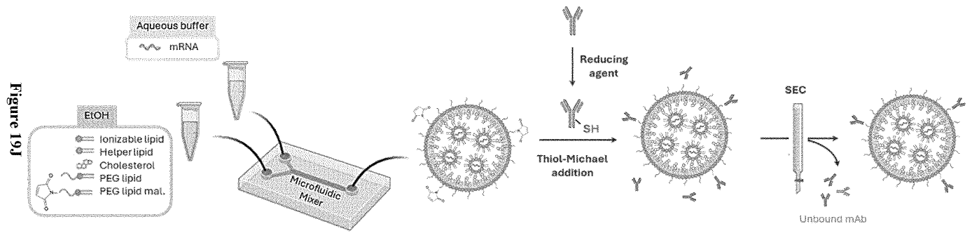

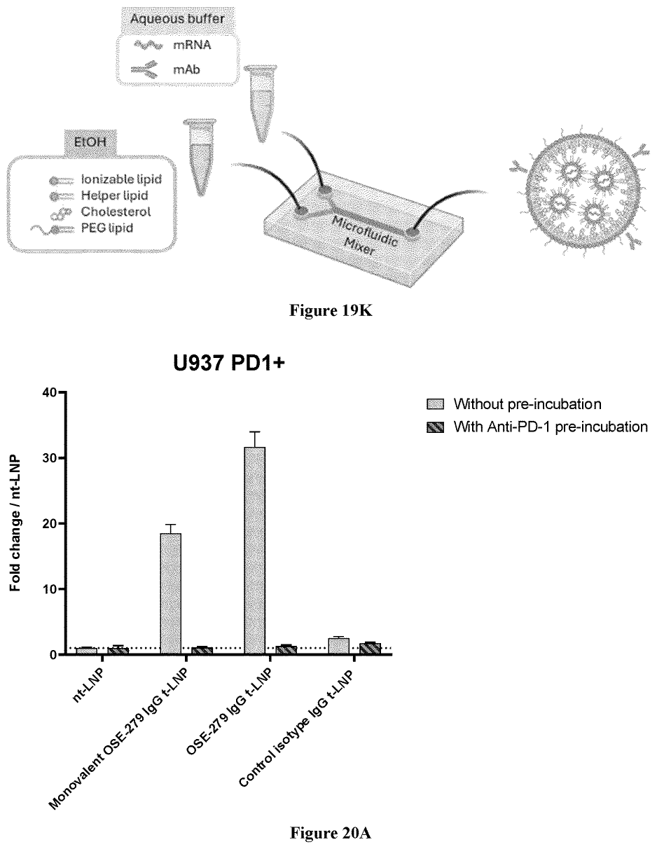

A stepwise approach was adopted to compare the preparation and efficacy of targeted-LNPs using an alternative method disclosed herein (see FIG. 19, in the “Methods” section and in Examples 1-3, wherein the mAbs are uncoupled—i.e. the mAbs are not covalently bound to any of the lipids of the lipid-based nanoparticle or do not comprise any modification for coupling or grafting the antigen binding domain to a lipid) with the classical process for mAb grafting on the surface of LNP consisting of i) preparing an LNP with a PEG-lipid decorated with a maleimide moiety, ii) reducing the mAb to expose thiol residue and iii) coupling both moieties via Thiol-Michael addition to allow the mAb to be grafted on the extremity of the PEG-lipid (in which the mAbs are coupled—i.e. the mAbs are covalently bound to the lipids of the lipid-based nanoparticle). At first, the inventors optimized the experimental conditions for the Thiol/Maleimide coupling by studying different experimental conditions, by varying on one hand the amount of DSPE-PEG-Maleimide in the LNP and on the other varying the amount of mAbs to be reduced and added in the coupling step.

Selection of the optimized ratio of DSPE-PEG-Maleimide Non-targeted LNPs were prepared using microfluidic mixing. Briefly, lipids were dissolved in ethanol with the following molar ratios, at a total lipid concentration of 9.1 mM:
Formulation B1: ALC-0315:50%/DOPE:10%/Cholesterol:38.5%/DMG-PEG-2000:1.0%/DSPE-PEG-Maleimide:0.5%,
Formulation B2: ALC-0315:50%/DOPE:10%/Cholesterol:38.5%/DMG-PEG-2000:1.4%/DSPE-PEG-Maleimide:0.1%,
Formulation B3: ALC-0315:50%/DOPE:10%/Cholesterol:38.5%/DMG-PEG-2000:1.5%/DSPE-PEG-Maleimide:0% (formulation used in the alternative method disclosed herein, i.e., where mAbs are not covalently linked to lipids).

FLuc-mRNA (150 μL, 1 mg/mL) was diluted with 25 mM acetate buffer (pH 4.3) to a final concentration of 0.083 mg/mL.

The selected experimental conditions allow for a N/P ratio of 6.

The lipids (600 μL) and the mRNA solutions (1800 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich).

TABLE 11
Formulation and characteristics of LNP produced and tested

| Ref | Formulation | Z-Ave | PDI | % EE |
|---|---|---|---|---|
| 3205 | B1 | 135 | 0.111 | 86% |
| 3206 | B2 | 146 | 0.104 | 86% |
| 3209 | B3 | 143 | 0.090 | 89% |

### In Vitro Transfection

LNPs were diluted to 5.16 ng/μL with PBS 1× and added on cells previously prepared in a final volume of 60 μL per well in their culture medium (addition of 25 μL LNP/well corresponding to 129 ng RNA/well for U937, Jurkat and HPB-ALL cells).

The luciferase assay (ONE-Glo™ Luciferase Assay System, Promega) was performed 48 h post-treatment in a 96-wells white cell culture plate and TECAN Spark plate reader were used to quantify the luminescence.

Formulation B2 (0.1% DSPE-PEG-Maleimide) was selected in favor of formulation B1, as the transfection data for formulation B2 were closer to those obtained with formulation B3 (no DSPE-PEG-Maleimide) used as standard (FIGS. 19A-C).

### Selection of the Best Conditions for mAb Coupling by Thiol-Michael Addition

LNPs were prepared using microfluidic mixing. Briefly, lipids were dissolved in ethanol with the following molar ratios ALC-0315: 50%/DOPE: 10%/Cholesterol: 38.5%/DMG-PEG-2000: 1.4%/DSPE-PEG-Maleimide: 0.1%, at a total lipid concentration of 9.1 mM.

FLuc-mRNA (150 μL, 1 mg/mL) was diluted with 25 mM acetate buffer (pH 4.3) to a final concentration of 0.083 mg/mL. The selected experimental conditions allow for a N/P ratio of 6.

The lipids (600 μL) and the mRNA solutions (1800 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the Maleimide-LNP solution was used for coupling with the reduced mAbs.

OSE-279 mAbs were diluted with PBS to obtain a 5 mg/mL solution, before addition of EDTA (5 mM) and TCEP (50 mM, 3 molar equivalent vs OSE-279). The mixture was heated for 30 min at 37 °C and stirred for 30 min at room temperature. Excess TCEP was removed by using 7K desalting columns (ThermoFisher) according to manufacturer protocol.

The following quantities of OSE-279 were assessed:
Formulation C1: 3 molar equivalent of OSE-279 vs DSPE-PEG-Maleimide
Formulation C2: 1 molar equivalent of OSE-279 vs DSPE-PEG-Maleimide
Formulation C3: 0.5 molar equivalent of OSE-279 vs DSPE-PEG-Maleimide
Formulation C4: 0.1 molar equivalent of OSE-279 vs DSPE-PEG-Maleimide

The reduced antibody was then immediately conjugated with Maleimide-LNP by incubation for 1 h at room temperature and overnight at 4 °C. The conjugated LNP was then purified by gel filtration chromatography (HiLoad 16/600 Superdex 200 column, Cytiva) using PBS as a mobile phase (flow rate: 1 mL/min). LNPs fractions were collected and concentrated to 1 mL by 100K Amicon tubes (Millipore).

TABLE 12
Formulation, targeting agent and
characteristics of LNP produced and tested

| Ref | Formulation | Targeting agent | Z-Ave | PDI | % EE |
|---|---|---|---|---|---|
| 3206 | B2 (no Ab) | No | 146 | 0.104 | 86% |
| 3208A | C1 (coupled Ab) | OSE-279 | 141 | 0.130 | 81% |
| 3208B | C2(coupled Ab) | OSE-279 | 141 | 0.094 | 79% |
| 3208C | C3(coupled Ab) | OSE-279 | 141 | 0.116 | 84% |
| 3208D | C4(coupled Ab) | OSE-279 | 146 | 0.115 | 82% |

### In Vitro Transfection (FIGS. 19D-F)

LNPs were diluted to 5.16 ng/μL with PBS 1× and added on cells previously prepared in a final volume of 60 μL per well in their culture medium (addition of 25 μL LNP/well corresponding to 129 ng RNA/well for U937 and HPB-ALL cells; addition of 2.5 μL LNP/well corresponding to 12.9 ng RNA/well for Jurkat cells).

The luciferase assay (ONE-Glo™ Luciferase Assay System, Promega) was performed 48 h post-treatment in a 96-wells white cell culture plate and TECAN Spark plate reader were used to quantify the luminescence.

Formulation C3 (0.5 eq of OSE-279 vs DSPE-PEG-Maleimide) was selected in favor of other formulations, as the transfection of PD-1 positive cells for formulation C3 were higher in the 3 cell lines tested (FIGS. 19 D-F).

### Preparation of OSE-279 Targeted LNP Using Optimized Conditions for Thiol-Michael Addition

LNPs were prepared using microfluidic mixing. Briefly, lipids were dissolved in ethanol with the following molar ratios ALC-0315: 50%/DOPE: 10%/Cholesterol: 38.5%/DMG-PEG-2000: 1.4%/DSPE-PEG-Maleimide: 0.1%, at a total lipid concentration of 9.1 mM. FLuc-mRNA (150 μL, 1 mg/mL) was diluted with 25 mM acetate buffer (pH 4.3) to a final concentration of 0.083 mg/mL. The selected experimental conditions allow for a N/P ratio of 6.

The lipids (600 μL) and the mRNA solutions (1800 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the Maleimide-LNP solution was used for coupling with the reduced mAbs.

OSE-279 mAbs (0.5 molar equivalent vs DSPE-PEG-Maleimide) were diluted with PBS to obtain a 5 mg/mL solution, before addition of EDTA (5 mM) and TCEP (50 mM, 3 molar equivalent vs OSE-279). The mixture was heated for 30 min at 37 °C and stirred for 30 min at room temperature. Excess TCEP was removed by using 7K desalting columns (ThermoFisher) according to manufacturer protocol.

The reduced antibody was then immediately conjugated with Maleimide-LNP by incubation for 1 h at room temperature and overnight at 4 °C. The conjugated LNP was then purified by gel filtration chromatography (HiLoad 16/600 Superdex 200 column, Cytiva) using PBS as a mobile phase (flow rate: 1 mL/min). LNPs fractions were collected and concentrated to 1 mL by 100K Amicon tubes (Millipore).

### Preparation of OSE-279 “Free” Targeted-LNP

LNPs were prepared using microfluidic mixing. Briefly, lipids were dissolved in ethanol with the following molar ratios ALC-0315: 50%/DOPE: 10%/Cholesterol: 38.5%/DMG-PEG-2000: 1.5%, at a total lipid concentration of 9.1 mM.

FLuc-mRNA (50 μL, 1 mg/mL) was diluted with 25 mM acetate buffer (pH 4.3) to a final concentration of 0.083 mg/mL. For the preparation of targeted-LNPs, the mAbs (0.95 μg/μL in PBS, 1.9×10⁻⁶ μmol mAb/μg RNA) were added to the mRNA solution prior to microfluidic mixing. In this experiment, the mAbs are not covalently bound to any of the lipids of the lipid-based nanoparticle or does not comprise any modification for coupling or grafting the antigen binding domain to a lipid. The selected experimental conditions allow for a N/P ratio of 6.

The lipids (200 μL) and the mRNA solutions (600 μL) were then injected into a microfluidic mixer (LNP Pack, Inside Therapeutics) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 13
Method of production, targeting agent and
characteristics of LNP produced and tested

| Tube | Method | Targeting agent | Z-Ave | PDI | % EE |
|---|---|---|---|---|---|
| 3206 | Thiol-maleimide | No | 146 | 0.104 | 86% |
| | (coupled Ab) | | | | |
| 3224D | Thiol-maleimide | OSE-279 | 147 | 0.098 | 61% |
| | (coupled Ab) | | | | |
| 3225 | alternative method | No | 149 | 0.083 | 82% |
| | (uncoupled Ab) | | | | |
| 3227A | alternative method | OSE-279 | 169 | 0.128 | 74% |
| | (uncoupled Ab) | | | | |

### In Vitro Transfection (FIGS. 19G-I)

LNPs were diluted to 2.57 ng/μL with PBS 1× and added on cells previously prepared in a final volume of 60 μL per well in their culture medium (addition of 10 μL LNP/well corresponding to 25.75 ng RNA/well for U937, Jurkat and HPB-ALL cells).

The luciferase assay (ONE-Glo™ Luciferase Assay System, Promega) was performed 48 h post-treatment in a 96-wells white cell culture plate and TECAN Spark plate reader were used to quantify the luminescence.

### ELISA Assay—Binding PD-1 (FIG. 19)

For activity ELISA assay, recombinant hPD-1 (Sino Biologicals, Beijing, China; reference 10377-H08H) was immobilized on plastic at 0.5 μg/mL in carbonate buffer (pH 9.2). Targeted-LNP (diluted to an initial concentration of 1.38 μg/mL of RNA) were then added to measure binding. After incubation and washing, peroxidase-labeled donkey anti-human IgG (Jackson Immunoresearch; USA; reference 709-035-149) was added and revealed by conventional methods.

Standard curves consist of i) OSE-279 alone at 5 μg/mL and ii) OSE-279 at different concentrations (5 to 0.04 μg/mL) spiked with non-targeted LNP.

The transfection data indicated different profiles for the OSE-279 targete 41d LNPs prepared by the 2 methods (each targeted LNPs being compared to the corresponding non-targeted LNPs prepared by the same method):
For Jurkat cells (FIG. 19G): the targeted LNPs obtained via the alternative method disclosed herein (uncoupled Abs) induced better targeting of PD-1 positive cells compared to targeted-LNPs prepared by thiol-Maleimide coupling (coupled Abs),
For HPB-ALL cells (FIG. 19H): similar targeting efficiency was observed for both types of targeted-LNPs

Comparing the efficacy of both targeted-LNP production process rely on assessing the efficacy of both LNP production and mAb insertion.

Targeted LNPs prepared with the chemical anchoring of mAb on Maleimide moieties positioned at the extremities of PEG-lipids on the LNPs (coupled Abs) rely on a multi-step process (FIG. 19J):
a. Formation of 5-constituents LNP with addition of DSPE-PEG-Maleimide compared to traditional LNPs,
b. Reduction of mAbs to expose thiol groups,
c. Thiol-Michael addition of reduced mAbs with Maleimide-LNPs,
d. Size-exclusion chromatography to remove unbound mAbs,
e. Concentration of SEC fractions.

This sequence led to the formation of OSE-279 targeted-LNPs
With an mRNA concentration of 6.90 μg/mL (calculated by Ribogreen assay according to manufacturer protocol), corresponding to a yield of 17% (expected concentration of 41.5 μg/mL),
With an OSE-279 mAb concentration of 1 μg/mL (calculated by ELISA binding assay on PD-1, FIG. 19I), corresponding to a yield of <1% of OSE-279 mAbs able to bind to their PD-1 receptors (expected mAb concentration of 118 μg/mL).

On the other hand, the alternative method disclosed herein allows (uncoupled Abs) for the preparation of targeted LNPs in a single step (and without the need for adding a 5^{th} lipid in the formulation, FIG. 19K). With the tested conditions, this process led to the formation of OSE-279 targeted-LNPs:
With an mRNA concentration of 14.75 μg/mL (calculated by Ribogreen assay according to manufacturer protocol), corresponding to a yield of 93% (expected concentration of 15.94 μg/mL),
With an OSE-279 mAb concentration of 1 μg/mL (calculated by ELISA binding assay on PD-1, FIG. 19I), corresponding to a yield of 58% of OSE-279 mAbs able to bind to their PD-1 receptors (expected mAb concentration of 3.64 μg/mL).

### Example 21: OSE-279 Targeted-LNPs Prepared with Either Full IgG or Monovalent IgG Format are Both Efficient in Preferentially Transfecting PD-1 Positive Cells

The influence of the OSE-279 mAb format was assessed by preparing OSE-279 targeted-LNP with mAb in full IgG or Monovalent IgG format.

### Preparation of LNP

LNPs were prepared using microfluidic mixing. Briefly, lipids were dissolved in ethanol with the following molar ratios ALC-0315: 50%/DOPE: 10%/Cholesterol: 38.5%/DMG-PEG-2000: 1.5%, at a total lipid concentration of 9.1 mM. FLuc-mRNA (50 μL, 1 mg/mL) was diluted with 25 mM acetate buffer (pH 4.3) to a final concentration of 0.083 mg/mL. For the preparation of targeted-LNPs, the mAb (0.95 μg/μL in PBS, 1.9×10⁻⁶ μmol mAb/μg RNA) was added to the mRNA solution prior to microfluidic mixing. The selected experimental conditions allow for a N/P ratio of 6.

The lipids (200 μL) and the mRNA solutions (600 μL) were then injected into a microfluidic mixer (LNP Pack, Inside Therapeutics) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C and characterized.

TABLE 14
Targeting agent and characteristics of LNP produced and tested

| Ref | Targeting agent | Z-Ave | PDI | % EE |
|---|---|---|---|---|
| 320 | No Ab | 140 | 0.161 | 91% |
| 322 | Monovalent OSE-279 | 178 | 0.191 | 76% |
| 323 | Bivalent OSE-279 | 171 | 0.178 | 71% |
| 325 | IgG Isotype | 211 | 0.225 | 68% |

### In Vitro Transfection (FIGS. 20A-C)

LNPs were diluted to 3.9 ng/μL with PBS 1× and added on cells previously prepared in a final volume of 60 μL per well in their culture medium (addition of 25 μL of LNP/well corresponding to 96 ng RNA/well).

The luciferase assay (ONE-Glo™ Luciferase Assay System, Promega) was performed 24 h post-treatment in a 96-wells white cell culture plate and TECAN Spark plate reader were used to quantify the luminescence.

In mAb pre-incubation experiments, 5 μL of mAb (9 mg/mL) were added on the cells for a final concentration of 500 μg/mL (saturating conditions) and incubated at room temperature for 30 minutes before treatment with LNPs.

Results indicate that both OSE-279 mAb formats (Bivalent IgG or Monovalent IgG) are suitable for producing targeted-LNPs able to preferentially transfect PD-1 positive cell lines.

### Example 22: OSE-279 Targeted-LNPs Prepared with Either Monovalent IgG or ScFv-Fc Format are Both Efficient in Preferentially Transfecting PD-1 Positive Cells

The influence of the OSE-279 mAb format was assessed by preparing OSE-279 targeted-LNP with mAb in Monovalent IgG or ScFv-Fc format.

### Preparation of LNP

LNPs were prepared using microfluidic mixing. Briefly, lipids were dissolved in ethanol with the following molar ratios ALC-0315: 50%/DOPE: 10%/Cholesterol: 38.5%/DMG-PEG-2000: 1.5%, at a total lipid concentration of 4.55 mM.

FLuc-mRNA (25 μL, 1 mg/mL) was diluted with 25 mM acetate buffer (pH 4.3) to a final concentration of 0.041 mg/mL. For the preparation of targeted-LNPs, the mAb (0.95 μg/μL in PBS, 1.3×10⁻⁶ μmol mAb/μg RNA) was added to the mRNA solution prior to microfluidic mixing. The selected experimental conditions allow for a N/P ratio of 6. The lipids (200 μL) and the mRNA solutions (600 μL) were then injected into a microfluidic mixer (LNP Pack, Inside Therapeutics, France) at a flow rate ratio of 1:3 and a combined flow rate of 4 mL/min. The resultant formulation was then immediately dialyzed against PBS 1M (pH 7.4) for at least 3.5 h using 3.5 MWCO dialysis cassettes (Pur-A-Lyzer, Sigma Aldrich). After dialysis, the LNPs were stored in a final volume of 1 to 1.5 mL in PBS at 4 °C for characterization.

TABLE 15
Targeting agent and characteristics of LNP produced and tested

| Ref | Targeting agent | Z-Ave | PDI | % EE |
|---|---|---|---|---|
| 3286 | No Ab | 118 | 0.120 | 86% |
| 3288 | Monovalent OSE-279 | 148 | 0.161 | 80% |
| 3289 | OSE-279 ScFv-Fc | 172 | 0.131 | 78% |
| 3290 | IgG Isotype | 156 | 0.247 | 77% |

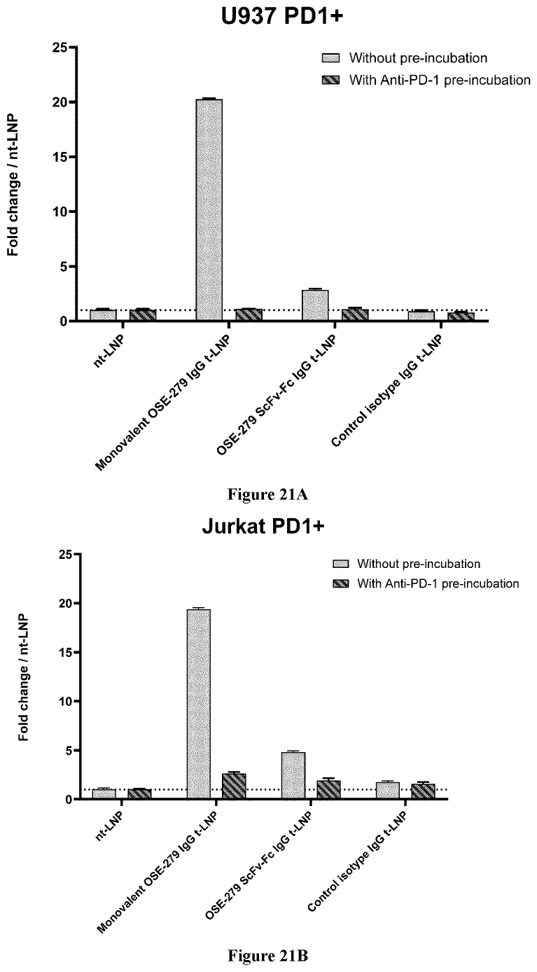

### In Vitro Transfection (FIGS. 21A-B)

LNPs were diluted to 4 ng/μL with PBS 1× and added on cells previously prepared in a final volume of 60 μL per well in their culture medium (addition of 25 μL LNP/well corresponding to 100 ng RNA/well for U937 cells and 2.5 μL LNP/well corresponding to 10 ng RNA/well for Jurkat cells).

The luciferase assay (ONE-Glo™ Luciferase Assay System, Promega) was performed 24 h post-treatment in a 96-wells white cell culture plate and TECAN Spark plate reader were used to quantify the luminescence.

In mAb pre-incubation experiments, 5 μL of mAb (9 mg/mL) were added on the cells for a final concentration of 500 μg/mL (saturating conditions) and incubated at room temperature for 30 minutes before treatment with LNPs.

Results indicate that both OSE-279 mAb formats (Monovalent IgG or ScFv-Fc) are suitable for producing targeted-LNPs able to preferentially transfect PD-1 positive cell lines, albeit in lower efficiency for the FcFv-Fc format.

1-33. (canceled)

34. A lipid-based nanoparticle comprising an antigen binding domain capable of specifically binding to a target expressed on activated immune cells surface and one or several mRNA molecule(s) encoding an activity-inhibiting protein of said activated immune cells, wherein the activity-inhibiting protein is i) an intracellular protein having an intracellular effect on the activated immune cell and/or ii) a transmembrane protein, wherein the antigen binding domain is an antibody or an antigen binding fragment thereof.

35. The lipid-based nanoparticle of claim 34, wherein the antigen binding domain comprises a Fc domain.

36. The lipid-based nanoparticle of claim 34, wherein the antigen binding domain is not covalently bound to any of the lipids of the lipid-based nanoparticle or does not comprise any modification for coupling or grafting the antigen binding domain to a lipid; and/or wherein the lipid-based nanoparticle does not comprise an anchoring moiety comprising a lipidation peptide or motif.

37. The lipid-based nanoparticle of claim 34, wherein the activated immune cells are selected from the group consisting of activated T cells, activated B cells, activated myeloid cells, activated macrophages and activated dendritic cells.

38. The lipid-based nanoparticle of claim 34, wherein the target expressed on activated immune cells surface is selected from the group consisting of PD-1, BCMA/TNFRSF17, BTLA, CD101/IGSF2, CD103, CD119, CD137/4-1BB/TNFRSF9, CD150, CD153, CD154, CD223, CD226, CD25, CD254, CD26, CD27, CD275, CD39/ENTPD1, CD40L, CD44, CD45RO, CD45RC, LGR6, CD69, GPR18, GPR35, FPR2, CD80, CD83, CD86, CD95, CMKLR1, CRTAM, CST7, CTLA4, CXCR3, CXCR4, CXCR5, CXCR6, FasL/TNFSF6, GITR/TNFRSF18, GPR32, TIM3/HAVCR2, ICOS, IL18R1/CXCR1/CD218a, ITGAE, LAG3, TRAILR, OX40L, LY108/SlamF6, NKG2D, OX40/TNFRSF4, PTPN22, RGS1, LOX1, SIGLEC 6, TACI/TNFRSF13B, TIGIT, CD163, CD206, LTBR/CD70, TNFSF14, SLAMF1, SLAMF7, NKG2A, KIR2DL2, CD96, CD112R, CD28H, IL2RB, TRAIL, CD48, CD53, CD164, CD138 (SDC1), CD38, CD39, FCRL4, CD30/TNFRSF8, CD78, TRAF1, TRAF2, TRAF3/CD40BP, TRAF3IP1, TRAF4, TRAF7, TRAP1, TNFR1/TNFRSF1A/CD120A, TRAP100/MED24, TNFR2/TNFRSF1811/CD120B, CDCR3/TNFRSF6B, TNFRSF12A/FN14/TWEAKR, BAFFR/TNFRSF13C/CD268, HVEM/TNFRSF14/CD270, GITR/TNFRSF8/CD357, RELT/TNFRSF19L, TNFRSF19/TROY, TNFRSF21/DR6, TNFRSF25/DR3/TNFRSF12, CD301, IL4R, CLEC-1A, CD21, CLEC-9A, CD180, CD59, CD54, CD71, CD35, CD218a, CD74, CD165, 4-1BBL/CD137L, ICOSL, CD160, CD127 and SIRPa.

39. The lipid-based nanoparticle of claim 38, wherein the target expressed on activated immune cells surface is PD-1.

40. The lipid-based nanoparticle of claim 39, wherein the antigen binding domain is an anti-PD-1 binding domain comprising a VH comprising a heavy chain CDR1 (HCDR1), CDR2 (HCDR2) and CDR3 (HCDR3), and (ii) a VL comprising a light chain CDR1 (LCDR1), CDR2 (LCDR2) and CDR3 (LCDR3),
wherein:
a) the HCDR1 comprises or consists of an amino acid sequence of SEQ ID NO: 1;
the HCDR2 comprises or consists of an amino acid sequence of SEQ ID NO: 2;
the HCDR3 comprises or consists of an amino acid sequence of SEQ ID NO: 3;
the LCDR1 comprises or consists of an amino acid sequence of SEQ ID NO: 4;
the LCDR2 comprises or consists of an amino acid sequence of SEQ ID NO: 5; and
the LCDR3 comprises or consists of an amino acid sequence of SEQ ID NO: 6; or
b) the HCDR1 comprises or consists of an amino acid sequence of SEQ ID NO: 23;
the HCDR2 comprises or consists of an amino acid sequence of SEQ ID NO: 24;
the HCDR3 comprises or consists of an amino acid sequence of SEQ ID NO: 25;
the LCDR1 comprises or consists of an amino acid sequence of SEQ ID NO: 26;
the LCDR2 comprises or consists of an amino acid sequence of SEQ ID NO: 27; and
the LCDR3 comprises or consists of an amino acid sequence of SEQ ID NO: 28; or
c) the HCDR1 comprises or consists of an amino acid sequence of SEQ ID NO: 31;
the HCDR2 comprises or consists of an amino acid sequence of SEQ ID NO: 32;
the HCDR3 comprises or consists of an amino acid sequence of SEQ ID NO: 33;
the LCDR1 comprises or consists of an amino acid sequence of SEQ ID NO: 34;
the LCDR2 comprises or consists of an amino acid sequence of SEQ ID NO: 35; and
the LCDR3 comprises or consists of an amino acid sequence of SEQ ID NO: 36; or
d) the antigen binding domain is an anti-PD-1 binding domain comprising:
i) a VH comprising or consisting of an amino acid sequence of SEQ ID NO: 15; and a VL comprising or consisting of an amino acid sequence of SEQ ID NO: 16;
ii) a VH comprising or consisting of an amino acid sequence of SEQ ID NO: 29; and a VL comprising or consisting of an amino acid sequence of SEQ ID NO: 30; or
iii) a VH comprising or consisting of an amino acid sequence of SEQ ID NO: 37; and a VL comprising or consisting of an amino acid sequence of SEQ ID NO: 38.

41. The lipid-based nanoparticle of claim 34, said lipid-based nanoparticle comprising an additional antigen binding domain capable of specifically binding to another target expressed on activated immune cells surface and, optionally, wherein the additional antigen binding domain is i) not covalently bound to any of the lipids of the lipid-based nanoparticle, ii) does not comprise any modification for coupling or grafting the antigen binding domain to a lipid and/or iii) is not covalently bound to a lipidation peptide or motif.

42. The lipid-based nanoparticle of claim 34, wherein the mRNA molecule encodes an intracellular protein having an intracellular effect on the activated immune cell selected from the group consisting of a cytoplasmic protein, an intracellular signalling protein, an enzyme, a transcription factor, an intrabody, a dominant negative receptor or an antagonist engineered protein.

43. The lipid-based nanoparticle of claim 42, wherein the one or several mRNA molecule(s) encode(s) for a molecule selected from the group consisting of:
a) a protein selected from the group consisting of TIM3, ENTPD1, LAG3, PD-1, PD-L1, TIGIT, FOXO1, MLH1, MSH2, MSH6, APC, CDKN2A, CASPASE, FAS, TRAIL, Bax, Bak, Bok, Bad, Bid et Bim, BIM, BIM-S, FADD, FASL, TRAILR, TNFR, ATG5, LC3, GABARAP, GATE16, ATG 5/7/10/12, NOXA, PUMA, P53, FOXP3, TOX, EOMES, BCL6 and BACH2, HIFa, PKCq and VHL, IDO-1, IDO-2, ARG1; TDO, mTOR/DAPTOR/RAPTOR, SHP, SMAD, SHIP-1, SHP-1/2, PTEN, PTP1B, IKAROS, EGR2/3, CREM, P27 (KIP1), CD107a, RAC1, AP2, RB7, M6P, MPR, BBSi10, BBS12, TCP1, HSP, APC, Laminin, Actin, Vimentin, DEF1, Dynein, Kinesin, TAPASIN, LMP7, Erp57, Cbl-b, CHEMR23, GPR37, GPR32, GPR18, FPR2, GPR35, HDAC, HMT, SIRT1, DOT1L, PRDME, PRMT1, PRMT3, PRMT5, PRMT6, SETD7, SETD8, SMYD2, SMYD3, SUV420H2and any combination thereof;
b) a cytokine selected from the group consisting of IFNG, IFNa, IL2, IL13, IL4, IL-10, and any combination thereof;
c) a cytokine receptor selected from the group consisting of IL-1R, IL-4R, IL-6R, IL-8R, IL-10R, IL-11R, IL-17R, IL-18R, IL-21R, IL-23R, IL-35R, and any combination thereof; and
d) a chemokine receptor selected from the group consisting of CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, XCR1, and any combination thereof.

44. The lipid-based nanoparticle of claim 43, wherein the one or several mRNA molecule(s) encode(s) for CCR4, CCR10, CXCR3, CCR5, CCR2, CX3CR1, CCR7, CXCR4, CXCR5, IL10R, TGFR, TNFR, ILR1A, GCSFR, IL4R, CXCR1, CXCR2, CXCR6, CCR1, CCR3, CCR6, CCR8, CCR9, CCR11, XCR1, CD72, DCIR, MICL, CLEC-1, TGFB, IL13, IL4, IL-10, IL-6R, IL17R, IL23R, IL35R, IL21R, IFNa R, BTLA, CTLA4, B7H1, B7H3, B7H4, PD1H, LAIR1, TIM1, TIM3, TIM4, 2B4, PD-1, PDL1, PDL2, galectin9, CD48, HVEM, B7DC, CD155, CD113, CD112, ILT2, ILT3, NKG2A, LILRB1, IRp60, KIR, CD22, CD5, CD66a, PIR-B, KIR2DL1, KIR2DL2/3, ILT4, KIR2DL5, KIR3DL1, KIR3DL2, KIR3DL3, TIGIT, LAG3 or A2AR, SOCS, RIPK1 and any member of the STAT family or any combination thereof; or
the one or several mRNA molecule(s) encode(s) for BIM-S and/or PUMA.

45. The lipid-based nanoparticle of claim 34, wherein the lipid-based nanoparticle comprises at least two mRNA molecules, wherein one of the least two mRNA molecules encodes a transmembrane protein that is a receptor and another of the least two mRNA molecules encodes a secreted protein that is a ligand of said receptor.

46. The lipid-based nanoparticle of claim 34, wherein the lipid-based composition of the lipid based nanoparticle comprises a cationic or ionizable lipid, a helper lipid, a sterol and a PEG-lipid.

47. The lipid-based nanoparticle of claim 46, wherein the ionizable lipid is selected from the group consisting of [(4-hydroxybutyl)azanediyl]di(hexane-6,1-diyl) bis(2-hexyldecanoate) (ALC-0315), 1,2-dioleoyl-3-trimethylammonium propane (DOTAP); N,N-dimethyl-2,3-dioleyloxypropylamine (DODMA), 1,2-di-O-octadecenyl-3-trimethylammoniumpropane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammoniumpropanes; 1,2-dialkyloxy-3-dimethylammoniumpropanes; dioctadecyldimethylammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (+)—N-(3-aminopropyl)-N,N-dimethyl-2,3-bis(cis-9-tetradecenyloxy)-1-propanaminium bromide (GAP-DMORIE), (+)—N-(3-aminopropyl)-N,N-dimethyl-2,3-bis(dodecyloxy)-1-propanaminium bromide (GAP-DLRIE), (+)—N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminium (DOBAQ), 2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), Ni-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98Niz-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), 9-Heptadecanyl 8-{(2-hydroxyethyl)[6-oxo-6-(undecyloxy)hexyl]amino}octanoate (SM-102), bis[2-(4-{2-[4-(cis-9-octadecenoyloxy)phenylacetoxy]ethyl}piperidinyl)ethyl]disulfide (SS-OP), bis{2-[4-(α-D-tocopherolhemisuccinateethyl)piperidyl]ethyl}disulfide (SS-EC) and any mixtures thereof,
the sterol is selected from the group consisting of cholesterol, fecosterol, sitosterol, ergosterol, campesterol, stigmasterol, brassicasterol, tomatidine, ursolic acid, alpha-tocopherol, and any mixtures thereof;
the helper lipid is selected from DOPE, DOPS, DODMA, DOTAP, DODAP, DDAB, POPE, DSPC, DOPC, DEPC and DSPE, and any mixtures thereof, and
the PEG-lipid is selected from PEG-DMG, PEG-DSPE, PEG-c-DOMG, DMG-PEG-2000, PEG-DLPE, PEG-DMPE, PEG-DPPC, PEG-DPPE, PEG-DAG and PEG-c-DMA, ALC-0159, and any mixtures thereof.

48. The lipid-based nanoparticle of claim 46, wherein the lipid-based composition of the lipid-based nanoparticle is selected from the group consisting of:
a) ALC-0315, DOPE, cholesterol and DMG-PEG;
b) ALC-0315, DDAB, cholesterol and DMG-PEG;
c) ALC-0315, POPE, cholesterol and DMG-PEG;
d) ALC-0315, DOPE, cholesterol and DSPE-PEG;
e) ALC-0315, DSPC, cholesterol and DMG-PEG;
f) ALC-0315, DSPC, cholesterol and ALC-0159;
g) SM-102, DSPC, cholesterol and DMG-PEG;
h) Dlin-MC3-DMA, DSPC, cholesterol and DMG-PEG;
i) ALC-0315, DOPE, cholesterol, DMG-PEG and DSPE-PEG;
j) SS-OP, DOPE, cholesterol and DMG-PEG;
k) SS-OP, DSPC, cholesterol and DSPE-PEG; and
l) SS-OP, DOPC, cholesterol and DMG-PEG.

49. The lipid-based nanoparticle of claim 46, wherein the lipid-based nanoparticle comprises from about 35 mol % to about 55 mol % of a cationic or ionizable lipid, from about 5 mol % to about 20 mol % of a helper lipid, from about 30 mol % to about 60 mol % of a sterol, and from about 0.5 mol % to about 4 mol % of a PEG-lipid.

50. A pharmaceutical composition comprising at least one lipid-based nanoparticle of claim 34 and a pharmaceutically acceptable carrier or excipient.

51. The pharmaceutical composition of claim 50, wherein the composition further comprises an additional lipid-based nanoparticle comprising an antigen binding domain capable of specifically binding to a target expressed on activated immune cells surface and comprising one or several mRNA molecule(s) encoding an immune cell activity inhibiting protein.

52. A method for treating an autoimmune disease or an inflammatory disease, wherein the method comprises administering the lipid-based nanoparticle of claim 34 or a pharmaceutical composition comprising said lipid-based nanoparticle.

53. An in vitro method for inhibiting immune cells activity, comprising the step of contacting activated immune cells with a lipid-based nanoparticle of claim 34 or a pharmaceutical composition comprising said lipid-based nanoparticle.
